SEQUENCE LISTING (12 sequences)

SEQ ID NO: 1 - DNA, 9674 bp, synthetic construct
  misc_feature 1..9674: note = Description of Artificial Sequence: Synthetic polynucleotide
  source 1..9674: mol_type = other DNA
aaccatctcgcaaataaataagtattttactgttttcgtaacagttttgtaataaaaaaacctataaatattccggattattcataccgtcccaccatcgggcgccatggcttctcaccaccatcaccatcaccatcatcatcacgctcagcacgacgaggctgtggacaacaagttcaacaaggagcagcagaacgctttctacgagatcctgcacctccctaacctgaacgaggagcagcgtaacgctttcatccagtccctgaaggacgacccttctcagtctgctaacctgctggctgaggctaagaagctgaacgacgctcaggctcctaaggtggacaacaagttcaacaaggagcagcagaacgctttctacgagatcctgcacctccctaacctgaacgaggagcagcgtaacgctttcatccagtccctgaaggacgacccttctcagtctgctaacctgctggctgaggctaagaagctgaacgacgctcaggctcctaaggtggacgctaacggtggcggcggttccggcggtggtggctctctcgaggttctgttccagggtccgaatgaaggcgaggaagatgatgacaaagacttcctgtggccagctccaaacgaagaacaggtgacttgtctcaagatgtacttcggtcatagcagcttcaaaccagtgcagtggaaagttatccacagcgttcttgaagaacgtcgtgataatgtggctgtgatggctactggctatggtaagagcctgtgtttccagtacccgccagtttacgttggtaagatcggtctggtgattagcccgctgatctctctgatggaagaccaggtgctgcaacttaagatgagcaacatcccggcttgtttcctgggttctgcacaaagcgagaacgtgctcaccgatatcaagctgggtaagtaccgtatcgtgtacgtgacgccagaatactgtagcggcaacatgggtcttctgcaacagctcgaagctgatattggcatcaccctcattgcagtggacgaagctcactgtatcagcgagtggggtcatgatttccgcgactctttccgtaaactgggttctctgaagactgcacttccgatggttccaattgtggcactgaccgcaactgcttctagctctattcgtgaagacatcgttcgttgcctgaacctccgtaacccacaaattacctgcaccggctttgaccgtccgaacctgtacctggaggttcgtcgtaagaccggtaatatccttcaggacctgcaaccattcctggttaagaccagcagccactgggagttcgaaggtccgactatcatctactgcccaagccgtaagatgacccagcaggttactggtgaactgcgtaaactgaacctgagctgtggcacttaccacgcaggcatgtctttctctacccgtaaagacatccatcatcgtttcgtgcgtgatgaaatccagtgcgttatcgctaccattgcattcggcatgggtatcaacaaagctgacatccgtcaagtgattcactacggtgcaccgaaagacatggaaagctactaccaggaaatcggccgtgcaggtcgtgatggtctgcaaagctcttgtcatgtgctgtgggcaccagcagatattaacctgaaccgtcacctgctgactgaaattcgtaacgagaaattccgtctgtacaaactgaagatgatggcgaagatggagaaatacctgcatagctcccgttgtcgtcgtcaaatcattctgagccatttcgaggataaacaggtgcagaaagcttctctgggtatcatgggcactgagaagtgctgcgataactgtcgtagccgtcttgatcactgctacagcatggacgatagcgaagacacttcttgggatttcggtccacaagcattcaaactgctgagcgcagttgatatcctgggtgagaaattcggcatcggcctcccaatcctgtttctgcgcggttctaactctcagcgtcttgctgatcaataccgtcgtcactctctgttcggcactggtaaagaccagaccgaatcttggtggaaagcattcagccgtcaacttatcaccgaaggctttctggtggaagtgtctcgttacaacaagttcatgaagatctgcgcactgactaagaaaggtcgtaactggctgcacaaggcaaataccgagtctcagtctcttatccttcaggctaacgaagaactgtgcccgaagaagcttctgctgccatcttctaagaccgtgagctctggtactaaagagcattgctacaaccaggtgccggttgaactgtctaccgagaagaagtccaacctggagaagctgtactcctacaaaccgtgcgacaagatctcctccggttctaatatcagcaagaagtccatcatggtgcagtctccggagaaagcttacagcagctctcagccagttatctctgcacaggaacaggaaactcagattgtgctgtacggtaaactggtggaagcacgtcagaaacacgctaacaagatggacgtgccgccagcaattcttgcaaccaacaagattctggtggacatggctaagatgcgcccaactactgttgagaacgtgaaacgtatcgacggtgttagcgaaggtaaagctgcaatgctggcaccactgcttgaagttatcaagcatttctgccagaccaactctgttcagaccgacctgttctcttctaccaaaccataatggtaccgaattcgcggccgcagagctcgctctggtgccacgcggtagttccgcttggagccacccgcagttcgaaaagtaagtgattaacctcaggttatacatatattttgaatttaattaattatacatatattttatattatttttgtcttttattatcgaggggccgttgttggtgtggggttttgcatagaaataacaatgggagttggcgacgttgctgcgccaacaccacctcccttccctcctttcatcatgtatctgtagataaaataaaatattaaacctaaaaacaagaccgcgcctatcaacaaaatgataggcattaacttgccgctgacgctgtcactaacgttggacgatttgccgactaaaccttcatcgcccagtaaccaatctaggtagctgagcgcatgcaagctgatccgggttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaatagagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctaagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcgatcccgcacgccgataacaagccttttcatttttactacagcattgtagtggcgagacacttcgctgtcgtcgaggtttaaacgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccaggaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcttcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaataagacgcacaaactaatatcacaaactggaaatgtctatcaatatatagttgctgatcagatctacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatcgtcgagatcccgggcgtttaaattgtgtaatttatgtagctgtaatttttaccttattaatattttttacgctttgcattcgacgactgaactcccaaatatatgtttaactcgtcttggtcgtttgaatttttgttgctgtgtttcctaatattttccatcaccttaaatatgttattgtaatcctcaatgttgaacttgcaattggacacggcatagttttccatagtcgtgtaaaacatggtattggctgcattgtaatacatccgactgagcgggtacggatctatgtgtttgagcagcctgttcaaaaactctgcatcgtcgcaaaacggaatttggtacccgggcgtatactccggaatattaatagatcatggagataattaaaatgat

SEQ ID NO: 2 - DNA, 45 bp, synthetic construct
  misc_feature 1..45: note = Description of Artificial Sequence: Synthetic oligonucleotide
  source 1..45: mol_type = other DNA
ttttttttttttttttttttttccaagtaaaacgacggccagtgc

SEQ ID NO: 3 - protein, 445 aa, synthetic construct
  REGION 1..445: note = Description of Artificial Sequence: Synthetic polypeptide
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 4 - protein, 214 aa, synthetic construct
  REGION 1..214: note = Description of Artificial Sequence: Synthetic polypeptide
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 118 aa, synthetic construct
  REGION 1..118: note = Description of Artificial Sequence: Synthetic polypeptide
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 6 - protein, 107 aa, synthetic construct
  REGION 1..107: note = Description of Artificial Sequence: Synthetic polypeptide
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 7 - protein, 10 aa, synthetic construct
  REGION 1..10: note = Description of Artificial Sequence: Synthetic peptide
GFSLTSYGVH

SEQ ID NO: 8 - protein, 16 aa, synthetic construct
  REGION 1..16: note = Description of Artificial Sequence: Synthetic peptide
VIYADGSTNYNPSLKS

SEQ ID NO: 9 - protein, 12 aa, synthetic construct
  REGION 1..12: note = Description of Artificial Sequence: Synthetic peptide
ARAYGNYWYIDV

SEQ ID NO: 10 - protein, 11 aa, synthetic construct
  REGION 1..11: note = Description of Artificial Sequence: Synthetic peptide
KSSESVSNDVA

SEQ ID NO: 11 - protein, 7 aa, synthetic construct
  REGION 1..7: note = Description of Artificial Sequence: Synthetic peptide
YAFHRFT

SEQ ID NO: 12 - protein, 9 aa, synthetic construct
  REGION 1..9: note = Description of Artificial Sequence: Synthetic peptide
HQAYSSPYT

(19) United States
(12) Patent Application Publication
(10) Pub. No.: US 2026/0250289 A1
(43) Pub. Date: Aug. 27, 2026

(54) BICYCLIC HETEROCYCLES AND THEIR USE AS WRN INHIBITORS

(71) Applicant: Novartis AG, Basel (CH)

(72) Inventors: Markus Reto FUREGATI, Allschwil (CH); Wanben GONG, Suzhou city (CN); Jacques HAMON, Habsheim (FR); Juergen Hans-Hermann HINRICHS, Schopfheim (DE); Henrik MOEBITZ, Arlesheim (CH); Sandro NOCITO, Wölflinswil (CH); Niko SCHMIEDEBERG, Riehen (CH); Joseph SCHOEPFER, Ependes (FR); Ross Sinclair STRANG, Hagenthal-le-Bas (FR); Huangchao YU, Changshu (CN); Frédéric Jean ZECRI, Brookline, MA (US)

(21) Appl. No.: 19/130,558

(22) PCT Filed: Nov. 14, 2023
(86) PCT No.: PCT/IB2023/061469

§ 371 Date: May 16, 2025

(30) Foreign Application Priority Data
Nov. 16, 2022 (WO) PCT/CN2022/132256

(51) Int. Cl. C07D 487/04; A61K 31/519; A61K 31/5377; A61K 45/06; A61P 35/00; C07B 59/00; C07D 519/00; C07K 16/28

(52) U.S. Cl. CPC C07D 487/04; A61K 31/519; A61K 31/5377; A61K 45/06; A61P 35/00; C07B 59/002; C07D 519/00; C07K 16/2818; C07K 2317/565; C07K 2317/76

(57) ABSTRACT

The present invention provides a compound, or a pharmaceutically acceptable salt thereof, of formula (I): wherein R₁, R₂, R₃, R₄, R₅, R₂₆, R₂₇, R, M, L, W, T, V, Y, K, J, A and y are as described in the Summary of the Invention, therapeutic uses, research uses and a method for manufacturing the compounds of the invention. The present invention further provides a combination of pharmacologically active agents and a pharmaceutical composition.

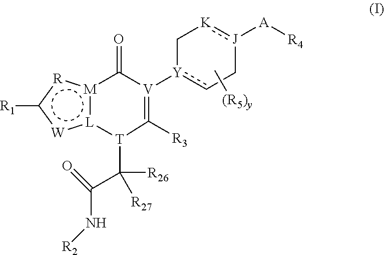

### FIELD OF INVENTION

The invention provides bicyclic heterocyclic compounds, the use thereof for inhibiting Werner Syndrome RecQ DNA helicase (WRN) and methods of treating disease using said compounds, in particular the use in treating cancer, and in particular the treatment of cancer characterized as microsatellite instability-high (MSI-H) or mismatch repair deficient (dMMR), including colorectal, gastric or endometrial cancer, their use as research chemicals, synthesis of said compounds, intermediates, formulations and combinations.

### BACKGROUND OF THE INVENTION

Loss of DNA mismatch repair is a common initiating event in cancer development occurring in 10-30% of colorectal, endometrial, ovarian and gastric cancers (Aaltonen, L. A. et al. Clues to the pathogenesis of familial colorectal cancer, Science 260, 812-816 (1993), Bonneville R et al., Landscape of Microsatellite Instability Across 39 Cancer Types. JCO Precis Oncol. 1: PO.17.00073 (2017)). Cancers that have lost competence in mismatch repair (MMR) have a high mutational burden, and frequent deletion and insertion events in repetitive DNA tracts, a phenotype known as microsatellite instability (MSI). While progress has been made in the treatment of microsatellite instability high (MSI-H) cancers, and the demonstration that pembrolizumab (anti-PD1) treatment led to significantly longer progression-free survival than chemotherapy when received as first-line therapy for MSI-H-dMMR metastatic colorectal cancer resulted in the recent approval of pembrolizumab as first-line treatment of these cancers, there is still a significant unmet medical need in CRC and other MSI-H indications (André T., et al. Pembrolizumab in Microsatellite-Instability-High Advanced Colorectal Cancer. N Engl J Med 383(23):2207-2218 (2020)). Several large-scale functional genomics screens across large panels of cell lines, including Novartis with 398 cell lines from the Cancer Cell Line Encyclopedia (CCLE) (McDonald E. R. et al., Project DRIVE: A Compendium of Cancer Dependencies and Synthetic Lethal Relationships Uncovered by Large-Scale, Deep RNAi Screening.

Cell 170(3):577-592 (2017)), have identified the Werner Syndrome RecQ helicase (WRN) as being selectively required for the survival of cell lines with defective mismatch repair that have become MSI-H (Behan, F. M. et al. Prioritization of cancer therapeutic targets using CRISPR-Cas9 screens. Nature 568, 511-516 (2019), Chan, E. M. et al. WRN helicase is a synthetic lethal target in microsatellite unstable cancers. Nature 568, 551-556 (2019). Kategaya, L., Perumal, S. K., Hager, J. H. & Belmont, L. D. Werner syndrome helicase is required for the survival of cancer cells with microsatellite instability. iScience 13, 488-497 (2019), Lieb, S. et al. Werner syndrome helicase is a selective vulnerability of microsatellite instability-high tumor cells. eLife 8, e43333 (2019)). WRN is synthetic lethal with MSI cancers. Depletion of WRN leads to anti-proliferative effects and results in activation of multiple DNA damage signaling markers, induction of cell cycle arrest and apoptosis in MMR cancer models but not cancer cells with an intact MMR pathway.

These findings indicate that WRN provides a DNA repair and maintenance function that is essential for cell survival in MSI cancers. Recently, the mechanism of WRN dependence has been elucidated. It has been shown that dinucleotide TA repeats are selectively unstable in MSI cells and undergo large scale expansions. These expanded TA repeats form secondary DNA structures that require the WRN helicase for unwinding (van Wietmarschen, N. et al. Repeat expansions confer WRN dependence in microsatellite-unstable cancers. Nature 586, 292-298, 2020). In the absence of WRN (or upon WRN helicase inhibition), expanded TA repeats in MSI cells are subject to nuclease cleavage and chromosome breakage. Thus, inhibiting the WRN helicase is an attractive strategy for the treatment of mismatch repair defective cancers.

### SUMMARY OF THE INVENTION

There remains a need for new treatments and therapies for the treatment of cancer, and in particular cancers characterized as microsatellite instability-high (MSI-H) or mismatch repair deficient (dMMR), including colorectal, gastric or endometrial cancer. The invention provides compounds, pharmaceutically acceptable salts thereof, pharmaceutical compositions thereof and combinations thereof, said compounds being inhibitors of Werner Syndrome RecQ DNA Helicase (WRN). The invention further provides methods of treating, preventing, or ameliorating a disease or condition, comprising administering to a subject in need thereof an effective amount of a WRN inhibitor. The invention further provides WRN inhibitor compounds as research chemicals. Various embodiments of the invention are described herein.

Within certain aspects, provided herein is a compound of formula (I) or a pharmaceutically acceptable salt thereof:

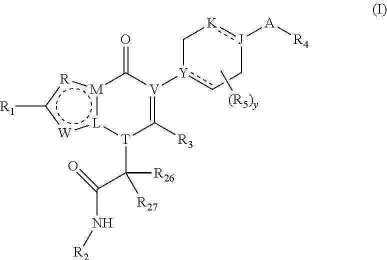

wherein
R, M, W, L, V and T are independently selected from C, CH and N,
to form subformulae 1a, 1b, 1c, 1d, 1e and 1f:

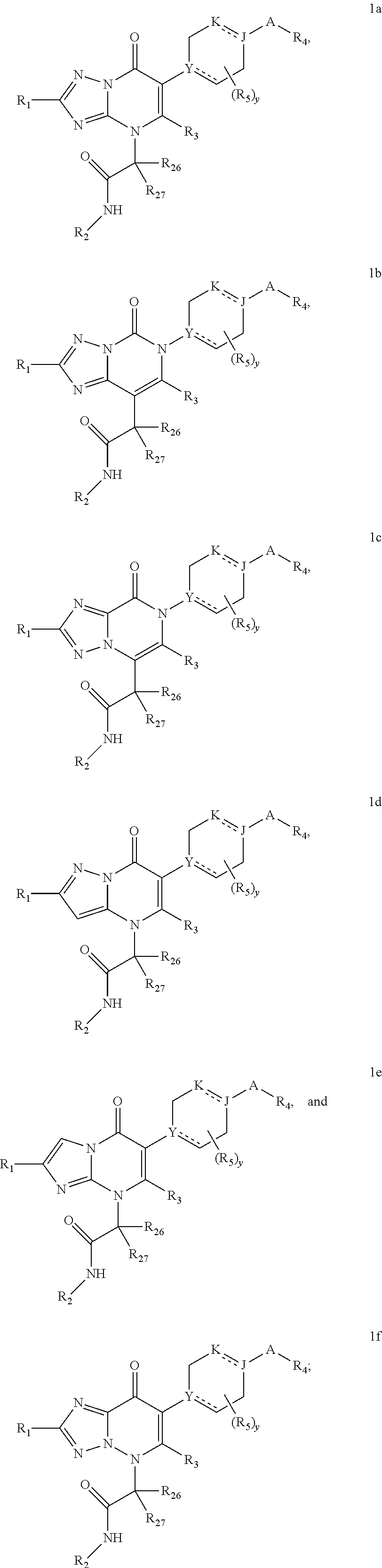

A is a linker which is —C(O)—;
Y is N, C or CH;
y is 0, 1, 2, 3 or 4;
Y [symbol] means Y is linked via a single bond to the adjacent carbon atom when Y is CH, or Y is linked via a double bond to the adjacent atom when Y is C, and when Y [symbol] is a single bond, Y is carbon unsubstituted or substituted by OH or F;
when Y is N, Y [symbol] is a single bond;
K [symbol] means K is linked via a single or double bond to the adjacent atom;
wherein:
   when K [symbol] is a double bond, Y [symbol] is a single bond, K is CH and J is C, or
   when K [symbol] is a single bond, K is selected from —CH₂—, —CH₂CH₂—, —NH— and a
   bond (to form a 5-membered ring:

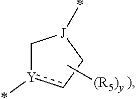

and J is N;
R₅ is independently selected from:
   —(C₁-C₄)alkyl,
   —(C₃-C₅)cycloalkyl,
   and wherein two R₅ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S,
   when K [symbol] J is a carbon-nitrogen single bond, a R₅ substituent on K and on the adjacent carbon atom may join to form ring C:

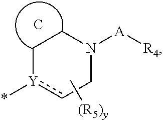

wherein ring C is a fused (C₃-C₆)cycloalkyl ring, a fused (C₃-C₆)heterocyclyl ring or a fused phenyl ring, wherein said fused (C₃-C₆)heterocyclyl ring contains ring carbon atoms and one ring heteroatom selected from O, N and S,
   and wherein when ring C is a fused (C₃-C₆)cycloalkyl ring, said fused (C₃-C₆)cycloalkyl ring is unsubstituted or substituted with 1 or 2 R₄₀ groups, wherein said R₄₀ is selected from:
      (C₁-C₂)alkyl, wherein each (C₁-C₂)alkyl is independently unsubstituted or substituted by OH or 1, 2 or 3 halo,
      halo, in particular F,
      or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
      or wherein two R₄₀ substituents on adjacent carbon atoms join together with the carbon atoms to which they are attached, to form a fused cyclopropyl ring;
and wherein when K is —CH₂— and J is N, two R₅ substituents may join to form a (C₁-C₃)alkylene bridge or a heteroalkylene bridge, wherein said heteroalkylene bridge is one heteroatom selected from N and O, or is —CH₂—O—CH₂—;
R₁ is:
cycloalkenyl, wherein said cycloalkenyl is a partially unsaturated monocyclic ring containing 5 or 6 ring carbon atoms, and said cycloalkenyl is unsubstituted or substituted by 1, 2, 3 or 4, preferably 1 or 2, R₃₃, wherein R₃₃ is halo, and wherein said cycloalkenyl or halo-substituted cycloalkenyl is substituted by 0, 1 or 2 R₁₅ substituents, or said cycloalkenyl or halo-substituted cycloalkenyl has 2 substitutents at the same ring carbon atom which join to form an oxetanyl spiro ring,
or R₁ is heterocyclyl, wherein said heterocyclyl is a 5 or 6 membered fully saturated or partially unsaturated group comprising ring carbon atoms and 1 or 2 ring heteroatoms independently selected from N, O and S, and wherein said heterocyclyl is unbridged or bridged, and said bridge is 1 or 2 carbon atoms, wherein said heterocyclyl is unsubstituted or substituted by 1, 2, 3 or 4, preferably 1 or 2, R₃₃, wherein R₃₃ is halo, and wherein said heterocyclyl or halo-substituted heterocyclyl is substituted by 0, 1 or 2 substituents independently selected from R₁₅, R₁₆, R₁₇, R₁₈, Rig, R₂₀, R₂₂ and R₂₃,
or said heterocyclyl or halo-substituted heterocyclyl is fused to a cyclopropyl ring, wherein said cyclopropyl ring is unsubstituted or substituted by 1, 2 or 3 F,
or said heterocyclyl or halo-substituted heterocyclyl has 2 substitutents at the same ring carbon atom which join to form a cyclopropyl spiro ring, or tetrahydrofuranyl spiro ring,
or said heterocyclyl or halo-substituted heterocyclyl is fused with a (C₃-C₅)heterocycloalkyl ring, wherein said (C₃-C₅)heterocycloalkyl ring contains ring carbon atoms and 1 ring O atom;
or R₁ is heteroaryl, wherein said heteroaryl is a 5 or 6 membered fully unsaturated monocyclic group comprising ring carbon atoms and 1, 2, 3 or 4 ring heteroatoms independently selected from N, O and S, preferably 1 or 2 ring heteraoms, preferably wherein the total number of ring S atoms does not exceed 1 and preferably the total number of ring O atoms does not exceed 1,
and wherein said heteroaryl is unsubstituted or substituted by 1, 2 or 3 substituents independently selected from R₂₁ and R₃₀, wherein R₂₁ and R₃₀ are independently selected from halo and (C₁-C₄)alkyl, wherein said (C₁-C₄)alkyl is unsubstituted or substituted by 1, 2 or 3 halo,
or R₁ is phenyl, wherein said phenyl is unsubstituted or substituted by 1, 2, 3 or 4, preferably 1 or 2, R₃₃, wherein R₃₃ is halo, and wherein said phenyl or halo-substituted phenyl is substituted by 0, 1 or 2 R₁₅ substituents,
or R₁ is (C₂-C₄)alkynyl or (C₂-C₄)alkenyl, wherein said (C₂-C₄)alkynyl and (C₂-C₄)alkenyl are unsubstituted or substituted by (C₁-C₄)alkyl-O—C(O)—, or morpholinyl;
each R₁₅, R₁₆, R₁₇, R₁₈, Rig, R₂₀, R₂₂ and R₂₃ is independently selected from:
   halo
   (C₁-C₄)alkyl-O—(CH₂)ₙ unsubstituted or substituted by 1, 2 or 3 halo;
   (C₁-C₄)alkyl unsubstituted or substituted by OH, —O—(C₁-C₂)alkyl or 1, 2 or 3 halo,
   HOC(O)—(CH₂)ₙ—,
   (C₁-C₄)alkyl-C(O)(CH₂)ₙ—,
   (E)-cyclooct-4-en-1-yl-O—C(O)—,
   (C₁-C₄)alkyl-O—C(O)(CH₂)ₙ,
   ═O
   azetidinyl or pyrrolidinyl, wherein said azetidinyl and pyrrolidinyl are linked to the rest of the molecule via the N atom, and are each unsubstituted or substituted by 1 or 2 F,
   R₂₅(R₂₄)N—(CH₂)ₙ, wherein R₂₄ is H or (C₁-C₄)alkyl unsubstituted or substituted by 1, 2 or 3 halo, and R₂₅ is:
      H,
      (C₁-C₄)alkyl-C(O)(CH₂)ₙ—, wherein said (C₁-C₄)alkyl of (C₁-C₄)alkyl-C(O)(CH₂)ₙ— is unsubstituted or substituted by halo or —N₃,
      (C₁-C₄)alkyl-O—C(O)(CH₂)ₙ₋,
      (C₁-C₄)alkyl unsubstituted or substituted by 1, 2 or 3 halo, or
      (E)-cyclooct-4-en-1-yl-O—C(O)—,
   OH
wherein n is 0, 1 or 2,
R₂₆ is CH₃, H or deuterium;
R₂₇ is CH₃, H or deuterium;
or R₂₆ and R₂₇ join, together with the carbon atom to which they are attached, to form a cyclopropyl ring;
R₂ is the moiety:

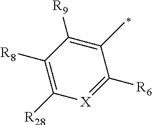

R₆ is selected from:
   H,
   halo,
   (C₁-C₄)alkyl unsubstituted or substituted by 1, 2 or 3 halo,
   (C₃-C₅)cycloalkyl unsubstituted or substituted by 1, 2 or 3 halo,
   —O—(C₁-C₄)alkyl unsubstituted or substituted by 1, 2 or 3 halo,
   OH, and
   CN;
R₈ is selected from H, halo, and (C₁-C₄)alkyl unsubstituted or substituted by 1, 2 or 3 halo,
R₉ is selected from H, O—CH₃, OH, CN, CH₃ and halo;
R₂₈ is selected from:
   SF₅,
   H,
   —C(O)H,
   halo,
   (C₁-C₄)alkyl unsubstituted or substituted by 1, 2 or 3 halo,
   (C₁-C₄)alkynyl,
   (C₁-C₄)alkenyl,
   (C₃-C₅)cycloalkyl unsubstituted or substituted by 1, 2 or 3 halo, and
   OCF₃;
X is selected from C—R₇ and N, wherein R₇ is H, CF₃ or halo, or R₇ can join, together with R₂₈ or R₆, and the atoms to which they are attached, to form a fused (C₄-C₆)cycloalkyl ring, wherein said fused (C₄-C₆)cycloalkyl ring is unsubstituted or substituted by 1, 2 or 3 halo,
or
R₂ is selected from:

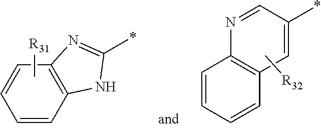

wherein
R₃₁ is selected from H, halo and CH₃,
R₃₂ is selected from H, halo and CH₃,
R₃ is:
   cyclopropyl,
   O—CH₃,
   N(CH₃)₂,
   S—CH₃,
   (C₁-C₄)alkyl unsubstituted or substituted by 1, 2 or 3 substituents independently selected from halo and OH;
R₄ is selected from:
—(C₁-C₄)alkyl, unsubstituted or substituted by NH₂;
—O—CH₂phenyl;
—O—CH₂CH₂phenyl;
—NH—NH—C(O)—CF₃;
-heteroaryl1, wherein said heteroaryl1 is a 5 or 6 membered, fully unsaturated, monocyclic ring comprising ring carbon atoms and 1, 2, 3 or 4 ring heteroatoms independently selected from N, O and S;
-heteroaryl2, wherein said heteroaryl2 is a 9 or 10 membered fused bicyclic ring comprising ring carbon atoms and 1, 2, 3 or 4 ring heteroatoms independently selected from N, O and S, and wherein both rings are fully unsaturated, or one ring is fully unsaturated, and the other is saturated or partially unsaturated, and wherein the heteroatoms may be in one or both rings;
-phenyl;
-heterocyclyl2, wherein said heterocyclyl2 is a 5 or 6 membered fully saturated or partially unsaturated group comprising ring carbon atoms and 1 or 2 ring heteroatoms independently selected from N, O and S,
and

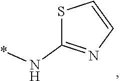 [SMILES: *Nc1nccs1]

wherein heteroaryl1, heteroaryl2 and phenyl are each substituted by 1, 2 or 3 substituents independently selected from R₁₀, R₁₁, R₁₂, R₁₃ and R₁₄, wherein each R₁₀, R₁₁, R₁₂, R₁₃ and R₁₄ is independently selected from:
   H,
   halo,
   (C₁-C₄)alkyl unsubstituted or substituted by 1, 2 or 3 halo substituents,
   (C₁-C₂)alkyl substituted by —O—(C₁-C₂)alkyl or OH,
   —S—(C₁-C₃)alkyl,
   —O—(C₁-C₄)alkyl unsubstituted or substituted by 1, 2 or 3 halo substituents,
   OH,
   (C₃-C₅)cycloalkyl, wherein said (C₃-C₅)cycloalkyl is unsubstituted or substituted by 1 or 2 halo,
   —O—(C₃-C₅)cycloalkyl,
   —NR₃₄R₃₅ wherein R₃₄ and R₃₅ are independently selected from:
      H,
      (C₁-C₄)alkyl, wherein said (C₁-C₄)alkyl is unsubstituted or substituted by OH or —O(C₁-C₂)alkyl,
      and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
   CN,
   —(C₂-C₄)alkenyl,
   —(C₂-C₄)alkynyl,
   ═O
   —C(O)H, and
   —C(O)(C₁-C₄)alkyl;
with the proviso that R₄ is not:

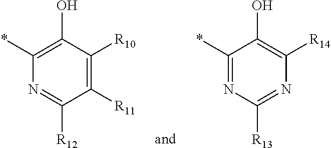

wherein
R₁₀, R₁₁, R₁₂, R₁₃ and R₁₄ are independently selected from:
   H,
   halo,
   (C₁-C₄)alkyl unsubstituted or substituted by 1, 2 or 3 halo substituents,
   (C₁-C₂)alkyl substituted by —O—(C₁-C₂)alkyl or OH,
   —S—(C₁-C₃)alkyl,
   —O—(C₁-C₄)alkyl unsubstituted or substituted by 1, 2 or 3 halo substituents,
   OH,
   (C₃-C₅)cycloalkyl, wherein said (C₃-C₅)cycloalkyl is unsubstituted or substituted by 1 or 2 halo,
   —O—(C₃-C₅)cycloalkyl,
   —NR₃₄R₃₅ wherein R₃₄ and R₃₅ are independently selected from:
      H,
      (C₁-C₄)alkyl, wherein said (C₁-C₄)alkyl is unsubstituted or substituted by OH or —O(C₁-C₂)alkyl,
      and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
   CN,
   —(C₂-C₄)alkenyl,
   —(C₂-C₄)alkynyl,
   —C(O)H, and
   —C(O)(C₁-C₄)alkyl;
and
* indicates a point of attachment.

In another aspect, the invention provides a pharmaceutical composition comprising a compound of the present invention and one or more pharmaceutically acceptable carriers.

In another aspect, the invention provides a combination, in particular a pharmaceutical combination, comprising a compound of the present invention and one or more therapeutically active agents.

In another aspect, the invention provides a compound of the present invention for use as a medicament, in particular for the treatment of a disorder or disease which can be treated by WRN inhibition.

In another aspect, the invention provides a compound of the present invention for use in the treatment of cancer, particularly wherein the cancer is characterized as microsatellite instability-high (MSI-H) or mismatch repair deficient (dMMR).

In another aspect, the invention provides a method of treating a disorder or disease which can be treated by WRN inhibition in a subject, comprising administering to the subject a therapeutically effective amount of a compound of the present invention.

In another aspect, the invention provides a method of treating cancer in a subject, more particularly wherein the cancer is characterized as microsatellite instability-high (MSI-H) or mismatch repair deficient (dMMR), comprising administering to the subject a therapeutically effective amount of a compound of the present invention.

In another aspect, the invention provides the use of a compound of the present invention in the manufacture of a medicament for the treatment of a disorder or disease which can be treated by WRN inhibition.

In another aspect, the invention provides a research use of a compound of the present invention.

### DETAILED DESCRIPTION

The invention therefore provides a compound of formula (I):

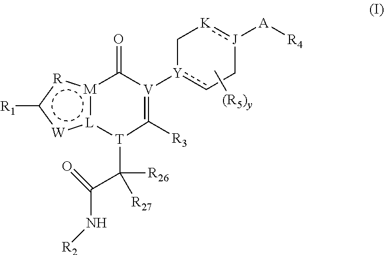

wherein R₁, R₂, R₃, R₄, R₅, R₂₆, R₂₇, R, M, L, W, T, V, Y, K, J, A and y are as described in the Summary of the Invention, supra.

Unless specified otherwise, the term “compounds of the present invention” or “compound of the present invention” refers to compounds of formula (I) subformulae thereof, and exemplified compounds, and salts thereof, as well as all zwitterions, stereoisomers (including diastereoisomers and enantiomers), rotamers, tautomers and isotopically labeled compounds (including deuterium substitutions), as well as inherently formed moieties.

Various (enumerated) embodiments of the invention are described herein. It will be recognized that features specified in each embodiment may be combined with other specified features to provide further embodiments of the present invention.

Embodiment 1. A compound of formula (I) or a pharmaceutically acceptable salt thereof, as described above.

Embodiment 2. A compound of formula (I) or a pharmaceutically acceptable salt thereof, according to Embodiment 1, wherein when R₁ is a ring, then:
each R₁ ring atom adjacent to the R₁ ring atom to which said R₁ ring is joined to the remainder of the molecule, is independently unsubstituted or substituted by halo only, in particular, independently unsubstituted or substituted with one F substituent, and
preferably, said R₁ ring is linked to the remainder of the molecule via a R₁ ring nitrogen atom, or a R₁ ring carbon atom which is double-bonded to an adjacent R₁ ring atom.

Embodiment 3. A compound of formula (I) or a pharmaceutically acceptable salt thereof, according to Embodiments 1 or 2, wherein R₁ is:
cycloalkenyl, wherein said cycloalkenyl is a partially unsaturated monocyclic ring containing 5 or 6 ring carbon atoms, and said cycloalkenyl is unsubstituted or substituted by 1, 2, 3 or 4, preferably 1 or 2, R₃₃, wherein R₃₃ is halo, and wherein said cycloalkenyl or halo-substituted cycloalkenyl is substituted by 0, 1 or 2 R₁₅ substituents, preferably 1 substituent, or said cycloalkenyl or halo-substituted cycloalkenyl has 2 substitutents at the same ring carbon atom which join to form an oxetanyl spiro ring,
or R₁ is heterocyclyl, wherein said heterocyclyl is a 5 or 6 membered fully saturated or partially unsaturated group comprising ring carbon atoms and 1 or 2 ring heteroatoms independently selected from N, NH, O and S, and wherein said heterocyclyl is unbridged or bridged, and said bridge is 1 or 2 carbon atoms, wherein said heterocyclyl is unsubstituted or substituted by 1, 2, 3 or 4, for example 1, 2 or 3, in particular 1 or 2 R₃₃, wherein R₃₃ is halo, and wherein said heterocyclyl or halo-substituted heterocyclyl is substituted by 0, 1 or 2 substituents, preferably 0 or 1 substituent, independently selected from R₁₅, R₁₆, R₁₇, R₁₈, R₁₉, R₂₀, R₂₂ and R₂₃,
or said heterocyclyl or halo-substituted heterocyclyl is fused to a cyclopropyl ring, wherein said cyclopropyl ring is unsubstituted or substituted by 1, 2 or 3 F,
or said heterocyclyl or halo-substituted heterocyclyl has 2 substitutents at the same ring carbon atom which join to form a tetrahydrofuranyl spiro ring,
or R₁ is heteroaryl, wherein said heteroaryl is a 5 or 6 membered fully unsaturated monocyclic group comprising ring carbon atoms and 1, 2, 3 or 4 ring heteroatoms independently selected from N, O and S, preferably 1 or 2 ring heteraoms, wherein the total number of ring S atoms does not exceed 1, and the total number of ring O atoms does not exceed 1, wherein said heteroaryl is unsubstituted or substituted by 1, 2 or 3 substituents independently selected from R₂₁ and R₃₀, wherein R₂₁ and R₃₀ are independently selected from halo and (C₁-C₄)alkyl, wherein said (C₁-C₄)alkyl is unsubstituted or substituted by 1, 2 or 3 halo,
or R₁ is phenyl, wherein said phenyl is unsubstituted or substituted by 1, 2, 3 or 4, preferably 1 or 2, R₃₃, wherein R₃₃ is halo, and wherein said phenyl or halo-substituted phenyl is substituted by 0 or 1 R₁₅ substituents,
or R₁ is (C₂-C₄)alkynyl, unsubstituted or substituted by (C₁-C₄)alkyl-O—C(O)—;
and each R₁₅, R₁₆, R₁₇, R₁₈, Rig, R₂₀, R₂₂ and R₂₃ is independently selected from:
   halo
   (C₁-C₄)alkyl-O—(CH₂)ₙ unsubstituted or substituted by 1, 2 or 3 halo;
   (C₁-C₄)alkyl unsubstituted or substituted by OH, —O—(C₁-C₂)alkyl or 1, 2 or 3 halo,
   HOC(O)—(CH₂)ₙ—,
   H₃C—C(O)(CH₂)ₙ—,
   (C₁-C₄)alkyl-O—C(O)(CH₂)ₙ,
   ═O
   azetidinyl or pyrrolidinyl, wherein said azetidinyl and pyrrolidinyl are linked to the rest of the molecule via the N atom, and are each unsubstituted or substituted by 1 or 2 F,
   R₂₅(R₂₄)N—(CH₂)ₙ, wherein R₂₄ is H or (C₁-C₂)alkyl unsubstituted or substituted by 1, 2 or 3 halo, R₂₅ is H, (C₁-C₄)alkyl-C(O)—, (C₁-C₄)alkyl-O—C(O)—, or (C₁-C₄)alkyl unsubstituted or substituted by 1, 2 or 3 halo,
   OH
wherein n is 0, 1 or 2,

Embodiment 4. A compound of formula (I) or a pharmaceutically acceptable salt thereof, according to any of Embodiments 1, 2 or 3, wherein R₁ is:
cycloalkenyl, wherein said cycloalkenyl is a partially unsaturated monocyclic ring containing 5 or 6 ring carbon atoms, and said cycloalkenyl is unsubstituted or substituted by 1 or 2 R₃₃, wherein R₃₃ is halo, preferably F, and wherein said cycloalkenyl or halo-substituted cycloalkenyl is substituted by 0 or 1 R₁₅ substituents, wherein R₁₅ is selected from:
   a) (C₁-C₂)alkyl-O— unsubstituted or substituted by 1, 2 or 3 halo;
   b) (C₁-C₂)alkyl unsubstituted or substituted by 1, 2 or 3 halo,
   c) HOC(O)—(CH₂)ₙ—,
   d) H₃C—C(O)(CH₂)ₙ—,
   e) H₃C—O—C(O)(CH₂)ₙ,
   f) ═O, and
   g) R₂₅(R₂₄)N—, H, wherein R₂₄ is H or (C₁-C₂)alkyl unsubstituted or substituted by 1, 2 or 3 halo, R₂₅ is H or (C₁-C₂)alkyl unsubstituted or substituted by 1, 2 or 3 halo,
n is 0 or 1,
wherein
   the R₁₅ substituent a) to g) of said cycloalkenyl or halo-substituted cycloalkenyl is not present on the ring atoms adjacent to the ring atom to which the cycloalkenyl or halo-substituted cycloalkenyl is joined to the remainder of the molecule, and preferably, said cycloalkenyl or halo-substituted cycloalkenyl is a 6 membered ring, with 1 R₁₅ substituent in the ring para position relative to the remainder of the molecule; and
   said cycloalkenyl or halo-substituted cycloalkenyl is linked to the remainder of the compound via a R₁ ring carbon atom which is double bonded to an adjacent R₁ ring carbon atom;
or R₁ is heterocyclyl, wherein said heterocyclyl is a 5 or 6 membered fully saturated or partially unsaturated group comprising ring carbon atoms and 1 or 2 ring heteroatoms independently selected from N, NH, O and S, and wherein said heterocyclyl is unbridged or bridged, and said bridge is 1 or 2 carbon atoms, wherein said heterocyclyl is unsubstituted or substituted by 1 or 2 R₃₃, wherein R₃₃ halo, is preferably F, and wherein said heterocyclyl or halo-substituted heterocyclyl is substituted by 0 or 1 substituents independently selected from R₁₅, R₁₆, R₁₇, R₁₈, Rig, R₂₀, R₂₂ and R₂₃, wherein said R₁₅, R₁₆, R₁₇, R₁₈, Rig, R₂₀, R₂₂ and R₂₃ are independently selected from:
   a) (C₁-C₄)alkyl-O— unsubstituted or substituted by 1, 2 or 3 halo;
   b) (C₁-C₄)alkyl unsubstituted or substituted by OH, —O—(C₁-C₂)alkyl or 1, 2 or 3 halo,
   c) HOC(O)—(CH₂)ₙ—,
   d) H₃C—C(O)(CH₂)ₙ—,
   e) H₃C—O—C(O)(CH₂)ₙ,
   f) ═O
   g) R₂₅(R₂₄)N—, wherein R₂₄ is H, (C₁-C₂)alkyl unsubstituted or substituted by 1, 2 or 3 halo, R₂₅ is H, (C₁-C₂)alkyl unsubstituted or substituted by 1, 2 or 3 halo,
   h) OH
wherein n is 0 or 1,
and wherein:
   substituent a) to h) of said heterocyclyl or halo-substituted heterocyclyl is not present on the ring atoms adjacent to the ring atom to which the heterocyclyl or halo-substituted heterocyclyl is joined to the remainder of the molecule, and preferably, when said heterocyclyl or halo-substituted heterocyclyl is a 6 membered ring, it has 0 or 1 substituent selected from a) to h) in the meta or para position, preferably para, relative to the remainder of the molecule; and
   said heterocyclyl is linked to the remainder of the compound via a R₁ ring nitrogen atom, or a R₁ ring carbon atom which is double bonded to an adjacent ring atom;
or R₁ is heteroaryl, wherein said heteroaryl is a 5 or 6 membered fully unsaturated monocyclic group comprising ring carbon atoms and 1 or 2 ring heteroatoms independently selected from N, O and S, preferably N, wherein the total number of ring S atoms does not exceed 1, and the total number of ring O atoms does not exceed 1, wherein said heteroaryl is unsubstituted or substituted by 1 or 2 substituents independently selected from R₂₁ and R₃₀, wherein R₂₁ and R₃₀ are independently selected from (C₁-C₂)alkyl, and said (C₁-C₂)alkyl is unsubstituted or substituted by 1, 2 or 3 halo, and wherein preferably, said alkyl or halo-alkyl substituent is not present on the R₁ ring atoms adjacent to the R₁ ring atom to which the heteroaryl is joined to the remainder of the molecule, and more preferably, when heteroaryl is a 6-membered ring, said alkyl or halo-alkyl substituent is in the ring para position relative to the rest of the molecule.

Embodiment 5. A compound of formula (I) or a pharmaceutically acceptable salt thereof, according to any of Embodiments 1 to 4, wherein R₁ is selected from:

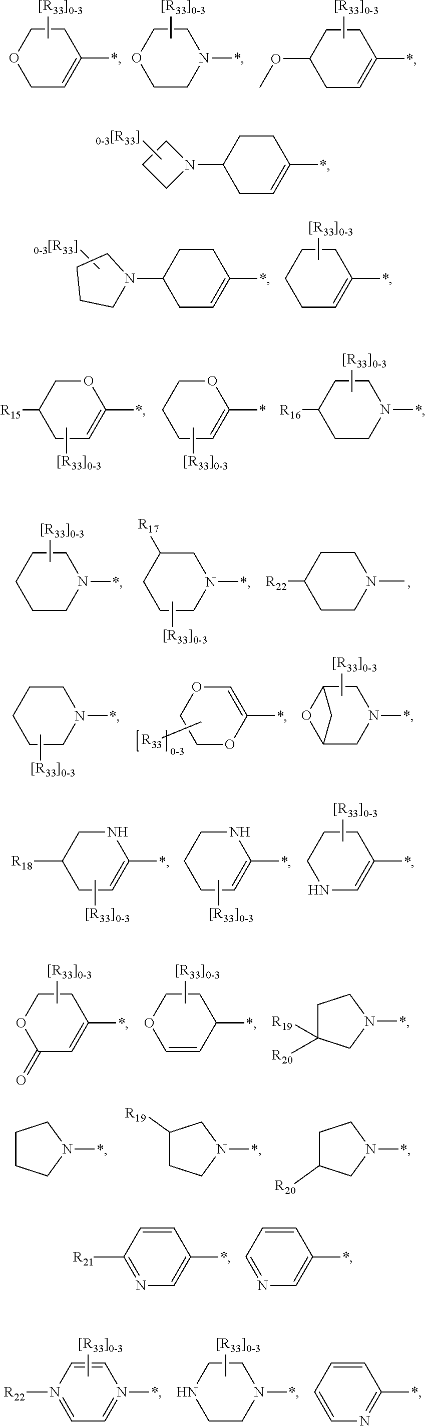

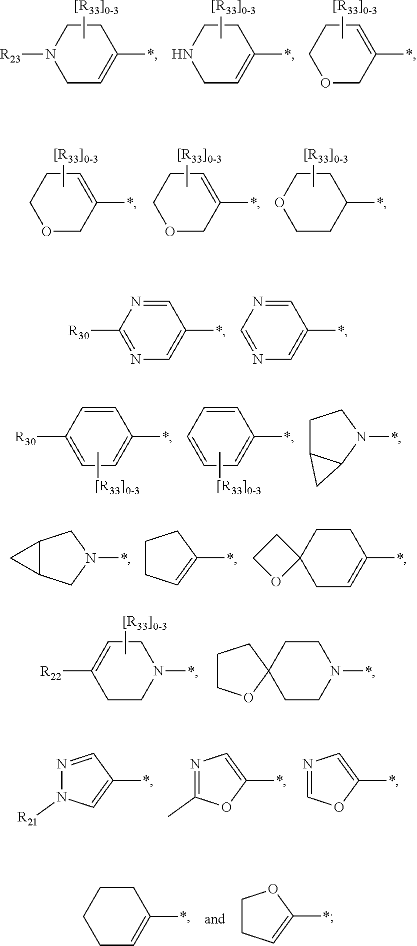

alternatively, there are 0-2 R₃₃ substituents, in each of the moieties above,
R₃₃ is F;
R₁₅ is:
   halo,
   R₂₅(R₂₄)N—(CH₂)ₙ, wherein R₂₄ is H or CH₃ unsubstituted or substituted by 1, 2 or 3 halo, R₂₅ is H, (C₁-C₄)alkyl-C(O)—, (C₁-C₄)alkyl-O—C(O)—, or (C₁-C₄)alkyl unsubstituted or substituted by 1, 2 or 3 halo, or
   azetidinyl or pyrrolidinyl, wherein said azetidinyl and pyrrolidinyl are linked to the rest of the molecule via the N atom, and are unsubstituted or substituted by 1 or 2 F;
R₁₆ is R₂₅(R₂₄)N—, wherein R₂₄ is H or (C₁-C₂)alkyl, R₂₅ is H or (C₁-C₂)alkyl unsubstituted or substituted by 1, 2 or 3 halo, in particular F;
R₁₇ is halo
R₁₈ is halo;
R₁₉ is:
   halo
   (C₁-C₄)alkyl unsubstituted or substituted by 1, 2 or 3 halo,
   (C₁-C₄)alkyl-O—(CH₂)ₙ—;
R₂₀ is halo;
R₂₁ is (C₁-C₂)alkyl, unsubstituted or substituted by 1, 2 or 3 F;
R₂₂ and R₂₃ are each independently selected from:
   (C₁-C₄)alkyl unsubstituted or substituted by 1, 2 or 3 halo,
   (C₁-C₄)alkyl-O—(CH₂)ₙ—
   HOC(O)—(CH₂)ₙ—,
   H₃C—C(O)(CH₂)ₙ—,
   (H₃C)₃C—O—C(O)(CH₂)ₙ—;
   wherein n is 0, 1 or 2;
and
R₃₀ is CH₃.

Embodiment 6. A compound of formula (I) or a pharmaceutically acceptable salt thereof, according to any of Embodiments 1 to 5, wherein R₁ is selected from:

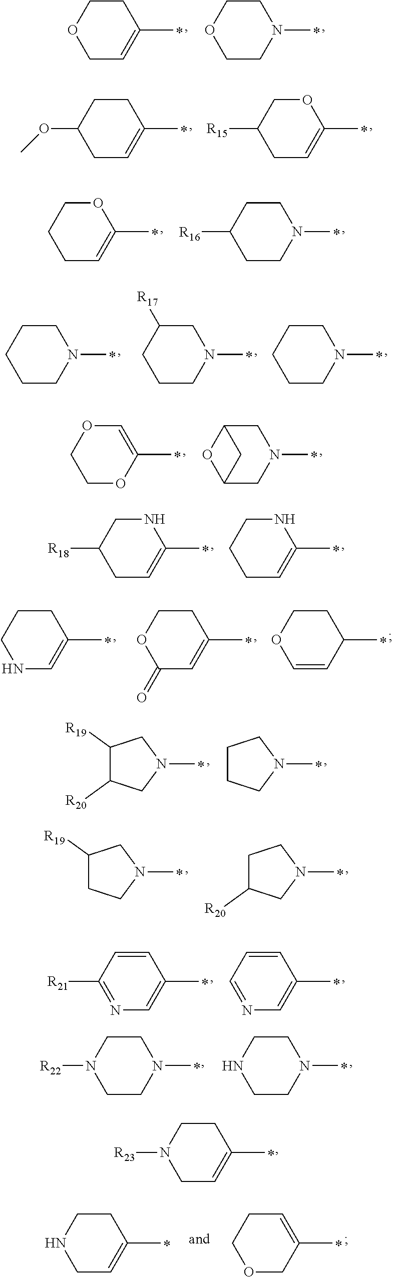

R₁₅ is F;
R₁₆ is R₂₅(R₂₄)N—;
R₁₇ is F;
R₁₈ is F;
R₁₉ is F;
R₂₀ is F;
R₂₁ is CH₃;
R₂₂ is CF₃, CHF₂CH₂, HOC(O)—CH₂—, H₃C—C(O)—, (H₃C)₃C—O—C(O)—;
R₂₃ is CF₃, CHF₂CH₂—, (H₃C)₃C—O—C(O)—;
R₂₄ is CH₃; and
R₂₅ is CHF₂CH₂—.

Embodiment 7. A compound of formula (I), or a pharmaceutically acceptable salt thereof, according to any of Embodiments 1 to 6, wherein R₁ is selected from:

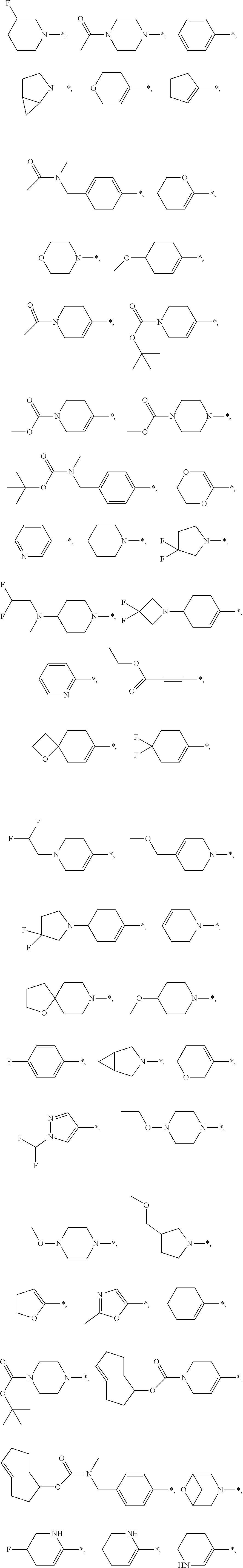

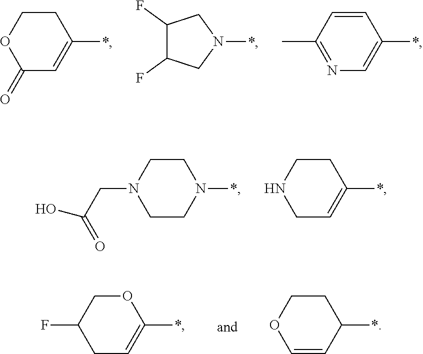

Embodiment 8. A compound of formula (I), or a pharmaceutically acceptable salt thereof, according to any of embodiments 1 to 7, wherein R₁ is selected from:

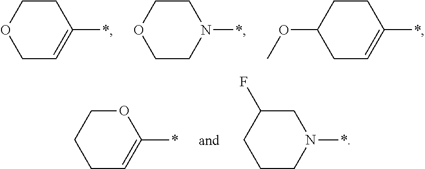

Embodiment 9. A compound of formula (I), or a pharmaceutically acceptable salt thereof, according to any of embodiments 1 to 8, wherein R₁ is selected from:

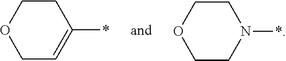

Embodiment 10. A compound of formula (I), or a pharmaceutically acceptable salt thereof, according to any of embodiments 1 to 9, wherein R₁ is:

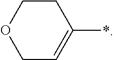

Embodiment 11. A compound of formula (I) or a pharmaceutically acceptable salt thereof, according to any of Embodiments 1 to 10, wherein R₂ is the moiety:

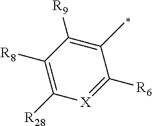

wherein
R₆ is selected from H, halo, (C₁-C₄)alkyl unsubstituted or substituted by 1, 2 or 3 halo;
R₈ is selected from H, halo, (C₁-C₄)alkyl unsubstituted or substituted by 1, 2 or 3 halo;
R₉ is selected from H, O—CH₃, OH, CN, CH₃ and halo;
R₂₈ is selected from SF₅, halo, (C₁-C₄)alkyl unsubstituted or substituted by 1, 2 or 3 halo, and —C(O)H;
X is selected from C—R₇ and N; and
R₇ is selected from H and halo.

Embodiment 12. A compound of formula (I) or a pharmaceutically acceptable salt thereof, according to any of Embodiments 1 to 11, wherein R₂ is the moiety:

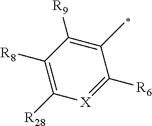

R₆ is selected from H, C, CH₃, F and Br;
R₈ is selected from H, C, F and CF₃;
R₉ is selected from H, CH₃ and Cl;
R₂₈ is selected from CF₃, CF₂H, —CH₂CH₃, Cl, SF₅, Br and —C(O)H;
X is selected from C—R₇ and N; and
R₇ is selected from H and F.

Embodiment 13. A compound of formula (I), or a pharmaceutically acceptable salt thereof, according to any of embodiments 1 to 12, wherein R₂₈ is selected from CF₃, CHF₂, Cl, —CH₂CH₃, CH₃, SF₅ and Br.

Embodiment 14. A compound of formula (I), or a pharmaceutically acceptable salt thereof, according to any of embodiments 1 to 13, wherein R₂₈ is selected from CF₃, Cl and SF₅, in particular CF₃.

Embodiment 15. A compound of formula (I), or a pharmaceutically acceptable salt thereof, according to any of embodiments 1 to 14, wherein X is CR₇.

Embodiment 16. A compound of formula (I), or a pharmaceutically acceptable salt thereof, according to any of embodiments 1 to 15, wherein R₇ is H.

Embodiment 17. A compound of formula (I), or a pharmaceutically acceptable salt thereof, according to any of embodiments 1 to 16, wherein R₆ is H, F, Cl or CH₃.

Embodiment 18. A compound of formula (I), or a pharmaceutically acceptable salt thereof, according to any of embodiments 1 to 17, wherein R₆ is Cl.

Embodiment 19. A compound of formula (I), or a pharmaceutically acceptable salt thereof, according to any of embodiments 1 to 18, wherein R₈ is F, CF₃ or H.

Embodiment 20. A compound of formula (I), or a pharmaceutically acceptable salt thereof, according to any of embodiments 1 to 19, wherein R₈ is H.

Embodiment 21. A compound of formula (I), or a pharmaceutically acceptable salt thereof, according to any of embodiments 1 to 20, wherein R₉ is H.

Embodiment 22. A compound of formula (I), or a pharmaceutically acceptable salt thereof, according to any of embodiments 1 to 21, wherein R₂ is selected from

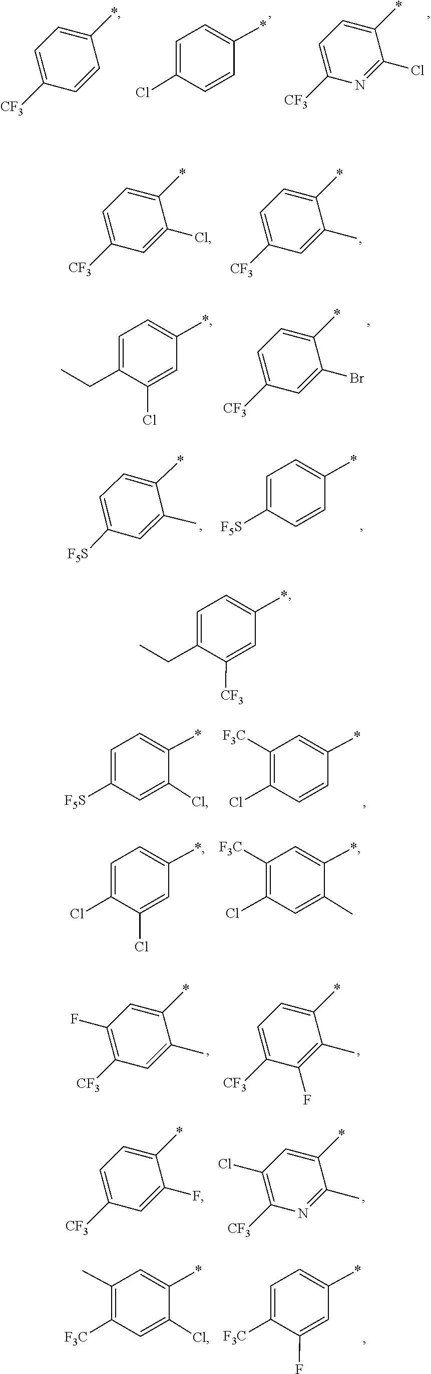

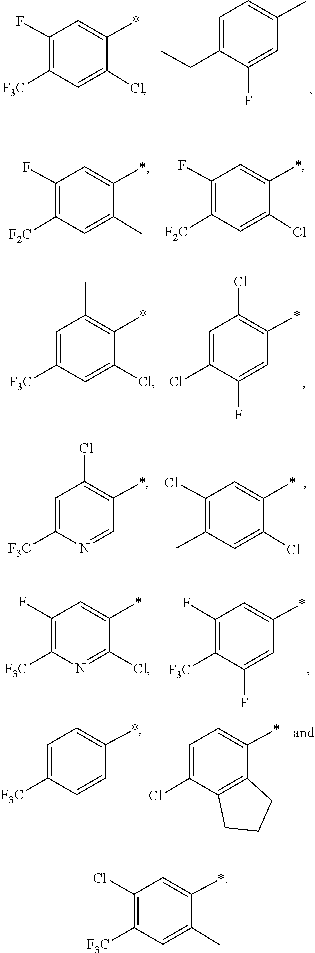

Embodiment 23. A compound of formula (I) or a pharmaceutically acceptable salt thereof, according to any of Embodiments 1 to 22, wherein R₃ is (C₁-C₄)alkyl unsubstituted or substituted by 1, 2 or 3 substituents independently selected from halo and OH.

Embodiment 24. A compound of formula (I) or a pharmaceutically acceptable salt thereof, according to any of Embodiments 1 to 23, wherein R₃ is (C₁-C₂)alkyl unsubstituted or substituted by 1, 2 or 3 substituents independently selected from halo and OH, preferably —CH₂CH₃ or CH₃, more preferably —CH₂CH₃.

Embodiment 25. A compound of formula (I) or a pharmaceutically acceptable salt thereof, according to any of Embodiments 1 to 24, wherein R₃ is selected from —CH₃, —CH₂CH₃, —CH(CH₃)₂, and cyclopropyl.

Embodiment 26. A compound of formula (I), or a pharmaceutically acceptable salt thereof, according to any of embodiments 1 to 25, wherein R₃ is —CH₃ or —CH₂—CH₃.

Embodiment 27. A compound of formula (I), or a pharmaceutically acceptable salt thereof, according to any of embodiments 1 to 26, wherein R₂₆ is H.

Embodiment 28 A compound of formula (I), or a pharmaceutically acceptable salt thereof, according to any of embodiments 1 to 27, wherein R₂₇ is H.

Embodiment 29. A compound of formula (I) or a pharmaceutically acceptable salt thereof, according to any of Embodiments 1 to 28, wherein R₄ is selected from:

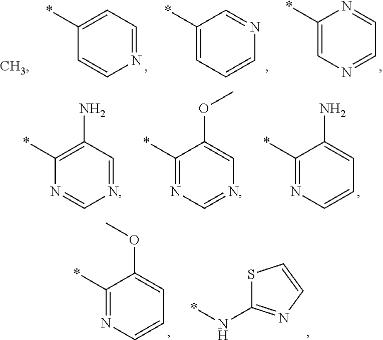

-heteroaryl1, wherein said heteroaryl1 is a 5 membered, fully unsaturated, monocyclic ring comprising ring carbon atoms and 1, 2, 3 or 4 ring heteroatoms independently selected from N, O and S;
-heteroaryl2, wherein said heteroaryl2 is a 9 or 10 membered fused bicyclic ring comprising ring carbon atoms and 1, 2, 3 or 4 ring heteroatoms independently selected from N, O and S, and wherein both rings are fully unsaturated, or one ring is fully unsaturated, and the other is saturated or partially unsaturated, and wherein the heteroatoms may be in one or both rings;
-phenyl; or
-heterocyclyl2, wherein said heterocyclyl2 is a 5 or 6 membered fully saturated or partially unsaturated group comprising ring carbon atoms and 1 or 2 ring heteroatoms independently selected from N, O and S,
wherein heteroaryl1, heteroaryl2, phenyl, and the moiety selected from:

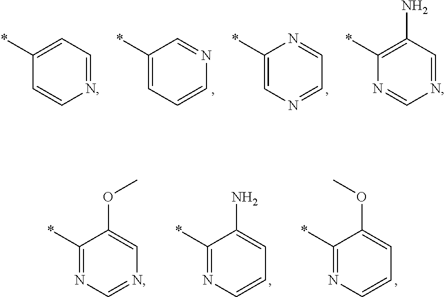

are each substituted by 1, 2 or 3 substituents, in particular 1 or 2 substituents, independently selected from R₁₀, R₁₁, R₁₂, R₁₃ and R₁₄, wherein each R₁₀, R₁₁, R₁₂, R₁₃ and R₁₄ is independently selected from:
   H,
   halo,
   (C₁-C₄)alkyl unsubstituted or substituted by 1, 2 or 3 halo substituents,
   (C₁-C₂)alkyl substituted by —O—(C₁-C₂)alkyl or OH,
   —S—(C₁-C₃)alkyl,
   —O—(C₁-C₄)alkyl unsubstituted or substituted by 1, 2 or 3 halo substituents,
   OH,
   (C₃-C₅)cycloalkyl, wherein said (C₃-C₅)cycloalkyl is unsubstituted or substituted by 1 or 2 halo,
   —O—(C₃-C₅)cycloalkyl,
   —NR₃₄R₃₅ wherein R₃₄ and R₃₅ are independently selected from:
      H,
      (C₁-C₄)alkyl, wherein said (C₁-C₄)alkyl is unsubstituted or substituted by OH or —O(C₁-C₂)alkyl,
      and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
   CN,
   —(C₂-C₄)alkenyl,
   —(C₂-C₄)alkynyl,
   ═O
   —C(O)H, and
   —C(O)(C₁-C₄)alkyl;

Embodiment 30. A compound of formula (I) or a pharmaceutically acceptable salt thereof, according to any of Embodiments 1 to 29, wherein R₄ is as described in embodiment 1 and other embodiments herein, with the proviso that at least one OH, CN, ═O, or NH₂ substituent is present on each heteroaryl1, heteroaryl2, phenyl,

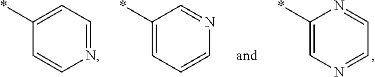

and the remaining R₁₀, R₁₁, R₁₂, R₁₃ and R₁₄ are as defined herein.

Embodiment 31. A compound of formula (I) or a pharmaceutically acceptable salt thereof, according to any of Embodiments 1 to 30, wherein R₄ is as described in embodiment 1 and other embodiments herein, with the proviso that one OH substituent is present on each heteroaryl1, heteroaryl2, phenyl,

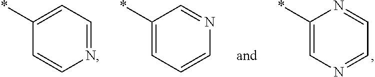

and the remaining R₁₀, R₁₁, R₁₂, R₁₃ and R₁₄ are as defined herein.

Embodiment 32. A compound of formula (I) or a pharmaceutically acceptable salt thereof, according to any of Embodiments 1 to 31, wherein R₄ is as described in embodiment 1 and other embodiments herein, with the proviso that one OH substituent is present on each heteroaryl1, heteroaryl2, phenyl,

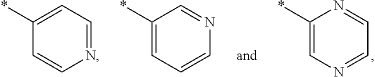

and said OH substituent is in the ortho position of the R₄ ring, relative to the position linking R₄ to linker —C(O)—, and the remaining R₁₀, R₁₁, R₁₂, R₁₃ and R₁₄ are as defined herein.

Embodiment 33. A compound of formula (I) or a pharmaceutically acceptable salt thereof, according to any of Embodiments 1 to 32, wherein R₄ is as described in embodiment 1 and other embodiments herein, and each R₁₀, R₁₁, R₁₂, R₁₃ and R₁₄ is independently selected from:
H,
halo (preferably F),
(C₁-C₂)alkyl (preferably CH₃), said (C₁-C₂)alkyl being unsubstituted or substituted by 1, 2 or 3 halo,
═O,
CN,
NH₂, and
—O—(C₁-C₂)alkyl unsubstituted or substituted by 1, 2 or 3 halo.

Embodiment 34. A compound of formula (I), or a pharmaceutically acceptable salt thereof, according to any of embodiments 1 to 33 wherein R₄ is selected from:

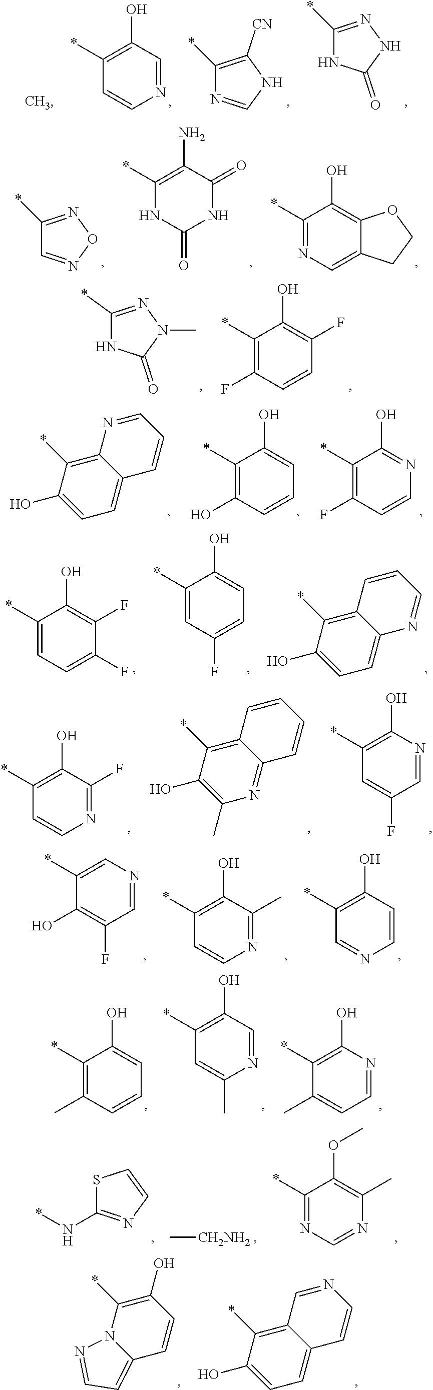

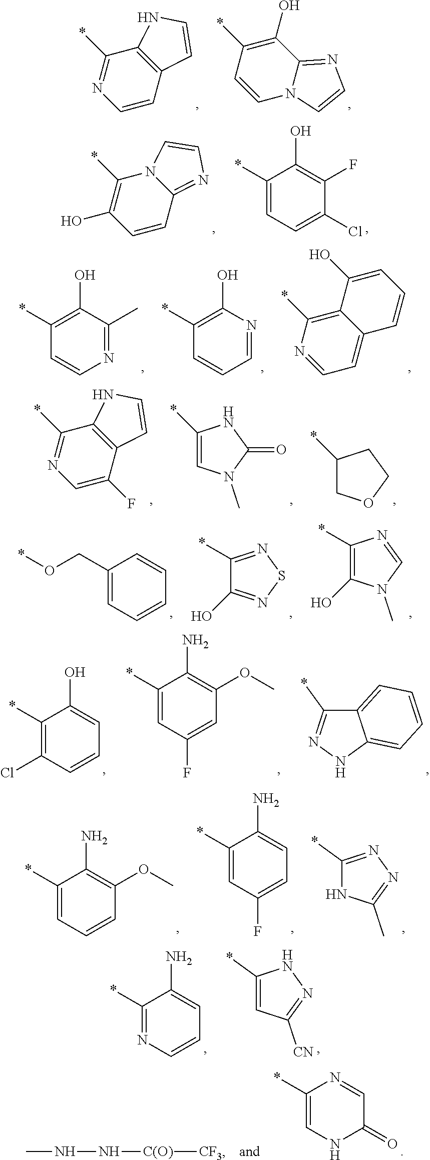

Embodiment 35. A compound of formula (I) or a pharmaceutically acceptable salt thereof, according to any of Embodiments 1 to 34, wherein Y is N and Y [symbol] is Y linked by a single bond.

Embodiment 36. A compound of formula (I) or a pharmaceutically acceptable salt thereof, according to any of Embodiments 1 to 35, wherein K [symbol] is K linked by a single bond, and K is selected from —CH₂—, —CH₂CH₂—, —NH— and a bond (to form a 5-membered ring:

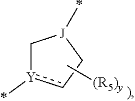

### and J is N.

Embodiment 37. A compound of formula (I) or a pharmaceutically acceptable salt thereof, according to any of Embodiments 1 to 36, wherein K [symbol] is K linked by a single bond, K is —CH₂— and J is N.

Embodiment 38. A compound of formula (I) or a pharmaceutically acceptable salt thereof, according to any of Embodiments 1 to 37, wherein R₅ is independently selected from:
—(C₁-C₄)alkyl, preferably methyl,
and wherein two R₅ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S,
when K [symbol] is a carbon-nitrogen single bond, a R₅ substituent on K and on the adjacent carbon atom may join to form ring C:

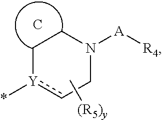

wherein ring C is a fused (C₃-C₆)cycloalkyl ring, in particular a fused cyclobutyl ring, a fused (C₃-C₆)heterocyclyl ring or a fused phenyl ring, wherein said fused (C₃-C₆)heterocyclyl ring contains ring carbon atoms and one ring heteroatom selected from O, N and S,
   and wherein when ring C is a fused (C₃-C₆)cycloalkyl ring, in particular fused cyclobutyl ring, said fused (C₃-C₆)cycloalkyl ring is unsubstituted or substituted with 1 or 2 R₄₀ groups, wherein said R₄₀ is selected from:
      (C₁-C₂)alkyl, wherein each (C₁-C₂)alkyl is independently unsubstituted or substituted by OH or 1, 2 or 3 halo,
      halo, in particular F,
      or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
      or wherein two R₄₀ substituents on adjacent carbon atoms join together with the carbon atoms to which they are attached, to form a fused cyclopropyl ring;
and wherein when K is —CH₂— and J is N, two R₅ substituents may join to form a (C₁-C₃)alkylene bridge or a heteroalkylene bridge, wherein said heteroalkylene bridge is one heteroatom selected from N and O, or is —CH₂—O—CH₂—.

Embodiment 39. A compound of formula (I) or a pharmaceutically acceptable salt thereof, according to any of Embodiments 1 to 38, wherein R₅ is independently selected from:
—(C₁-C₄)alkyl, preferably methyl,
when K [symbol] J is a carbon-nitrogen single bond, a R₅ substituent on K and on the adjacent carbon atom may join to form ring C:

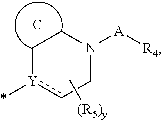

wherein ring C is a fused (C₃-C₆)cycloalkyl ring, in particular a fused cyclobutyl ring, or a fused (C₃-C₆)heterocyclyl ring, wherein said fused (C₃-C₆)heterocyclyl ring contains ring carbon atoms and one ring heteroatom selected from O, N and S,
   and wherein when ring C is a fused (C₃-C₆)cycloalkyl ring, in particular fused cyclobutyl ring, said fused (C₃-C₆)cycloalkyl ring is unsubstituted or substituted with 1 or 2 R₄₀ groups, wherein said R₄₀ is selected from:
      (C₁-C₂)alkyl, wherein each (C₁-C₂)alkyl is independently unsubstituted or substituted by OH or 1, 2 or 3 halo,
      halo, in particular F,
      or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
      or wherein two R₄₀ substituents on adjacent carbon atoms join together with the carbon atoms to which they are attached, to form a fused cyclopropyl ring;
and wherein when K is —CH₂— and J is N, two R₅ substituents may join to form a (C₁-C₃)alkylene bridge or a heteroalkylene bridge, wherein said heteroalkylene bridge is one heteroatom selected from N and O, or is —CH₂—O—CH₂—.

Embodiment 40. A compound of formula (I) or a pharmaceutically acceptable salt thereof, according to any of Embodiments 1 to 39, wherein R₅ is independently selected from:
—(C₁-C₂)alkyl, preferably methyl, and
when K [symbol] J is a carbon-nitrogen single bond, a R₅ substituent on K and on the adjacent carbon atom may join to form ring C:

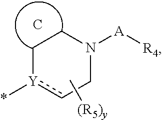

wherein ring C is a fused (C₃-C₄)cycloalkyl ring, in particular a fused cyclobutyl ring, and said fused (C₃-C₄)cycloalkyl ring, in particular fused cyclobutyl ring, is unsubstituted or substituted with 1 or 2 R₄₀ groups as described in the embodiments herein.

Embodiment 41. A compound of formula (I) or a pharmaceutically acceptable salt thereof, according to any of Embodiments 1 to 40, wherein y is 0, 1, 2 or 3, preferably 0, 1, or 2.

Embodiment 42. A compound of formula (I), or a pharmaceutically acceptable salt thereof, according to any of embodiments 1 to 41, wherein y is 0.

Embodiment 43. A compound of formula (I) or a pharmaceutically acceptable salt thereof, according to any of Embodiments 1 to 42, wherein R₅ is independently selected from:
CH₃, and y is 1 or 2, and
when K [symbol] J is a carbon-nitrogen single bond, a R₅ substituent on K and on the adjacent carbon atom may join to form ring C:

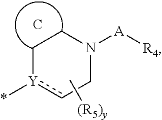

wherein ring C is a fused cyclobutyl ring.

Embodiment 44. A compound of formula (I) or a pharmaceutically acceptable salt thereof, according to any of Embodiments 1 to 43, wherein the compound of formula (I) includes the moiety:

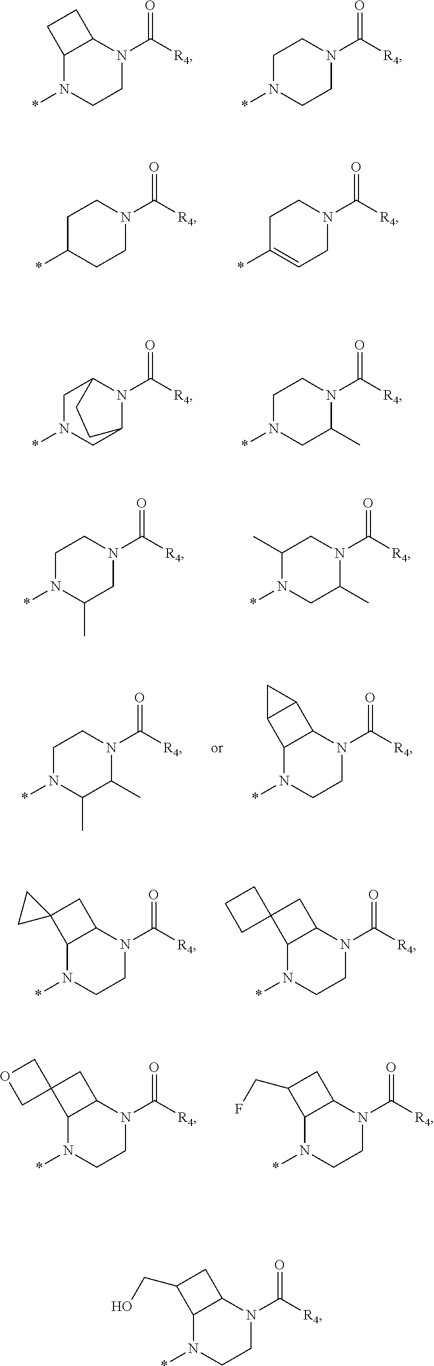

in particular
A:

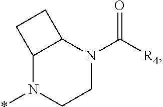

more particularly

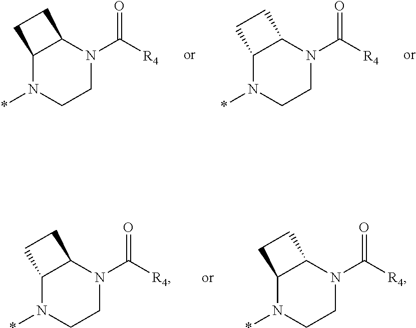

or B:

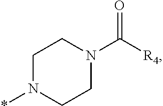

or C:

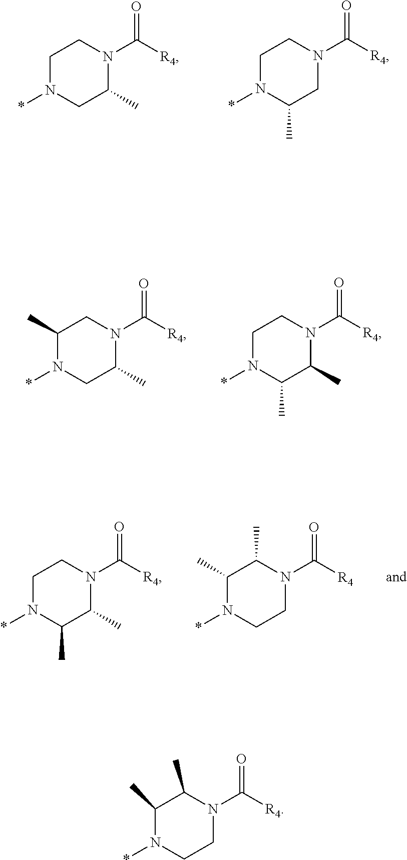

Embodiment 45. A compound of formula (I) or a pharmaceutically acceptable salt thereof, according to any of Embodiments 1 to 44, wherein the compound of formula (I) includes the moiety:

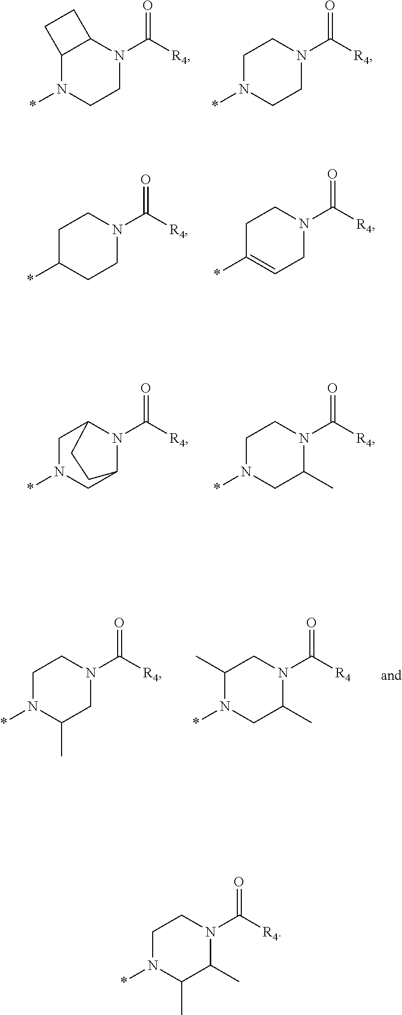

Embodiment 46. A compound of formula (I) or a pharmaceutically acceptable salt thereof, according to any of Embodiments 1 to 45, wherein formula (I) is formula 1a, wherein A is —C(O)—:

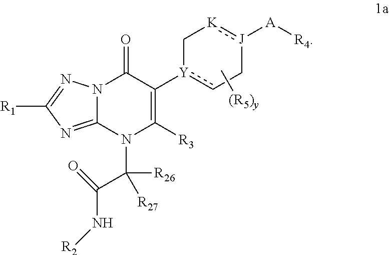

(Preferably, formula (I) is formula 1a)

Embodiment 47. A compound of formula (I) or a pharmaceutically acceptable salt thereof, according to any of Embodiments 1 to 45, wherein formula (I) is formula 1b, wherein A is —C(O)—:

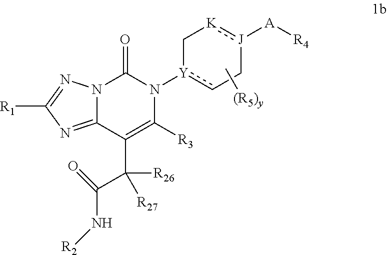

Embodiment 48. A compound of formula (I) or a pharmaceutically acceptable salt thereof, according to any of Embodiments 1 to 45, wherein formula (I) is formula 1c, wherein A is —C(O)—:

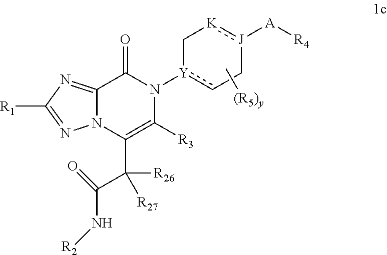

Embodiment 49. A compound of formula (I) or a pharmaceutically acceptable salt thereof, according to any of Embodiments 1 to 45, wherein formula (I) is formula 1d, wherein A is —C(O)—:

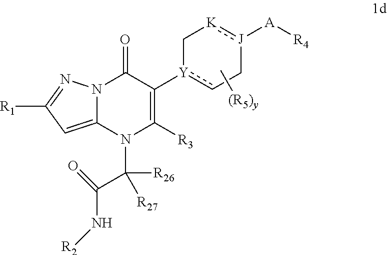

Embodiment 50. A compound of formula (I) or a pharmaceutically acceptable salt thereof, according to any of Embodiments 1 to 45, wherein formula (I) is formula 1e, wherein A is —C(O)—:

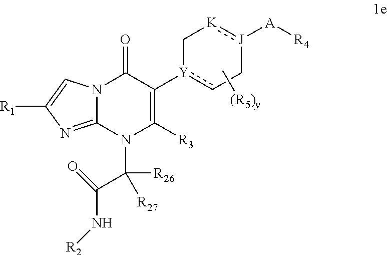

Embodiment 51. A compound of formula (I) or a pharmaceutically acceptable salt thereof, according to any of Embodiments 1 to 45, wherein formula (I) is formula 1f, wherein A is —C(O)—:

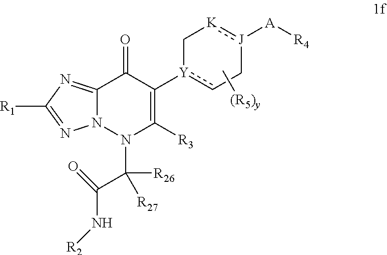

Embodiment 52. A compound of formula (I) or a pharmaceutically acceptable salt thereof, according to any of Embodiments 1 to 45, wherein formula (I) is formula 1g:

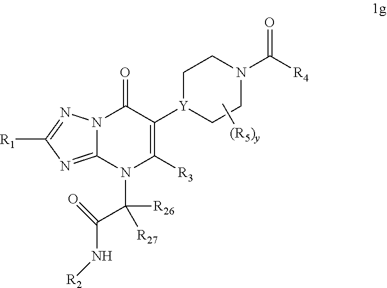

More preferably, formula (I) is formula 1g.

Embodiment 53. A compound of formula (I) or a pharmaceutically acceptable salt thereof, according to any of Embodiments 1 to 45 wherein formula (I) is formula 1 h:

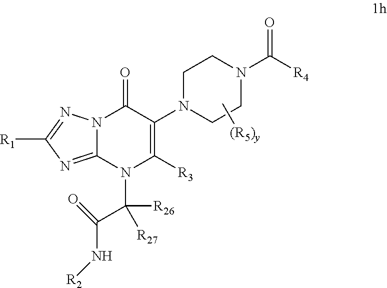

Most preferably, formula (I) is formula 1 h.

Embodiment 54. A compound of formula (1a) or a pharmaceutically acceptable salt thereof, according to any of Embodiments 1 to 46, wherein A is —C(O)—:

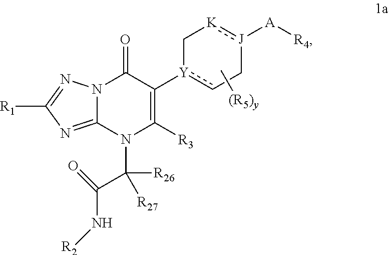

wherein
Y is N, C or CH;
Y [symbol] means is linked via a single bond to the adjacent carbon atom when Y is CH, or Y is linked via a double bond to the adjacent atom when Y is C;
y is 0, 1, 2 or 3;
K [symbol] is K linked by a single bond, K is —CH₂— and J is N;
R₅ is independently selected from:
   —(C₁-C₄)alkyl, preferably methyl,
   and wherein two R₅ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S,
   when K [symbol] J is a carbon-nitrogen single bond, a R₅ substituent on K and on the adjacent carbon atom may join to form ring C:

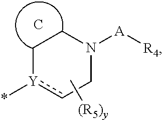

wherein ring C is a fused (C₃-C₆)cycloalkyl ring, in particular a fused cyclobutyl ring, a fused (C₃-C₆)heterocyclyl ring or a fused phenyl ring, wherein said fused (C₃-C₆)heterocyclyl ring contains ring carbon atoms and one ring heteroatom selected from O, N and S,
   and wherein when ring C is a fused (C₃-C₆)cycloalkyl ring, in particular fused cyclobutyl ring, said fused (C₃-C₆)cycloalkyl ring is unsubstituted or substituted with 1 or 2 R₄₀ groups, wherein said R₄₀ is selected from:
      (C₁-C₂)alkyl, wherein each (C₁-C₂)alkyl is independently unsubstituted or substituted by OH or 1, 2 or 3 halo,
      halo, in particular F,
      or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
      or wherein two R₄₀ substituents on adjacent carbon atoms join together with the carbon atoms to which they are attached, to form a fused cyclopropyl ring;
and wherein when K is —CH₂— and J is N, two R₅ substituents may join to form a (C₁-C₃)alkylene bridge or a heteroalkylene bridge, wherein said heteroalkylene bridge is one heteroatom selected from N and O, or is —CH₂—O—CH₂—;
R₁ is selected from:

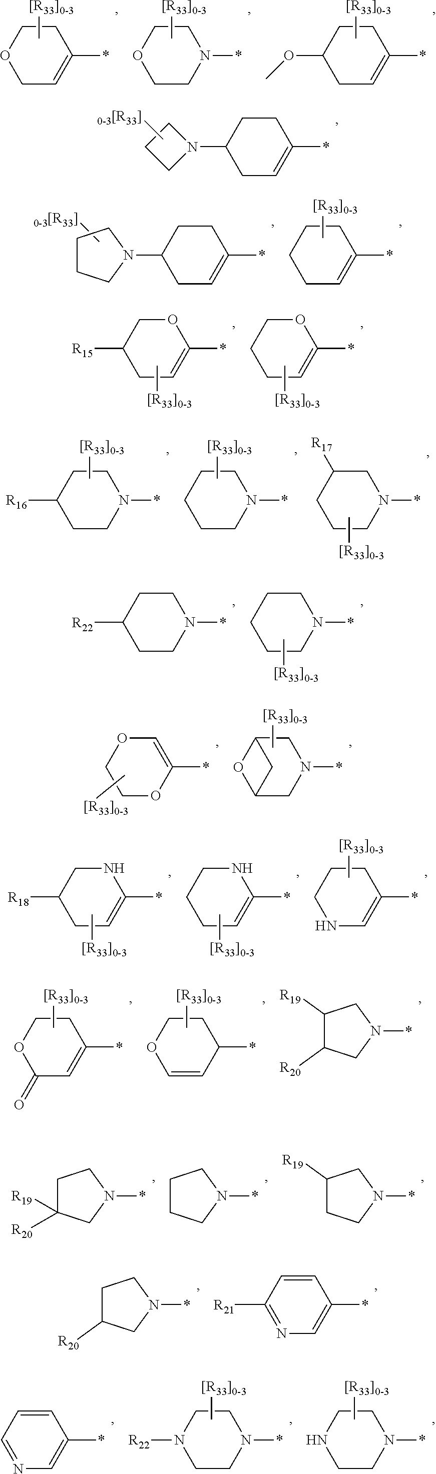

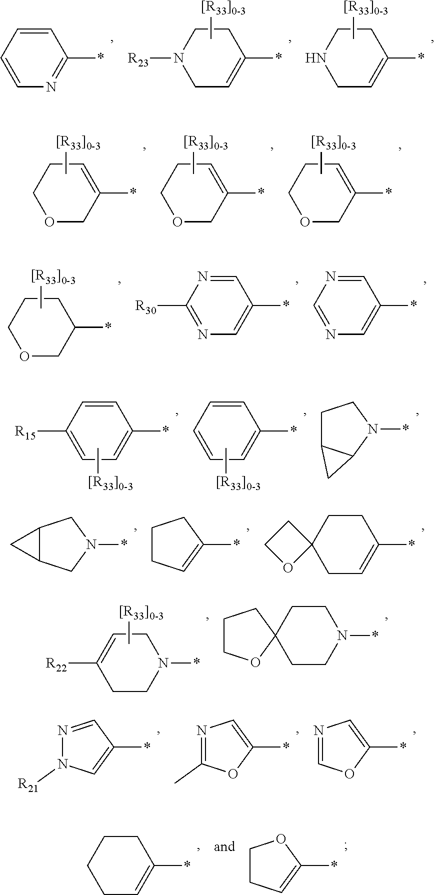

alternatively, there are 0-2 R₃₃ substituents, in each of the moieties above,
R₃₃ is F;
R₁₅ is:
   halo,
   R₂₅(R₂₄)N—(CH₂)ₙ, wherein R₂₄ is H or CH₃ unsubstituted or substituted by 1, 2 or 3 halo, R₂₅ is H, (C₁-C₄)alkyl-C(O)—, (C₁-C₄)alkyl-O—C(O)—, or (C₁-C₄)alkyl unsubstituted or substituted by 1, 2 or 3 halo, or
   azetidinyl or pyrrolidinyl, wherein said azetidinyl and pyrrolidinyl are linked to the rest of the molecule via the N atom, and are unsubstituted or substituted by 1 or 2 F;
R₁₆ is R₂₅(R₂₄)N—, wherein R₂₄ is H or (C₁-C₂)alkyl, R₂₅ is H or (C₁-C₂)alkyl unsubstituted or substituted by 1, 2 or 3 halo, in particular F;
R₁₇ is halo
R₁₈ is halo;
R₁₉ is:
   halo
   (C₁-C₄)alkyl unsubstituted or substituted by 1, 2 or 3 halo,
   (C₁-C₄)alkyl-O—(CH₂)ₙ—;
R₂₀ is halo;
R₂₁ is (C₁-C₂)alkyl, unsubstituted or substituted by 1, 2 or 3 F;
R₂₂ and R₂₃ are each independently selected from:
   (C₁-C₄)alkyl unsubstituted or substituted by 1, 2 or 3 halo,
   (C₁-C₄)alkyl-O—(CH₂)ₙ—
   HOC(O)—(CH₂)ₙ—,
   H₃C—C(O)(CH₂)ₙ—,
   (H₃C)₃C—O—C(O)(CH₂)ₙ—;
   wherein n is 0, 1 or 2;
and
R₃₀ is CH₃;
R₂ is the moiety:

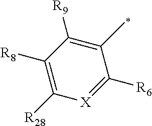

wherein
R₆ is selected from H, halo, (C₁-C₄)alkyl unsubstituted or substituted by 1, 2 or 3 halo;
R₈ is selected from H, halo, (C₁-C₄)alkyl unsubstituted or substituted by 1, 2 or 3 halo;
R₉ is selected from H, O—CH₃, OH, CN, CH₃ and halo;
R₂₈ is selected from SF₅, halo, (C₁-C₄)alkyl unsubstituted or substituted by 1, 2 or 3 halo, and —C(O)H;
X is selected from C—R₇ and N; and
R₇ is selected from H and halo;
R₃ is selected from —CH₃, —CH₂CH₃, —CH(CH₃)₂, and cyclopropyl, in particular —CH₃, and —CH₂CH₃;
R₂₆ is H;
R₂₆ is H;
R₄ is selected from:
—(C₁-C₄)alkyl, unsubstituted or substituted by NH₂, —O—(CH₂)₁₋₂-phenyl, —NH—NH—C(O)—CF₃,
-heteroaryl1, wherein said heteroaryl1 is a 5 or 6 membered, fully unsaturated, monocyclic ring comprising ring carbon atoms and 1, 2, 3 or 4 ring heteroatoms independently selected from N, O and S;
-heteroaryl2, wherein said heteroaryl2 is a 9 or 10 membered fused bicyclic ring comprising ring carbon atoms and 1, 2, 3 or 4 ring heteroatoms independently selected from N, O and S, and wherein both rings are fully unsaturated, or one ring is fully unsaturated, and the other is saturated or partially unsaturated, and wherein the heteroatoms may be in one or both rings;
-phenyl;
-heterocyclyl2, wherein said heterocyclyl2 is a 5 or 6 membered fully saturated or partially unsaturated group comprising ring carbon atoms and 1 or 2 ring heteroatoms independently selected from N, O and S,
wherein heteroaryl1, heteroaryl2 and phenyl are each substituted by 1, 2 or 3 substituents independently selected from R₁₀, R₁₁, R₁₂, R₁₃ and R₁₄, wherein each R₁₀, R₁₁, R₁₂, R₁₃ and R₁₄ is independently selected from:
   H,
   halo,
   (C₁-C₄)alkyl unsubstituted or substituted by 1, 2 or 3 halo substituents,
   (C₁-C₂)alkyl substituted by —O—(C₁-C₂)alkyl or OH,
   —S—(C₁-C₃)alkyl,
   —O—(C₁-C₄)alkyl unsubstituted or substituted by 1, 2 or 3 halo substituents,
   OH,
   (C₃-C₅)cycloalkyl, wherein said (C₃-C₅)cycloalkyl is unsubstituted or substituted by 1 or 2 halo,
   —O—(C₃-C₅)cycloalkyl,
   —NR₃₄R₃₅ wherein R₃₄ and R₃₅ are independently selected from:
      H,
      (C₁-C₄)alkyl, wherein said (C₁-C₄)alkyl is unsubstituted or substituted by OH or —O(C₁-C₂)alkyl,
      and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
      CN,
      —(C₂-C₄)alkenyl,
      —(C₂-C₄)alkynyl,
      ═O
      —C(O)H, and
      —C(O)(C₁-C₄)alkyl;
with the proviso that R₄ is not:

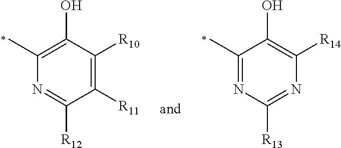

wherein
R₁₀, R₁₁, R₁₂, R₁₃ and R₁₄ are independently selected from:
   H,
   halo,
   (C₁-C₄)alkyl unsubstituted or substituted by 1, 2 or 3 halo substituents,
   (C₁-C₂)alkyl substituted by —O—(C₁-C₂)alkyl or OH,
   —S—(C₁-C₃)alkyl,
   —O—(C₁-C₄)alkyl unsubstituted or substituted by 1, 2 or 3 halo substituents,
   OH,
   (C₃-C₅)cycloalkyl, wherein said (C₃-C₅)cycloalkyl is unsubstituted or substituted by 1 or 2 halo,
   —O—(C₃-C₅)cycloalkyl,
   —NR₃₄R₃₅ wherein R₃₄ and R₃₅ are independently selected from:
      H,
      (C₁-C₄)alkyl, wherein said (C₁-C₄)alkyl is unsubstituted or substituted by OH or —O(C₁-C₂)alkyl,
      and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
   CN,
   —(C₂-C₄)alkenyl,
   —(C₂-C₄)alkynyl,
   —C(O)H, and
   —C(O)(C₁-C₄)alkyl;
and
* indicates a point of attachment.

In particular, R₄ is as described in any of the embodiments herein. For example, R₄ is selected from:

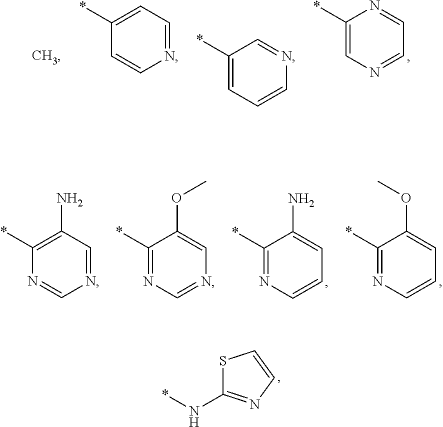

-heteroaryl1, wherein said heteroaryl1 is a 5 membered, fully unsaturated, monocyclic ring comprising ring carbon atoms and 1, 2, 3 or 4 ring heteroatoms independently selected from N, O and S;
-heteroaryl2, wherein said heteroaryl2 is a 9 or 10 membered fused bicyclic ring comprising ring carbon atoms and 1, 2, 3 or 4 ring heteroatoms independently selected from N, O and S, and wherein both rings are fully unsaturated, or one ring is fully unsaturated, and the other is saturated or partially unsaturated, and wherein the heteroatoms may be in one or both rings;
-phenyl; or
-heterocyclyl2, wherein said heterocyclyl2 is a 5 or 6 membered fully saturated or partially unsaturated group comprising ring carbon atoms and 1 or 2 ring heteroatoms independently selected from N, O and S,
wherein heteroaryl1, heteroaryl2, phenyl, and the moiety selected from:

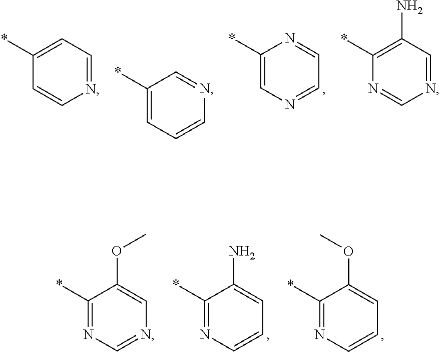

are each substituted by 1, 2 or 3 substituents, in particular 1 or 2 substituents, independently selected from R₁₀, R₁₁, R₁₂, R₁₃ and R₁₄, wherein each R₁₀, R₁₁, R₁₂, R₁₃ and R₁₄ is independently selected from:
   H,
   halo,
   (C₁-C₄)alkyl unsubstituted or substituted by 1, 2 or 3 halo substituents,
   (C₁-C₂)alkyl substituted by —O—(C₁-C₂)alkyl or OH,
   —S—(C₁-C₃)alkyl,
   —O—(C₁-C₄)alkyl unsubstituted or substituted by 1, 2 or 3 halo substituents,
   OH,
   (C₃-C₅)cycloalkyl, wherein said (C₃-C₅)cycloalkyl is unsubstituted or substituted by 1 or 2 halo,
   —O—(C₃-C₅)cycloalkyl,
   —NR₃₄R₃₅ wherein R₃₄ and R₃₅ are independently selected from:
      H,
      (C₁-C₄)alkyl, wherein said (C₁-C₄)alkyl is unsubstituted or substituted by OH or —O(C₁-C₂)alkyl,
      and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
   CN,
   —(C₂-C₄)alkenyl,
   —(C₂-C₄)alkynyl,
   ═O
   —C(O)H, and
   —C(O)(C₁-C₄)alkyl.

More particularly, R₁ is selected from:

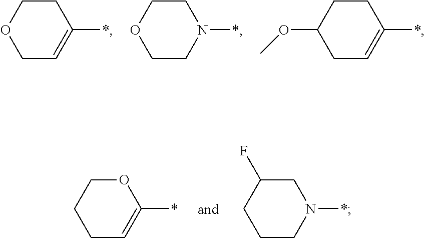

Embodiment 55. A compound of formula (1b) or a pharmaceutically acceptable salt thereof, according to any of Embodiments 1 to 45 or 47, wherein A is —C(O)—:

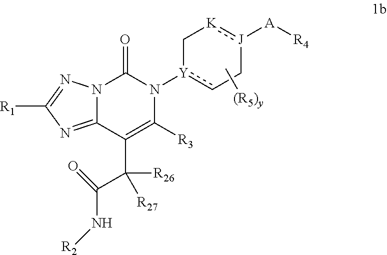

and wherein R₁, R₂, R₃, R₄, R₅, R₂₆, R₂₇, Y, K, J, and y are as defined in embodiment 54.

Embodiment 56. A compound of formula (1c) or a pharmaceutically acceptable salt thereof, according to any of Embodiments 1 to 45 or 48, wherein A is —C(O)—:

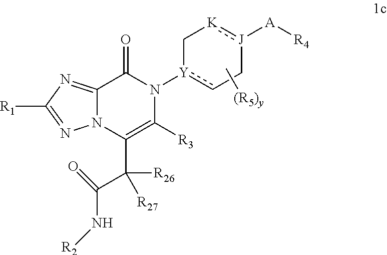

and wherein R₁, R₂, R₃, R₄, R₅, R₂₆, R₂₇, Y, K, J, and y are as defined in embodiment 54.

Embodiment 57. A compound of formula (1d) or a pharmaceutically acceptable salt thereof, according to any of Embodiments 1 to 45 or 49, wherein A is —C(O)—:

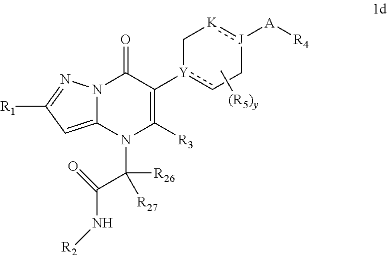

and wherein R₁, R₂, R₃, R₄, R₅, R₂₆, R₂₇, Y, K, J, and y are as defined in embodiment 54.

Embodiment 58. A compound of formula (1e) or a pharmaceutically acceptable salt thereof, according to any of Embodiments 1 to 45 or 50, wherein A is —C(O)—:

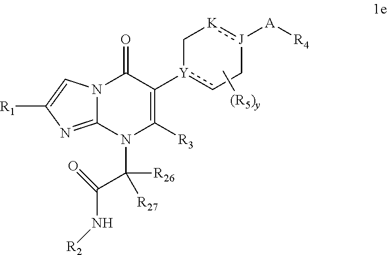

and wherein H₁, H₂, H₃, H₄, H₅, H₂₆, H₂₇, Y, K, J, and y are as defined in embodiment 54.

Embodiment 59. A compound of formula (1f) or a pharmaceutically acceptable salt thereof, according to any of Embodiments 1 to 45 or 51, wherein A is —C(O)—:

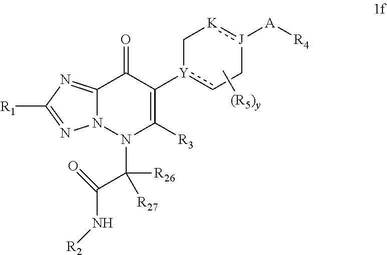

and wherein R₁, R₂, R₃, R₄, R₅, R₂₆, R₂₇, Y, K, J, and y are as defined in embodiment 54.

Embodiment 60. A compound of formula (1 g) or a pharmaceutically acceptable salt thereof, according to any of Embodiments 1 to 45 or 54, wherein formula 1 or 1a is formula 1g:

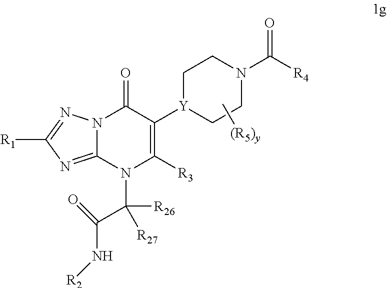

wherein R₁, R₂, R₃, R₄, R₅, R₂₆, R₂₇, Y and y are as defined in embodiment 54.

Embodiment 61. A compound of formula (1 h) or a pharmaceutically acceptable salt thereof, according to any of Embodiments 1 to 45, 54 or 60, wherein formula I or 1a or 1g, is formula 1h:

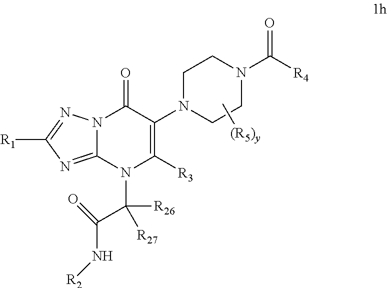

wherein R₁, R₂, R₃, R₄, R₅, R₂₆, R₂₇ and y are as defined in embodiment 54.

Embodiment 62. A compound of Formula (I) or a pharmaceutically acceptable salt thereof, selected from an exemplified compound structure herein.

Embodiment 63. A compound of formula (I), or a pharmaceutically acceptable salt thereof, according to any of embodiments 1-62, wherein the compound is in non-zwitterionic form.

Embodiment 64. A compound of formula (I), or a pharmaceutically acceptable salt thereof, according to any of embodiments 1-62, wherein the compound is in zwitterionic form.

Embodiment 65. A compound of formula (I), or a pharmaceutically acceptable salt thereof, according to any of embodiments 1-62, wherein the compound is a mixture of zwitterionic and non-zwitterionic forms.

Embodiment 66. A compound of formula (I), or a pharmaceutically acceptable salt thereof, according to any of embodiments 1-62, wherein the compound is a sodium salt.

Embodiment 67. A compound of formula (I), or a pharmaceutically acceptable salt thereof, according to any of embodiments 1-62, wherein the compound is in amorphous form. For example, the compound is the sodium salt in amorphous form.

Embodiment 68. A compound of formula (I), or a pharmaceutically acceptable salt thereof, according to any of embodiments 1-62, in crystalline form.

Embodiment 69. A compound of formula (I) according to any of embodiments 1-62, wherein the compound is in substantially pure form.

Embodiment 70. A combination comprising a compound of formula (I), or a pharmaceutically acceptable salt thereof, according to any of claims 1 to 62, and one or more additional therapeutically active agents.

Embodiment 71. A combination according to embodiment 70, wherein an additional therapeutically active agent is an anti-cancer agent.

Embodiment 72. A combination according to embodiment 70 or 71, wherein an additional therapeutically active agent is a chemotherapy.

Embodiment 73. A combination according to embodiment 72, wherein an additional therapeutically active agent is a chemotherapy selected from anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®), in particular irinotecan.

Embodiment 74. A combination according to embodiment 71, wherein an additional therapeutically active agent is a PD-1 inhibitor.

Embodiment 75. A combination according to embodiment 70 or 71, wherein an additional therapeutically active agent is an anti-PD-1 antibody molecule.

Embodiment 76. A combination according to embodiment 75, wherein an additional therapeutically active agent is a PD-1 inhibitor selected from PDR001 (Novartis), Nivolumab (Bristol-Myers Squibb), Pembrolizumab (Merck & Co), Pidilizumab (CureTech), MEDI0680 (Medimmune), Cemiplimab (REGN2810, Regeneron), Dostarlimab (TSR-042, Tesaro), PF-06801591 (Pfizer), Tislelizumab (BGB-A317, Beigene), BGB-108 (Beigene), INCSHR1210 (Incyte), Balstilimab (AGEN2035, Agenus), Sintilimab (InnoVent), Toripalimab (Shanghai Junshi Bioscience), Camrelizumab (Jiangsu Hengrui Medicine Co.), and AMP-224 (Amplimmune), in particular PDR001, Pembrolizumab or Tislelizumab, more particularly Tislelizumab.

Embodiment 77. A pharmaceutical composition comprising a compound of formula (I) or a pharmaceutically acceptable salt thereof, according to any of embodiments 1 to 62, and one or more pharmaceutically acceptable carriers.

Embodiment 78. A compound of formula (I), or a pharmaceutically acceptable salt thereof, according to embodiments 1-62, for use as a medicament.

Embodiment 79. A compound of formula (I), or a pharmaceutically acceptable salt thereof, according to embodiments 1-62, for use according to embodiment 78, wherein the use is for the treatment of a disease that is treated by WRN inhibition.

Embodiment 80. A compound of formula (I), or a pharmaceutically acceptable salt thereof, according to embodiments 1-62, for use according to embodiment 78, wherein the use is for the treatment of cancer.

Embodiment 81. A compound of formula (I), or a pharmaceutically acceptable salt thereof, according to embodiments 1-62, for use according to embodiment 80, wherein the cancer is characterized as microsatellite instability-high (MSI-H) or mismatch repair deficient (dMMR).

Embodiment 82. A compound of formula (I), or a pharmaceutically acceptable salt thereof, according to embodiments 1-62, for use according to embodiment 81, wherein the cancer characterized as microsatellite instability-high (MSI-H) or mismatch repair deficient (dMMR) is selected from colorectal, gastric, and endometrial, adrenocortical, uterine, cervical, esophageal, breast, kidney and ovarian cancer.

Embodiment 83. A compound of formula (I), or a pharmaceutically acceptable salt thereof, according to embodiments 1-62, for use according to embodiment 82, wherein the cancer characterized as microsatellite instability-high (MSI-H) or mismatch repair deficient (dMMR) is selected from colorectal, gastric and endometrial cancer.

Embodiment 84. A compound of formula (I), or a pharmaceutically acceptable salt thereof, according to embodiments 1-62, for use according to embodiment 81, wherein the cancer characterized as microsatellite instability-high (MSI-H) or mismatch repair deficient (dMMR) is selected from uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma and ovarian serous cystadenocarcinoma.

Embodiment 85. A method of modulating WRN activity in a subject, wherein the method comprises administering to the subject a therapeutically effective amount of the compound of formula (I), or a pharmaceutically acceptable salt thereof, according to embodiments 1-62.

Embodiment 86. A method of inhibiting WRN in a subject, wherein the method comprises administering to the subject a therapeutically effective amount of the compound of formula (I), or a pharmaceutically acceptable salt thereof, according to embodiments 1-62.

Embodiment 87. A method of treating a disorder or disease which can be treated by WRN inhibition in a subject, comprising administering to the subject a therapeutically effective amount of the compound of formula (I), or a pharmaceutically acceptable salt thereof, according to embodiments 1-62.

Embodiment 88. A method of treating cancer in a subject, comprising administering to the subject a therapeutically effective amount of the compound of formula (I), or a pharmaceutically acceptable salt thereof, according to embodiments 1-62.

Embodiment 89. A method of treating cancer in a subject, comprising administering a compound of formula (I), or a pharmaceutically acceptable salt thereof, according to embodiments 1-62, wherein the cancer is characterized as microsatellite instability-high (MSI-H) or mismatch repair deficient (dMMR).

Embodiment 90. The method according to embodiment 89, wherein the cancer characterized as microsatellite instability-high (MSI-H) or mismatch repair deficient (dMMR) is selected from colorectal, gastric, and endometrial, adrenocortical, uterine, cervical, esophageal, breast, kidney and ovarian cancer.

Embodiment 91. The method according to embodiment 90, wherein the cancer characterized as microsatellite instability-high (MSI-H) or mismatch repair deficient (dMMR) is selected from colorectal, gastric and endometrial cancer.

Embodiment 92. The method according to embodiment 89, wherein the cancer characterized as microsatellite instability-high (MSI-H) or mismatch repair deficient (dMMR) is selected from uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma and ovarian serous cystadenocarcinoma.

Embodiment 93. The use of a compound, or pharmaceutically acceptable salt thereof, according to any of embodiments 1 to 62, in the manufacture of a medicament for the treatment of cancer.

Embodiment 94. The use according to claim 93, of a compound or pharmaceutically acceptable salt thereof according to any of embodiments 1 to 62, wherein the cancer is characterized as microsatellite instability-high (MSI-H) or mismatch repair deficient (dMMR).

Embodiment 95. The use of a compound, or salt thereof, according to any of embodiments 1 to 62 as a research chemical, a chemical probe, or as a tool compound.

Embodiment 96. A process to manufacture a compound according to any of embodiments 1 to 62, or a pharmaceutically acceptable salt thereof.

Embodiment 97. An intermediate compound as defined herein.

There is also provided a compound of formula (I), or a pharmaceutically acceptable salt thereof, as described herein,
and in particular, when R₁ is a ring, then:
   each R₁ ring atom adjacent to the R₁ ring atom to which said R₁ ring is joined to the remainder of the molecule, is independently unsubstituted or substituted by halo only, in particular, independently unsubstituted or substituted with one F substituent, and
   preferably, said R₁ ring is linked to the remainder of the molecule via a R₁ ring nitrogen atom, or a R₁ ring carbon atom which is double-bonded to an adjacent R₁ ring atom.

More particularly, R₁ is:
cycloalkenyl, wherein said cycloalkenyl is a partially unsaturated monocyclic ring containing 5 or 6 ring carbon atoms, and said cycloalkenyl is unsubstituted or substituted by 1, 2, 3 or 4, preferably 1 or 2, R₃₃, wherein R₃₃ is halo, and wherein said cycloalkenyl or halo-substituted cycloalkenyl is substituted by 0, 1 or 2 R₁₅ substituents, preferably 1 substituent, or said cycloalkenyl or halo-substituted cycloalkenyl has 2 substitutents at the same ring carbon atom which join to form an oxetanyl spiro ring, or R₁ is heterocyclyl, wherein said heterocyclyl is a 5 or 6 membered fully saturated or partially unsaturated group comprising ring carbon atoms and 1 or 2 ring heteroatoms independently selected from N, NH, O and S, and wherein said heterocyclyl is unbridged or bridged, and said bridge is 1 or 2 carbon atoms, wherein said heterocyclyl is unsubstituted or substituted by 1, 2, 3 or 4, for example 1, 2 or 3, in particular 1 or 2 R₃₃, wherein R₃₃ is halo, and wherein said heterocyclyl or halo-substituted heterocyclyl is substituted by 0, 1 or 2 substituents, preferably 0 or 1 substituent, independently selected from R₁₅, R₁₆, R₁₇, R₁₈, R₁₉, R₂₀, R₂₂ and R₂₃,
or said heterocyclyl or halo-substituted heterocyclyl is fused to a cyclopropyl ring, wherein said cyclopropyl ring is unsubstituted or substituted by 1, 2 or 3 F,
or said heterocyclyl or halo-substituted heterocyclyl has 2 substitutents at the same ring carbon atom which join to form a tetrahydrofuranyl spiro ring,
or R₁ is heteroaryl, wherein said heteroaryl is a 5 or 6 membered fully unsaturated monocyclic group comprising ring carbon atoms and 1, 2, 3 or 4 ring heteroatoms independently selected from N, O and S, preferably 1 or 2 ring heteraoms, wherein the total number of ring S atoms does not exceed 1, and the total number of ring O atoms does not exceed 1, wherein said heteroaryl is unsubstituted or substituted by 1, 2 or 3 substituents independently selected from R₂₁ and R₃₀, wherein R₂₁ and R₃₀ are independently selected from halo and (C₁-C₄)alkyl, wherein said (C₁-C₄)alkyl is unsubstituted or substituted by 1, 2 or 3 halo,
or R₁ is phenyl, wherein said phenyl is unsubstituted or substituted by 1, 2, 3 or 4, preferably 1 or 2, R₃₃, wherein R₃₃ is halo, and wherein said phenyl or halo-substituted phenyl is substituted by 0 or 1 R₁₅ substituents,
or R₁ is (C₂-C₄)alkynyl, unsubstituted or substituted by (C₁-C₄)alkyl-O—C(O)—;
and each R₁₅, R₁₆, R₁₇, R₁₈, Rig, R₂₀, R₂₂ and R₂₃ is independently selected from:
   halo
   (C₁-C₄)alkyl-O—(CH₂)ₙ unsubstituted or substituted by 1, 2 or 3 halo;
   (C₁-C₄)alkyl unsubstituted or substituted by OH, —O—(C₁-C₂)alkyl or 1, 2 or 3 halo,
   HOC(O)—(CH₂)ₙ—,
   H₃C—C(O)(CH₂)ₙ—,
   (C₁-C₄)alkyl-O—C(O)(CH₂)ₙ,
   ═O
   azetidinyl or pyrrolidinyl, wherein said azetidinyl and pyrrolidinyl are linked to the rest of the molecule via the N atom, and are each unsubstituted or substituted by 1 or 2 F,
   R₂₅(R₂₄)N—(CH₂)ₙ, wherein R₂₄ is H or (C₁-C₂)alkyl unsubstituted or substituted by 1, 2 or 3 halo, R₂₅ is H, (C₁-C₄)alkyl-C(O)—, (C₁-C₄)alkyl-O—C(O)—, or (C₁-C₄)alkyl unsubstituted or substituted by 1, 2 or 3 halo,
   OH
wherein n is 0, 1 or 2,
even more particularly, R₁ is:
cycloalkenyl, wherein said cycloalkenyl is a partially unsaturated monocyclic ring containing 5 or 6 ring carbon atoms, and said cycloalkenyl is unsubstituted or substituted by 1 or 2 R₃₃, wherein R₃₃ is halo, preferably F, and wherein said cycloalkenyl or halo-substituted cycloalkenyl is substituted by 0 or 1 R₁₅ substituents, wherein R₁₅ is selected from:
   h) (C₁-C₂)alkyl-O— unsubstituted or substituted by 1, 2 or 3 halo;
   i) (C₁-C₂)alkyl unsubstituted or substituted by 1, 2 or 3 halo,
   j) HOC(O)—(CH₂)ₙ—,
   k) H₃C—C(O)(CH₂)ₙ—,
   l) H₃C—O—C(O)(CH₂)ₙ,
   m) ═O, and
   n) R₂₅(R₂₄)N—, H, wherein R₂₄ is H or (C₁-C₂)alkyl unsubstituted or substituted by 1, 2 or 3 halo, R₂₅ is H or (C₁-C₂)alkyl unsubstituted or substituted by 1, 2 or 3 halo,
n is 0 or 1,
wherein
   the R₁₅ substituent a) to g) of said cycloalkenyl or halo-substituted cycloalkenyl is not present on the ring atoms adjacent to the ring atom to which the cycloalkenyl or halo-substituted cycloalkenyl is joined to the remainder of the molecule, and preferably, said cycloalkenyl or halo-substituted cycloalkenyl is a 6 membered ring, with 1 R₁₅ substituent in the ring para position relative to the remainder of the molecule; and
   said cycloalkenyl or halo-substituted cycloalkenyl is linked to the remainder of the compound via a R₁ ring carbon atom which is double bonded to an adjacent R₁ ring carbon atom;
or R₁ is heterocyclyl, wherein said heterocyclyl is a 5 or 6 membered fully saturated or partially unsaturated group comprising ring carbon atoms and 1 or 2 ring heteroatoms independently selected from N, NH, O and S, and wherein said heterocyclyl is unbridged or bridged, and said bridge is 1 or 2 carbon atoms, wherein said heterocyclyl is unsubstituted or substituted by 1 or 2 R₃₃, wherein R₃₃ halo, is preferably F, and wherein said heterocyclyl or halo-substituted heterocyclyl is substituted by 0 or 1 substituents independently selected from R₁₅, R₁₆, R₁₇, R₁₈, Rig, R₂₀, R₂₂ and R₂₃, wherein said R₁₅, R₁₆, R₁₇, R₁₈, Rig, R₂₀, R₂₂ and R₂₃ are independently selected from:
   i) (C₁-C₄)alkyl-O— unsubstituted or substituted by 1, 2 or 3 halo;
   j) (C₁-C₄)alkyl unsubstituted or substituted by OH, —O—(C₁-C₂)alkyl or 1, 2 or 3 halo,
   k) HOC(O)—(CH₂)ₙ—,
   l) H₃C—C(O)(CH₂)ₙ—,
   m) H₃C—O—C(O)(CH₂)ₙ,
   n) ═O
   o) R₂₅(R₂₄)N—, wherein R₂₄ is H, (C₁-C₂)alkyl unsubstituted or substituted by 1, 2 or 3 halo, R₂₅ is H, (C₁-C₂)alkyl unsubstituted or substituted by 1, 2 or 3 halo,
   p) OH
wherein n is 0 or 1,
and wherein:
   substituent a) to h) of said heterocyclyl or halo-substituted heterocyclyl is not present on the ring atoms adjacent to the ring atom to which the heterocyclyl or halo-substituted heterocyclyl is joined to the remainder of the molecule, and preferably, when said heterocyclyl or halo-substituted heterocyclyl is a 6 membered ring, it has 0 or 1 substituent selected from a) to h) in the meta or para position, preferably para, relative to the remainder of the molecule; and
   said heterocyclyl is linked to the remainder of the compound via a R₁ ring nitrogen atom, or a R₁ ring carbon atom which is double bonded to an adjacent ring atom;
or R₁ is heteroaryl, wherein said heteroaryl is a 5 or 6 membered fully unsaturated monocyclic group comprising ring carbon atoms and 1 or 2 ring heteroatoms independently selected from N, O and S, preferably N, wherein the total number of ring S atoms does not exceed 1, and the total number of ring O atoms does not exceed 1, wherein said heteroaryl is unsubstituted or substituted by 1 or 2 substituents independently selected from R₂₁ and R₃₀, wherein R₂₁ and R₃₀ are independently selected from (C₁-C₂)alkyl, and said (C₁-C₂)alkyl is unsubstituted or substituted by 1, 2 or 3 halo, and wherein preferably, said alkyl or halo-alkyl substituent is not present on the R₁ ring atoms adjacent to the R₁ ring atom to which the heteroaryl is joined to the remainder of the molecule, and more preferably, when heteroaryl is a 6-membered ring, said alkyl or halo-alkyl substituent is in the ring para position relative to the rest of the molecule.

In particular, R₁, is selected from:

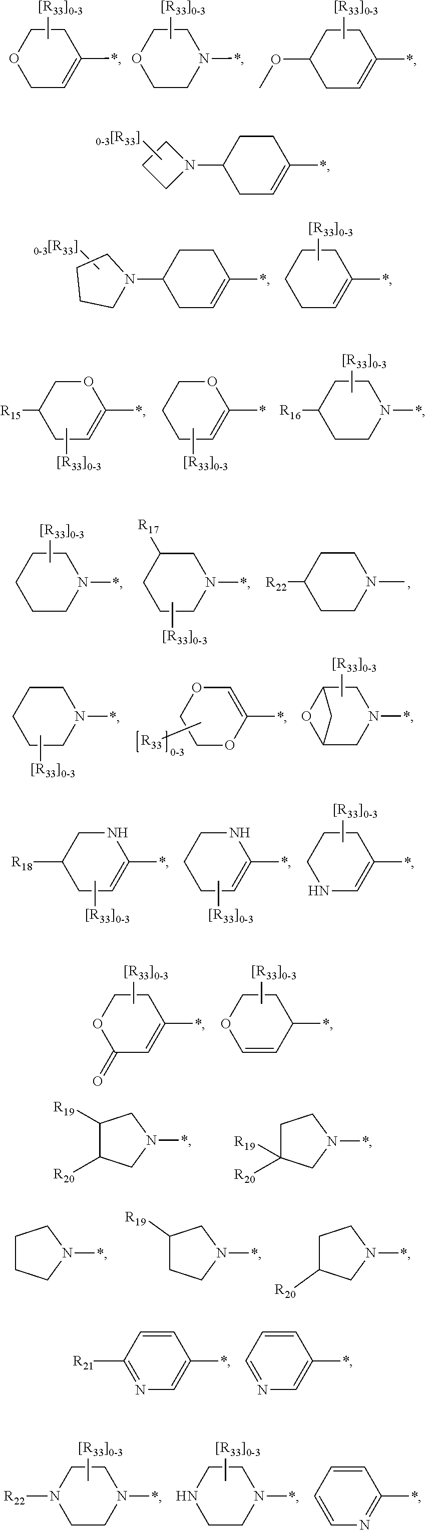

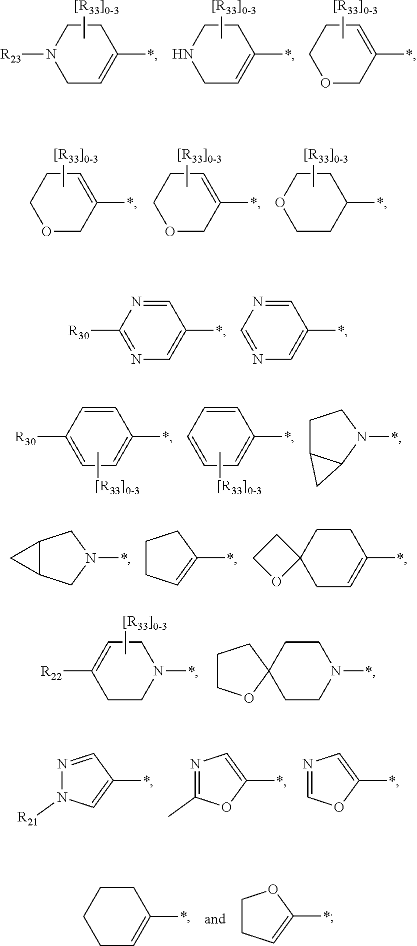

alternatively, there are 0-2 R₃₃ substituents, in each of the moieties above,
R₃₃ is F;
R₁₅ is:
   halo,
   R₂₅(R₂₄)N—(CH₂)ₙ, wherein R₂₄ is H or CH₃ unsubstituted or substituted by 1, 2 or 3 halo, R₂₅ is H, (C₁-C₄)alkyl-C(O)—, (C₁-C₄)alkyl-O—C(O)—, or (C₁-C₄)alkyl unsubstituted or substituted by 1, 2 or 3 halo, or
   azetidinyl or pyrrolidinyl, wherein said azetidinyl and pyrrolidinyl are linked to the rest of the molecule via the N atom, and are unsubstituted or substituted by 1 or 2 F;
R₁₆ is R₂₅(R₂₄)N—, wherein R₂₄ is H or (C₁-C₂)alkyl, R₂₅ is H or (C₁-C₂)alkyl unsubstituted or substituted by 1, 2 or 3 halo, in particular F;
R₁₇ is halo
R₁₈ is halo;
R₁₉ is:
   halo
   (C₁-C₄)alkyl unsubstituted or substituted by 1, 2 or 3 halo,
   (C₁-C₄)alkyl-O—(CH₂)ₙ—;
R₂₀ is halo;
R₂₁ is (C₁-C₂)alkyl, unsubstituted or substituted by 1, 2 or 3 F;
R₂₂ and R₂₃ are each independently selected from:
   (C₁-C₄)alkyl unsubstituted or substituted by 1, 2 or 3 halo,
   (C₁-C₄)alkyl-O—(CH₂)ₙ—
   HOC(O)—(CH₂)ₙ—,
   H₃C—C(O)(CH₂)ₙ—,
   (H₃C)₃C—O—C(O)(CH₂)ₙ—;
   wherein n is 0, 1 or 2;
and
R₃₀ is CH₃.

R₁ is preferably selected from:

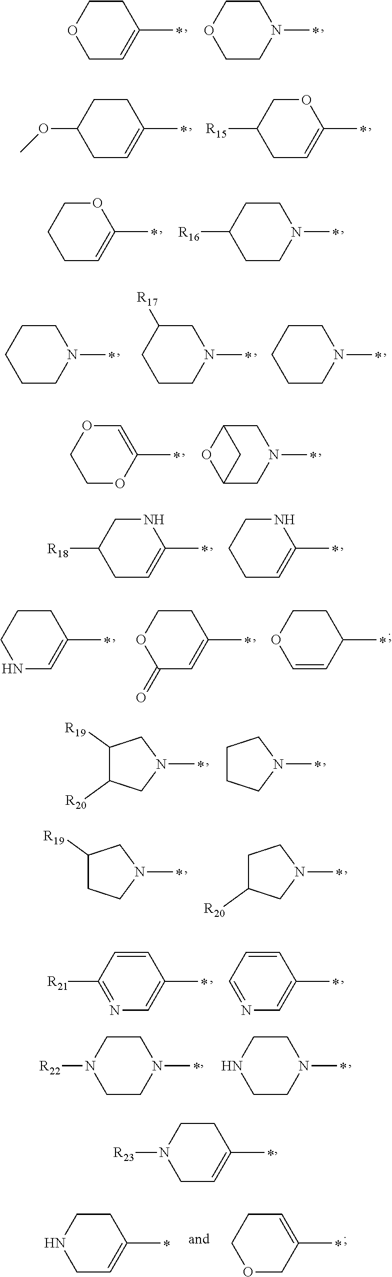

R₁₅ is F;
R₁₆ is R₂₅(R₂₄)N—;
R₁₇ is F;
R₁₈ is F;
R₁₉ is F;
R₂₀ is F;
R₂₁ is CH₃;
R₂₂ is CF₃, CHF₂CH₂, HOC(O)—CH₂—, H₃C—C(O)—, (H₃C)₃C—O—C(O)—;
R₂₃ is CF₃, CHF₂CH₂—, (H₃C)₃C—O—C(O)—;
R₂₄ is CH₃; and
R₂₅ is CHF₂CH₂—.

In particular, R₁ is selected from:

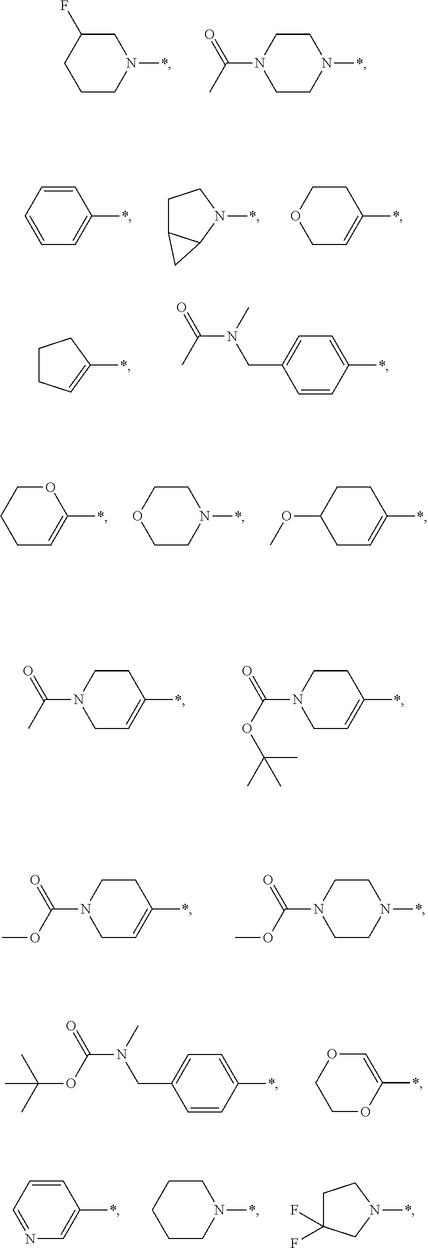

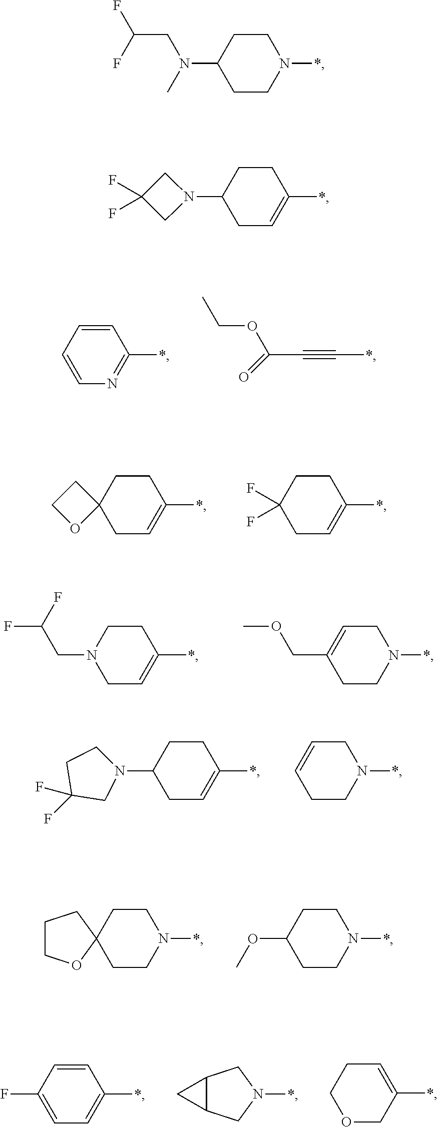

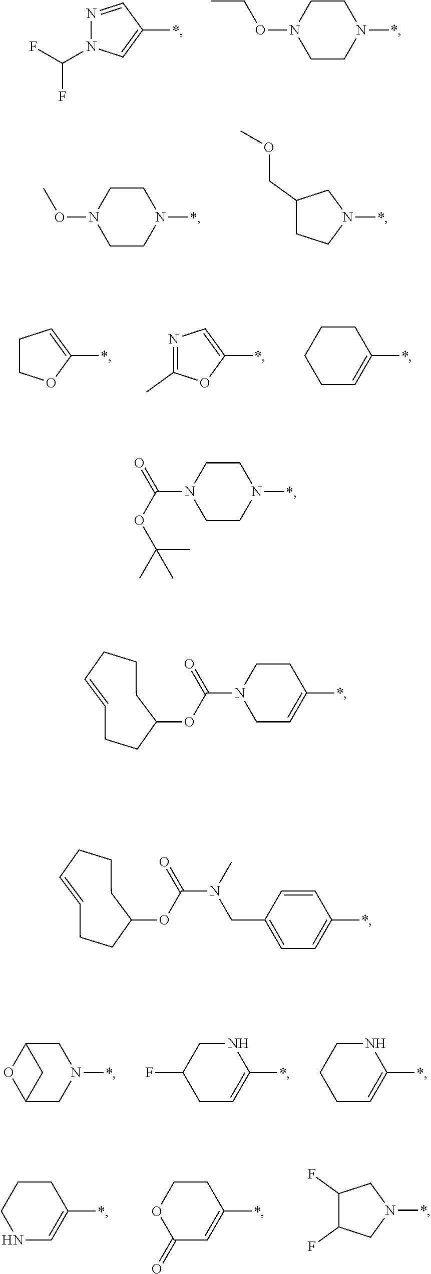

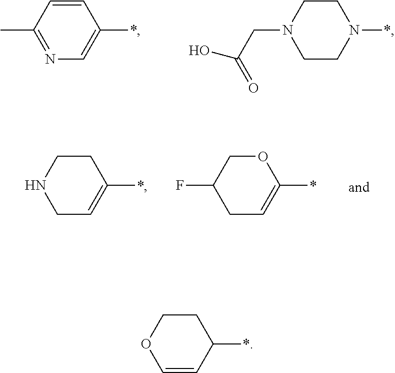

More preferably, R₁ is selected from:

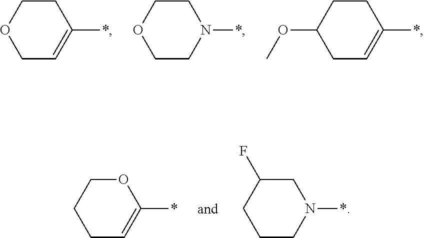

especially

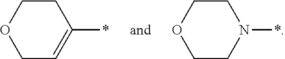

More preferably

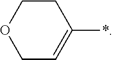

In particular R₂ is the moiety:

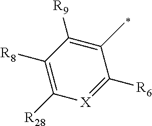

wherein
R₆ is selected from H, halo, (C₁-C₄)alkyl unsubstituted or substituted by 1, 2 or 3 halo;
R₈ is selected from H, halo, (C₁-C₄)alkyl unsubstituted or substituted by 1, 2 or 3 halo;
R₉ is selected from H, O—CH₃, OH, CN, CH₃ and halo;
R₂₈ is selected from SF₅, halo, (C₁-C₄)alkyl unsubstituted or substituted by 1, 2 or 3 halo, and —C(O)H;
X is selected from C—R₇ and N; and
R₇ is selected from H and halo.

More particularly, R₂ is the moiety:

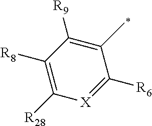

R₆ is selected from H, C, CH₃, F and Br;
R₈ is selected from H, C, F and CF₃;
R₉ is selected from H, CH₃ and Cl;
R₂₈ is selected from CF₃, CF₂H, —CH₂CH₃, Cl, SF₅, Br and —C(O)H;
X is selected from C—R₇ and N; and
R₇ is selected from H and F.

In particular, R₂₈ is selected from CF₃, CHF₂, Cl, —CH₂CH₃, OH₃, SF₅ and Br. More particularly, R₂₈ is selected from CF₃, Cl and SF₅, in particular CF₃.

In particular, X is CR₇. More particularly, R₇ is H.

In particular, R₆ is H, F, Cl or OH₃. More particularly, R₆ is Cl.

In particular, R₈ is F, CF₃ or H. More particularly, R₈ is H.

In particular, R₉ is H.

In particular, R₂ is selected from

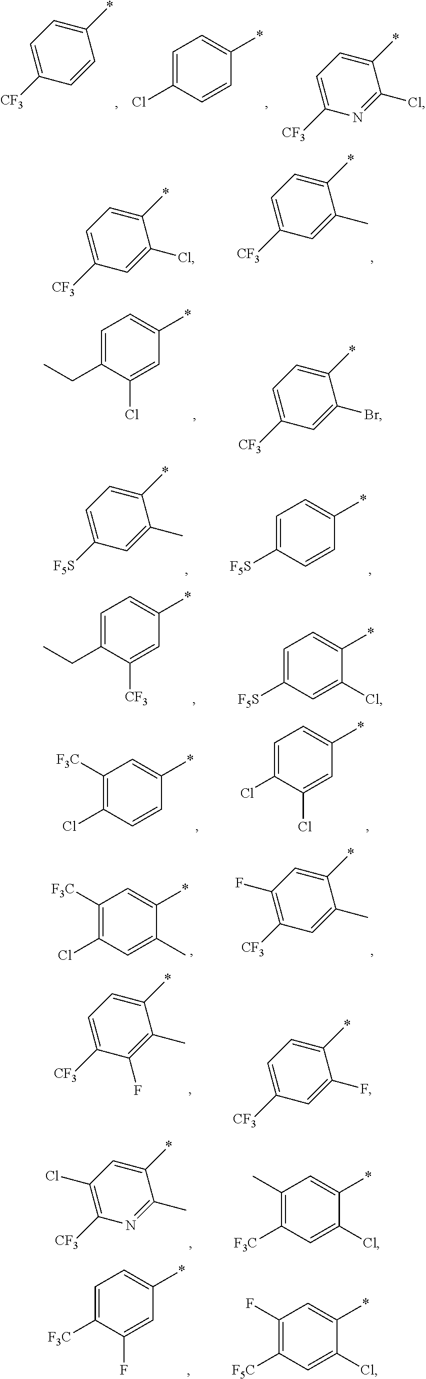

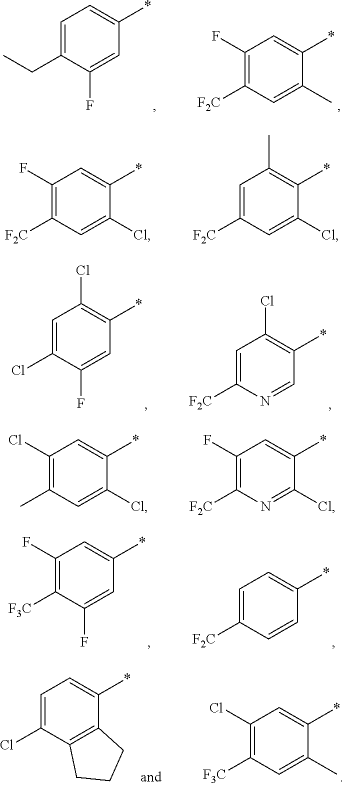

In particular, R₃ is (C₁-C₄)alkyl unsubstituted or substituted by 1, 2 or 3 substituents independently selected from halo and OH, or R₃ is selected from —CH₃, —CH₂CH₃, —CH(CH₃)₂, and cyclopropyl. More particularly, R₃ is (C₁-C₂)alkyl unsubstituted or substituted by 1, 2 or 3 substituents independently selected from halo and OH, preferably —CH₂CH₃ or CH₃, more preferably —CH₂CH₃.

In particular, R₂₆ is H.

In particular, R₂₇ is H.

In particular, R₄ is selected from:

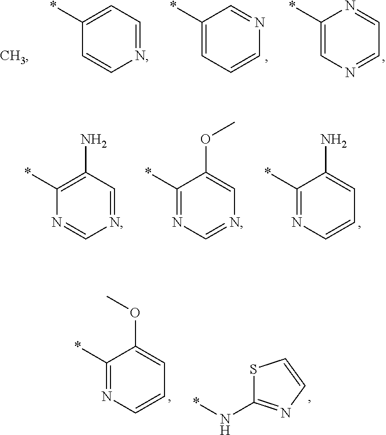

-heteroaryl1, wherein said heteroaryl1 is a 5 membered, fully unsaturated, monocyclic ring comprising ring carbon atoms and 1, 2, 3 or 4 ring heteroatoms independently selected from N, O and S;
-heteroaryl2, wherein said heteroaryl2 is a 9 or 10 membered fused bicyclic ring comprising ring carbon atoms and 1, 2, 3 or 4 ring heteroatoms independently selected from N, O and S, and wherein both rings are fully unsaturated, or one ring is fully unsaturated, and the other is saturated or partially unsaturated, and wherein the heteroatoms may be in one or both rings;
-phenyl; or
-heterocyclyl2, wherein said heterocyclyl2 is a 5 or 6 membered fully saturated or partially unsaturated group comprising ring carbon atoms and 1 or 2 ring heteroatoms independently selected from N, O and S,
wherein heteroaryl1, heteroaryl2, phenyl, and the moiety selected from:

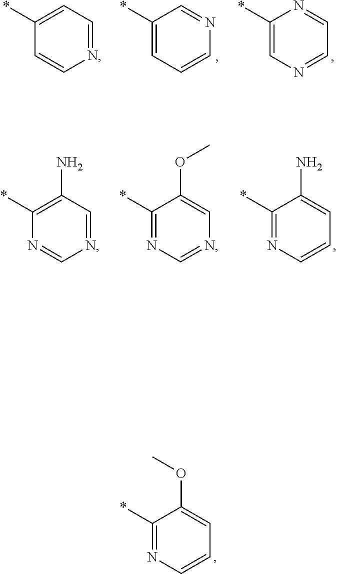

are each substituted by 1, 2 or 3 substituents, in particular 1 or 2 substituents, independently selected from R₁₀, R₁₁, R₁₂, R₁₃ and R₁₄, wherein each R₁₀, R₁₁, R₁₂, R₁₃ and R₁₄ is independently selected from:
   H,
   halo,
   (C₁-C₄)alkyl unsubstituted or substituted by 1, 2 or 3 halo substituents,
   (C₁-C₂)alkyl substituted by —O—(C₁-C₂)alkyl or OH,
   —S—(C₁-C₃)alkyl,
   —O—(C₁-C₄)alkyl unsubstituted or substituted by 1, 2 or 3 halo substituents,
   OH,
   (C₃-C₅)cycloalkyl, wherein said (C₃-C₅)cycloalkyl is unsubstituted or substituted by 1 or 2 halo,
   —O—(C₃-C₅)cycloalkyl,
   —NR₃₄R₃₅ wherein R₃₄ and R₃₅ are independently selected from:
      H,
      (C₁-C₄)alkyl, wherein said (C₁-C₄)alkyl is unsubstituted or substituted by OH or —O(C₁-C₂)alkyl,
      and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
   CN,
   —(C₂-C₄)alkenyl,
   —(C₂-C₄)alkynyl,
   ═O
   —C(O)H, and
   —C(O)(C₁-C₄)alkyl;

More particularly, R₄ is as described in embodiment 1 and other embodiments herein, with the proviso that at least one OH, CN, ═O, or NH₂ substituent is present on each heteroaryl1, heteroaryl2, phenyl,

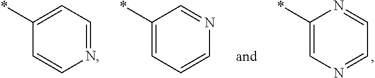

and the remaining R₁₀, R₁₁, R₁₂, R₁₃ and R₁₄ are as defined herein.

Even more particularly, R₄ is as described in embodiment 1 and other embodiments herein, with the proviso that one OH substituent is present on each heteroaryl1, heteroaryl2, phenyl,

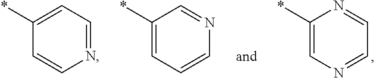

and the remaining R₁₀, R₁₁, R₁₂, R₁₃ and R₁₄ are as defined herein.

In another embodiment, R₄ is as described herein, with the proviso that one OH substituent is present on each heteroaryl1, heteroaryl2, phenyl,

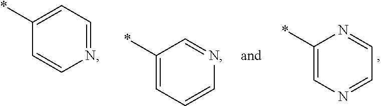

and said OH substituent is in the ortho position of the R₄ ring, relative to the position linking R₄ to linker —C(O)—, and the remaining R₁₀, R₁₁, R₁₂, R₁₃ and R₁₄ are as defined herein.

More particularly, R₄ is as described herein, and each R₁₀, R₁₁, R₁₂, R₁₃ and R₁₄ is independently selected from:
H,
halo (preferably F),
(C₁-C₂)alkyl (preferably CH₃), said (C₁-C₂)alkyl being unsubstituted or substituted by 1, 2 or 3 halo,
═O,
CN,
NH₂, and
—O—(C₁-C₂)alkyl unsubstituted or substituted by 1, 2 or 3 halo.

More particularly, R₄ is selected from:

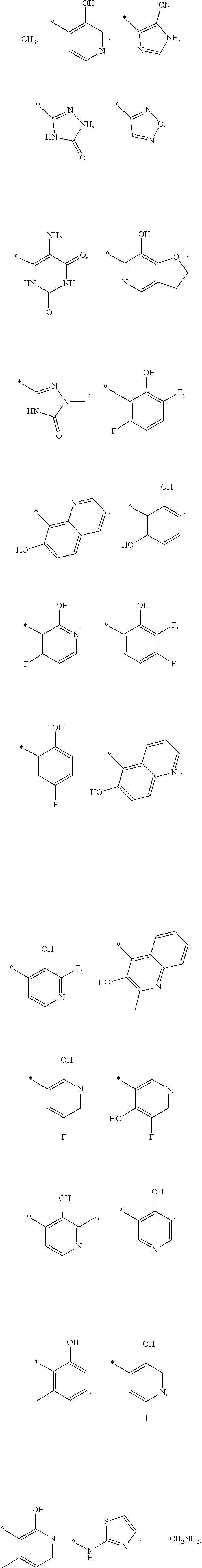

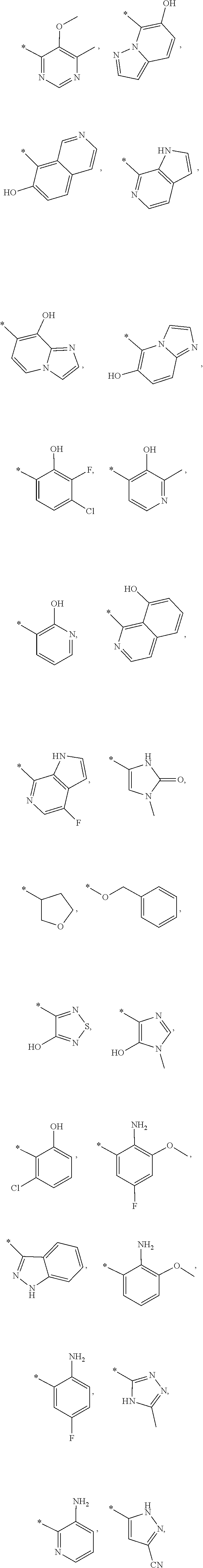

—NH—NH—C(O)—CF₃, and

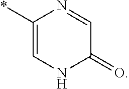 [SMILES: *c1c[nH]c(=O)cn1]

In particular, Y is N and Y is Y [symbol] linked by a single bond.
   In particular, K [symbol] is K linked by a single bond, and K is selected from —CH₂—, —CH₂CH₂—,
—NH— and a bond (to form a 5-membered ring:

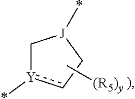

and J is N. More particularly, K [symbol] is K linked by a single bond, K is —CH₂— and J is N.

In particular, R₅ is independently selected from:
—(C₁-C₄)alkyl, preferably methyl,
and wherein two R₅ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S,
when K [symbol] J is a carbon-nitrogen single bond, a R₅ substituent on K and on the adjacent carbon atom may join to form ring C:

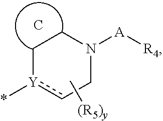

wherein ring C is a fused (C₃-C₆)cycloalkyl ring, in particular a fused cyclobutyl ring, a fused (C₃-C₆)heterocyclyl ring or a fused phenyl ring, wherein said fused (C₃-C₆)heterocyclyl ring contains ring carbon atoms and one ring heteroatom selected from O, N and S,
   and wherein when ring C is a fused (C₃-C₆)cycloalkyl ring, in particular fused cyclobutyl ring, said fused (C₃-C₆)cycloalkyl ring is unsubstituted or substituted with 1 or 2 R₄₀ groups, wherein said R₄₀ is selected from:
      (C₁-C₂)alkyl, wherein each (C₁-C₂)alkyl is independently unsubstituted or substituted by OH or 1, 2 or 3 halo,
      halo, in particular F,
      or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
      or wherein two R₄₀ substituents on adjacent carbon atoms join together with the carbon atoms to which they are attached, to form a fused cyclopropyl ring;
and wherein when K is —CH₂— and J is N, two R₅ substituents may join to form a (C₁-C₃)alkylene bridge or a heteroalkylene bridge, wherein said heteroalkylene bridge is one heteroatom selected from N and O, or is —CH₂—O—CH₂—.

More particularly, R₅ is independently selected from:
—(C₁-C₄)alkyl, preferably methyl,
when K [symbol] J is a carbon-nitrogen single bond, a R₅ substituent on K and on the adjacent carbon atom may join to form ring C:

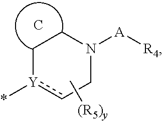

wherein ring C is a fused (C₃-C₆)cycloalkyl ring, in particular a fused cyclobutyl ring, or a fused (C₃-C₆)heterocyclyl ring, wherein said fused (C₃-C₆)heterocyclyl ring contains ring carbon atoms and one ring heteroatom selected from O, N and S,
   and wherein when ring C is a fused (C₃-C₆)cycloalkyl ring, in particular fused cyclobutyl ring, said fused (C₃-C₆)cycloalkyl ring is unsubstituted or substituted with 1 or 2 R₄₀ groups, wherein said R₄₀ is selected from:
      (C₁-C₂)alkyl, wherein each (C₁-C₂)alkyl is independently unsubstituted or substituted by OH or 1, 2 or 3 halo,
      halo, in particular F,
      or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
      or wherein two R₄₀ substituents on adjacent carbon atoms join together with the carbon atoms to which they are attached, to form a fused cyclopropyl ring;
and wherein when K is —CH₂— and J is N, two R₅ substituents may join to form a (C₁-C₃)alkylene bridge or a heteroalkylene bridge, wherein said heteroalkylene bridge is one heteroatom selected from N and O, or is —CH₂—O—CH₂—.

Even more particularly, R₅ is independently selected from:
—(C₁-C₂)alkyl, preferably methyl, and
when K [symbol] J is a carbon-nitrogen single bond, a R₅ substituent on K and on the adjacent carbon atom may join to form ring C:

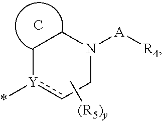

wherein ring C is a fused (C₃-C₄)cycloalkyl ring, in particular a fused cyclobutyl ring, and said fused (C₃-C₄)cycloalkyl ring, in particular fused cyclobutyl ring, is unsubstituted or substituted with 1 or 2 R₄₀ groups as described in the embodiments herein.

In particular, y is 0, 1, 2 or 3, preferably 0, 1, or 2.

More particularly, R₅ is independently selected from:
CH₃, and y is 1 or 2, and
when K [symbol] J is a carbon-nitrogen single bond, a R₅ substituent on K and on the adjacent carbon atom may join to form ring C:

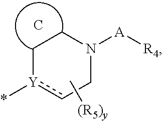

wherein ring C is a fused cyclobutyl ring.

In particular, the compound of formula (I) includes the moiety:

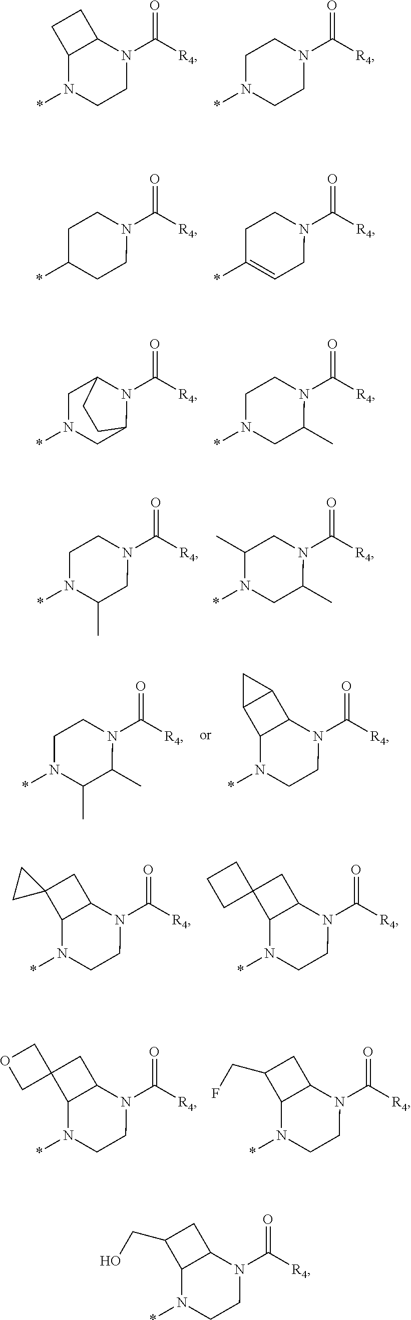

in particular
A:

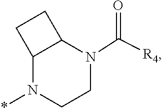

more particularly

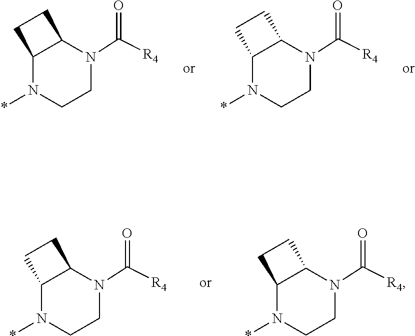

or B:

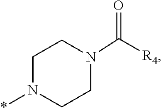

or C:

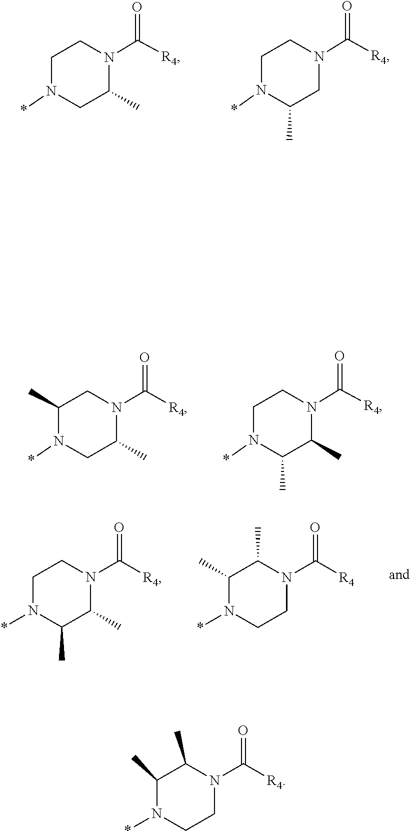

More particularly, the compound of formula (I) includes the moiety:

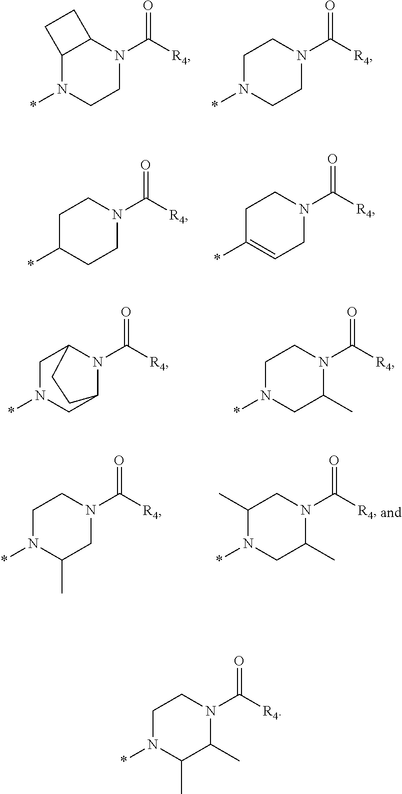

### Forms

Depending on the choice of the starting materials and procedures, the compounds can be present in the form of one of the possible stereoisomers or as mixtures thereof, for example as pure optical isomers, or as stereoisomer mixtures, such as racemates and diastereoisomer mixtures, depending on the number of asymmetric carbon atoms. The present invention is meant to include all such possible stereoisomers, including racemic mixtures, diasteriomeric mixtures and optically pure forms. Optically active (R)- and (S)-stereoisomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the terms “salt” or “salts” refers to an acid addition or base addition salt of a compound of the present invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In another aspect, the present invention provides compounds of the present invention in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form.

Any formula given herein is intended to represent unlabeled forms as well as isotopically labeled forms of the compounds, in addition to the deuteration specifically claimed in formula (I). Isotopically labeled compounds have structures depicted by the formulae given herein except that one or more atoms are replaced by an atom having a selected atomic mass or mass number. Isotopes that can be incorporated into compounds of the invention include, for example, isotopes of hydrogen. For example, the invention includes deuterated forms of the exemplified compounds disclosed herein. In compounds of Formula (I), for example, one or more H atoms on the ring:

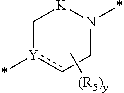

may be replaced by deuterium, and for example one or more atoms on the R₁ moiety may be replaced by deuterium:

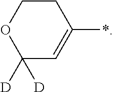

Further, incorporation of certain isotopes, particularly deuterium (i.e., ²H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of a compound of the present invention. The concentration of deuterium, may be defined by the isotopic enrichment factor. The term “isotopic enrichment factor” as used herein means the ratio between the isotopic abundance and the natural abundance of a specified isotope. If a substituent in a compound of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into compounds of the invention include isotopes of hydrogen, carbon, nitrogen, oxygen, phosphorous, fluorine, and chlorine, such as ³H, ¹¹C ¹³C, ¹⁴C ¹⁵N, ¹⁸F ³¹P, ³²P, ³⁵S, ³⁶Cl, ¹²³I, ¹²⁴I ¹²⁵I respectively. Accordingly it should be understood that the invention includes compounds that incorporate one or more of any of the aforementioned isotopes, including for example, radioactive isotopes, such as ³H and ¹⁴C or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of the present invention can generally be prepared by conventional techniques known to those skilled in the art or by processes analogous to those described in the accompanying Examples and Preparations using an appropriate isotopically-labeled reagents in place of the non-labeled reagent previously employed.

### Definitions

A ‘compound of the present invention’ or a ‘compound of formula (I)’ or a ‘compound of formula 1a’ etc., includes a zwitterion thereof, a non-zwitterion thereof (non-charged form), or a pharmaceutically acceptable salt of said zwitterionic or non-zwitterionic form thereof. ‘zwitterion’ or ‘zwitterionic form’ means a compound containing both positive and negatively charged functional groups.

halo means fluoro, chloro or bromo, particularly fluoro or chloro, unless otherwise stated.

Alkyl, and alkoxy groups, containing the requisite number of carbon atoms, can be unbranched or branched. Examples of alkyl include, but are not limited to, methyl, ethyl, n-propyl, i-propyl, n-butyl, i-butyl, sec-butyl and t-butyl. Examples of alkoxy include methoxy, ethoxy, n-propoxy, i-propoxy, n-butoxy, i-butoxy, sec-butoxy and t-butoxy.

‘═O’ means an oxo substituent.

When R₁ is substituted or unsubstituted cycloalkenyl, said cycloalkenyl includes, but is not limited to, groups such as cyclohexenyl, in particular cyclohex-1-en-1-yl.

When R₁ is substituted or unsubstituted heterocyclyl, said heterocyclyl includes, but is not limited to, groups such as morpholinyl, piperidinyl, pyrrolidinyl, 6-oxa-3-azabicyclo[3.1.1]heptan-3-yl, 5,6-dihydro-1,4-dioxin-2-yl, dihydropyranyl, in particular 3,4-dihydro-2H-pyran-6-yl, 5,6-dihydro-2H-pyran-3-yl and 3,6-dihydro-2H-pyran-4-yl, piperazinyl, tetrahydropyridinyl, such as 1,4,5,6-tetrahydropyridin-3-yl and 1,2,3,6-tetrahydropyridin-4-yl and dihydropyridinyl, such as 3,6-dihydropyridinyl.

When R₁ is heteroaryl, said heteroaryl is a 5 or 6 membered fully unsaturated (which includes aromatic), monocyclic group comprising ring carbon atoms and 1, 2, 3 or 4 ring heteroatoms independently selected from N, O and S, preferably 1 or 2 ring heteroatoms, preferably wherein the total number of ring S atoms does not exceed 1 and the total number of ring O atoms does not exceed 1. When R₁ is substituted or unsubstituted heteroaryl, said heteroaryl includes, but is not limited to, substituted or unsubstituted groups such as pyridinyl, in particular pyridin-3-yl.

-‘heteroaryl1’ is a 5 or 6 membered, fully unsaturated (which includes aromatic) monocyclic ring comprising ring carbon atoms and 1, 2, 3 or 4 ring heteroatoms independently selected from N, O and S. Preferably, the total number of ring S atoms does not exceed 1 and the total number of ring O atoms does not exceed 1. In particular, said heteraryl1 comprises ring carbon atoms and one or two nitrogen atoms only. Heteroaryl1 includes, but is not limited to, pyrrolyl, furanyl, thiophenyl, pyrazolyl, imidazolyl, isoxazolyl, oxadiazolyl, oxazolyl, isothiazolyl, thiazolyl, thiadiazolyl, tetrazolyl, pyridinyl, pyridazinyl, pyrimidinyl, triazolyl and pyrazinyl, in particular pyridyl, pyrimidinyl and triazolyl.

-‘heteroaryl2′, wherein said heteroaryl2 is a 9 or 10 membered fused bicyclic ring comprising ring carbon atoms and 1, 2, 3 or 4 ring heteroatoms independently selected from N, O and S, and wherein both rings are fully unsaturated (which includes aromatic), or one ring is fully unsaturated (which includes aromatic), and the other is saturated or partially unsaturated, and wherein the heteroatoms may be in one or both rings. Preferably, the total number of ring S atoms does not exceed 1 and the total number of ring O atoms does not exceed 1. In particular, the ring which is linked to the rest of the molecule via linker -A- is fully unsaturated. Heteroaryl2 includes, but is not limited to, benzofuranyl, benzothiophenyl, indolyl, benzimidazolyl, indazolyl, benzotriazolyl, pyrrolopyridinyl, imidazopyridinyl, pyrazololpyridinyl, isoindolyl, indazolyl, purinyl, indolininyl, imidazopyridinyl, pyrazolopyridinyl, pyrrolopyridazinyl, pyrrolopyridinyl, imidazopyrimidinyl, quinolinyl, isoquinolinyl, cinnolinyl, quinazolinyl, quinoxalinyl, phthalazinyl, naphthyridinyl, pyridopyrimidinyl, pyridopyrazinyl, pyrimidopyrimidinyl, pyrazinopyrazinyl, hydropyranopyridinyl, in particular hydrofuropyridinyl especially dihydrofuropyridinyl, and imidazopyridinyl.

The invention includes all tautomeric forms of the compounds of formula (I). For example, when heteroaryl1 and heteroaryl2 are substituted by ═O they may form tautomers, for example as follows:

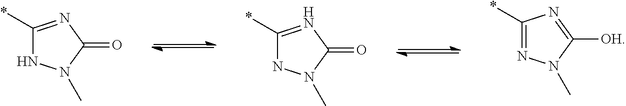

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to colorectal, gastric, endometrial, adrenocortical, uterine, cervical, esophageal, breast, kidney, ovarian cancer and the like.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors. ‘WRN inhibitor’ or ‘WRN helicase inhibitor’ as used herein means a compound that inhibits Werner Syndrome RecQ DNA helicase (WRN). The term “WRN” as used herein refers to the protein of Werner Syndrome RecQ DNA helicase. The term “WRN” includes mutants, fragments, variants, isoforms, and homologs of full-length wild-type WRN. In one embodiment, the protein is encoded by the WRN gene (Entrez gene ID 7486; Ensembl ID ENSG00000165392). Exemplary WRN sequences are available at the Uniprot database under accession number Q14191.

‘disease or condition mediated by WRN’ includes a disease or condition, such as cancer, which is treated by WRN inhibition. In particular this can include cancers characterized as microsatellite instability-high (MSI-H) or mismatch repair deficient (dMMR).

‘microsatellite unstable cancer’, microsatellite instability-high cancer’, ‘microsatellite high cancer’ and ‘MSI-high cancer’ ‘MSI^{hi}’ and ‘MSI-H’ when used herein, are used interchangeably, and describe cancers that have a high number of alterations in the length of simple repetitive genomic sequences within microsatellites.

The determination of MSI-H or dMMR tumor status for patients can be performed using, e.g., polymerase chain reaction (PCR) tests for MSI-H status or immunohistochemistry (IHC) tests for dMMR. Methods for identification of MSI-H or dMMR tumor status are described, e.g., in Ryan et al. Crit Rev Oncol Hematol. 2017; 116:38-57; Dietmaier and Hofstadter. Lab Invest 2001, 81:1453-1456; and Kawakami et al. Curr Treat Options Oncol. 2015; 16(7): 30).

Microsatellite instability can be found in colorectal cancer, gastric cancer and endometrial cancer in particular, but also in adrenocortical, uterine, cervical, esophageal, breast, kidney and ovarian cancers. Examples of microsatellite high cancers include uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma and ovarian serous cystadenocarcinoma.

A cancer that has “defective mismatch repair” (dMMR) or “dMMR character” includes cancer types associated with documented MLH1, PMS2, MSH2, MSH3, MSH6, MLH3, and PMS1 mutations or epigenetic silencing, microsatellite fragile sites, or other gene inactivation mechanisms, including but not limited to cancers of the lung, breast, kidney, large intestine, ovary, prostate, upper aerodigestive tract, stomach, endometrium, liver, pancreas, haematopoietic and lymphoid tissue, skin, thyroid, pleura, autonomic ganglia, central nervous system, soft tissue, pediatric rhabdoid sarcomas, melanomas and other cancers. A cell or cancer with “defective” mismatch repair has a significantly reduced (e.g., at least about 25%, 30%, 40%, 50%, 60%, 70%, 80% or 90% decrease) amount of mismatch repair. In some cases, a cell or cancer which is defective in mismatch repair will perform no mismatch repair.

As used herein, the terms “synthetic lethality,” and “synthetic lethal” are used to refer to reduced cell viability and/or a reduced rate of cell proliferation caused by a combination of mutations or approaches to cause loss of function (e.g., RNA interference or protein function inhibition) in two or more genes but not by the loss of function of only one of these genes.

he term “pharmaceutical composition” refers to a compound of the invention, or a pharmaceutically acceptable salt thereof, together with at least one pharmaceutically acceptable carrier, in a form suitable for oral or parenteral administration.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22nd Ed. Pharmaceutical Press, 2013, pp. 1049-1070).

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc.

In one embodiment, the term “a therapeutically effective amount” refers to the amount of the compound of the present invention that, when administered to a subject, is effective to (1) at least partially alleviate, prevent and/or ameliorate a condition, or a disorder or a disease (i) mediated by WRN, or (ii) associated with WRN activity, or (iii) characterized by activity (normal or abnormal) of WRN; or (2) reduce or inhibit the activity of WRN.

In another embodiment, the term “a therapeutically effective amount” refers to the amount of the compound of the present invention that, when administered to a cell, or a tissue, or a non-cellular biological material, or a medium, is effective to at least partially reducing or inhibiting the activity of WRN, or reducing WRN protein levels.

As used herein, the term “subject” refers to primates (e.g., humans, male or female), dogs, rabbits, guinea pigs, pigs, rats and mice. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing” or “prevention” of any disease or disorder refers to the prophylactic treatment of the disease or disorder; or delaying the onset or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

### Isomeric Forms

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible stereoisomers, rotamers, atropisomers, tautomers or mixtures thereof, for example, as substantially pure geometric (cis or trans) stereoisomers, diastereomers, optical isomers (antipodes), racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of compounds of the present invention or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′p-toluoyl tartaric acid, mandelic acid, malic acid or camphor-10-sulfonic acid. Racemic compounds of the present invention or racemic intermediates can also be resolved by chiral chromatography, e.g., high pressure liquid chromatography (HPLC) using a chiral adsorbent.

Compounds of the invention, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

### Formulations

In another aspect, the present invention provides a pharmaceutical composition comprising a compound of the present invention, or a pharmaceutically acceptable salt thereof, and a pharmaceutically acceptable carrier. In a further embodiment, the composition comprises at least two pharmaceutically acceptable carriers, such as those described herein. The pharmaceutical composition can be formulated for particular routes of administration such as oral administration, parenteral administration (e.g. by injection, infusion, transdermal or topical administration), and rectal administration, in particular oral administration. Topical administration may also pertain to inhalation or intranasal application. The pharmaceutical compositions of the present invention can be made up in a solid form (including, without limitation, capsules, tablets, pills, granules, powders or suppositories), or in a liquid form (including, without limitation, solutions, suspensions or emulsions). Tablets may be either film coated or enteric coated according to methods known in the art. Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and
e) absorbents, colorants, flavors and sweeteners.

Compounds intended for parenteral or oral administration can be solubilized using various methods including nano-suspensions, solid dispersions and liposomes (van Hoogevest P., Xiangli L., and Alfred F. “Drug delivery strategies for poorly water-soluble drugs: the industrial perspective” Expert Opinion on Drug Delivery 2011, 8(11), 1481-1500).

Solid dispersion technologies have been used to improve the dissolution characteristics and bioavailability of orally administered drugs (Dhirendra K et al: ‘Solid dispersions: A review”, Pakistan Journal of Pharmaceutical Sciences, Faculty of Pharmacy, University of Karachi, Pakistan, vol. 22, no.2. 30 April 200, pages 234-246).

Typical approaches to solubilize compounds for parenteral administration are the optimization of the pH or the use of co-solvents (e.g. PEG300, PEG400, propylene glycol, or ethanol). If these approaches are, for any reason, not feasible, the use of surfactants may be considered (e.g. Tween®80 or Cremophor EL®). Cyclodextrins are established as safe solubilizing agents. Compounds with a high solubility in natural oils (e.g. propofol) may be solubilized in parenteral fat emulsions.

There is also provided a pharmaceutical composition comprising a compound of formula (I) as described herein, or a pharmaceutically acceptable salt thereof, and one or more pharmaceutically acceptable carriers.

### Uses

The compounds of the present invention in free form or in pharmaceutically acceptable salt form, exhibit valuable pharmacological properties, e.g. WRN inhibiting properties, e.g. as indicated in vitro tests as provided herein, and are therefore indicated for therapy, or for use as research chemicals, such as laboratory research chemicals, for example as a chemical probe, or as tool compounds.

In another aspect of the invention there is provided a compound of formula (I), or a salt thereof, as described herein, which can be used as a research chemical, for example a tool compound or chemical probe, in particular for research on WRN or for example, MSI high cancers. In another embodiment there is provided the use of a compound of formula (I), or a salt thereof, as described herein, as a research chemical, for example tool compound or chemical probe, in particular for research on WRN or for example, MSI high cancers.

There is also provided a compound of formula (I) as described herein, or a pharmaceutically acceptable salt thereof, for use in the treatment of cancer. Cancers that may be treated by WRN inhibition include cancers that are characterized as microsatellite instability-high (MSI-H) or mismatch repair deficient (dMMR). In particular, a compound of formula (I) as described herein, or a pharmaceutically acceptable salt thereof, may be useful in the treatment of a cancer that is characterized as microsatellite instability-high (MSI-H) or mismatch repair deficient (dMMR).

There is also provided a compound of formula (I) as described herein, or a pharmaceutically acceptable salt thereof, for use as a medicament. In particular, said use is:
for the treatment of a disease that is treated by WRN inhibition,
for the treatment of cancer,
for the treatment of cancer that is characterized as microsatellite instability-high (MSI-H) or mismatch repair deficient (dMMR),
for the treatment of cancer that is characterized as microsatellite instability-high (MSI-H) or mismatch repair deficient (dMMR), such as colorectal, gastric, prostate, endometrial, adrenocortical, uterine, cervical, esophageal, breast, kidney and ovarian cancer,
for the treatment of cancer that is characterized as microsatellite instability-high (MSI-H) or mismatch repair deficient (dMMR) is selected from colorectal, gastric, prostate and endometrial cancer, or
for the treatment of cancer wherein the cancer characterized as microsatellite instability-high (MSI-H) or mismatch repair deficient (dMMR) is selected from uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma, prostate cancer and ovarian serous cystadenocarcinoma.

There is also provided a method of:
modulating WRN activity in a subject, wherein the method comprises administering to the subject a therapeutically effective amount of the compound of formula (I) as described herein, or a pharmaceutically acceptable salt thereof,
inhibiting WRN in a subject, wherein the method comprises administering to the subject a therapeutically effective amount of the compound of formula (I) as described herein, or a pharmaceutically acceptable salt thereof,
treating a disorder or disease which can be treated by WRN inhibition in a subject, comprising administering to the subject a therapeutically effective amount of the compound of formula (I) as described herein, or a pharmaceutically acceptable salt thereof,
treating cancer in a subject, comprising administering to the subject a therapeutically effective amount of the compound of formula (I) as described herein, or a pharmaceutically acceptable salt thereof,
treating cancer in a subject, comprising administering a compound of formula (I) as described herein, or a pharmaceutically acceptable salt thereof, wherein the cancer is characterized as microsatellite instability-high (MSI-H) or mismatch repair deficient (dMMR). In particular, the cancer characterized as microsatellite instability-high (MSI-H) or mismatch repair deficient (dMMR) is selected from colorectal, gastric, prostate, endometrial, adrenocortical, uterine, cervical, esophageal, breast, kidney and ovarian cancer. More particularly, the cancer characterized as microsatellite instability-high (MSI-H) or mismatch repair deficient (dMMR) is selected from colorectal, gastric, prostate and endometrial cancer. Examples include uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma, prostate cancer and ovarian serous cystadenocarcinoma.

There is also provided the use of a compound of formula (I) as described herein, or a pharmaceutically acceptable salt thereof:
in therapy,
in the manufacture of a medicament,
in the manufacture of a medicament for the treatment of cancer. In particular, said cancer is characterized as microsatellite instability-high (MSI-H) or mismatch repair deficient (dMMR),
in the manufacture of a medicament for treatment of a disease which may be treated by WRN inhibition, wherein in particular, the cancer is characterized by microsatellite instability-high (MSI-H) or mismatch repair deficient (dMMR), for example colorectal, gastric, prostate, endometrial, adrenocortical, uterine, cervical, esophageal, breast, kidney and ovarian cancer, in particular, colorectal, gastric, prostate or endometrial cancer, or uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma and ovarian serous cystadenocarcinoma.

In some embodiments, the subject has or is identified as having a microsatellite instable (MSI-H) cancer, e.g., in reference to a control, e.g., a normal, subject. In one embodiment, the subject has MSI-H advanced solid tumors, a colorectal cancer (CRC), endometrial, uterine, stomach or other MSI-H cancer. In some embodiments, the subject has a colorectal (CRC), endometrial or stomach cancer, which cancer has or is identified as having a microsatellite instability (MSI-H), e.g., in reference to a control, e.g., a normal, subject. Such identification techniques are known in the art.

### Dosage Forms

The pharmaceutical composition or combination of the present invention may, for example, be in unit dosage of about 1-1000 mg of active ingredient(s) for a subject of about 50-70 kg.

### Combinations

“Combination” refers to either a fixed combination in one dosage unit form, or a combined administration where a compound of formula (I), or a pharmaceutically acceptable salt thereof, and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. a compound of the present invention and a combination partner, are both administered to a patient simultaneously in the form of a single entity or dosage. The term “non-fixed combination” means that the therapeutic agents, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agents.

The combinations described herein can include a compound of formula (I) and one or more additional therapeutic agents, e.g., one or more anti-cancer agents, cytotoxic or cytostatic agents, hormone treatment, vaccines, and/or other immunotherapies. In other embodiments, the combination is further administered or used in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the treatment.

The additional therapeutic agent is, for example, a chemical compound, peptide, antibody, antibody fragment or nucleic acid, which is therapeutically active or enhances the therapeutic activity when administered to a patient in combination with a compound of the present disclosure.

In one embodiment, the additional therapeutically active agent is a chemotherapy. General Chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

In another embodiment, the additional therapeutically active agent is an anti-cancer agent.

Combination partners of particular interest for combinations with the compounds of the present invention include fluorouracil (5-FU) and irinotecan (Camptosar®).

In a further embodiment, the additional therapeutically active agent is the chemotherapy irinotecan (Camptosar®).

In another embodiment, the additional therapeutically active agent is an inhibitor of PD-1, e.g., human PD-1. In another embodiment, the immunomodulator is an inhibitor of PD-L1, e.g., human PD-L1. In one embodiment, the inhibitor of PD-1 or PD-L1 is an antibody molecule to PD-1 or PD-L1. In another embodiment, the additional therapeutically active agent is an anti-PD-1 antibody molecule.

In another embodiment, the PD-1 inhibitor is selected from PDR001 (Novartis), Nivolumab (Bristol-Myers Squibb), Pembrolizumab (Merck & Co), Pidilizumab (CureTech), MEDI0680 (Medimmune), Cemiplimab (REGN2810, Regeneron), Dostarlimab (TSR-042, Tesaro), PF-06801591 (Pfizer), Tislelizumab (BGB-A317, Beigene), BGB-108 (Beigene), INCSHR1210 (Incyte), Balstilimab (AGEN2035, Agenus), Sintilimab (InnoVent), Toripalimab (Shanghai Junshi Bioscience), Camrelizumab (Jiangsu Hengrui Medicine Co.), and AMP-224 (Amplimmune), in particular PDR001 or tislelizumab.

In a further embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety.

In another embodiment, there is provided a combination of a compound of formula (I) or a pharmaceutically acceptable salt thereof, and a chemotherapy, and a PD-1 inhibitor.

In particular, the chemotherapy and PD-1 inhibitor are selected from those described above. More particularly, the chemotherapy is irinotecan (Camptosar®) and the PD-1 inhibitor is PHR001 or Tislelizumab. Tislelizumab can have a heavy chain of SEQ ID NO: 3 and a light chain of SEQ ID NO: 4. In some embodiments, the ani-PD-1 antibody is dosed at 100 mg per week. In some embodiments, tislelizumab and is dosed at 300 mg IV on day 1 of each 28 day cycle. In some embodiments, tislelizumab can be dosed at 500 mg once every four (4) weeks.

In another embodiment, the anti-PD-1 antibody molecule, e.g., tislelizumab, and comprises a heavy chain and/or light chain, VH, VL, HCDR1, HCDR2, HCDR3, LCDR1, LCDR2, and LCDR3 of the following:

| | AMINO ACID SEQUENCE | |
|---|---|---|
| Heavy | QVQLQESGPGLVKPSETLSLTCTVSGFSLTSYGVHWI | SEQ ID NO: 3 |
| Chain | RQPPGKGLEWIGVIYADGSTNYNPSLKSRVTISKDTSK | |
| | NQVSLKLSSVTAADTAVYYCARAYGNYWYIDVWGQG | |
| | TTVTVSSASTKGPSVFPLAPCSRSTSESTAALGCLVKD | |
| | YFPEPVTVSWNSGALTSGVHTFPAVLQSSGLYSLSSV | |
| | VTVPSSSLGTKTYTCNVDHKPSNTKVDKRVESKYGPP | |
| | CPPCPAPPVAGGPSVFLFPPKPKDTLMISRTPEVTCVV | |
| | VAVSQEDPEVQFNWYVDGVEVHNAKTKPREEQFNST | |
| | YRVVSVLTVVHQDWLNGKEYKCKVSNKGLPSSIEKTIS | |
| | KAKGQPREPQVYTLPPSQEEMTKNQVSLTCLVKGFYP | |
| | SDIAVEWESNGQPENNYKTTPPVLDSDGSFFLYSKLT | |
| | VDKSRWQEGNVFSCSVMHEALHNHYTQKSLSLSLGK | |
| Light | DIVMTQSPDSLAVSLGERATINCKSSESVSNDVAWYQ | SEQ ID NO: 4 |
| Chain | QKPGQPPKLLINYAFHRFTGVPDRFSGSGYGTDFTLTI | |
| | SSLQAEDVAVYYCHQAYSSPYTFGQGTKLEIKRTVAA | |
| | PSVFIFPPSDEQLKSGTASVVCLLNNFYPREAKVQWK | |
| | VDNALQSGNSQESVTEQDSKDSTYSLSSTLTLSKADY | |
| | EKHKVYACEVTHQGLSSPVTKSFNRGEC | |
| Variable | QVQLQESGPGLVKPSETLSLTCTVSGFSLTSYGVHWI | SEQ ID NO: 5 |
| Heavy | RQPPGKGLEWIGVIYADGSTNYNPSLKSRVTISKDTSK | |
| Chain | NQVSLKLSSVTAADTAVYYCARAYGNYWYIDVWGQG | |
| | TTVTVSS | |
| Variable | DIVMTQSPDSLAVSLGERATINCKSSESVSNDVAWYQ | SEQ ID NO: 6 |
| Light | QKPGQPPKLLINYAFHRFTGVPDRFSGSGYGTDFTLTI | |
| Chain | SSLQAEDVAVYYCHQAYSSPYTFGQGTKLEIK | |
| HCDR1 | GFSLTSYGVH | SEQ ID NO: 7 |
| HCDR2 | VIYADGSTNYNPSLKS | SEQ ID NO: 8 |
| HCDR3 | ARAYGNYWYIDV | SEQ ID NO: 9 |
| LCDR1 | KSSESVSNDVA | SEQ ID NO: 10 |
| LCDR2 | YAFHRFT | SEQ ID NO: 11 |
| LCDR3 | HQAYSSPYT | SEQ ID NO: 12 |

In some embodiments, the PD-1 inhibitor comprises the HCDRs and LCDRs of tislelizumab as set forth in SEQ ID NOs: 7-12.

In some embodiments, the PD-1 inhibitor (e.g., tislelizumab) is administered at a flat dose of between about 100 mg to about 600 mg. In some embodiments, the PD-1 inhibitor is administered at a dose of between about 100 mg to about 500 mg. In some embodiments, the PD-1 inhibitor is administered at a dose of between about 100 mg to about 400 mg. In some embodiments, the PD-1 inhibitor is administered at a dose of between about 100 mg to about 300 mg. In some embodiments, the PD-1 inhibitor is administered at a dose of between about 100 mg to about 200 mg. In some embodiments, the PD-1 inhibitor is administered at a dose of between about 200 mg to about 600 mg. In some embodiments, the PD-1 inhibitor is administered at a dose of between about 200 mg to about 500 mg. In some embodiments, the PD-1 inhibitor is administered at a dose of between about 200 mg to about 400 mg. In some embodiments, the PD-1 inhibitor is administered at a dose of between about 200 mg to about 300 mg. In some embodiments, the PD-1 inhibitor is administered at a dose of between about 300 mg to about 600 mg. In some embodiments, the PD-1 inhibitor is administered at a dose of between about 300 mg to about 500 mg. In some embodiments, the PD-1 inhibitor is administered at a dose of between about 300 mg to about 400 mg. In some embodiments, the PD-1 inhibitor is administered at a dose of between about 400 mg to about 600 mg. In some embodiments, the PD-1 inhibitor is administered at a dose of between about 400 mg to about 500 mg. In some embodiments, the PD-1 inhibitor is administered at a dose of between about 500 mg to about 600 mg. In some embodiments, the PD-1 inhibitor is administered at a dose of between about 600 mg to about 700 mg. In some embodiments, the PD-1 inhibitor is administered at a dose of between about 700 mg to about 800 mg. In some embodiments, the PD-1 inhibitor is administered at a dose of between about 800 mg to about 900 mg. In some embodiments, the PD-1 inhibitor is administered at a dose of between about 900 mg to about 1000 mg.

In some embodiments, the PD-1 inhibitor (e.g., tislelizumab) is administered at a flat dose of about 100 mg. In some embodiments, the PD-1 inhibitor is administered at a dose of about 200 mg. In some embodiments, the PD-1 inhibitor is administered at a dose of about 300 mg. In some embodiments, the PD-1 inhibitor is administered at a dose of about 400 mg. In some embodiments, the PD-1 inhibitor is administered at a dose of about 500 mg.

In some embodiments, the PD-1 inhibitor is administered at a dose of about 600 mg. In some embodiments, the PD-1 inhibitor is administered at a dose of about 700 mg. In some embodiments, the PD-1 inhibitor is administered at a dose of about 800 mg. In some embodiments, the PD-1 inhibitor is administered at a dose of about 900 mg. In some embodiments, the PD-1 inhibitor is administered at a dose of about 1000 mg.

In some embodiments, the PD-1 inhibitor (e.g., tislelizumab) is administered once every ten weeks. In some embodiments, the PD-1 inhibitor is administered once every nine weeks.

In some embodiments, the PD-1 inhibitor is administered once every eight weeks. In some embodiments, the PD-1 inhibitor is administered once every seven weeks. In some embodiments, the PD-1 inhibitor is administered once every six weeks. In some embodiments, the PD-1 inhibitor is administered once every five weeks. In some embodiments, the PD-1 inhibitor is administered once every four weeks. In some embodiments, the PD-1 inhibitor is administered once every three weeks. In some embodiments, the PD-1 inhibitor is administered once every two weeks. In some embodiments, the PD-1 inhibitor is administered once every week.

In some embodiments, the PD-1 inhibitor (e.g., tislelizumab) is administered intravenously.

In some embodiments, the PD-1 inhibitor (e.g., tislelizumab) is administered over a period of about 20 minutes to 40 minutes (e.g., about 30 minutes). In some embodiments, the PD-1 inhibitor is administered over a period of about 30 minutes. In some embodiments, the PD-1 inhibitor is administered over a period of about an hour. In some embodiments, the PD-1 inhibitor is administered over a period of about two hours. In some embodiments, the PD-1 inhibitor is administered over a period of about three hours. In some embodiments, the PD-1 inhibitor is administered over a period of about four hours. In some embodiments, the PD-1 inhibitor is administered over a period of about five hours. In some embodiments, the PD-1 inhibitor is administered over a period of about six hours.

In some embodiments, the PD-1 inhibitor (e.g., tislelizumab) is administered at a dose between about 300 mg to about 500 mg (e.g., about 400 mg), intravenously, once every four weeks. In some embodiments, the PD-1 inhibitor is administered at a dose between about 200 mg to about 400 mg (e.g., about 300 mg), intravenously, once every three weeks. In some embodiments, tislelizumab is administered at a dose of 400 mg, once every four weeks. In some embodiments, tislelizumab is administered at a dose of 300 mg, once every three weeks.

In some embodiments, the PD-1 inhibitor (e.g., tislelizumab) is administered at a dose between about 300 mg to about 500 mg (e.g., about 400 mg), intravenously, over a period of about 20 minutes to about 40 minutes (e.g., about 30 minutes), once every two weeks. In some embodiments, the PD-1 inhibitor is administered at a dose between about 200 mg to about 400 mg (e.g., about 300 mg), intravenously, over a period of about 20 minutes to about 40 minutes (e.g., about 30 minutes), once every three weeks.

In some embodiments, the PD-1 inhibitor (e.g., tislelizumab) is administered at a dose of about 100 mg per week. For example, if a 10-week dose is given to a patient, then the PD-1 inhibitor (e.g., tislelizumab) can be given at 1000 mg. If a 9-week dose is given, then the PD-1 inhibitor (e.g., tislelizumab) can be given at 900 mg. If an 8-week dose is given, then the PD-1 inhibitor (e.g., tislelizumab) can be given at 800 mg. If a 7-week dose is given, then the PD-1 inhibitor (e.g., tislelizumab) can be given at 700 mg. If a 6-week dose is given, then the PD-1 inhibitor (e.g., tislelizumab) can be given at 600 mg. If a 5-week dose is given, then the PD-1 inhibitor (e.g., tislelizumab) can be given at 500 mg. If a 4-week dose is given, then the PD-1 inhibitor (e.g., tislelizumab) can be given at 400 mg. If a 3-week dose is given, then the PD-1 inhibitor (e.g., tislelizumab) can be given at 300 mg. If a 2-week dose is given, then the PD-1 inhibitor (e.g., tislelizumab) can be given at 200 mg. If a 1-week dose is given, then the PD-1 inhibitor (e.g., tislelizumab) can be given at 100 mg.

For example, if an anti-PD-1 antibody, such as tislelizumab is used, it can be administered at a dose of 200 mg as an intravenous infusion, once every three week. Alternatively, tislelizumab can be administered at a dose of 300 mg as an intravenous infusion, once every four weeks. If an anti-PD-1 antibody, such as tislelizumab is used, it can be administered at a dose of 300 mg as an intravenous infusion, once every three week. Alternatively, tislelizumab can be administered at a dose of 400 mg as an intravenous infusion, once every four weeks.

The structure of the active compounds identified by code numbers, generic or trade names may be taken from the actual edition of the standard compendium “The Merck Index” or from databases, e.g. Patents International (e.g. IMS World Publications). The above-mentioned compounds, which can be used in combination with a compound of the present invention, can be prepared and administered as described in the art, such as in the documents cited above.

In one embodiment, the invention provides a product comprising a compound of the present invention and at least one other therapeutic agent as a combined preparation for simultaneous, separate or sequential use in therapy. In one embodiment, the therapy is the treatment of a disease or condition mediated by WRN. Products provided as a combined preparation include a composition comprising the compound of the present invention and the other therapeutic agent(s) together in the same pharmaceutical composition, or the compound of the present invention and the other therapeutic agent(s) in separate form, e.g. in the form of a kit.

In one embodiment, the invention provides a pharmaceutical composition comprising a compound of the present invention and another therapeutic agent(s). Optionally, the pharmaceutical composition may comprise a pharmaceutically acceptable carrier, as described above.

In one embodiment, the invention provides a kit comprising two or more separate pharmaceutical compositions, at least one of which contains a compound of the present invention. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of the present invention and the other therapeutic agent may be manufactured and/or formulated by the same or different manufacturers. Moreover, the compound of the present invention and the other therapeutic may be brought together into a combination therapy: (i) prior to release of the combination product to physicians (e.g. in the case of a kit comprising the compound of the present invention and the other therapeutic agent); (ii) by the physician themselves (or under the guidance of the physician) shortly before administration; (iii) in the patient themselves, e.g. during sequential administration of the compound of the present invention and the other therapeutic agent.

Accordingly, the invention provides the use of a compound of the present invention for treating a disease or condition mediated by WRN, wherein the medicament is prepared for administration with another therapeutic agent. The invention also provides the use of another therapeutic agent for treating a disease or condition mediated by WRN, wherein the medicament is administered with a compound of the present invention.

The invention also provides a compound of the present invention for use in treating a disease or condition mediated by WRN, wherein the compound of the present invention is prepared for administration with another therapeutic agent. The invention also provides another therapeutic agent for use in treating a disease or condition mediated by WRN, wherein the other therapeutic agent is prepared for administration with a compound of the present invention. The invention also provides a compound of the present invention for use in treating a disease or condition mediated by WRN, wherein the compound of the present invention is administered with another therapeutic agent. The invention also provides another therapeutic agent for use in a method of treating a disease or condition mediated by WRN, wherein the other therapeutic agent is administered with a compound of the present invention.

The invention also provides the use of a compound of the present invention for treating a disease or condition mediated by WRN, wherein the patient has previously (e.g. within 24 hours) been treated with another therapeutic agent. The invention also provides the use of another therapeutic agent for treating a disease or condition mediated by WRN, wherein the patient has previously (e.g. within 24 hours) been treated with compound of the present invention.

### Biological Assays and Data

The activity of a compound according to the present invention can be assessed by the following in vitro methods.

### Material and Methods

Molecular Biology and virus production. The DNA encoding human Werner helicase (UniProt Q14191, WRN, amino acids S2-S1432) was designed as four DNA strings which were codon-optimized for expression in E. coli. The strings were either ordered from GeneArt (LifeTechnologies, Regensburg, Germany) or made with subcloning overlapping oligonucleotides.

The baculovirus from expression plasmid pLAF1202 (SEQ ID NO: 1) encoding His-ZZ-3C-WRN (aa N517-P1238, encoded by nucleotides 578-2743 in the sequence) was generated with the FlashBac Ultra system (Oxford Expression Technologies 100302) using 540 ng of plasmid DNA, 5.4 μg Flashbac Ultra DNA, and 5.4 microliters Lipofectin (LifeTechnologies 18292-011) for transfection following the manufacturer's instructions. After 5 hours incubation the solution was diluted with 500 microliters TC100 medium (LifeTechnologies 13055-025) and incubated for 7 days at 27° C.

The cells were harvested by centrifugation at 800×g for 10 minutes and the supernatant containing the virus was transferred into a new sterile tube. For the first virus amplification, 500 microliters of the virus was added to 25 mL of SF9 cells at one million cells/mL and incubated for 5 days at 27° C. (200 rpm). The cell viability, density, and diameter was measured and the virus, upon signs of infection, was harvested by centrifugation at 3000 rpm for 15 minutes.

Baculovirus infected insect cells (BIICs) were generated as described by Wasilko et al., 2009, DOI: 10.1016/j.pep.2009.01.002.

In brief, in an Erlenmeyer flask 100 million SF9 cells (one million cells/mL) in 100 mL ESF921 medium (Expression Systems—96-001-01, supplemented with 0.5× Streptomycin/Penicillin) were infected with 300 million baculovirus particles of the respective construct (estimated MOI=3) and incubated at 27° C. for 24 hours at 130 rpm. The infected cells were transferred to 50 mL tubes and harvested by centrifugation at 100×g for 10 minutes at RT.

The cells were resuspended to 10 million/mL in ESF921 (0.5× Streptomycin/Penicillin) medium with BSA (final 10 mg/mL) and 10% DMSO. 500 μL aliquots of cells were transferred to 1.8 mL cryotubes and frozen in Nunc Cryo 1° C. freezing container overnight at −80° C.

SEQ ID NO: 1
AACCATCTCGCAAATAAATAAGTATTTTACTGTTTTCGTAACAGTTTTGTAATAAAAAAACCTATAAATATT
CCGGATTATTCATACCGTCCCACCATCGGGCGCCATGGCTTCTCACCACCATCACCATCACCATCATCATCA
CGCTCAGCACGACGAGGCTGTGGACAACAAGTTCAACAAGGAGCAGCAGAACGCTTTCTACGAGATCCTGCA
CCTCCCTAACCTGAACGAGGAGCAGCGTAACGCTTTCATCCAGTCCCTGAAGGACGACCCTTCTCAGTCTGC
TAACCTGCTGGCTGAGGCTAAGAAGCTGAACGACGCTCAGGCTCCTAAGGTGGACAACAAGTTCAACAAGGA
GCAGCAGAACGCTTTCTACGAGATCCTGCACCTCCCTAACCTGAACGAGGAGCAGCGTAACGCTTTCATCCA
GTCCCTGAAGGACGACCCTTCTCAGTCTGCTAACCTGCTGGCTGAGGCTAAGAAGCTGAACGACGCTCAGGC
TCCTAAGGTGGACGCTAACGGTGGCGGCGGTTCCGGCGGTGGTGGCTCTCTCGAGGTTCTGTTCCAGGGTCC
GAATGAAGGCGAGGAAGATGATGACAAAGACTTCCTGTGGCCAGCTCCAAACGAAGAACAGGTGACTTGTCT
CAAGATGTACTTCGGTCATAGCAGCTTCAAACCAGTGCAGTGGAAAGTTATCCACAGCGTTCTTGAAGAACG
TCGTGATAATGTGGCTGTGATGGCTACTGGCTATGGTAAGAGCCTGTGTTTCCAGTACCCGCCAGTTTACGT
TGGTAAGATCGGTCTGGTGATTAGCCCGCTGATCTCTCTGATGGAAGACCAGGTGCTGCAACTTAAGATGAG
CAACATCCCGGCTTGTTTCCTGGGTTCTGCACAAAGCGAGAACGTGCTCACCGATATCAAGCTGGGTAAGTA
CCGTATCGTGTACGTGACGCCAGAATACTGTAGCGGCAACATGGGTCTTCTGCAACAGCTCGAAGCTGATAT
TGGCATCACCCTCATTGCAGTGGACGAAGCTCACTGTATCAGCGAGTGGGGTCATGATTTCCGCGACTCTTT
CCGTAAACTGGGTTCTCTGAAGACTGCACTTCCGATGGTTCCAATTGTGGCACTGACCGCAACTGCTTCTAG
CTCTATTCGTGAAGACATCGTTCGTTGCCTGAACCTCCGTAACCCACAAATTACCTGCACCGGCTTTGACCG
TCCGAACCTGTACCTGGAGGTTCGTCGTAAGACCGGTAATATCCTTCAGGACCTGCAACCATTCCTGGTTAA
GACCAGCAGCCACTGGGAGTTCGAAGGTCCGACTATCATCTACTGCCCAAGCCGTAAGATGACCCAGCAGGT
TACTGGTGAACTGCGTAAACTGAACCTGAGCTGTGGCACTTACCACGCAGGCATGTCTTTCTCTACCCGTAA
AGACATCCATCATCGTTTCGTGCGTGATGAAATCCAGTGCGTTATCGCTACCATTGCATTCGGCATGGGTAT
CAACAAAGCTGACATCCGTCAAGTGATTCACTACGGTGCACCGAAAGACATGGAAAGCTACTACCAGGAAAT
CGGCCGTGCAGGTCGTGATGGTCTGCAAAGCTCTTGTCATGTGCTGTGGGCACCAGCAGATATTAACCTGAA
CCGTCACCTGCTGACTGAAATTCGTAACGAGAAATTCCGTCTGTACAAACTGAAGATGATGGCGAAGATGGA
GAAATACCTGCATAGCTCCCGTTGTCGTCGTCAAATCATTCTGAGCCATTTCGAGGATAAACAGGTGCAGAA
AGCTTCTCTGGGTATCATGGGCACTGAGAAGTGCTGCGATAACTGTCGTAGCCGTCTTGATCACTGCTACAG
CATGGACGATAGCGAAGACACTTCTTGGGATTTCGGTCCACAAGCATTCAAACTGCTGAGCGCAGTTGATAT
CCTGGGTGAGAAATTCGGCATCGGCCTCCCAATCCTGTTTCTGCGCGGTTCTAACTCTCAGCGTCTTGCTGA
TCAATACCGTCGTCACTCTCTGTTCGGCACTGGTAAAGACCAGACCGAATCTTGGTGGAAAGCATTCAGCCG
TCAACTTATCACCGAAGGCTTTCTGGTGGAAGTGTCTCGTTACAACAAGTTCATGAAGATCTGCGCACTGAC
TAAGAAAGGTCGTAACTGGCTGCACAAGGCAAATACCGAGTCTCAGTCTCTTATCCTTCAGGCTAACGAAGA
ACTGTGCCCGAAGAAGCTTCTGCTGCCATCTTCTAAGACCGTGAGCTCTGGTACTAAAGAGCATTGCTACAA
CCAGGTGCCGGTTGAACTGTCTACCGAGAAGAAGTCCAACCTGGAGAAGCTGTACTCCTACAAACCGTGCGA
CAAGATCTCCTCCGGTTCTAATATCAGCAAGAAGTCCATCATGGTGCAGTCTCCGGAGAAAGCTTACAGCAG
CTCTCAGCCAGTTATCTCTGCACAGGAACAGGAAACTCAGATTGTGCTGTACGGTAAACTGGTGGAAGCACG
TCAGAAACACGCTAACAAGATGGACGTGCCGCCAGCAATTCTTGCAACCAACAAGATTCTGGTGGACATGGC
TAAGATGCGCCCAACTACTGTTGAGAACGTGAAACGTATCGACGGTGTTAGCGAAGGTAAAGCTGCAATGCT
GGCACCACTGCTTGAAGTTATCAAGCATTTCTGCCAGACCAACTCTGTTCAGACCGACCTGTTCTCTTCTAC
CAAACCATAATGGTACCGAATTCGCGGCCGCAGAGCTCGCTCTGGTGCCACGCGGTAGTTCCGCTTGGAGCC
ACCCGCAGTTCGAAAAGTAAGTGATTAACCTCAGGTTATACATATATTTTGAATTTAATTAATTATACATAT
ATTTTATATTATTTTTGTCTTTTATTATCGAGGGGCCGTTGTTGGTGTGGGGTTTTGCATAGAAATAACAAT
GGGAGTTGGCGACGTTGCTGCGCCAACACCACCTCCCTTCCCTCCTTTCATCATGTATCTGTAGATAAAATA
AAATATTAAACCTAAAAACAAGACCGCGCCTATCAACAAAATGATAGGCATTAACTTGCCGCTGACGCTGTC
ACTAACGTTGGACGATTTGCCGACTAAACCTTCATCGCCCAGTAACCAATCTAGGTAGCTGAGCGCATGCAA
GCTGATCCGGGTTATTAGTACATTTATTAAGCGCTAGATTCTGTGCGTTGTTGATTTACAGACAATTGTTGT
ACGTATTTTAATAATTCATTAAATTTATAATCTTTAGGGTGGTATGTTAGAGCGAAAATCAAATGATTTTCA
GCGTCTTTATATCTGAATTTAAATATTAAATCCTCAATAGATTTGTAAAATAGGTTTCGATTAGTTTCAAAC
AAGGGTTGTTTTTCCGAACCGATGGCTGGACTATCTAATGGATTTTCGCTCAACGCCACAAAACTTGCCAAA
TCTTGTAGCAGCAATCTAGCTTTGTCGATATTCGTTTGTGTTTTGTTTTGTAATAAAGGTTCGACGTCGTTC
AAAATATTATGCGCTTTTGTATTTCTTTCATCACTGTCGTTAGTGTACAATTGACTCGACGTAAACACGTTA
AATAGAGCTTGGACATATTTAACATCGGGCGTGTTAGCTTTATTAGGCCGATTATCGTCGTCGTCCCAACCC
TCGTCGTTAGAAGTTGCTTCCGAAGACGATTTTGCCATAGCCACACGACGCCTATTAATTGTGTCGGCTAAC
ACGTCCGCGATCAAATTTGTAGTTGAGCTTTTTGGAATTATTTCTGATTGCGGGCGTTTTTGGGCGGGTTTC
AATCTAACTGTGCCCGATTTTAATTCAGACAACACGTTAGAAAGCGATGGTGCAGGCGGTGGTAACATTTCA
GACGGCAAATCTACTAATGGCGGCGGTGGTGGAGCTGATGATAAATCTACCATCGGTGGAGGCGCAGGCGGG
GCTGGCGGCGGAGGCGGAGGCGGAGGTGGTGGCGGTGATGCAGACGGCGGTTTAGGCTCAAATGTCTCTTTA
GGCAACACAGTCGGCACCTCAACTATTGTACTGGTTTCGGGCGCCGTTTTTGGTTTGACCGGTCTAAGACGA
GTGCGATTTTTTTCGTTTCTAATAGCTTCCAACAATTGTTGTCTGTCGTCTAAAGGTGCAGCGGGTTGAGGT
TCCGTCGGCATTGGTGGAGCGGGCGGCAATTCAGACATCGATGGTGGTGGTGGTGGTGGAGGCGCTGGAATG
TTAGGCACGGGAGAAGGTGGTGGCGGCGGTGCCGCCGGTATAATTTGTTCTGGTTTAGTTTGTTCGCGCACG
ATTGTGGGCACCGGCGCAGGCGCCGCTGGCTGCACAACGGAAGGTCGTCTGCTTCGAGGCAGCGCTTGGGGT
GGTGGCAATTCAATATTATAATTGGAATACAAATCGTAAAAATCTGCTATAAGCATTGTAATTTCGCTATCG
TTTACCGTGCCGATATTTAACAACCGCTCAATGTAAGCAATTGTATTGTAAAGAGATTGTCTCAAGCTCGGA
TCGATCCCGCACGCCGATAACAAGCCTTTTCATTTTTACTACAGCATTGTAGTGGCGAGACACTTCGCTGTC
GTCGAGGTTTAAACGCTTCCTCGCTCACTGACTCGCTGCGCTCGGTCGTTCGGCTGCGGCGAGCGGTATCAG
CTCACTCAAAGGCGGTAATACGGTTATCCACAGAATCAGGGGATAACGCAGGAAAGAACATGTGAGCAAAAG
GCCAGCAAAAGGCCAGGAACCGTAAAAAGGCCGCGTTGCTGGCGTTTTTCCATAGGCTCCGCCCCCCTGACG
AGCATCACAAAAATCGACGCTCAAGTCAGAGGTGGCGAAACCCGACAGGACTATAAAGATACCAGGCGTTTC
CCCCTGGAAGCTCCCTCGTGCGCTCTCCTGTTCCGACCCTGCCGCTTACCGGATACCTGTCCGCCTTTCTCC
CTTCGGGAAGCGTGGCGCTTTCTCATAGCTCACGCTGTAGGTATCTCAGTTCGGTGTAGGTCGTTCGCTCCA
AGCTGGGCTGTGTGCACGAACCCCCCGTTCAGCCCGACCGCTGCGCCTTATCCGGTAACTATCGTCTTGAGT
CCAACCCGGTAAGACACGACTTATCGCCACTGGCAGCAGCCACTGGTAACAGGATTAGCAGAGCGAGGTATG
TAGGCGGTGCTACAGAGTTCTTGAAGTGGTGGCCTAACTACGGCTACACTAGAAGGACAGTATTTGGTATCT
GCGCTCTGCTGAAGCCAGTTACCTTCGGAAAAAGAGTTGGTAGCTCTTGATCCGGCAAACAAACCACCGCTG
GTAGCGGTGGTTTTTTTGTTTGCAAGCAGCAGATTACGCGCAGAAAAAAAGGATCTCAAGAAGATCCTTTGA
TCTTTTCTACGGGGTCTGACGCTCAGTGGAACGAAAACTCACGTTAAGGGATTTTGGTCATGAGATTATCAA
AAAGGATCTTCACCTAGATCCTTTTAAATTAAAAATGAAGTTTTAAATCAATCTAAAGTATATATGAGTAAA
CTTGGTCTGACAGTTACCAATGCTTAATCAGTGAGGCACCTATCTCAGCGATCTGTCTATTTCGTTCATCCA
TAGTTGCCTGACTCCCCGTCGTGTAGATAACTACGATACGGGAGGGCTTACCATCTGGCCCCAGTGCTGCAA
TGATACCGCGAGACCCACGCTCACCGGCTCCAGATTTATCAGCAATAAACCAGCCAGCCGGAAGGGCCGAGC
GCAGAAGTGGTCCTGCAACTTTATCCGCCTCCATCCAGTCTATTAATTGTTGCCGGGAAGCTAGAGTAAGTA
GTTCGCCAGTTAATAGTTTGCGCAACGTTGTTGCCATTGCTACAGGCATCGTGGTGTCACGCTCGTCGTTTG
GTATGGCTTCATTCAGCTCCGGTTCCCAACGATCAAGGCGAGTTACATGATCCCCCATGTTGTGCAAAAAAG
CGGTTAGCTCCTTCGGTCCTCCGATCGTTGTCAGAAGTAAGTTGGCCGCAGTGTTATCACTCATGGTTATGG
CAGCACTGCATAATTCTCTTACTGTCATGCCATCCGTAAGATGCTTTTCTGTGACTGGTGAGTACTCAACCA
AGTCATTCTGAGAATAGTGTATGCGGCGACCGAGTTGCTCTTGCCCGGCGTCAATACGGGATAATACCGCGC
CACATAGCAGAACTTTAAAAGTGCTCATCATTGGAAAACGTTCTTCGGGGCGAAAACTCTCAAGGATCTTAC
CGCTGTTGAGATCCAGTTCGATGTAACCCACTCGTGCACCCAACTGATCTTCAGCATCTTTTACTTTCACCA
GCGTTTCTGGGTGAGCAAAAACAGGAAGGCAAAATGCCGCAAAAAAGGGAATAAGGGCGACACGGAAATGTT
GAATACTCATACTCTTCCTTTTTCAATATTATTGAAGCATTTATCAGGGTTATTGTCTCATGAGCGGATACA
TATTTGAATGTATTTAGAAAAATAAACAAATAGGGGTTCCGCGCACATTTCCCCGAAAAGTGCCACCTGACG
CGCCCTGTAGCGGCGCATTAAGCGCGGCGGGTGTGGTGGTTACGCGCAGCGTGACCGCTACACTTGCCAGCG
CCCTAGCGCCCGCTCCTTTCGCTTTCTTCCCTTCCTTTCTCGCCACGTTCGCCGGCTTTCCCCGTCAAGCTC
TAAATCGGGGGCTCCCTTTAGGGTTCCGATTTAGTGCTTTACGGCACCTCGACCCCAAAAAACTTGATTAGG
GTGATGGTTCACGTAGTGGGCCATCGCCCTGATAGACGGTTTTTCGCCCTTTGACGTTGGAGTCCACGTTCT
TTAATAGTGGACTCTTGTTCCAAACTGGAACAACACTCAACCCTATCTCGGTCTATTCTTTTGATTTATAAG
GGATTTTGCCGATTTCGGCCTATTGGTTAAAAAATGAGCTGATTTAACAAAAATTTAACGCGAATTTTAACA
AAATATTAACGTTTACAATTTCCCATTCGCCATTCAGGCTGCGCAACTGTTGGGAAGGGCGATCGGTGCGGG
CCTCTTCGCTATTACGCCAGGAACGGCTCCGCCCACTATTAATGAAATTAAAAATTCCAATTTTAAAAAACG
CAGCAAGAGAAACATTTGTATGAAAGAATGCGTAGAAGGAAAGAAAAATGTCGTCGACATGCTGAACAACAA
GATTAATATGCCTCCGTGTATAAAAAAAATATTGAACGATTTGAAAGAAAACAATGTACCGCGCGGCGGTAT
GTACAGGAAGAGGTTTATACTAAACTGTTACATTGCAAACGTGGTTTCGTGTGCCAAGTGTGAAAACCGATG
TTTAATCAAGGCTCTGACGCATTTCTACAACCACGACTCCAAGTGTGTGGGTGAAGTCATGCATCTTTTAAT
CAAATCCCAAGATGTGTATAAACCACCAAACTGCCAAAAAATGAAAACTGTCGACAAGCTCTGTCCGTTTGC
TGGCAACTGCAAGGGTCTCAATCCTATTTGTAATTATTGAATAATAAAACAATTATAAATGCTAAATTTGTT
TTTTATTAACGATACAAACCAAACGCAACAAGAACATTTGTAGTATTATCTATAATTGAAAACGCGTAGTTA
TAATCGCTGAGGTAATATTTAAAATCATTTTCAAATGATTCACAGTTAATTTGCGACAATATAATTTTATTT
TCACATAAACTAGACGCCTTGTCGTCTTCTTCTTCGTATTCCTTCTCTTTTTCATTTTTCTCTTCATAAAAA
TTAACATAGTTATTATCGTATCCATATATGTATCTATCGTATAGAGTAAATTTTTTGTTGTCATAAATATAT
ATGTCTTTTTTAATGGGGTGTATAGTACCGCTGCGCATAGTTTTTCTGTAATTTACAACAGTGCTATTTTCT
GGTAGTTCTTCGGAGTGTGTTGCTTTAATTATTAAATTTATATAATCAATGAATTTGGGATCGTCGGTTTTG
TACAATATGTTGCCGGCATAGTACGCAGCTTCTTCTAGTTCAATTACACCATTTTTTAGCAGCACCGGATTA
ACATAACTTTCCAAAATGTTGTACGAACCGTTAAACAAAAACAGTTCACCTCCCTTTTCTATACTATTGTCT
GCGAGCAGTTGTTTGTTGTTAAAAATAACAGCCATTGTAATAAGACGCACAAACTAATATCACAAACTGGAA
ATGTCTATCAATATATAGTTGCTGATCAGATCTACCCGTAGTGGCTATGGCAGGGCTTGCCGCCCCGACGTT
GGCTGCGAGCCCTGGGCCTTCACCCGAACTTGGGGGTTGGGGTGGGGAAAAGGAAGAAACGCGGGCGTATTG
GTCCCAATGGGGTCTCGGTGGGGTATCGACAGAGTGCCAGCCCTGGGACCGAACCCCGCGTTTATGAACAAA
CGACCCAACACCCGTGCGTTTTATTCTGTCTTTTTATTGCCGTCATAGCGCGGGTTCCTTCCGGTATTGTCT
CCTTCCGTGTTTCAGTTAGCCTCCCCCATCTCCCGGTACCGCATGCTATGCATCGGCCGCTTTACTTGTACA
GCTCGTCCATGCCGAGAGTGATCCCGGCGGCGGTCACGAACTCCAGCAGGACCATGTGATCGCGCTTCTCGT
TGGGGTCTTTGCTCAGGGCGGACTGGGTGCTCAGGTAGTGGTTGTCGGGCAGCAGCACGGGGCCGTCGCCGA
TGGGGGTGTTCTGCTGGTAGTGGTCGGCGAGCTGCACGCTGCCGTCCTCGATGTTGTGGCGGATCTTGAAGT
TCACCTTGATGCCGTTCTTCTGCTTGTCGGCCATGATATAGACGTTGTGGCTGTTGTAGTTGTACTCCAGCT
TGTGCCCCAGGATGTTGCCGTCCTCCTTGAAGTCGATGCCCTTCAGCTCGATGCGGTTCACCAGGGTGTCGC
CCTCGAACTTCACCTCGGCGCGGGTCTTGTAGTTGCCGTCGTCCTTGAAGAAGATGGTGCGCTCCTGGACGT
AGCCTTCGGGCATGGCGGACTTGAAGAAGTCGTGCTGCTTCATGTGGTCGGGGTAGCGGCTGAAGCACTGCA
CGCCGTAGGTCAGGGTGGTCACGAGGGTGGGCCAGGGCACGGGCAGCTTGCCGGTGGTGCAGATGAACTTCA
GGGTCAGCTTGCCGTAGGTGGCATCGCCCTCGCCCTCGCCGGACACGCTGAACTTGTGGCCGTTTACGTCGC
CGTCCAGCTCGACCAGGATGGGCACCACCCCGGTGAACAGCTCCTCGCCCTTGCTCACCATCGTCGAGATCC
CGGGCGTTTAAATTGTGTAATTTATGTAGCTGTAATTTTTACCTTATTAATATTTTTTACGCTTTGCATTCG
ACGACTGAACTCCCAAATATATGTTTAACTCGTCTTGGTCGTTTGAATTTTTGTTGCTGTGTTTCCTAATAT
TTTCCATCACCTTAAATATGTTATTGTAATCCTCAATGTTGAACTTGCAATTGGACACGGCATAGTTTTCCA
TAGTCGTGTAAAACATGGTATTGGCTGCATTGTAATACATCCGACTGAGCGGGTACGGATCTATGTGTTTGA
GCAGCCTGTTCAAAAACTCTGCATCGTCGCAAAACGGAATTTGGTACCCGGGCGTATACTCCGGAATATTAA
TAGATCATGGAGATAATTAAAATGAT

### Protein Expression and Purification

BIICs aliquots for Werner helicase protein His-ZZ-3C-WRN (aa N517-P1238, pLAF1202) were diluted 1/100 into ESF921 medium and further diluted 1/100 into the expression/production flasks with Sf21 cells (one million cells/mL) in 1 L ESF921 medium and incubated for protein expression for 96 h (27° C., 130 rpm).

The WRN protein was purified using the following protocol. The cell pellets were thawed and resuspended in 80 mL buffer A (50 mM Tris, 300 mM NaCl, 20 mM imidazole, 1 mM TCEP, 10% glycerol, pH 7.8) supplemented with Turbonuclease (final concentration 40 units/mL, Merck) and cOmplete protease inhibitor tablets (1 tablet/50 mL, Roche). The cells were lysed by three passages through a homogenizer (Avestin, Emulsiflex C3) at 800-1000 bar. The lysed sample was centrifuged at 48000×g for 40 minutes (Sorvall RC5B, SS-34 rotor) and the supernatant was passed through a 0.45 μm filter.

The lysate was loaded onto a HisTrap crude FF 5 mL column (GE Healthcare) mounted on an ÄKTA Pure 25 chromatography system (GE Healthcare). Contaminating proteins were washed away with buffer A and bound protein was eluted with a linear gradient over 10 column volumes to 100% of buffer B (50 mM Tris, 300 mM NaCl, 300 mM imidazole, 1 mM TCEP, 10% glycerol, pH 7.8). 1% (w/w) HRV 3C protease (His-MBP-tagged, produced in-house) was added to the eluted protein. The N-terminal purification tag was cleaved off by the protease during dialysis overnight at 5° C. against 2 L buffer (50 mM Tris pH 7.0, 150 mM NaCl, 1 mM TCEP, 10% glycerol, 0.02% CHAPS). The protein solution was then carefully diluted with adding two volume parts of 20 mM Tris pH 7.0, 10% glycerol, 0.02% CHAPS. The slightly turbid protein solution was passed over a 0.45 μm filter. The cleaved protein was loaded onto a Resource S 6 mL column (GE Healthcare) pre-equilibrated with 20 mM Tris, 20 mM NaCl, 1 mM TCEP, 10% glycerol, pH 7.0. Cleaved tag and contaminating proteins were washed away with the equilibration buffer. The bound target protein was eluted with a linear gradient over 20 column volumes of the same buffer containing 1 M sodium chloride and then injected onto a HiLoad 16/600 Superdex 75 pg column (GE Healthcare) pre-equilibrated with 50 mM Tris pH 7.4, 300 mM NaCl, 10% glycerol. Fractions containing pure protein were identified by SDS-PAGE and pooled. The purified protein was finally split into aliquots and frozen on dry ice. The purity, quantity, and identity of the protein was determined by RP-HPLC and LC-MS.

### In Vitro Enzymatic Activity Assay on WRN Helicase

An ATPase assay was set up to measure the DNA dependent ATP hydrolysis activity of WRN helicase. This assay was used also to assess the inhibition properties of compounds of the invention on DNA dependent WRN ATPase activity.

The core helicase motif of the WRN protein (aa N517-P1238) was produced for this assay (protein production as described above). A 45 oligonucleotide sequence called “FLAP26” as described by Brosh et al., 2009, DOI: 10.1074/jbc.M 111446200 (TTTTTTTTTTTTTTTTTTTTTTCCAAGTAAAACGACGGCCAGTGC; SEQ ID NO: 2) was purchased from IDT (Integrated DNA Technologies, Leuven, Belgium) and used as single strand DNA substrate. The ADP-Glo assay kit (Promega, Madison, WI) allowing the quantification of ADP produced in ATP hydrolysis reactions was used for setting up this assay.

Time course experiments were first performed in order to determine the best enzymatic assay conditions (including buffer conditions, reaction time and concentrations of protein, ATP and DNA substrates). A typical reaction consists of 10 nM WRN protein, 0.2 nM FLAP26, and 300 micromolar ATP in the following assay buffer: 30 mM Tris pH7.5, 2 mM MgCl₂, 0.02% BSA, 50 mM NaCl, 0.1% pluronic F127 prepared in DNAse free water.

To evaluate the inhibition properties of compounds of the invention, serial dilutions were prepared in DMSO (10 half log dilutions from a 10 mM DMSO solution). 50 nanoliters of each concentration was pre-incubated for 3 hours in a 384 small volume assay plate (Greiner #784075) with 2.5 microliters of a 20 nM WRN helicase protein in assay buffer with 600 micromolar ATP. Control wells were included with a “high control” (no inhibition), containing DMSO with no test compound, and “low controls” (maximal inhibition), containing buffer without protein. The reaction was started by addition of 2.5 microliters of FLAP26 at 0.4 nM and incubated for 30 minutes at room temperature. The reaction was stopped with the addition of 5 microliters of the first ADP-Glo reagent and incubated for one hour to remove the excess amount of ATP. Afterwards, 10 microliters of ATP detection reagent was added and incubated for an additional hour before reading. Luminescence output was recorded using Tecan 1000 reader, with 5 minutes delay before reading. Each concentration of compound was tested in duplicates in the assay plate.

Data analysis was carried out using an in-house developed software (Novartis Helios software application, Novartis Institutes for BioMedical Research, unpublished) using the methods described by Formenko et al., 2006, DOI: 10.1016/j.cmpb.2006.01.008. Following normalization of activity values for the wells to % inhibition (% inhibition=[(high control-sample)/(high control-low control)]×100), IC₅₀ fitting was carried out from the duplicate determinations present on each plate according to [4]. Data analysis can also be carried out using commercially available software designed to derive IC₅₀ values using 4-parameter fits (e.g. GraphPad Prism, XL fit). The reported IC₅₀ values are the geometrical means of at least 2 independent replicates.

### Method for Detecting Effects on Cellular Proliferation

The colon carcinoma cell lines SW48 (RRID: CVCL_1724), HCT 116 (RRID: CVCL_0291) and SNU-407 (RRID: CVCL_5058) were obtained from ATCC. The WRN-knockdown insensitive colon carcinoma cell line DLD-1 (RRID: CVCL_0248) was obtained from the Korean Cell Line Bank (KCLB), and used to generate a derivative in which the endogenous WRN gene copies were knocked out by CRISPR-mediated editing using standard CRISPR methods. The resulting cell line, DLD1-WRN-KO, was used to assess potential off-target compound effects.

SW48, SNU-407 and DLD1-WRN-KO cells were cultured in growth medium composed of RPMI-1640 (Amimed Cat #1-41F22-1), 2 mM L-Glutamine (Amimed Cat #5-10K50), 10 mM HEPES (Gibco Cat #15630-056), 1 mM sodium pyruvate (Amimed Cat #5-60F00-H), 1× Penicillin-Streptomycin (Amimed Cat #4-01F00-H) and 10% fetal calf serum (Amimed Cat #2-01F30-G, Lot #LB11566P). HCT 116 cells were cultured in growth medium composed of McCoys 5A (Amimed catalog #1-18F01-1), 2 mM L-Glutamine (Amimed Cat #5-10K50), 1× Penicillin-Streptomycin (Amimed Cat #4-01F00-H) and 10% fetal calf serum (Amimed Cat #2-01F30-G, Lot #LB11566P). All cells were maintained at 37° C. in a humidified 5% CO₂ incubator.

Following filtration through a Steriflip-NY 20 μm filter (Millipore Cat #SCNY00020), trypsinized cells were seeded in 100 microliters growth medium at 2,000 (SW48) or 1,500 (SNU-407, DLD1-WRN-KO, HCT 116) cells/well into white, clear-bottom 96-well plates (Costar Cat #3903). Three replicate plates were prepared for each compound treatment condition. In addition, one plate (termed “day 0”) was prepared to quantify the number of viable cells at the time of compound addition. Following overnight incubation at 37° C. in a humidified 5% CO₂ atmosphere, eight 3-fold serial dilutions of a given compound stock (obtained at a concentration of 10 mM in DMSO and stored at 4° C.) were dispensed directly into each of the triplicate assay plates using a HP 300D non-contact Digital Dispenser (TECAN). The final concentration of DMSO was normalized to 0.1% in all wells. 96 hours after compound addition, cellular ATP levels as a surrogate for cell viability was assessed following addition of 50 microliters CellTiterGlo (Promega Cat #G7573) reagent and luminescence quantification on a MPLEX multi-mode plate-reader (TECAN) following a 10 minute incubation at room temperature. The number of viable cells in the “day 0” plate were quantified identically on the day of compound addition.

For data analysis, the assay background signal that was determined in wells containing medium, but no cells, was subtracted from all other data points prior to further calculations. The extent of growth inhibition and potential cell kill was assessed by comparing the ATP levels (measured using CellTiterGlo, Promega) in compound-treated cells with those present at the time of compound addition. To this end, the following conditional concept was programmatically applied in HELIOS, an in-house software applying a multi-step decision tree to arrive at optimal concentration response curve fits (Gubler et al, SLAS DOI: 10.1177/2472555217752140) to calculate % growth (% G) for each compound-treated well: % G=(T−V0)/V0))*100 when T<V0, and % G=(T−V0)/(V−V0)))*100 when T≥V0, where V0 is the viability level at time of compound addition, while V and T represent vehicle-control and compound-treated viability levels, respectively, at the end of the compound incubation. 100%, 0% and −100% signify absence of growth inhibition, growth stasis, and complete cell kill, respectively. Compound concentrations leading to half-maximal growth inhibition (GI50) and residual cell viability at the highest tested compound concentration (Data (cmax), expressed in percent) were routinely calculated. Data analysis can also be carried out using commercially available software designed to derive IC50 values using 4-parameter fits (e.g. GraphPad Prism, XL fit). The reported GI₅₀ values are the geometrical means of at least 2 independent replicates.

The following table shows the IC₅₀ data in the WRN ATPase assay and the GI₅₀ data for the proliferation assays using SW48 and DLD1-WRN-KO cell lines for compounds of the invention. For example, Example 11 is a WRN ATPase inhibitor with a biochemical IC₅₀ of 0.05 μM and a proliferation GI₅₀ of 0.06 μM in SW48 and greater than 10 μM in the DLD1 WRN-KO cell lines.

| | | | Proliferation |
|---|---|---|---|
| | WRN ATPase | Proliferation | Assay |
| | activity | Assay SW48 | DLD1-WRN-KO |
| Example | IC₅₀ [μM] | GI₅₀ [μM] | GI₅₀ [μM] |
| 1 | 1 | 5 | >10 |
| 2 | 0.3 | >10 | >10 |
| 3 | 0.1 | 3 | 10 |
| 4 | intentionally | | |
| | blank | | |
| 5 | 2 | 8 | >10 |
| 6 | 2 | 7 | >10 |
| 7 | 0.04 | | >10 |
| 8 | 0.05 | | >10 |
| 9 | 0.08 | | >10 |
| 10 | 0.09 | | >10 |
| 11 | 0.05 | 0.06 | >10 |
| 12 | 0.01 | 0.8 | >10 |
| 13 | 0.03 | 0.3 | >10 |
| 14 | 0.04 | 0.2 | >10 |
| 15 | 0.05 | 4 | >10 |
| 16 | 0.05 | 0.5 | >10 |
| 17 | 0.05 | 0.9 | 8 |
| 18 | 0.06 | 0.2 | >10 |
| 19 | 0.06 | >10 | >10 |
| 20 | 0.09 | 1 | >10 |
| 21 | 0.1 | >10 | >10 |
| 22 | 0.1 | >10 | >10 |
| 23 | 0.2 | 0.3 | >10 |
| 24 | 0.2 | >10 | >10 |
| 25 | 0.2 | 4 | >10 |
| 26 | 0.3 | >10 | >10 |
| 27 | 0.3 | >10 | >10 |
| 28 | 0.5 | >10 | >10 |
| 29 | 0.1 | 2 | >10 |
| 30 | 0.1 | 2 | >10 |
| 31 | 0.2 | 6 | >10 |
| 32 | 0.8 | 3 | >10 |
| 33 | 1 | 8 | >10 |
| 34 | 5 | 7 | >10 |
| 35 | 0.04 | 0.3 | >10 |
| 36 | 0.1 | >10 | >10 |
| 37 | 0.09 | 0.09 | >10 |
| 38 | 0.03 | 0.1 | >10 |
| 39 | 0.03 | 0.3 | 4 |
| 40 | 0.04 | 0.6 | >10 |
| 41 | 0.05 | 0.1 | 6 |
| 42 | 0.07 | 0.5 | >10 |
| 43 | 0.08 | 1.0 | >10 |
| 44 | 0.08 | 0.9 | >10 |
| 45 | 0.09 | 0.3 | >10 |
| 46 | 0.09 | 0.2 | >10 |
| 47 | 0.09 | 0.9 | >10 |
| 48 | 0.1 | >10 | >10 |
| 49 | 0.2 | 0.5 | >10 |
| 50 | 0.3 | 1.0 | >10 |
| 51 | 0.6 | 2 | 7 |
| 52 | 0.05 | 3 | >10 |
| 53 | 0.06 | 0.8 | >10 |
| 54 | 0.1 | 1 | >10 |
| 55 | 0.1 | 0.8 | >10 |
| 56 | 0.1 | 1 | >10 |
| 57 | 0.1 | 1 | >10 |
| 58 | 0.1 | 2 | >10 |
| 59 | 0.3 | 1.0 | >10 |
| 60 | 0.3 | 3 | >10 |
| 61 | 0.4 | 4 | >10 |
| 62 | 0.5 | 1 | >10 |
| 63 | 0.5 | 4 | >10 |
| 64 | 0.9 | 4 | >10 |
| 65 | 8 | 4 | 4 |
| 66 | 0.03 | 0.3 | >10 |
| 67 | 0.3 | 3 | >10 |
| 68 | 0.02 | 0.1 | >10 |
| 69 | 0.5 | 4 | >10 |
| 70 | 0.07 | 0.9 | >10 |
| 71 | 0.09 | 5 | >10 |
| 72 | 0.1 | 2 | >10 |
| 73 | 0.1 | 2 | >10 |
| 74 | 0.2 | | 0.6 |
| 75 | 0.2 | | 0.9 |
| 76 | intentionally | | |
| | blank | | |
| 77 | 0.2 | 4 | >10 |
| 78 | 0.2 | 3 | >10 |
| 79 | 0.2 | 5 | >10 |
| 80 | 0.3 | 0.8 | >10 |
| 81 | 0.3 | 4 | >10 |
| 82 | 0.3 | 2 | >10 |
| 83 | 0.3 | 2 | >10 |
| 84 | 0.3 | 1 | >10 |
| 85 | 0.5 | 4 | >10 |
| 86 | 0.07 | | >10 |
| 87 | 0.5 | 4 | >10 |
| 88 | 0.5 | 3 | >10 |
| 89 | 0.6 | 3 | >10 |
| 90 | 0.6 | 4 | >10 |
| 91 | 0.7 | 3 | >10 |
| 92 | 0.7 | 4 | >10 |
| 93 | 0.7 | | 5 |
| 94 | 0.7 | 3 | >10 |
| 95 | 0.8 | 2 | >10 |
| 96 | 0.8 | 3 | >10 |
| 97 | 1 | 4 | >10 |
| 98 | 1 | 6 | >10 |
| 99 | 2 | 6 | >10 |
| 100 | 2 | 4 | >10 |
| 101 | 2 | 10 | >10 |
| 102 | >3 | 7 | 5 |
| 103 | 4 | 4 | >10 |
| 104 | 0.8 | 8 | >10 |
| 105 | 0.1 | 0.8 | 5 |
| 106 | 0.1 | 0.2 | >10 |
| 107 | 0.2 | 3 | >10 |
| 108 | 0.2 | 0.9 | >10 |
| 109 | 0.2 | 4 | >10 |
| 110 | 0.2 | 5 | >10 |
| 111 | 0.2 | | >10 |
| 112 | 0.2 | 0.9 | >10 |
| 113 | 0.3 | 5 | >10 |
| 114 | 0.4 | 2 | >10 |
| 115 | 0.5 | 2 | 5 |
| 116 | 0.5 | | 5 |
| 117 | 0.6 | | >10 |
| 118 | 0.6 | 9 | >10 |
| 119 | 0.6 | 6 | >10 |
| 120 | 0.6 | 6 | >10 |
| 121 | 0.7 | 5 | >10 |
| 122 | 0.7 | 5 | >10 |
| 123 | 0.7 | 9 | >10 |
| 124 | 0.7 | 4 | >10 |
| 125 | 0.8 | 3 | >10 |
| 126 | 0.8 | 4 | >10 |
| 127 | 0.02 | >10 | 7 |
| 128 | 0.8 | 7 | >10 |
| 129 | 0.8 | 5 | >10 |
| 130 | 0.9 | 2 | 7 |
| 131 | 1 | 6 | >10 |
| 132 | 2 | 10 | >10 |
| 133 | 2 | 6 | >10 |
| 134 | 4 | 5 | >10 |
| 135 | 0.4 | >10 | >10 |
| 136 | 0.02 | 0.5 | >10 |
| 137 | 0.05 | 0.3 | >10 |
| 138 | 0.08 | 1 | >10 |
| 139 | 0.1 | 1 | >10 |
| 140 | 0.1 | 4 | >10 |
| 141 | 0.4 | 8 | >10 |
| 142 | 0.6 | 5 | >10 |
| 143 | | | >10 |
| 144 | | 3 | |
| 145 | | 2 | |
| 146 | 0.09 | 4 | >10 |
| 147 | 0.6 | 6 | >10 |
| 148 | 0.06 | 0.3 | >10 |
| 149 | 0.09 | 2 | >10 |
| 150 | 0.09 | 2 | >10 |
| 151 | 0.2 | 4 | |
| 152 | 0.5 | >10 | >10 |
| 153 | 0.10 | | 5 |
| 154 | 0.1 | | 8 |
| 155 | 0.1 | 2 | >10 |
| 156 | 0.2 | 3 | >10 |
| 157 | 0.2 | 3 | >10 |
| 158 | 0.3 | 3 | 1 |
| 159 | 0.6 | >10 | >10 |
| 160 | 0.6 | 6 | >10 |
| 161 | 0.7 | 5 | >10 |
| 162 | 0.9 | 3 | >10 |
| 163 | 1.0 | 7 | >10 |
| 164 | 0.05 | >10 | >10 |
| 165 | 0.09 | >10 | |
| 166 | 0.1 | >10 | >10 |

Data are geometric means of at least 2 duplicate determinations.

### Preparation of Compounds

Compounds of the present invention can be prepared as described in the following Examples. The Examples are intended to illustrate the invention and are not to be construed as being limitations thereof.

### Instrumentation

Microwave: All microwave reactions were conducted in a Biotage Initiator or an Anton Paar monowave 450, irradiating at 0-400 W from a magnetron at 2.45 GHz with Robot Eight/Robot Sixty/Robot twentyfour processing capacity, unless otherwise stated.

UPLC-MS Methods: Using Waters Acquity UPLC with Waters SQ detector, unless stated otherwise.

UPLC-MS Methods: Using Waters Acquity UPLC with Waters SQ detector, unless stated otherwise.

UPLC-MS 1:

| Column | CORTECS ™ C18+ 2.7 μm, | |
|---|---|---|
| Column Dimension | 2.1 × 50 | mm |
| Column Temperature | 80° | C. |
| Eluents | A: water + 4.76% isopropanol + 0.05% FA + 3.75 mM AA | |
| | B: isopropanol + 0.05% FA | |
| Flow Rate | 1.0 | mL/min |

| | |
|---|---|
| Gradient | 1 to 50% B in 1.4 min; 50 to 98% B in 0.3 min |

UPLC-MS 3:

| Column | ACQUITY UPLC ® BEH C18 1.7 μm | |
|---|---|---|
| Column Dimension | 2.1 × 50 | mm |
| Column Temperature | 80° | C. |
| Eluents | A: water + 4.76% isopropanol + 0.05% FA + 3.75 mM AA | |
| | B: isopropanol + 0.05% FA | |
| Flow Rate | 0.6 | mL/min |

| | |
|---|---|
| Gradient | 1 to 98% B in 1.7 min |

UPLC-MS 4:

| Column | ACQUITY UPLC ® BEH C18 1.7 μm | |
|---|---|---|
| Column Dimension | 2.1 × 50 | mm |
| Column Temperature | 80° | C. |
| Eluents | A: water + 0.05% FA + 3.75 mM AA | |
| | B: isopropanol + 0.05% FA | |
| Flow Rate | 0.6 / 0.7 | mL/min |

| | |
|---|---|
| Gradient | 5 to 98% B in 1.7 min |

UPLC-MS 7:

| Column | Acquity UPLC ® HSS T3 1.8 μm | |
|---|---|---|
| Column Dimension | 2.1 × 50 | mm |
| Column Temperature | 60° | C. |
| Eluents | A: water + 0.05% formic acid + 3.75 mM ammonium acetate | |
| | B: acetonitrile + 0.04% FA | |
| Flow Rate | 1.0 | mL/min |

| | |
|---|---|
| Gradient | 2 to 98% B in 1.4 min |

UPLC-MS 8:

| Column | Acquity UPLC ® HSS T3 1.8 μm | |
|---|---|---|
| Column Dimension | 2.1 × 50 | mm |
| Column Temperature | 60° | C. |
| Eluents | A: water + 0.05% formic acid + 3.75 mM ammonium acetate | |
| | B: acetonitrile + 0.04% FA | |
| Flow Rate | 1.0 | mL/min |

| | |
|---|---|
| Gradient | 5 to 98% B in 1.4 min |

UPLC-MS 10:

| Column | CORTECS ™ C18+ 2.7 μm, | |
|---|---|---|
| Column Dimension | 2.1 × 50 | mm |
| Column Temperature | 80° | C. |
| Eluents | A: water + 0.05% FA + 3.75 mM AA | |
| | B: isopropanol + 0.05% FA | |
| Flow Rate | 1.0 | mL/min |

| | |
|---|---|
| Gradient | concave from 1 to 98% B in 1.4 min |

UPLC-MS 11:

| Instrument | Shimadzu NEXERA UPLC PDA with Shimadzu LCMS 2020 | |
|---|---|---|
| | as MSD | |
| Column | Mercury MS Synergi C12 2.5 μm | |
| Column Dimension | 20 × 4.0 | mm |
| Column Temperature | 40° | C. |
| Eluents | A: water + 0.1% FA | |
| | B: acetonitrile | |
| Flow Rate | 2.0 | mL/min |

| | |
|---|---|
| Gradient | Time/% B: 0.01/5, 0.5/5, 1.0/95, 1.5/95, 2.0/5, 3.0/5 |

UPLC-MS 12:

| Instrument | Agilent 1200 HPLC PDA with AB Sciex API2000 TQ as MSD | |
|---|---|---|
| Column | Mercury MS Synergi C12 2.5 μm | |
| Column Dimension | 20 × 4.0 | mm |
| Column Temperature | 30° | C. |
| Eluents | A: water + 0.1% FA | |
| | B: acetonitrile | |
| Flow Rate | 2.0 | mL/min |

| | |
|---|---|
| Gradient | Time/% B: 0.01/30, 0.5/30, 1.0/95, 2.4/95, 2.5/30, 3.0/30 |

UPLC-MS 15:

| Column | Acquity UPLC ® HSS T3 1.8 μm | |
|---|---|---|
| Column Dimension | 2.1 × 50 | mm |
| Column Temperature | 50° | C. |
| Eluents | A: water + 0.05% formic acid + 3.75 mM ammonium acetate | |
| | B: acetonitrile + 0.04% FA | |
| Flow Rate | 1.0 | mL/min |

| | |
|---|---|
| Gradient | 5 to 98% B in 1.4 min |

UPLC-MS 17:

| Column | Acquity UPLC ® BEH C18 1.7 μm | |
|---|---|---|
| Column Dimension | 2.1 × 100 | mm |
| Column Temperature | 80° | C. |
| Eluents | A: water + 4.76% isopropanol + 0.05% FA + 3.75 mM AA | |
| | B: isopropanol + 0.05% FA | |
| Flow Rate | 0.4 | mL/min |

| | |
|---|---|
| Gradient | 1 to 60% B in 8.4 min; 60 to 98% B in 1.0 min |

UPLC-MS 18:

| Column | Acquity UPLC ® BEH C18 1.7 μm | |
|---|---|---|
| Column Dimension | 2.1 × 50 | mm |
| Column Temperature | 60° | C. |
| Eluents | A: water + 0.05% FA + 3.75 mM AA | |
| | B: acetonitrile + 0.04% FA | |
| Flow Rate | 1.0 | mL/min |

| | |
|---|---|
| Gradient | 2 to 98% B in 0.8 min |

HPLC Methods:
HPLC 1:

| Instrument | Agilent 1100 series with PDA Detector | |
|---|---|---|
| Column | Kinetex C-18, 5 μm | |
| Column Dimension | 150 × 4.6 | mm |
| Column Temperature | 40° | C. |
| Eluents | A: water + 0.01% TFA | |
| | B: acetonitrile | |
| Flow Rate | 1.0 | mL/min |

| | |
|---|---|
| Gradient | Time/B: 0/30, 2/40, 5/90, 8/100, 10/100, 11/30, 12/30 |

HPLC 4:

| Instrument | Agilent 1260 | |
|---|---|---|
| Column | Agilent Poroshell 120 EC-C18, 2.7 μm | |
| Column Dimension | 4.6 × 50 | mm |
| Column Temperature | 40° | C. |
| Eluents | A: water + 0.1% TFA | |
| | B: acetonitrile + 0.1% TFA | |
| Flow Rate | 1.2 | mL/min |

| | |
|---|---|
| Gradient | 5% B to 95% B in 5 min, hold 2 min |

HPLC 5:

| Instrument | Agilent 1260 HPLC | |
|---|---|---|
| Column | InertSustain C18, 5 μm | |
| Column Dimension | 4.6 × 150 | mm |
| Column Temperature | 30° | C. |
| Eluents | A: water + 5 mmol (NH₄)₂CO₃ | |
| | B: acetonitrile | |
| Flow Rate | 1.0 | mL/min |

| | |
|---|---|
| Gradient | 10% B to 90% B in 8 min, hold 2 min |

HPLC 6:

| Instrument | Agilent 1260 infinity series HPLC system with DAD/ELSD | |
|---|---|---|
| Column | Atlantis dC18, 5 μm | |
| Column Dimension | 4.6 × 250 | mm |
| Column Temperature | 25° | C. |
| Eluents | A: water + 0.1% TFA | |
| | B: acetonitrile | |
| Flow Rate | 1.0 | mL/min |

| | |
|---|---|
| Gradient | 10% B to 100% B in 15 min, hold 5 min |

### Preparative Methods:

### Column Chromatography:

Column chromatography was run on silica gel using prepacked columns, as detailed below, or using glass columns following standard flash chromatography methodology, unless otherwise stated.

| | |
|---|---|
| System 1 | Teledyne ISCO, CombiFlash Rf, |
| | CombiFlash Rf+ |
| System 2 | Biotage Isolera |
| Column | pre-packed RediSep Rf cartridges, |
| | or SNAP cartridges |
| Sample adsorption | onto Isolute, or on silica gel, |
| | or applied as solutions |

### Supercritical Fluid Chromatography (SFC)

Purifications were achieved on a Waters Preparative SFC-100-MS system with ABSYS update, with a Waters 2998 Photodiode Array Detector and a Waters MS Single Quadrupole Detector.

### SFC 8:

Instrument: WATERS SFC 100 with ABSYS update
Mobile phase: A: CO₂, B: MeOH
Flow rate: 150 mL/min MeOH+30 mL/min CO₂, constant flow of 180 mL/min
Column: 100×30 Reprosil NH₂ 100A 3 μm
Temperature: 50° C.
Back pressure: 100 bar
Detection UV: 210-400 nm
Gradient: 16% B to 24% B in 4 min

### Reversed Phase HPLC:

RP-HPLC acidic 1:

| | |
|---|---|
| System | Gilson |
| Column | Waters SunFire Prep C18 OBD |
| | (100 mm × 30 mm), 5 μm |
| Eluents | A: water + 0.1% TFA, B: acetonitrile |
| Flow rate | 40 mL/min |

### Preparation of Compounds

The following examples are intended to illustrate the invention and are not to be construed as being limitations thereon. Temperatures are given in degrees Celsius. If not mentioned otherwise, all evaporations are performed under reduced pressure, typically between about 15 mm Hg and 100 mm Hg (=20-133 mbar). Abbreviations used are those conventional in the art.

All starting materials, building blocks, reagents, acids, bases, dehydrating agents, solvents, and catalysts utilized to synthesize the compounds of the present invention are either commercially available or can be produced by organic synthesis methods known to one of ordinary skill in the art. Further, the compounds of the present invention can be produced by organic synthesis methods known to one of ordinary skill in the art as shown in the following examples.

The structures of all final products, intermediates and starting materials are confirmed by standard analytical spectroscopic characteristics, e.g., MS, IR, NMR. The absolute stereochemistry of representative examples of the preferred (most active) isomers has been determined by analyses of X-ray crystal structures of complexes in which the respective compounds are bound to WRN or by small molecule xray crystal structures of a precursor or the final compound.

Amines synthetized via acidic deprotection of the Boc-precursor were often obtained as HCl or TFA salt. The corresponding free base can be isolated by partitioning between DCM and aq sat NaHCO₃ as described for Intermediate D.

### General Conditions:

Mass spectra were acquired on LC-MS systems using electrospray, chemical and electron impact ionization methods with a range of instruments of the following configurations: Waters Acquity UPLC with Waters SQ detector, Shimadzu NEXERA UPLC PDA with Shimadzu LCMS 2020 as MSD, Agilent 1200 HPLC PDA with AB Sciex AP12000 TQ as MSD and Agilent 1200 HPLC PDA with AB Sciex AP13200 QTRAP as MSD. [M+H]⁺ refers to the protonated molecular ion of the chemical species.

NMR spectra were run with Bruker Ultrashield™400 (400 MHz), Bruker Ultrashield™400 Plus (400 MHz), Bruker Ultrashield™600 (600 MHz) and Bruker Ascend™400 (400 MHz) spectrometers, all with and without tetramethylsilane as an internal standard. Chemical shifts (d-values) are reported in ppm downfield from tetramethylsilane, spectra splitting pattern are designated as singlet (s), doublet (d), triplet (t), multiplet, unresolved or more overlapping signals (m), broad signal (br). Solvents are given in parentheses.
Celite: Celite^{R} (the Celite corporation)=filtering aid based on diatomaceous earth
Phase separator: Biotage—Isolute phase separator—(Part number: 120-1906-D for 15 mL, Part number: 120-1908-F for 70 mL and Part number: 120-1909-J for 150 mL)
SiliaMetS® Thiol: SiliCYCLE thiol metal scavenger—(Part number: R51030B, Loading: 1.31 mmol/g Particle Size: 40-63 μm)
ISOLUTE® Si-Thiol: Biotage thiol metal scavenger—(Part number: 9180-0100, Loading: 1.3 mmol/g)
PL-BnSH MP-Resin: Agilent thiol metal scavenger—(Part number: PL3582-6689, 2.2 mmol/g 100 A 150-1 kg)
ISOLUTE® Si-TMT: Biotage thiol metal scavenger—(Part number: 9538)
Smopex®-301: Alfa Aesar thiol metal scavenger (Part number: 45902)
PL-HCO₃ MP SPE cartridge (500 mg per 6 mL)—(Part number: PL3540-C₆₀₃)
PL-HCO₃ MP SPE cartridge (100 mg per 6 mL)—(Part number: PL3540-A603)

### Sodium Salt Formation:

The compound can be suspended in tert-butanol and NaOH 0.1 M (1 eq) added. The mixture ca be stirred/sonicated at RT. If the suspension turns into a clear solution it can be lyophilized. If the suspension is still turbid, water can be added and the resulting solution lyophilized. If no change happens, NaOH 0.1 M up to 2 eq in total is added until a clear solution is observed, which is then lyophilized. If the NMR of the resulting solid still contains tert-butanol, the solid is dissolved in a small amount of water and lyophilized again.

### Abbreviations

| Abbreviation | Description |
|---|---|
| AA | ammonium acetate |
| ACN | acetonitrile |
| AcOH | acetic acid |
| AgBF₄ | silver tetrafluoroborate |
| aq | aqueous |
| BCl₃ | boron trichloride |
| BINAP | 2,2′-bis(diphenylphosphino)-1,1′-binaphthyl |
| Boc | tert-butyloxycarbonyl |
| Boc₂O | di-tert-butyl dicarbonate |
| br | broad signal |
| brine | saturated aqueous sodium chloride |
| ° C. | degrees Celsius |
| CaCl₂ | calcium chloride |
| CO | carbon monoxide |
| CO₂ | carbon dioxide |
| CPhos | 2-dicyclohexylphosphino-2′,6′-bis(N,N-dimethylamino)biphenyl |
| CPhos Pd G3 | [(2-dicyclohexylphosphino-2′,6′-bis(N,N-dimethylamino)-1,1′- |
| | biphenyl)-2-(2′-amino-1,1′-biphenyl)] palladium(II) |
| | methanesulfonate |
| Cs₂CO₃ | caesium carbonate |
| DAST | diethylaminosulfur trifluoride |
| DCM | dichloromethane |
| DIBAL-H | diisobutylaluminium hydride |
| DIPEA | N,N-diisopropylethylamine |
| DMAP | 4-dimethylaminopyridine |
| DME | 1,2-dimethoxyethane |
| DMF | N,N-dimethylformamide |
| DMSO | dimethyl sulfoxide |
| DMSO-d₆ | hexadeuterodimethyl sulfoxide |
| dppf | 1,1′-bis(diphenylphosphino)ferrocene |
| EDC•HCl | 3-(ethyliminomethyleneamino)-N,N-dimethylpropan-1-amine |
| | hydrochloride |
| EDCl | 3-(ethyliminomethyleneamino)-N,N-dimethylpropan-1-amine |
| ee | enantiomeric excess |
| eq | equivalent |
| Et₃N | triethylamine |
| Et₂O | diethyl ether |
| EtOAc | ethylacetate |
| EtOH | ethanol |
| FA | formic acid |
| H₂O | water |
| HATU | 1-[Bis(dimethylamino)methylene]-1H-1,2,3-triazolo[4,5- |
| | b]pyridinium 3-oxide hexafluorophosphate |
| HBr | hydrobromic acid |
| HCl | hydrochloric acid |
| HCOOH | formic acid |
| Hg | mercury |
| HI | hydroiodic acid |
| HNO₃ | nitric acid |
| HOAt | 3H-[1,2,3]triazolo[4,5-b]pyridin-3-ol |
| HOBt | benzotriazol-1-ol |
| HPLC | high-performance liquid chromatography |
| H₃PO₄ | phosphoric acid |
| HV | high vacuum |
| Hz, MHz, GHz | hertz, megahertz, gigahertz |
| IPA | isopropyl alcohol |
| IPAc | isopropyl acetate |
| J | coupling constant |
| K₂CO₃ | potassium carbonate |
| kg / g / mg | kilogram / gram / milligram |
| KHMDS | potassium bis(trimethylsilyl)amide |
| KI | potassium iodide |
| KMnO₄ | potassium permanganate |
| KOH | potassium hydroxide |
| K₃PO₄ | potassium phosphate |
| L / mL / μL | liter / milliliter / microliter |
| LC-MS | liquid chromatography and mass spectrometry |
| LDA | lithium diisopropylamide |
| LiCl | lithium chloride |
| M / mM | molar / millimolar |
| mbar | millibar |
| MCC pad | microcrystalline cellulose |
| MeOH | methanol |
| MeTHF | 2-methyloxolane / 2-methyltetrahydrofuran |
| MgSO₄ | magnesium sulfate |
| min | minutes |
| mm / nm / μm | millimeter / nanometer / micrometer |
| μM / mM | micromolar / millimolar |
| μmol / mmol / mol | micromol / millimol / mol |
| MS | mass spectrometry |
| MW, mw | microwave |
| m/z | mass to charge ratio |
| N | normality |
| NaCl | sodium chloride |
| NaCN | sodium cyanide |
| NaH | sodium hydride |
| NaHCO₃ | sodium bicarbonate |
| NaH₂PO₄ | sodium dihydrogen phosphate |
| NaHSO4 | sodium bisulfate |
| NaOH | sodium hydroxide |
| Na₂O₃S₂ | sodium thiosulfate |
| NaOtBu | sodium 2-methylpropan-2-olate |
| Na₂SO₄ | sodium sulfate |
| NBS | N-bromosuccinimide |
| NH₃ | ammonia |
| NH₄Cl | ammonium chloride |
| NH₄OH | ammonium hydroxide |
| (NH₄)₂CO₃ | ammonium carbonate |
| NIS | N-iodosuccinimide |
| NMP | 1-methylpyrrolidin-2-one |
| NMR | nuclear magnetic resonance |
| PdCl₂(PPh₃)₂ | bis(triphenylphosphine)palladium(II) dichloride |
| Pd(dppf)Cl₂ | [1,1′-bis(diphenylphosphino)ferrocene]palladium(II) dichloride |
| Pd(dppf)Cl₂•DCM | [1,1′-Bis(diphenylphosphino)ferrocene]palladium(II) dichloride, |
| | DCM adduct |
| Pd(OAc)₂ | 2 palladium(II) acetate |
| Pd(PPh₃)₄ | tetrakis(triphenylphosphine)palladium(0) |
| PMa | pressure maximum allowable |
| POCl₃ | phosphoryl chloride |
| PyAOP | (7-azabenzotriazol-1-yloxy)tripyrrolidinophosphonium |
| | hexafluorophosphate |
| R_{f} | retardation factor |
| RM or rm | reaction mixture |
| RP | reversed phase |
| RPM | revolutions per minute |
| Rt | retention time (if not indicated, in minutes) |
| RT | room temperature |
| RuPhos | 2-dicyclohexylphosphino-2′,6′-diisopropoxybiphenyl |
| s, d, dd, t, m | singulet, doublet, doublet of doublets, triplet, multiplet |
| sat | saturated |
| scCO₂ | supercritical CO₂ |
| SEM | β-(trimethylsilyl)ethoxymethyl |
| SFC | supercritical fluid chromatography |
| T₃P | propanephosphonic acid anhydride |
| TBAF | tetrabutylammonium fluoride |
| TBME | 2-methoxy-2-methylpropane |
| TFA | trifluoroacetic acid |
| THF | tetrahydrofuran |
| TLC | thin-layer chromatography |
| TMSOTf | trimethylsilyl trifluoromethanesulfonate |
| UPLC | ultra performance liquid chromatography |
| W | watt |
| XPhos Pd G2 | chloro(2-dicyclohexylphosphino-2′,4′,6′-triisopropyl-1,1′- |
| | biphenyl)[2-(2′-amino-1,1′-biphenyl)]palladium(II) |
| XPhos Pd G3 | (2-dicyclohexylphosphino-2′,4′,6′-triisopropyl-1,1′-biphenyl)[2-(2′- |
| | amino-1,1′-biphenyl)]palladium(II) methanesulfonate |
| XRPD | X-Ray Powder Diffraction |

### Preparation of Final Compounds

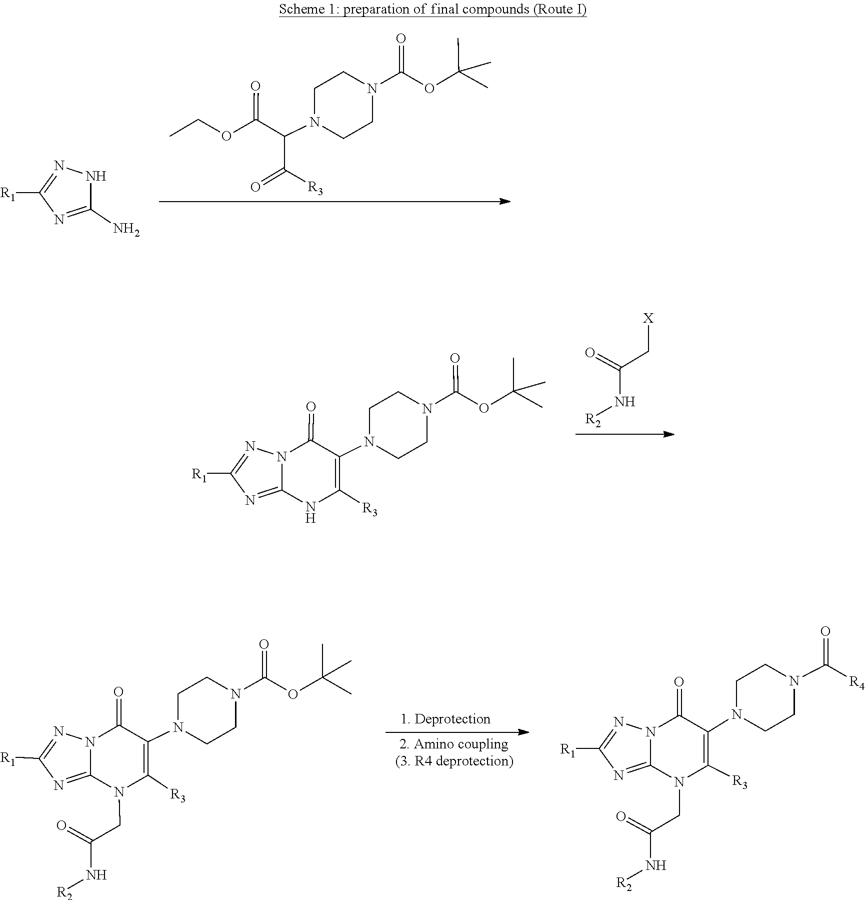

### Example 1: 2-(6-(4-acetylpiperazin-1-yl)-2-(3-fluoropiperidin-1-yl)-5-methyl-7-oxo-[1,2,4]triazolo[1,5-a]pyrimidin-4(7H)-yl)-N-(4-(trifluoromethyl)phenyl)acetamide

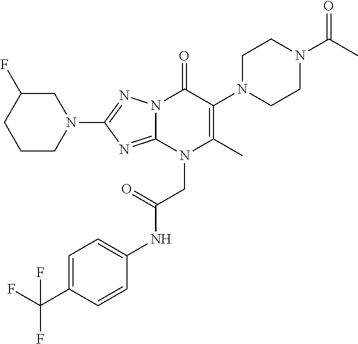 [SMILES: CC(=O)N1CCN(c2c(C)n(CC(=O)Nc3ccc(C(F)(F)F)cc3)c3nc(N4CCCC(F)C4)nn3c2=O)CC1]

6-(4-Acetylpiperazin-1-yl)-2-(3-fluoropiperidin-1-yl)-5-methyl-[1,2,4]triazolo[1,5-a]pyrimidin-7(4H)-one (Intermediate F) (550 mg, 1.45 mmol) was suspended in DMF (10 mL). 2-Bromo-N-(4-(trifluoromethyl)phenyl)acetamide (450 mg, 1.60 mmol) and DIPEA (760 μmol, 4.35 mmol) were added at 0° C. The RM was stirred at RT for 12 hours. Water was added and the resulting solid was filtered off. The crude product was purified by column chromatography (Combiflash column: 12 g, eluent DCM:MeOH 100:0 to 95:5). The product containing fractions were combined, concentrated and dried under HV to give the title compound.

LC-MS: Rt=1.50 min; MS m/z [M+H]⁺ 579.2; UPLC-MS 11

Examples 2 to 10 were made using analogous methods to Example 1, using methods known to the skilled chemist and using starting materials in the public domain. Examples 7 to 10 were analyzed from the crude reaction mixture.

| | | Rt | MS | LC-MS |
|---|---|---|---|---|
| Ex. | Compound | [min] | [M + H]+ | method |
| 2 | 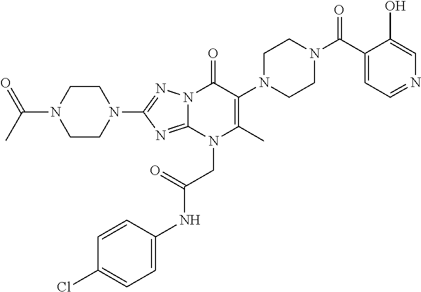 [SMILES: CC(=O)N1CCN(c2nc3n(CC(=O)Nc4ccc(Cl)cc4)c(C)c(N4CCN(C(=O)c5ccncc5O)CC4)c(=O)n3n2)CC1] | 1.44 | 649.3 | UPLC- MS 11 |
| | 2-(2-(4-acetylpiperazin-1-yl)-6-(4-(3- | | | |
| | hydroxyisonicotinoyl)piperazin-1-yl)-5-methyl-7- | | | |
| | oxo-[1,2,4]triazolo[1,5-a]pyrimidin-4(7H)-yl)-N-(4- | | | |
| | chlorophenyl)acetamide | | | |
| 3 | 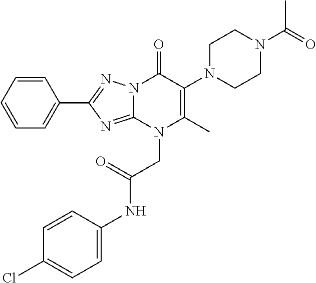 [SMILES: CC(=O)N1CCN(c2c(C)n(CC(=O)Nc3ccc(Cl)cc3)c3nc(-c4ccccc4)nn3c2=O)CC1] | 1.01 | 520.2 | UPLC- MS 8 |
| | 2-(6-(4-acetylpiperazin-1-yl)-5-methyl-7-oxo-2- | | | |
| | phenyl-[1,2,4]triazolo[1,5-a]pyrimidin-4(7H)-yl)-N- | | | |
| | (4-chlorophenyl)acetamide | | | |
| 4 | Intentionally blank | | | |
| 5 | 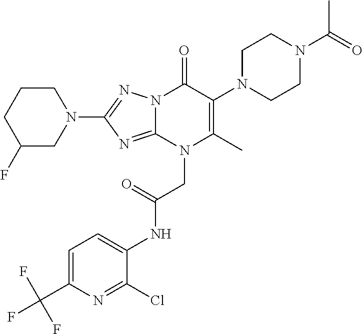 [SMILES: CC(=O)N1CCN(c2c(C)n(CC(=O)Nc3ccc(C(F)(F)F)nc3Cl)c3nc(N4CCCC(F)C4)nn3c2=O)CC1] | 1.49 | 614.2 | UPLC- MS 11 |
| | 2-(6-(4-acetylpiperazin-1-yl)-2-(3-fluoropiperidin-1- | | | |
| | yl)-5-methyl-7-oxo-[1,2,4]triazolo[1,5-a]pyrimidin- | | | |
| | 4(7H)-yl)-N-(2-chloro-6-(trifluoromethyl)pyridin-3- | | | |
| | yl)acetamide | | | |
| 6 | 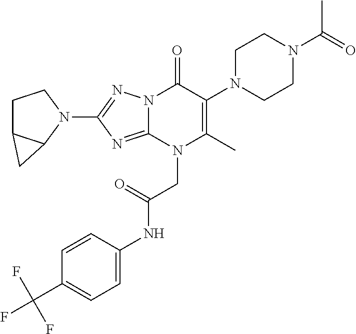 [SMILES: CC(=O)N1CCN(c2c(C)n(CC(=O)Nc3ccc(C(F)(F)F)cc3)c3nc(N4CCC5CC54)nn3c2=O)CC1] | 1.48 | 559.3 | UPLC- MS 11 |
| | 2-(6-(4-acetylpiperazin-1-yl)-2-(2- | | | |
| | azabicyclo[3.1.0]hexan-2-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 7 | 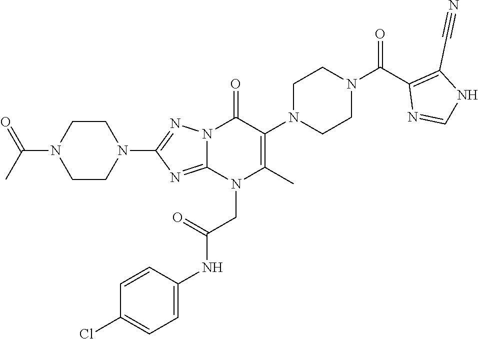 [SMILES: CC(=O)N1CCN(c2nc3n(CC(=O)Nc4ccc(Cl)cc4)c(C)c(N4CCN(C(=O)c5nc[nH]c5C#N)CC4)c(=O)n3n2)CC1] | 0.57 | 647.5 | UPLC- MS 18 |
| | 2-(2-(4-acetylpiperazin-1-yl)-6-(4-(5-cyano-1H- | | | |
| | imidazole-4-carbonyl)piperazin-1-yl)-5-methyl-7-oxo- | | | |
| | [1,2,4]triazolo[1,5-a]pyrimidin-4(7H)-yl)-N-(4- | | | |
| | chlorophenyl)acetamide | | | |
| 8 | 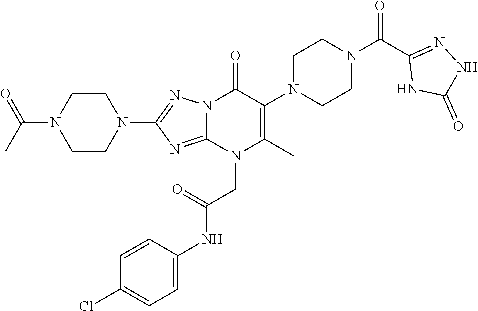 [SMILES: CC(=O)N1CCN(c2nc3n(CC(=O)Nc4ccc(Cl)cc4)c(C)c(N4CCN(C(=O)c5n[nH]c(=O)[nH]5)CC4)c(=O)n3n2)CC1] | 0.54 | 639.6 | UPLC- MS 18 |
| | 2-(2-(4-acetylpiperazin-1-yl)-5-methyl-7-oxo-6-(4-(5- | | | |
| | oxo-4,5-dihydro-1H-1,2,4-triazole-3- | | | |
| | carbonyl)piperazin-1-yl)-[1,2,4]triazolo[1,5- | | | |
| | a]pyrimidin-4(7H)-yl)-N-(4-chlorophenyl)acetamide | | | |
| 9 | 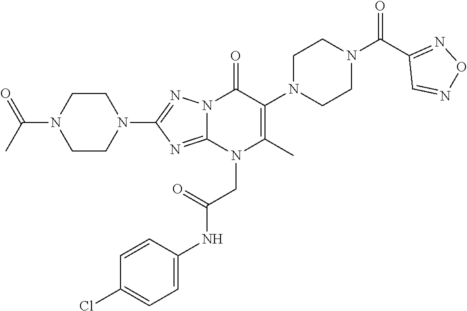 [SMILES: CC(=O)N1CCN(c2nc3n(CC(=O)Nc4ccc(Cl)cc4)c(C)c(N4CCN(C(=O)c5cnon5)CC4)c(=O)n3n2)CC1] | 0.59 | 624.4 | UPLC- MS 18 |
| | 2-(6-(4-(1,2,5-oxadiazole-3-carbonyl)piperazin-1-yl)- | | | |
| | 2-(4-acetylpiperazin-1-yl)-5-methyl-7-oxo- | | | |
| | [1,2,4]triazolo[1,5-a]pyrimidin-4(7H)-yl)-N-(4- | | | |
| | chlorophenyl)acetamide | | | |
| 10 | 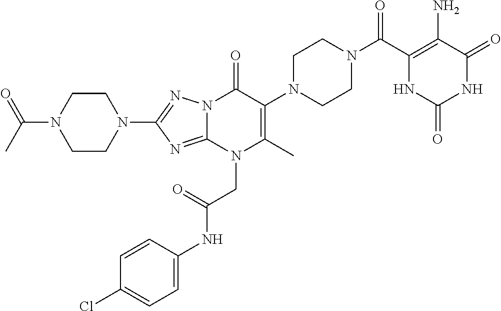 [SMILES: CC(=O)N1CCN(c2nc3n(CC(=O)Nc4ccc(Cl)cc4)c(C)c(N4CCN(C(=O)c5[nH]c(=O)[nH]c(=O)c5N)CC4)c(=O)n3n2)CC1] | 0.53 | 681.5 | UPLC- MS 18 |
| | 2-(2-(4-acetylpiperazin-1-yl)-6-(4-(5-amino-2,6-dioxo- | | | |
| | 1,2,3,6-tetrahydropyrimidine-4-carbonyl)piperazin-1- | | | |
| | yl)-5-methyl-7-oxo-[1,2,4]triazolo[1,5-a]pyrimidin- | | | |
| | 4(7H)-yl)-N-(4-chlorophenyl)acetamide | | | |

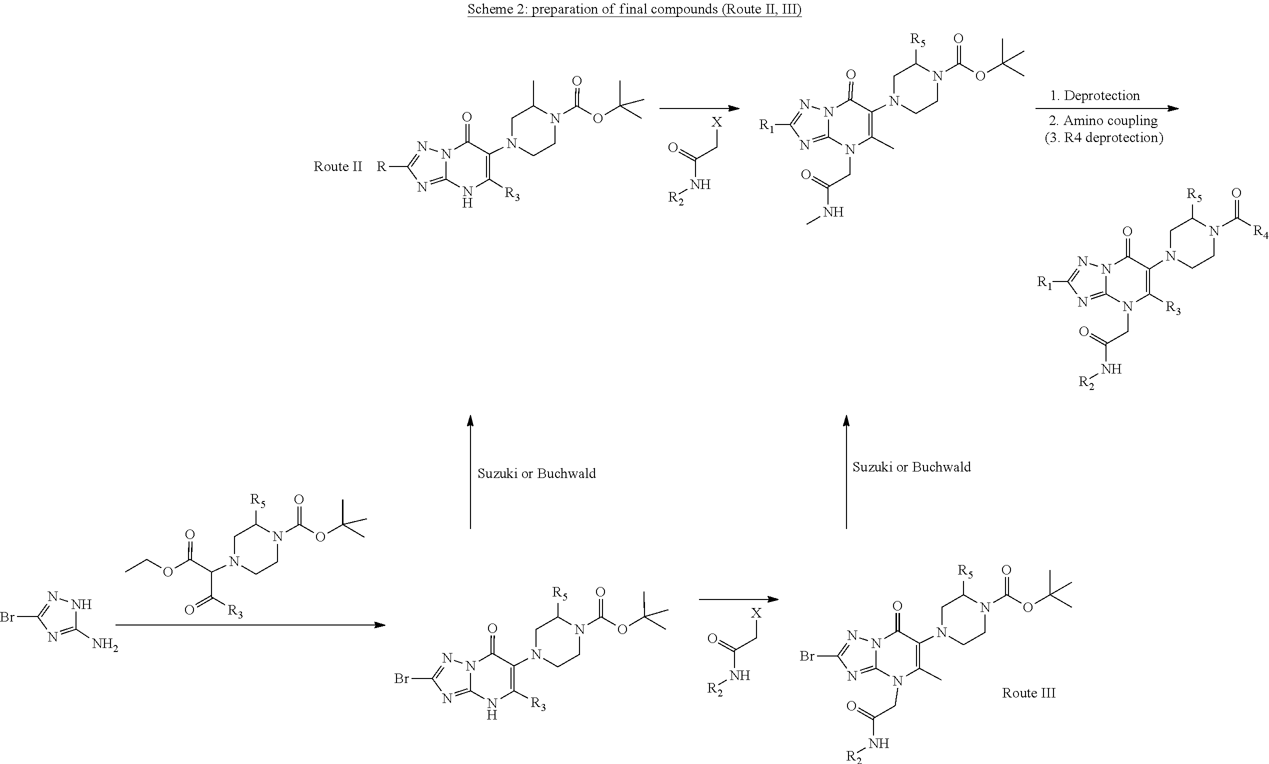

### Route II

### Example 11: N-(2-chloro-4-(trifluoromethyl)phenyl)-2-(2-(3,6-dihydro-2H-pyran-4-yl)-5-ethyl-6-(4-(7-hydroxy-2,3-dihydrofuro[3,2-c]pyridine-6-carbonyl)piperazin-1-yl)-7-oxo-[1,2,4]triazolo[1,5-a]pyrimidin-4(7H)-yl)acetamide

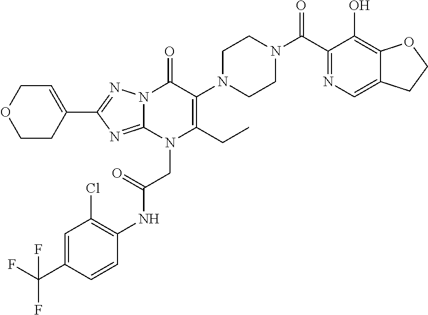 [SMILES: CCc1c(N2CCN(C(=O)c3ncc4c(c3O)OCC4)CC2)c(=O)n2nc(C3=CCOCC3)nc2n1CC(=O)Nc1ccc(C(F)(F)F)cc1Cl]

### Step 1: N-(2-chloro-4-(trifluoromethyl)phenyl)-2-(2-(3,6-dihydro-2H-pyran-4-yl)-5-ethyl-6-(4-(7-(methoxymethoxy)-2,3-dihydrofuro[3,2-c]pyridine-6-carbonyl)piperazin-1-yl)-7-oxo-[1,2,4]triazolo[1,5-a]pyrimidin-4(7H)-yl)acetamide

To a beige suspension of N-(2-chloro-4-(trifluoromethyl)phenyl)-2-(2-(3,6-dihydro-2H-pyran-4-yl)-5-ethyl-7-oxo-6-(piperazin-1-yl)-[1,2,4]triazolo[1,5-a]pyrimidin-4(7H)-yl)acetamide (Intermediate D) (80.0 mg, 134 μmol) and 7-(methoxymethoxy)-2,3-dihydrofuro[3,2-c]pyridine-6-carboxylic acid (Intermediate P) (36.8 mg, 141 μmol) in DMF (1.34 mL) were added DIPEA (117 μL, 671 μmol), then HATU (61.3 mg, 161 μmol). The RM was stirred at RT for 30 minutes. The RM was diluted with water and extracted 3 times with DCM. The combined organic phases were dried through a phase separator and concentrated under reduced pressure. The crude product was purified by column chromatography (RediSep column: Silica 4 g, eluent DCM:DCM/MeOH (8/2) 100:0 to 10:90) to give the title compound.

LC-MS: Rt=1.04 min; MS m/z [M+H]⁺ 773.6/775.6, m/z [M−H]⁻ 771.5/773.6; UPLC-MS 1

### Step 2: N-(2-chloro-4-(trifluoromethyl)phenyl)-2-(2-(3,6-dihydro-2H-pyran-4-yl)-5-ethyl-6-(4-(7-hydroxy-2,3-dihydrofuro[3,2-c]pyridine-6-carbonyl)piperazin-1-yl)-7-oxo-[1,2,4]triazolo[1,5-a]pyrimidin-4(7H)-yl)acetamide

To a light yellow solution of N-(2-chloro-4-(trifluoromethyl)phenyl)-2-(2-(3,6-dihydro-2H-pyran-4-yl)-5-ethyl-6-(4-(7-(methoxymethoxy)-2,3-dihydrofuro[3,2-c]pyridine-6-carbonyl)piperazin-1-yl)-7-oxo-[1,2,4]triazolo[1,5-a]pyrimidin-4(7H)-yl)acetamide (88.0 mg, 97.0 μmol) in anhydrous EtOH (1.94 mL) was added HCl 1.25 M in EtOH (155 μL, 193 μmol). The RM was stirred at RT for 7 days. The RM was concentrated to dryness under vacuum. The residue was triturated in Et₂O. The resulting light-yellow suspension was filtered. The cake was washed with Et₂O and dried to give a beige solid. The filtrate was concentrated under vacuum to give a light brown residue. The cake was dissolved in MeOH and filtered through a PL-HCO₃ MP SPE cartridge. The filtrate was concentrated under reduced pressure to give a beige solid. The crude product was purified by reverse phase preparative HPLC (RP-HPLC acidic 1:5 to 100% B in 20 min). The product containing fractions were combined, basified with aq sat NaHCO₃, extracted twice with DCM, dried through a phase separator and concentrated under reduced pressure to give the title compound.

LC-MS: Rt=0.93 min; MS m/z [M+H]⁺ 729.5/731.5, m/z [M−H]⁻ 727.5/729.4; UPLC-MS 1

Examples 12 to 34 were made using analogous methods to Example 11, using methods known to the skilled chemist and using starting materials in the public domain.

| | | Rt | MS | LC-MS |
|---|---|---|---|---|
| Ex. | Compound | [min] | [M + H]+ | method |
| 12 | 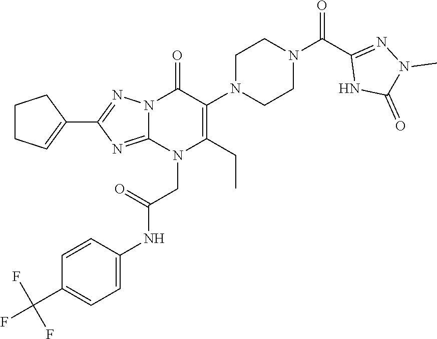 [SMILES: CCc1c(N2CCN(C(=O)c3nn(C)c(=O)[nH]3)CC2)c(=O)n2nc(C3=CCCC3)nc2n1CC(=O)Nc1ccc(C(F)(F)F)cc1] | 1.04 | 641.7 | UPLC- MS 15 |
| | 2-(2-(cyclopent-1-en-1-yl)-5-ethyl-6-(4-(1-methyl-5- | | | |
| | oxo-4,5-dihydro-1H-1,2,4-triazole-3- | | | |
| | carbonyl)piperazin-1-yl)-7-oxo-[1,2,4]triazolo[1,5- | | | |
| | a]pyrimidin-4(7H)-yl)-N-(4- | | | |
| | (trifluoromethyl)phenyl)acetamide | | | |
| 13 | 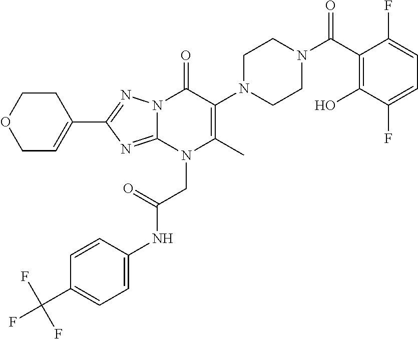 [SMILES: Cc1c(N2CCN(C(=O)c3c(F)ccc(F)c3O)CC2)c(=O)n2nc(C3=CCOCC3)nc2n1CC(=O)Nc1ccc(C(F)(F)F)cc1] | 1.03 | 674.2 | UPLC- MS 8 |
| | 2-(6-(4-(3,6-difluoro-2-hydroxybenzoyl)piperazin-1-yl)- | | | |
| | 2-(3,6-dihydro-2H-pyran-4-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 14 | 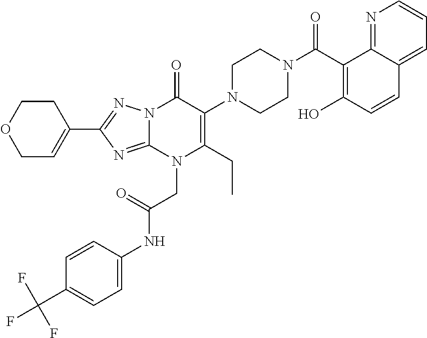 [SMILES: CCc1c(N2CCN(C(=O)c3c(O)ccc4cccnc34)CC2)c(=O)n2nc(C3=CCOCC3)nc2n1CC(=O)Nc1ccc(C(F)(F)F)cc1] | 0.99 | 703.4 | UPLC- MS 4 |
| | 2-(2-(3,6-dihydro-2H-pyran-4-yl)-5-ethyl-6-(4-(7- | | | |
| | hydroxyquinoline-8-carbonyl)piperazin-1-yl)-7-oxo- | | | |
| | [1,2,4]triazolo[1,5-a]pyrimidin-4(7H)-yl)-N-(4- | | | |
| | (trifluoromethyl)phenyl)acetamide | | | |
| 15 | 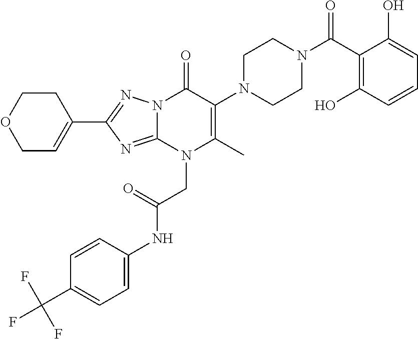 [SMILES: Cc1c(N2CCN(C(=O)c3c(O)cccc3O)CC2)c(=O)n2nc(C3=CCOCC3)nc2n1CC(=O)Nc1ccc(C(F)(F)F)cc1] | 0.94 | 654.7 | UPLC- MS 15 |
| | 2-(2-(3,6-dihydro-2H-pyran-4-yl)-6-(4-(2,6- | | | |
| | dihydroxybenzoyl)piperazin-1-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 16 | 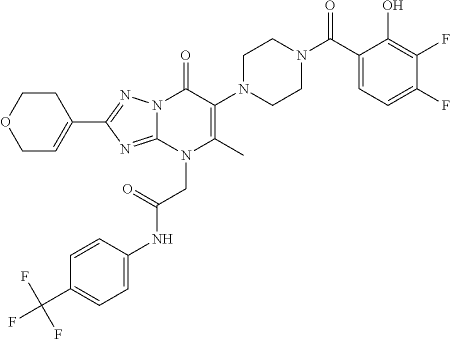 [SMILES: Cc1c(N2CCN(C(=O)c3ccc(F)c(F)c3O)CC2)c(=O)n2nc(C3=CCOCC3)nc2n1CC(=O)Nc1ccc(C(F)(F)F)cc1] | 1.04 | 674.2 | UPLC- MS 8 |
| | 2-(6-(4-(3,4-difluoro-2-hydroxybenzoyl)piperazin-1-yl)- | | | |
| | 2-(3,6-dihydro-2H-pyran-4-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 17 | 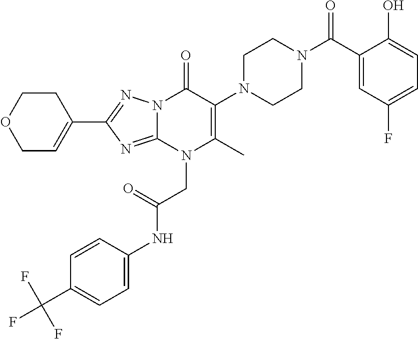 [SMILES: Cc1c(N2CCN(C(=O)c3cc(F)ccc3O)CC2)c(=O)n2nc(C3=CCOCC3)nc2n1CC(=O)Nc1ccc(C(F)(F)F)cc1] | 1.03 | 656.7 | UPLC- MS 15 |
| | 2-(2-(3,6-dihydro-2H-pyran-4-yl)-6-(4-(5-fluoro-2- | | | |
| | hydroxybenzoyl)piperazin-1-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 18 | 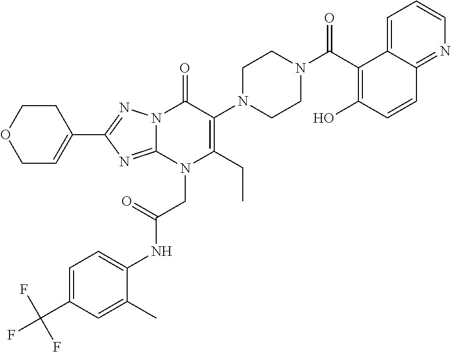 [SMILES: CCc1c(N2CCN(C(=O)c3c(O)ccc4ncccc34)CC2)c(=O)n2nc(C3=CCOCC3)nc2n1CC(=O)Nc1ccc(C(F)(F)F)cc1C] | 0.99 | 717.6 | UPLC- MS 3 |
| | 2-(2-(3,6-dihydro-2H-pyran-4-yl)-5-ethyl-6-(4-(6- | | | |
| | hydroxyquinoline-5-carbonyl)piperazin-1-yl)-7-oxo- | | | |
| | [1,2,4]triazolo[1,5-a]pyrimidin-4(7H)-yl)-N-(2-methyl-4- | | | |
| | (trifluoromethyl)phenyl)acetamide | | | |
| 19 | 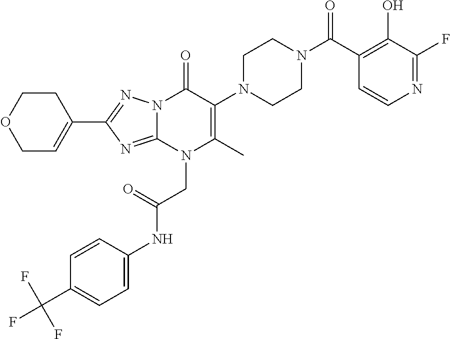 [SMILES: Cc1c(N2CCN(C(=O)c3ccnc(F)c3O)CC2)c(=O)n2nc(C3=CCOCC3)nc2n1CC(=O)Nc1ccc(C(F)(F)F)cc1] | 1.00 | 657.2 | UPLC- MS 3 |
| | 2-(2-(3,6-dihydro-2H-pyran-4-yl)-6-(4-(2-fluoro-3- | | | |
| | hydroxyisonicotinoyl)piperazin-1-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 20 | 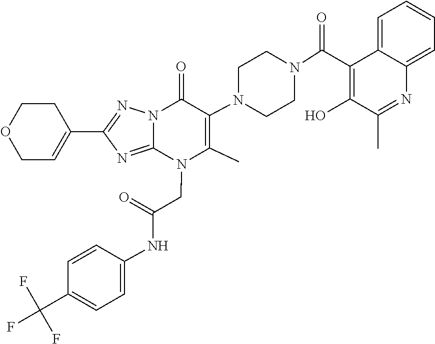 [SMILES: Cc1nc2ccccc2c(C(=O)N2CCN(c3c(C)n(CC(=O)Nc4ccc(C(F)(F)F)cc4)c4nc(C5=CCOCC5)nn4c3=O)CC2)c1O] | 1.05 | 703.3 | UPLC- MS 3 |
| | 2-(2-(3,6-dihydro-2H-pyran-4-yl)-6-(4-(3-hydroxy-2- | | | |
| | methylquinoline-4-carbonyl)piperazin-1-yl)-5-methyl- | | | |
| | 7-oxo-[1,2,4]triazolo[1,5-a]pyrimidin-4(7H)-yl)-N-(4- | | | |
| | (trifluoromethyl)phenyl)acetamide | | | |
| 21 | 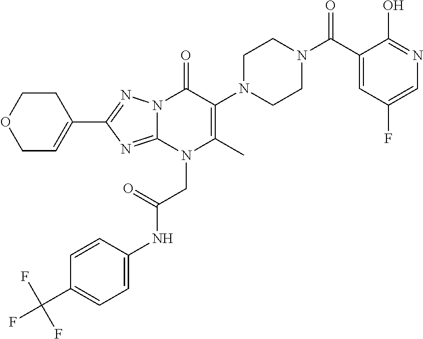 [SMILES: Cc1c(N2CCN(C(=O)c3cc(F)cnc3O)CC2)c(=O)n2nc(C3=CCOCC3)nc2n1CC(=O)Nc1ccc(C(F)(F)F)cc1] | 0.94 | 657.4 | UPLC- MS 3 |
| | 2-(2-(3,6-dihydro-2H-pyran-4-yl)-6-(4-(5-fluoro-2- | | | |
| | hydroxynicotinoyl)piperazin-1-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 22 | 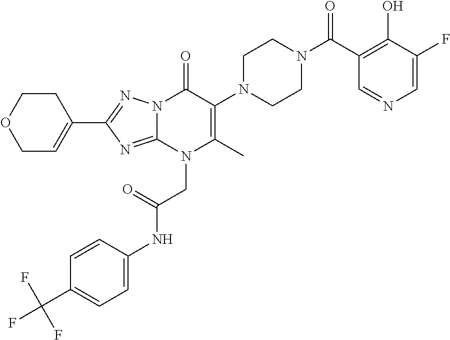 [SMILES: Cc1c(N2CCN(C(=O)c3cncc(F)c3O)CC2)c(=O)n2nc(C3=CCOCC3)nc2n1CC(=O)Nc1ccc(C(F)(F)F)cc1] | 0.85 | 657.3 | UPLC- MS 4 |
| | 2-(2-(3,6-dihydro-2H-pyran-4-yl)-6-(4-(5-fluoro-4- | | | |
| | hydroxynicotinoyl)piperazin-1-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 23 | 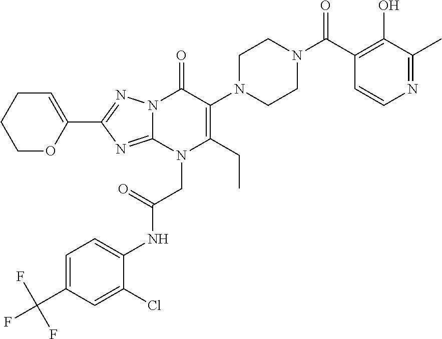 [SMILES: CCc1c(N2CCN(C(=O)c3ccnc(C)c3O)CC2)c(=O)n2nc(C3=CCCCO3)nc2n1CC(=O)Nc1ccc(C(F)(F)F)cc1Cl] | 1.42 | 701.3 | UPLC- MS 11 |
| | N-(2-chloro-4-(trifluoromethyl)phenyl)-2-(2-(3,4- | | | |
| | dihydro-2H-pyran-6-yl)-5-ethyl-6-(4-(3-hydroxy-2- | | | |
| | methylisonicotinoyl)piperazin-1-yl)-7-oxo- | | | |
| | [1,2,4]triazolo[1,5-a]pyrimidin-4(7H)-yl)acetamide | | | |
| 24 | 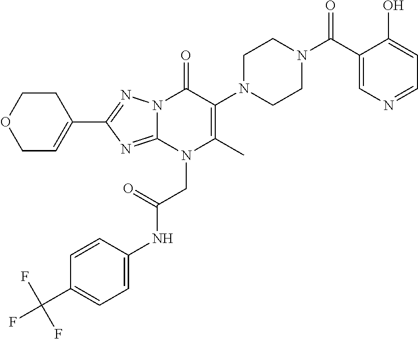 [SMILES: Cc1c(N2CCN(C(=O)c3cnccc3O)CC2)c(=O)n2nc(C3=CCOCC3)nc2n1CC(=O)Nc1ccc(C(F)(F)F)cc1] | 0.81 | 639.2 | UPLC- MS 8 |
| | 2-(2-(3,6-dihydro-2H-pyran-4-yl)-6-(4-(4- | | | |
| | hydroxynicotinoyl)piperazin-1-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 25 | 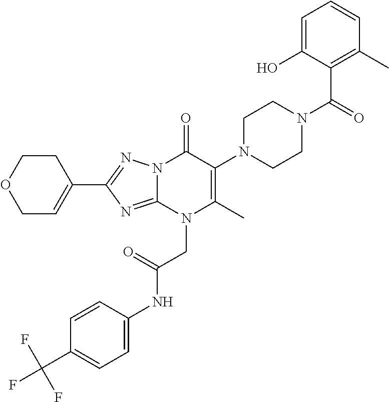 [SMILES: Cc1cccc(O)c1C(=O)N1CCN(c2c(C)n(CC(=O)Nc3ccc(C(F)(F)F)cc3)c3nc(C4=CCOCC4)nn3c2=O)CC1] | 1.02 | 652.4 | UPLC- MS 8 |
| | 2-(2-(3,6-dihydro-2H-pyran-4-yl)-6-(4-(2-hydroxy-6- | | | |
| | methylbenzoyl)piperazin-1-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 26 | 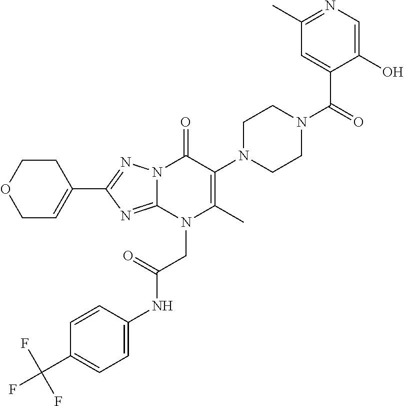 [SMILES: Cc1cc(C(=O)N2CCN(c3c(C)n(CC(=O)Nc4ccc(C(F)(F)F)cc4)c4nc(C5=CCOCC5)nn4c3=O)CC2)c(O)cn1] | 0.97 | 653.2 | UPLC- MS 4 |
| | 2-(2-(3,6-dihydro-2H-pyran-4-yl)-6-(4-(5-hydroxy-2- | | | |
| | methylisonicotinoyl)piperazin-1-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 27 | 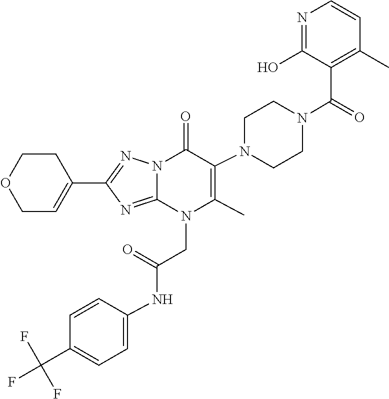 [SMILES: Cc1ccnc(O)c1C(=O)N1CCN(c2c(C)n(CC(=O)Nc3ccc(C(F)(F)F)cc3)c3nc(C4=CCOCC4)nn3c2=O)CC1] | 0.87 | 653.3 | UPLC- MS 8 |
| | 2-(2-(3,6-dihydro-2H-pyran-4-yl)-6-(4-(2-hydroxy-4- | | | |
| | methylnicotinoyl)piperazin-1-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 28 | 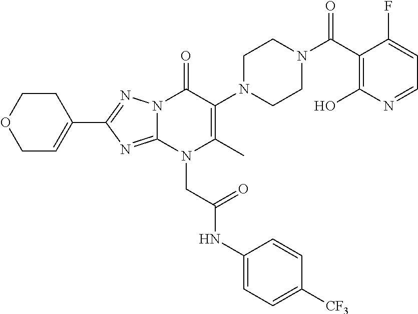 [SMILES: Cc1c(N2CCN(C(=O)c3c(F)ccnc3O)CC2)c(=O)n2nc(C3=CCOCC3)nc2n1CC(=O)Nc1ccc(C(F)(F)F)cc1] | 0.93 | 657.0 | UPLC- MS 1 |
| | 2-(2-(3,6-dihydro-2H-pyran-4-yl)-6-(4-(4-fluoro-2- | | | |
| | hydroxynicotinoyl)piperazin-1-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 29 | 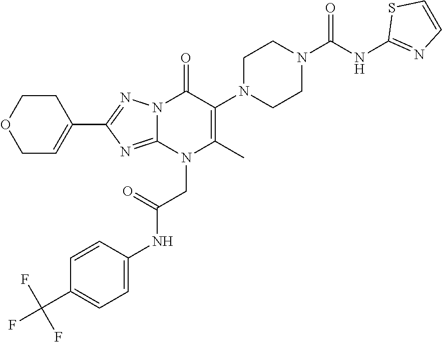 [SMILES: Cc1c(N2CCN(C(=O)Nc3nccs3)CC2)c(=O)n2nc(C3=CCOCC3)nc2n1CC(=O)Nc1ccc(C(F)(F)F)cc1] | 1.01 | 644.3 | UPLC- MS 4 |
| | 4-(2-(3,6-dihydro-2H-pyran-4-yl)-5-methyl-7-oxo-4-(2- | | | |
| | oxo-2-((4-(trifluoromethyl)phenyl)amino)ethyl)-4,7- | | | |
| | dihydro-[1,2,4]triazolo[1,5-a]pyrimidin-6-yl)-N-(thiazol- | | | |
| | 2-yl)piperazine-1-carboxamide | | | |
| 30 | 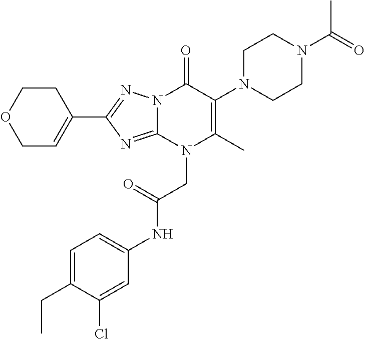 [SMILES: CCc1ccc(NC(=O)Cn2c(C)c(N3CCN(C(C)=O)CC3)c(=O)n3nc(C4=CCOCC4)nc23)cc1Cl] | 1.02 | 554.4 | UPLC- MS 8 |
| | 2-(6-(4-acetylpiperazin-1-yl)-2-(3,6-dihydro-2H-pyran- | | | |
| | 4-yl)-5-methyl-7-oxo-[1,2,4]triazolo[1,5-a]pyrimidin- | | | |
| | 4(7H)-yl)-N-(3-chloro-4-ethylphenyl)acetamide | | | |
| 31 | 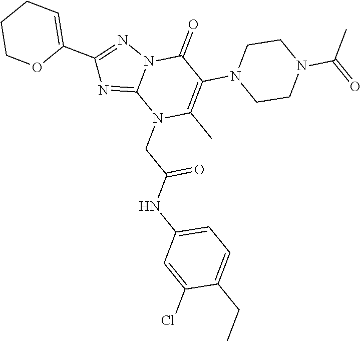 [SMILES: CCc1ccc(NC(=O)Cn2c(C)c(N3CCN(C(C)=O)CC3)c(=O)n3nc(C4=CCCCO4)nc23)cc1Cl] | 1.06 | 554.4 | UPLC- MS 8 |
| | 2-(6-(4-acetylpiperazin-1-yl)-2-(3,4-dihydro-2H-pyran- | | | |
| | 6-yl)-5-methyl-7-oxo-[1,2,4]triazolo[1,5-a]pyrimidin- | | | |
| | 4(7H)-yl)-N-(3-chloro-4-ethylphenyl)acetamide | | | |
| 32 | 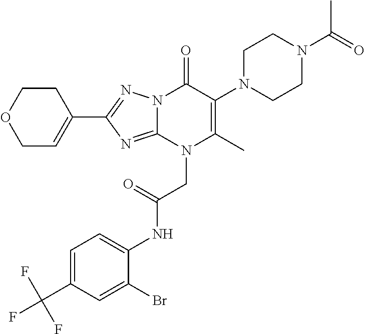 [SMILES: CC(=O)N1CCN(c2c(C)n(CC(=O)Nc3ccc(C(F)(F)F)cc3Br)c3nc(C4=CCOCC4)nn3c2=O)CC1] | 0.97 | 638.4 | UPLC- MS 1 |
| | 2-(6-(4-acetylpiperazin-1-yl)-2-(3,6-dihydro-2H-pyran- | | | |
| | 4-yl)-5-methyl-7-oxo-[1,2,4]triazolo[1,5-a]pyrimidin- | | | |
| | 4(7H)-yl)-N-(2-bromo-4- | | | |
| | (trifluoromethyl)phenyl)acetamide | | | |
| 33 | 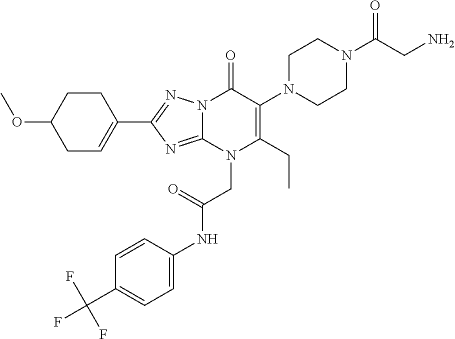 [SMILES: CCc1c(N2CCN(C(=O)CN)CC2)c(=O)n2nc(C3=CCC(OC)CC3)nc2n1CC(=O)Nc1ccc(C(F)(F)F)cc1] | 0.86 | 617.3 | UPLC- MS 8 |
| | 2-(5-ethyl-6-(4-glycylpiperazin-1-yl)-2-(4- | | | |
| | methoxycyclohex-1-en-1-yl)-7-oxo-[1,2,4]triazolo[1,5- | | | |
| | a]pyrimidin-4(7H)-yl)-N-(4- | | | |
| | (trifluoromethyl)phenyl)acetamide | | | |
| 34 | 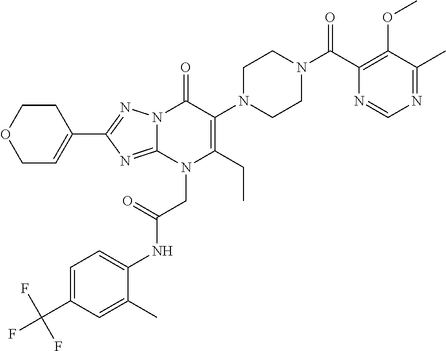 [SMILES: CCc1c(N2CCN(C(=O)c3ncnc(C)c3OC)CC2)c(=O)n2nc(C3=CCOCC3)nc2n1CC(=O)Nc1ccc(C(F)(F)F)cc1C] | 0.98 | 696.4 | UPLC- MS 1 |
| | 2-(2-(3,6-dihydro-2H-pyran-4-yl)-5-ethyl-6-(4-(5- | | | |
| | methoxy-6-methylpyrimidine-4-carbonyl)piperazin-1- | | | |
| | yl)-7-oxo-[1,2,4]triazolo[1,5-a]pyrimidin-4(7H)-yl)-N- | | | |
| | (2-methyl-4-(trifluoromethyl)phenyl)acetamide | | | |

### Route III

### Example 35: 2-(2-(3,6-dihydro-2H-pyran-4-yl)-6-(4-(6-hydroxypyrazolo[1,5-a]pyridine-7-carbonyl)piperazin-1-yl)-5-methyl-7-oxo-[1,2,4]triazolo[1,5-a]pyrimidin-4(7H)-yl)-N-(4-(trifluoromethyl)phenyl)acetamide

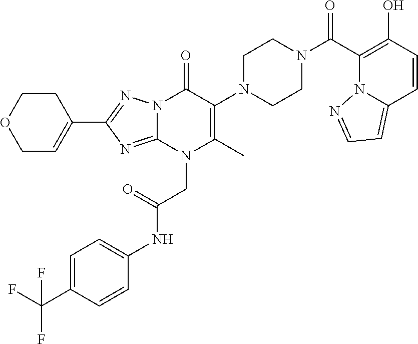 [SMILES: Cc1c(N2CCN(C(=O)c3c(O)ccc4ccnn34)CC2)c(=O)n2nc(C3=CCOCC3)nc2n1CC(=O)Nc1ccc(C(F)(F)F)cc1]

To a dark purple suspension of 6-hydroxypyrazolo[1,5-a]pyridine-7-carboxylic acid (Intermediate O) (16.5 mg, 85.0 μmol) in DCM (386 μL) were added HATU (44.1 mg, 116 μmol) then DIPEA (40.5 μL, 232 μmol). The RM was stirred for 5 minutes, then 2-(2-(3,6-dihydro-2H-pyran-4-yl)-5-methyl-7-oxo-6-(piperazin-1-yl)-[1,2,4]triazolo[1,5-a]pyrimidin-4(7H)-yl)-N-(4-(trifluoromethyl)phenyl)acetamide (Intermediate A) (40.0 mg, 77.0 μmol) was added. The RM was stirred at RT for 5 hours. The RM was diluted with DCM and washed with water. The aqueous phase was extracted once with DCM. The combined organic phases were dried through a phase separator, concentrated under reduced pressure, and dried under HV. The crude product was purified by column chromatography (RediSep Column: Silica 4 g, eluent DCM:DCM/MeOH (9/1) 100:0 to 0:100). The product containing fractions were combined, concentrated and dried under HV to give the title compound.

LC-MS: Rt=1.02 min; MS m/z [M+H]⁺ 678.1, m/z [M−H]⁻ 676.1; UPLC-MS 3

### Example 36: 2-(2-(3,6-dihydro-2H-pyran-4-yl)-6-(4-(3-hydroxyisonicotinoyl)piperazin-1-yl)-5-methyl-7-oxo-[1,2,4]triazolo[1,5-a]pyrimidin-4(7H)-yl)-N-(4-(trifluoromethyl)phenyl)acetamide

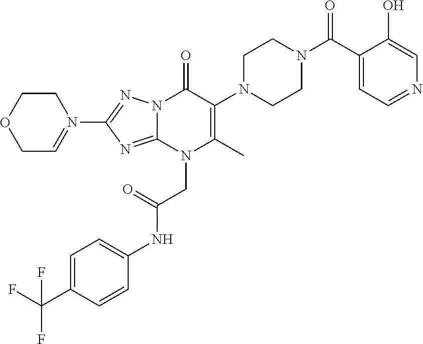

A solution of 3-hydroxyisonicotinic acid (53.8 mg, 386 μmol) in DCM (4 mL) was cooled to 0° C. Then 1-Chloro-N,N,2-trimethyl-1-propenylamine (56.0 μL, 425 μmol) was added and the RM was stirred at RT for 2 hours. The solution was cooled again to 0° C. and DIPEA (169 μL, 966 μmol) was added followed by 2-(2-(3,6-dihydro-2H-pyran-4-yl)-5-methyl-7-oxo-6-(piperazin-1-yl)-[1,2,4]triazolo[1,5-a]pyrimidin-4(7H)-yl)-N-(4-(trifluoromethyl)phenyl)acetamide (Intermediate A) (100 mg, 193 μmol). The RM was stirred at RT for 1 hour. The RM was diluted with DCM and washed with aq sat NaHCO₃. The organic layer was dried through a phase separator and concentrated under reduced pressure. The crude product was purified by reverse phase preparative HPLC (RP-HPLC acidic 15:20 to 50% B in 7 min, 50 to 100% B in 0.2 min) to give after lyophilization the title compound.

LC-MS: Rt=0.95 min; MS m/z [M+H]⁺ 639.4, m/z [M−H]⁻ 637.3; UPLC-MS 4

Examples 37 to 68 were made using analogous methods to Example 35 and 36, using methods known to the skilled chemist and using starting materials in the public domain.

| | | Rt | MS | LC-MS |
|---|---|---|---|---|
| Ex | Structure and name | [min] | [M + H]+ | method |
| 37 | 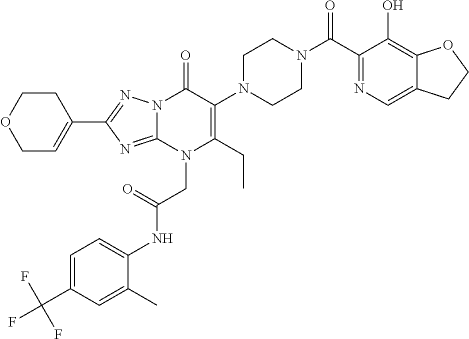 [SMILES: CCc1c(N2CCN(C(=O)c3ncc4c(c3O)OCC4)CC2)c(=O)n2nc(C3=CCOCC3)nc2n1CC(=O)Nc1ccc(C(F)(F)F)cc1C] | 0.91 | 709.3 | UPLC- MS 1 |
| | 2-(2-(3,6-dihydro-2H-pyran-4-yl)-5-ethyl-6-(4-(7-hydroxy-2,3- | | | |
| | dihydrofuro[3,2-c]pyridine-6-carbonyl)piperazin-1-yl)-7-oxo- | | | |
| | [1,2,4]triazolo[1,5-a]pyrimidin-4(7H)-yl)-N-(2-methyl-4- | | | |
| | (trifluoromethyl)phenyl)acetamide | | | |
| 38 | 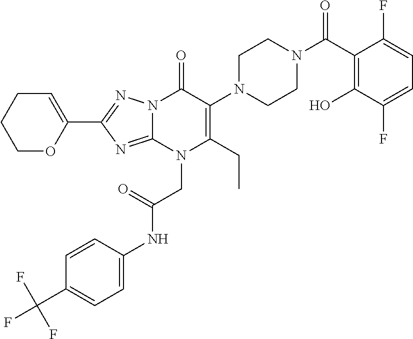 [SMILES: CCc1c(N2CCN(C(=O)c3c(F)ccc(F)c3O)CC2)c(=O)n2nc(C3=CCCCO3)nc2n1CC(=O)Nc1ccc(C(F)(F)F)cc1] | 1.09 | 688.3 | UPLC- MS 8 |
| | 2-(6-(4-(3,6-difluoro-2-hydroxybenzoyl)piperazin-1-yl)-2-(3,4- | | | |
| | dihydro-2H-pyran-6-yl)-5-ethyl-7-oxo-[1,2,4]triazolo[1,5-a] | | | |
| | pyrimidin-4(7H)-yl)-N-(4-(trifluoromethyl)phenyl)acetamide | | | |
| 39 | 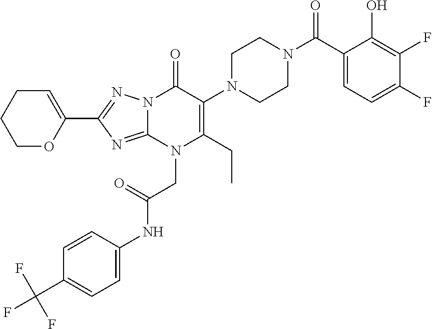 [SMILES: CCc1c(N2CCN(C(=O)c3ccc(F)c(F)c3O)CC2)c(=O)n2nc(C3=CCCCO3)nc2n1CC(=O)Nc1ccc(C(F)(F)F)cc1] | 1.13 | 688.7 | UPLC- MS 15 |
| | 2-(6-(4-(3,4-difluoro-2-hydroxybenzoyl)piperazin-1-yl)-2-(3,4- | | | |
| | dihydro-2H-pyran-6-yl)-5-ethyl-7-oxo-[1,2,4]triazolo[1,5-a] | | | |
| | pyrimidin-4(7H)-yl)-N-(4-(trifluoromethyl)phenyl)acetamide | | | |
| 40 | 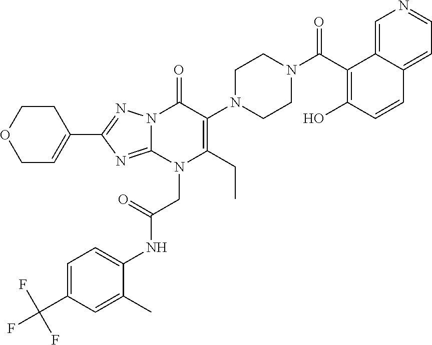 [SMILES: CCc1c(N2CCN(C(=O)c3c(O)ccc4ccncc34)CC2)c(=O)n2nc(C3=CCOCC3)nc2n1CC(=O)Nc1ccc(C(F)(F)F)cc1C] | 0.99 | 717.3 | UPLC- MS 3 |
| | 2-(2-(3,6-dihydro-2H-pyran-4-yl)-5-ethyl-6-(4-(7-hydroxyisoquin- | | | |
| | oline-8-carbonyl)piperazin-1-yl)-7-oxo-[1,2,4]triazolo[1,5-a]pyrim- | | | |
| | idin-4(7H)-yl)-N-(2-methyl-4-(trifluoromethyl)phenyl)acetamide | | | |
| 41 | 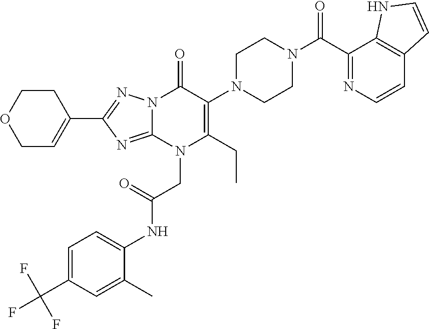 [SMILES: CCc1c(N2CCN(C(=O)c3nccc4cc[nH]c34)CC2)c(=O)n2nc(C3=CCOCC3)nc2n1CC(=O)Nc1ccc(C(F)(F)F)cc1C] | 1.01 | 690.5 | UPLC- MS 1 |
| | 2-(6-(4-(1H-pyrrolo[2,3-c]pyridine-7-carbonyl)piperazin-1-yl)-2- | | | |
| | (3,6-dihydro-2H-pyran-4-yl)-5-ethyl-7-oxo-[1,2,4]triazolo[1,5-a]pyr- | | | |
| | imidin-4(7H)-yl)-N-(2-methyl-4-(trifluoromethyl)phenyl)acetamide | | | |
| 42 | 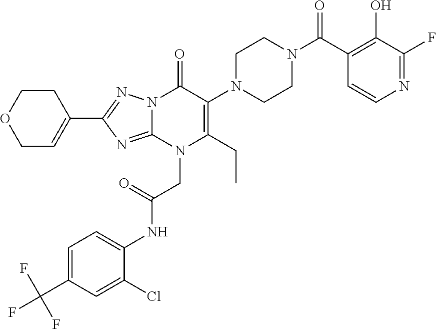 [SMILES: CCc1c(N2CCN(C(=O)c3ccnc(F)c3O)CC2)c(=O)n2nc(C3=CCOCC3)nc2n1CC(=O)Nc1ccc(C(F)(F)F)cc1Cl] | 1.51 | 705 | UPLC- MS 11 |
| | N-(2-chloro-4-(trifluoromethyl)phenyl)-2-(2-(3,6-dihydro-2H- | | | |
| | pyran-4-yl)-5-ethyl-6-(4-(2-fluoro-3-hydroxyisonicotinoyl)- | | | |
| | piperazin-1-yl)-7-oxo-[1,2,4]triazolo[1,5-a]pyrimidin-4(7H)- | | | |
| | yl)acetamide | | | |
| 43 | 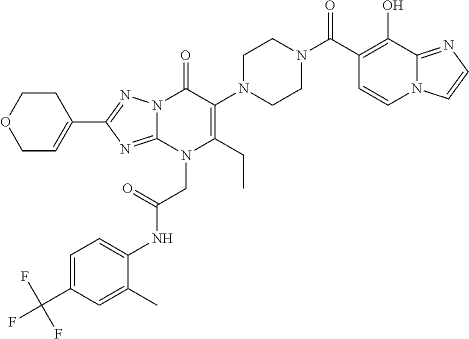 [SMILES: CCc1c(N2CCN(C(=O)c3ccn4ccnc4c3O)CC2)c(=O)n2nc(C3=CCOCC3)nc2n1CC(=O)Nc1ccc(C(F)(F)F)cc1C] | 1.35 | 706.3 | UPLC- MS 11 |
| | 2-(2-(3,6-dihydro-2H-pyran-4-yl)-5-ethyl-6-(4-(8-hydroxyimidazo | | | |
| | [1,2-a]pyridine-7-carbonyl)piperazin-1-yl)-7-oxo-[1,2,4]triazolo[1,5-a] | | | |
| | pyrimidin-4(7H)-yl)-N-(2-methyl-4-(trifluoromethyl)phenyl)acetamide | | | |
| 44 | 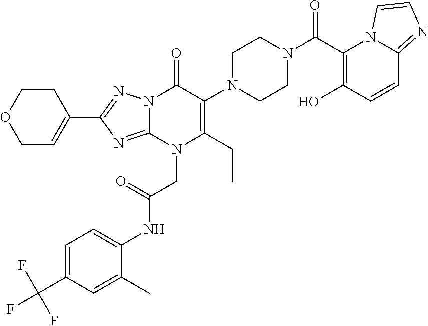 [SMILES: CCc1c(N2CCN(C(=O)c3c(O)ccc4nccn34)CC2)c(=O)n2nc(C3=CCOCC3)nc2n1CC(=O)Nc1ccc(C(F)(F)F)cc1C] | 0.91 | 706.3 | UPLC- MS 3 |
| | 2-(2-(3,6-dihydro-2H-pyran-4-yl)-5-ethyl-6-(4-(6-hydroxyimidazo | | | |
| | [1,2-a]pyridine-5-carbonyl)piperazin-1-yl)-7-oxo-[1,2,4]triazolo | | | |
| | [1,5-a]pyrimidin-4(7H)-yl)-N-(2-methyl-4-(trifluoromethyl)- | | | |
| | phenyl)acetamide | | | |
| 45 | 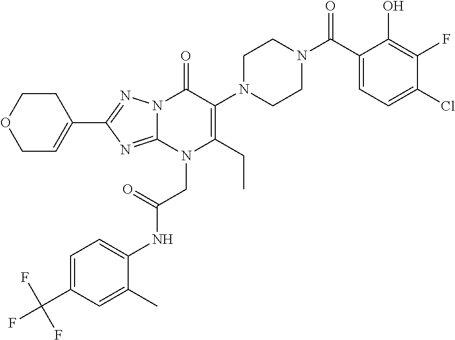 [SMILES: CCc1c(N2CCN(C(=O)c3ccc(Cl)c(F)c3O)CC2)c(=O)n2nc(C3=CCOCC3)nc2n1CC(=O)Nc1ccc(C(F)(F)F)cc1C] | 1.10 | 718.4 | UPLC- MS 3 |
| | 2-(6-(4-(4-chloro-3-fluoro-2-hydroxybenzoyl)piperazin-1-yl)-2- | | | |
| | (3,6-dihydro-2H-pyran-4-yl)-5-ethyl-7-oxo-[1,2,4]triazolo[1,5- | | | |
| | a]pyrimidin-4(7H)-yl)-N-(2-methyl-4-(trifluoromethyl)- | | | |
| | phenyl)acetamide | | | |
| 46 | 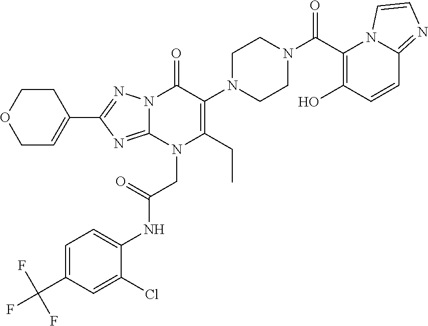 [SMILES: CCc1c(N2CCN(C(=O)c3c(O)ccc4nccn34)CC2)c(=O)n2nc(C3=CCOCC3)nc2n1CC(=O)Nc1ccc(C(F)(F)F)cc1Cl] | 0.91 | 726.3 | UPLC- MS 1 |
| | N-(2-chloro-4-(trifluoromethyl)phenyl)-2-(2-(3,6-dihydro-2H- | | | |
| | pyran-4-yl)-5-ethyl-6-(4-(6-hydroxyimidazo[1,2-a]pyridine- | | | |
| | 5-carbonyl)piperazin-1-yl)-7-oxo-[1,2,4]triazolo[1,5-a]pyrim- | | | |
| | idin-4(7H)-yl)acetamide | | | |
| 47 | 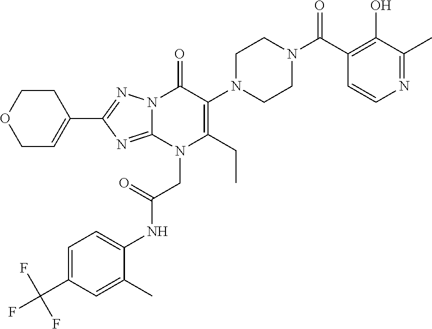 [SMILES: CCc1c(N2CCN(C(=O)c3ccnc(C)c3O)CC2)c(=O)n2nc(C3=CCOCC3)nc2n1CC(=O)Nc1ccc(C(F)(F)F)cc1C] | 0.96 | 681.4 | UPLC- MS 3 |
| | 2-(2-(3,6-dihydro-2H-pyran-4-yl)-5-ethyl-6-(4-(3-hydroxy-2- | | | |
| | methylisonicotinoyl)piperazin-1-yl)-7-oxo-[1,2,4]triazolo[1,5- | | | |
| | a]pyrimidin-4(7H)-yl)-N-(2-methyl-4-(trifluoromethyl)- | | | |
| | phenyl)acetamide | | | |
| 48 | 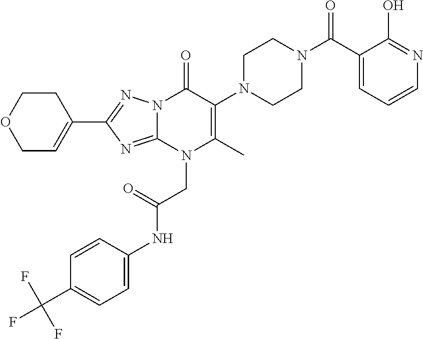 [SMILES: Cc1c(N2CCN(C(=O)c3cccnc3O)CC2)c(=O)n2nc(C3=CCOCC3)nc2n1CC(=O)Nc1ccc(C(F)(F)F)cc1] | 0.86 | 639.2 | UPLC- MS 15 |
| | 2-(2-(3,6-dihydro-2H-pyran-4-yl)-6-(4-(2-hydroxynicotinoyl)- | | | |
| | piperazin-1-yl)-5-methyl-7-oxo-[1,2,4]triazolo[1,5-a] | | | |
| | pyrimidin-4(7H)-yl)-N-(4-(trifluoromethyl)phenyl)acetamide | | | |
| 49 | 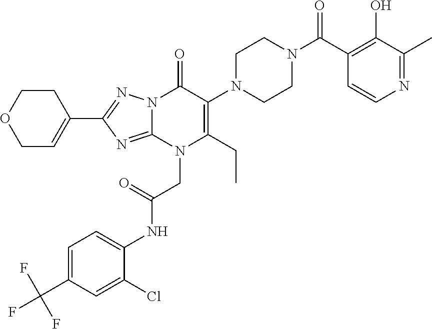 [SMILES: CCc1c(N2CCN(C(=O)c3ccnc(C)c3O)CC2)c(=O)n2nc(C3=CCOCC3)nc2n1CC(=O)Nc1ccc(C(F)(F)F)cc1Cl] | 0.94 | 701.4 | UPLC- MS 1 |
| | N-(2-chloro-4-(trifluoromethyl)phenyl)-2-(2-(3,6-dihydro-2H- | | | |
| | pyran-4-yl)-5-ethyl-6-(4-(3-hydroxy-2-methylisonicotinoyl)- | | | |
| | piperazin-1-yl)-7-oxo-[1,2,4]triazolo[1,5-a]pyrimidin-4(7H)- | | | |
| | yl)acetamide | | | |
| 50 | 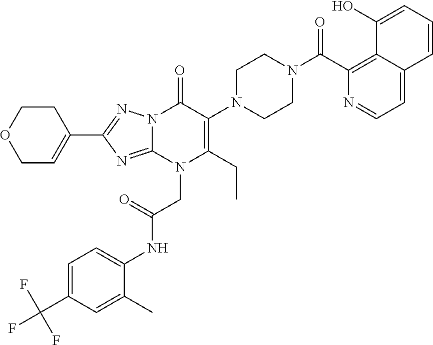 [SMILES: CCc1c(N2CCN(C(=O)c3nccc4cccc(O)c34)CC2)c(=O)n2nc(C3=CCOCC3)nc2n1CC(=O)Nc1ccc(C(F)(F)F)cc1C] | 1.06 | 717.6 | UPLC- MS 1 |
| | 2-(2-(3,6-dihydro-2H-pyran-4-yl)-5-ethyl-6-(4-(8-hydroxy- | | | |
| | isoquinoline-1-carbonyl)piperazin-1-yl)-7-oxo-[1,2,4]triazolo | | | |
| | [1,5-a]pyrimidin-4(7H)-yl)-N-(2-methyl-4-(trifluoromethyl)- | | | |
| | phenyl)acetamide | | | |
| 51 | 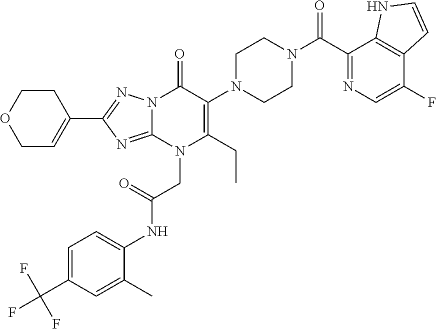 [SMILES: CCc1c(N2CCN(C(=O)c3ncc(F)c4cc[nH]c34)CC2)c(=O)n2nc(C3=CCOCC3)nc2n1CC(=O)Nc1ccc(C(F)(F)F)cc1C] | 1.17 | 708.4 | UPLC- MS 1 |
| | 2-(2-(3,6-dihydro-2H-pyran-4-yl)-5-ethyl-6-(4-(4-fluoro-1H- | | | |
| | pyrrolo[2,3-c]pyridine-7-carbonyl)piperazin-1-yl)-7-oxo- | | | |
| | [1,2,4]triazolo[1,5-a]pyrimidin-4(7H)-yl)-N-(2-methyl-4- | | | |
| | (trifluoromethyl)phenyl)acetamide | | | |
| 52 | 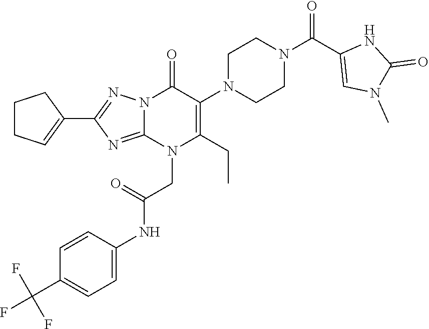 [SMILES: CCc1c(N2CCN(C(=O)c3cn(C)c(=O)[nH]3)CC2)c(=O)n2nc(C3=CCCC3)nc2n1CC(=O)Nc1ccc(C(F)(F)F)cc1] | 1.01 | 640.2 | UPLC- MS 4 |
| | 2-(2-(cyclopent-1-en-1-yl)-5-ethyl-6-(4-(1-methyl-2-oxo-2,3- | | | |
| | dihydro-1H-imidazole-4-carbonyl)piperazin-1-yl)-7-oxo-[1,2,4] | | | |
| | triazolo[1,5-a]pyrimidin-4(7H)-yl)-N-(4-(trifluoromethyl)- | | | |
| | phenyl)acetamide | | | |
| 53 | 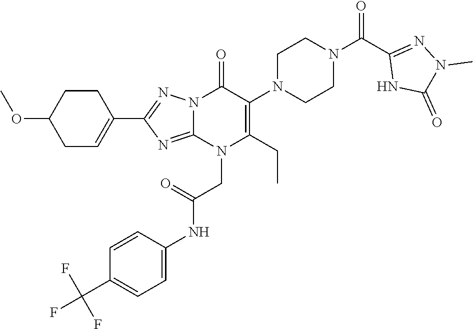 | 1.00 | 685.4 | UPLC- MS 15 |
| | 2-(5-ethyl-2-(4-methoxycyclohex-1-en-1-yl)-6-(4-(1-methyl-5-oxo- | | | |
| | 4,5-dihydro-1H-1,2,4-triazole-3-carbonyl)piperazin-1-yl)-7-oxo- | | | |
| | [1,2,4]triazolo[1,5-a]pyrimidin-4(7H)-yl)-N-(4-(trifluoromethyl)- | | | |
| | phenyl)acetamide | | | |
| 54 | 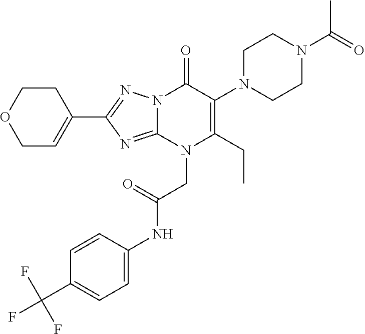 [SMILES: CCc1c(N2CCN(C(C)=O)CC2)c(=O)n2nc(C3=CCOCC3)nc2n1CC(=O)Nc1ccc(C(F)(F)F)cc1] | 0.92 | 574.2 | UPLC- MS 4 |
| | 2-(6-(4-acetylpiperazin-1-yl)-2-(3,6-dihydro-2H-pyran- | | | |
| | 4-yl)-5-ethyl-7-oxo-[1,2,4]triazolo[1,5-a]pyrimidin- | | | |
| | 4(7H)-yl)-N-(4-(trifluoromethyl)phenyl)acetamide | | | |
| 55 | 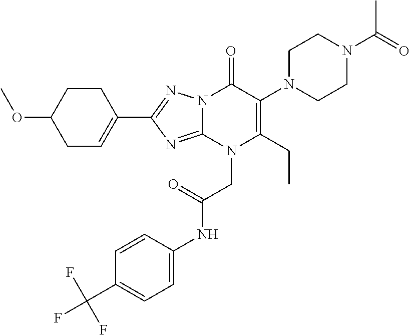 [SMILES: CCc1c(N2CCN(C(C)=O)CC2)c(=O)n2nc(C3=CCC(OC)CC3)nc2n1CC(=O)Nc1ccc(C(F)(F)F)cc1] | 1.03 | 602.3 | UPLC- MS 8 |
| | 2-(6-(4-acetylpiperazin-1-yl)-5-ethyl-2-(4-methoxycyclohex- | | | |
| | 1-en-1-yl)-7-oxo-[1,2,4]triazolo[1,5-a]pyrimidin-4(7H)- | | | |
| | yl)-N-(4-(trifluoromethyl)phenyl)acetamide | | | |
| 56 | 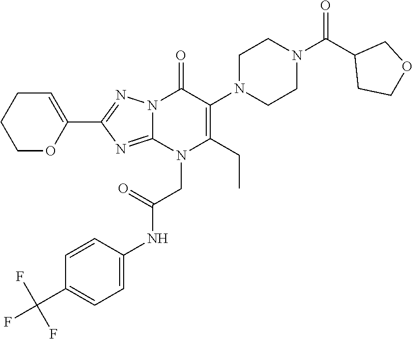 [SMILES: CCc1c(N2CCN(C(=O)C3CCOC3)CC2)c(=O)n2nc(C3=CCCCO3)nc2n1CC(=O)Nc1ccc(C(F)(F)F)cc1] | 1.03 | 630.3 | UPLC- MS 8 |
| | 2-(2-(3,4-dihydro-2H-pyran-6-yl)-5-ethyl-7-oxo-6-(4-(tetrahydro- | | | |
| | furan-3-carbonyl)piperazin-1-yl)-[1,2,4]triazolo[1,5-a]pyrimidin- | | | |
| | 4(7H)-yl)-N-(4-(trifluoromethyl)phenyl)acetamide | | | |
| 57 | 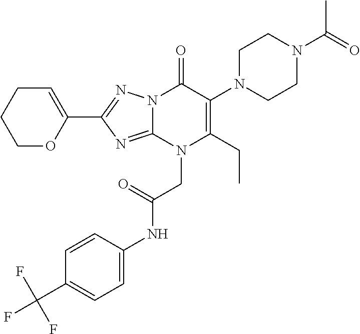 [SMILES: CCc1c(N2CCN(C(C)=O)CC2)c(=O)n2nc(C3=CCCCO3)nc2n1CC(=O)Nc1ccc(C(F)(F)F)cc1] | 1.01 | 574.3 | UPLC- MS 8 |
| | 2-(6-(4-acetylpiperazin-1-yl)-2-(3,4-dihydro-2H-pyran- | | | |
| | 6-yl)-5-ethyl-7-oxo-[1,2,4]triazolo[1,5-a]pyrimidin- | | | |
| | 4(7H)-yl)-N-(4-(trifluoromethyl)phenyl)acetamide | | | |
| 58 | 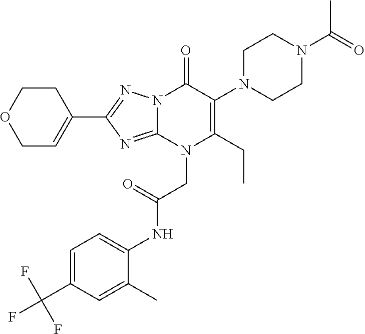 [SMILES: CCc1c(N2CCN(C(C)=O)CC2)c(=O)n2nc(C3=CCOCC3)nc2n1CC(=O)Nc1ccc(C(F)(F)F)cc1C] | 0.98 | 588.4 | UPLC- MS 4 |
| | 2-(6-(4-acetylpiperazin-1-yl)-2-(3,6-dihydro-2H-pyran-4- | | | |
| | yl)-5-ethyl-7-oxo-[1,2,4]triazolo[1,5-a]pyrimidin-4(7H)- | | | |
| | yl)-N-(2-methyl-4-(trifluoromethyl)phenyl)acetamide | | | |
| 59 | 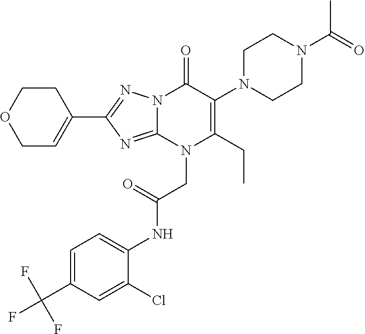 [SMILES: CCc1c(N2CCN(C(C)=O)CC2)c(=O)n2nc(C3=CCOCC3)nc2n1CC(=O)Nc1ccc(C(F)(F)F)cc1Cl] | 0.98 | 608.3 | UPLC- MS 1 |
| | 2-(6-(4-acetylpiperazin-1-yl)-2-(3,6-dihydro-2H-pyran-4- | | | |
| | yl)-5-ethyl-7-oxo-[1,2,4]triazolo[1,5-a]pyrimidin-4(7H)- | | | |
| | yl)-N-(2-chloro-4-(trifluoromethyl)phenyl)acetamide | | | |
| 60 | 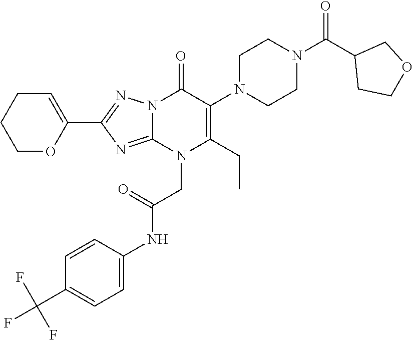 [SMILES: CCc1c(N2CCN(C(=O)C3CCOC3)CC2)c(=O)n2nc(C3=CCCCO3)nc2n1CC(=O)Nc1ccc(C(F)(F)F)cc1] | 1.03 | 630.3 | UPLC- MS 8 |
| | 2-(2-(3,4-dihydro-2H-pyran-6-yl)-5-ethyl-7-oxo-6-(4-(tetra- | | | |
| | hydrofuran-3-carbonyl)piperazin-1-yl)-[1,2,4]triazolo[1,5-a] | | | |
| | pyrimidin-4(7H)-yl)-N-(4-(trifluoromethyl)phenyl)acetamide | | | |
| 61 | 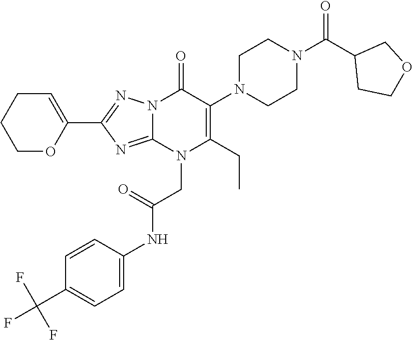 [SMILES: CCc1c(N2CCN(C(=O)C3CCOC3)CC2)c(=O)n2nc(C3=CCCCO3)nc2n1CC(=O)Nc1ccc(C(F)(F)F)cc1] | 1.03 | 630.3 | UPLC- MS 8 |
| | 2-(2-(3,4-dihydro-2H-pyran-6-yl)-5-ethyl-7-oxo-6-(4-(tetra- | | | |
| | hydrofuran-3-carbonyl)piperazin-1-yl)-[1,2,4]triazolo[1,5-a] | | | |
| | pyrimidin-4(7H)-yl)-N-(4-(trifluoromethyl)phenyl)acetamide | | | |
| 62 | 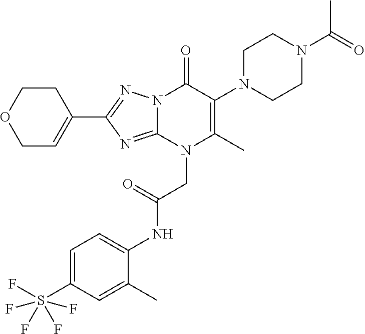 [SMILES: CC(=O)N1CCN(c2c(C)n(CC(=O)Nc3ccc(S(F)(F)(F)(F)F)cc3C)c3nc(C4=CCOCC4)nn3c2=O)CC1] | 1.00 | 632.3 | UPLC- MS 1 |
| | 2-(6-(4-acetylpiperazin-1-yl)-2-(3,6-dihydro-2H-pyran- | | | |
| | 4-yl)-5-methyl-7-oxo-[1,2,4]triazolo[1,5-a]pyrimidin- | | | |
| | 4(7H)-yl)-N-(2-methyl-4-(pentafluoro-l6-sulfaneyl)- | | | |
| | phenyl)acetamide | | | |
| 63 | 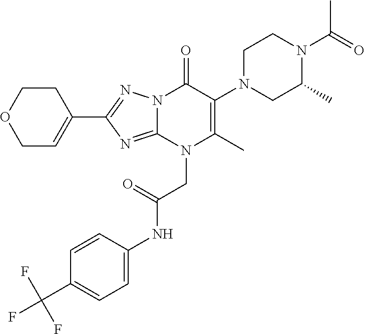 [SMILES: CC(=O)N1CCN(c2c(C)n(CC(=O)Nc3ccc(C(F)(F)F)cc3)c3nc(C4=CCOCC4)nn3c2=O)C[C@H]1C] | 0.94 | 574.2 | UPLC- MS 4 |
| | (R)-2-(6-(4-acetyl-3-methylpiperazin-1-yl)-2-(3,6- | | | |
| | dihydro-2H-pyran-4-yl)-5-methyl-7-oxo-[1,2,4]triazolo | | | |
| | [1,5-a]pyrimidin-4(7H)-yl)-N-(4-(trifluoromethyl)- | | | |
| | phenyl)acetamide | | | |
| 64 | 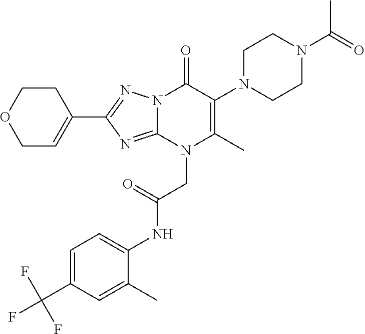 [SMILES: CC(=O)N1CCN(c2c(C)n(CC(=O)Nc3ccc(C(F)(F)F)cc3C)c3nc(C4=CCOCC4)nn3c2=O)CC1] | 0.96 | 574.3 | UPLC- MS 4 |
| | 2-(6-(4-acetylpiperazin-1-yl)-2-(3,6-dihydro-2H-pyran-4- | | | |
| | yl)-5-methyl-7-oxo-[1,2,4]triazolo[1,5-a]pyrimidin-4(7H)- | | | |
| | yl)-N-(2-methyl-4-(trifluoromethyl)phenyl)acetamide | | | |
| 65 | 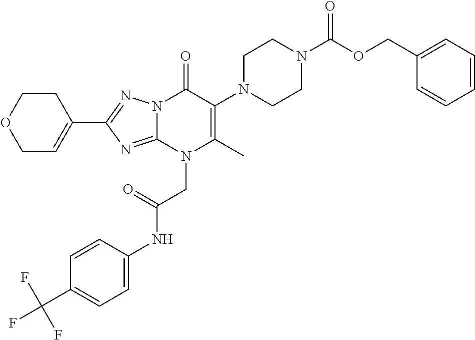 [SMILES: Cc1c(N2CCN(C(=O)OCc3ccccc3)CC2)c(=O)n2nc(C3=CCOCC3)nc2n1CC(=O)Nc1ccc(C(F)(F)F)cc1] | 1.22 | 652.3 | UPLC- MS 3 |
| | benzyl 4-(2-(3,6-dihydro-2H-pyran-4-yl)-5-methyl-7-oxo-4-(2-oxo- | | | |
| | 2-((4-(trifluoromethyl)phenyl)amino)ethyl)-4,7-dihydro-[1,2,4] | | | |
| | triazolo[1,5-a]pyrimidin-6-yl)piperazine-1-carboxylate | | | |
| 66 | 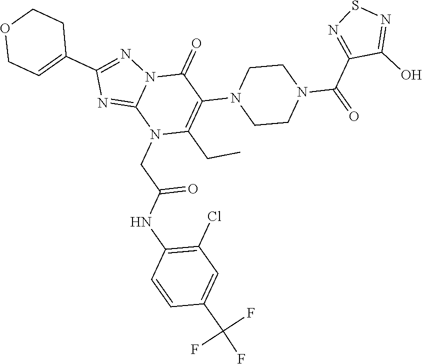 [SMILES: CCc1c(N2CCN(C(=O)c3nsnc3O)CC2)c(=O)n2nc(C3=CCOCC3)nc2n1CC(=O)Nc1ccc(C(F)(F)F)cc1Cl] | 1.08 | 694.1 | UPLC- MS 1 |
| | N-(2-chloro-4-(trifluoromethyl)phenyl)-2-(2-(3,6-dihydro-2H- | | | |
| | pyran-4-yl)-5-ethyl-6-(4-(4-hydroxy-1,2,5-thiadiazole-3- | | | |
| | carbonyl)piperazin-1-yl)-7-oxo-[1,2,4]triazolo[1,5-a] | | | |
| | pyrimidin-4(7H)-yl)acetamide | | | |
| 67 | 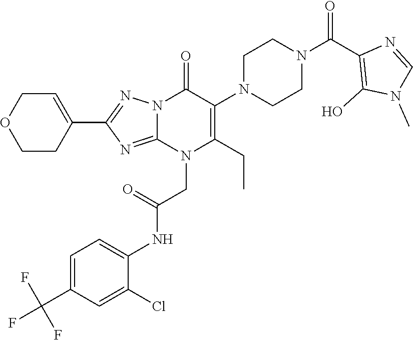 [SMILES: CCc1c(N2CCN(C(=O)c3ncn(C)c3O)CC2)c(=O)n2nc(C3=CCOCC3)nc2n1CC(=O)Nc1ccc(C(F)(F)F)cc1Cl] | 4.53 | 690.4 | UPLC- MS 17 |
| | N-(2-chloro-4-(trifluoromethyl)phenyl)-2-(2-(3,6-dihydro-2H- | | | |
| | pyran-4-yl)-5-ethyl-6-(4-(5-hydroxy-1-methyl-1H-imidazole- | | | |
| | 4-carbonyl)piperazin-1-yl)-7-oxo-[1,2,4]triazolo[1,5-a] | | | |
| | pyrimidin-4(7H)-yl)acetamide | | | |
| 68 | 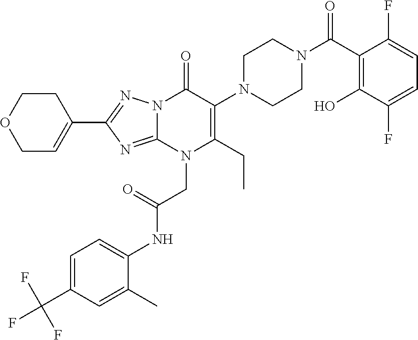 [SMILES: CCc1c(N2CCN(C(=O)c3c(F)ccc(F)c3O)CC2)c(=O)n2nc(C3=CCOCC3)nc2n1CC(=O)Nc1ccc(C(F)(F)F)cc1C] | 1.03 | 702.5 | UPLC- MS 3 |
| | 2-(6-(4-(3,6-difluoro-2-hydroxybenzoyl)piperazin-1-yl)-2-(3,6- | | | |
| | dihydro-2H-pyran-4-yl)-5-ethyl-7-oxo-[1,2,4]triazolo[1,5-a] | | | |
| | pyrimidin-4(7H)-yl)-N-(2-methyl-4-(trifluoromethyl)- | | | |
| | phenyl)acetamide | | | |

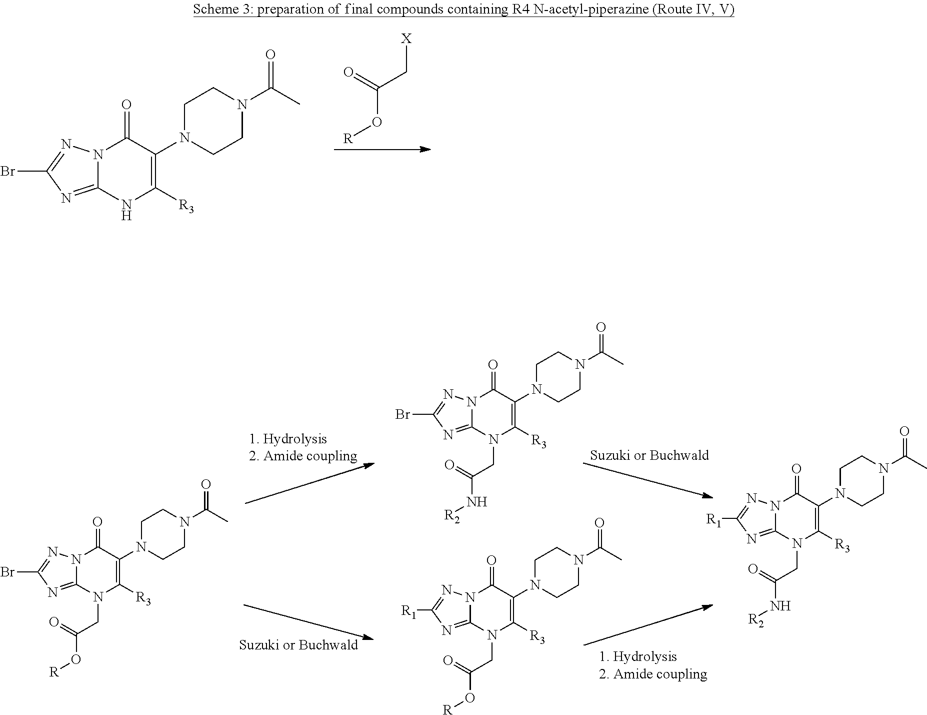

### Route IV

### Example 69: 2-(6-(4-acetylpiperazin-1-yl)-2-(3,6-dihydro-2H-pyran-4-yl)-5-methyl-7-oxo-[1,2,4]triazolo[1,5-a]pyrimidin-4(7H)-yl)-N-(4-(trifluoromethyl)phenyl)acetamide

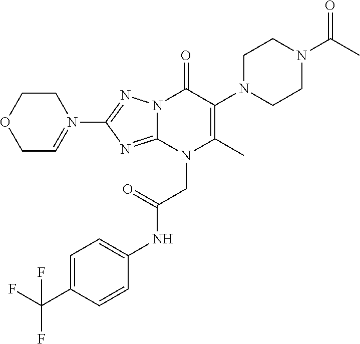

To a solution of 2-(6-(4-acetylpiperazin-1-yl)-2-(3,6-dihydro-2H-pyran-4-yl)-5-methyl-7-oxo-[1,2,4]triazolo[1,5-a]pyrimidin-4(7H)-yl)acetic acid (Intermediate H) (64.0 mg, 98.0 μmol) and 4-aminobenzotrifluoride (15.0 μL, 117 μmol) in DMF (500 μL) were added Et₃N (54.0 μL, 391 μmol) and T₃P 50% in DMF (116 μL, 195 μmol). The brown solution mixture was stirred at RT for 4.5 hours. 4-Aminobenzotrifluoride (15.0 μL, 117 μmol), T₃P 50% in DMF (116 μL, 195 μmol) and Et₃N (54.0 μL, 391 μmol) were added and the RM was stirred at RT for 2.5 days, then at 50° C. for 4 hours. The RM was purified by reverse phase preparative HPLC (RP-HPLC acidic 1:28 to 58% B in 20 min). The product containing fractions were combined and basified with a small amount of aq sat NaHCO₃. The ACN was removed under reduced pressure and the resulting solid was filtered off, washed with water, and dried under HV to give the title compound as a white solid.

LC-MS: Rt=0.93 min; MS m/z [M+H]⁺ 560.4, m/z [M−H]⁻ 558.4; UPLC-MS 8

Examples 70 to 103 were made using analogous methods to Example 69, using methods known to the skilled chemist and using starting materials in the public domain.

| | | Rt | MS | LC-MS |
|---|---|---|---|---|
| Ex. | Structure and name | [min] | [M + H]+ | method |
| 70 | 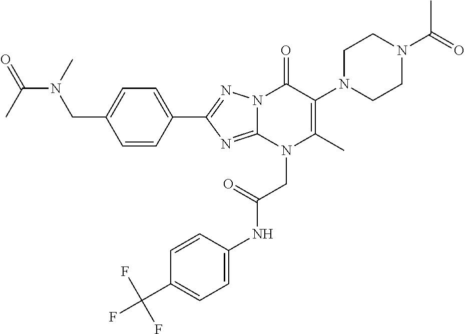 [SMILES: CC(=O)N(C)Cc1ccc(-c2nc3n(CC(=O)Nc4ccc(C(F)(F)F)cc4)c(C)c(N4CCN(C(C)=O)CC4)c(=O)n3n2)cc1] | 0.94 | 639.4 | UPLC- MS 15 |
| | 2-(6-(4-acetylpiperazin-1-yl)-5-methyl-2-(4-((N-methylacetamido)- | | | |
| | methyl)phenyl)-7-oxo-[1,2,4]triazolo[1,5-a]pyrimidin-4(7H)-yl)- | | | |
| | N-(4-(trifluoromethyl)phenyl)acetamide | | | |
| 71 | 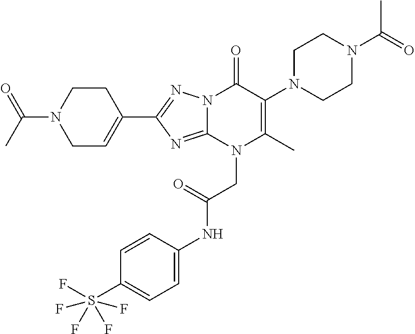 [SMILES: CC(=O)N1CC=C(c2nc3n(CC(=O)Nc4ccc(S(F)(F)(F)(F)F)cc4)c(C)c(N4CCN(C(C)=O)CC4)c(=O)n3n2)CC1] | 0.92 | 659.5 | UPLC- MS 8 |
| | 2-(2-(1-acetyl-1,2,3,6-tetrahydropyridin-4-yl)-6-(4-acetyl- | | | |
| | piperazin-1-yl)-5-methyl-7-oxo-[1,2,4]triazolo[1,5-a]pyrimidin- | | | |
| | 4(7H)-yl)-N-(4-(pentafluoro-l6-sulfaneyl)phenyl)acetamide | | | |
| 72 | 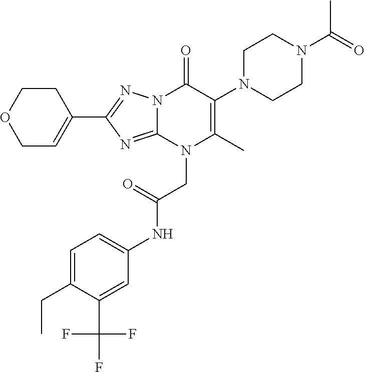 [SMILES: CCc1ccc(NC(=O)Cn2c(C)c(N3CCN(C(C)=O)CC3)c(=O)n3nc(C4=CCOCC4)nc23)cc1C(F)(F)F] | 1.05 | 588.2 | UPLC- MS 4 |
| | 2-(6-(4-acetylpiperazin-1-yl)-2-(3,6-dihydro-2H-pyran-4- | | | |
| | yl)-5-methyl-7-oxo-[1,2,4]triazolo[1,5-a]pyrimidin-4(7H)- | | | |
| | yl)-N-(4-ethyl-3-(trifluoromethyl)phenyl)acetamide | | | |
| 73 | 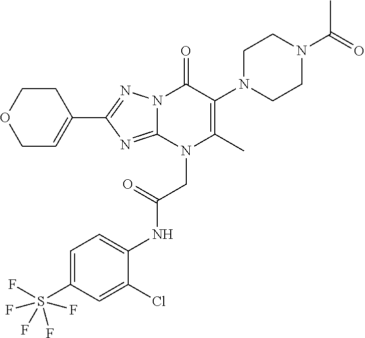 [SMILES: CC(=O)N1CCN(c2c(C)n(CC(=O)Nc3ccc(S(F)(F)(F)(F)F)cc3Cl)c3nc(C4=CCOCC4)nn3c2=O)CC1] | 1.03 | 652.3 | UPLC- MS 1 |
| | 2-(6-(4-acetylpiperazin-1-yl)-2-(3,6-dihydro-2H-pyran-4-yl)- | | | |
| | 5-methyl-7-oxo-[1,2,4]triazolo[1,5-a]pyrimidin-4(7H)-yl)- | | | |
| | N-(2-chloro-4-(pentafluoro-l6-sulfaneyl)phenyl)acetamide | | | |
| 74 | 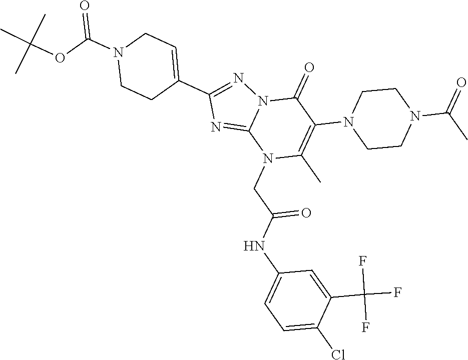 [SMILES: CC(=O)N1CCN(c2c(C)n(CC(=O)Nc3ccc(Cl)c(C(F)(F)F)c3)c3nc(C4=CCN(C(=O)OC(C)(C)C)CC4)nn3c2=O)CC1] | 1.19 | 693.4 | UPLC- MS 15 |
| | tert-butyl 4-(6-(4-acetylpiperazin-1-yl)-4-(2-((4-chloro-3-(trifluoro- | | | |
| | methyl)phenyl)amino)-2-oxoethyl)-5-methyl-7-oxo-4,7-dihydro- | | | |
| | [1,2,4]triazolo[1,5-a]pyrimidin-2-yl)-5,6-dihydropyridine- | | | |
| | 1(2H)-carboxylate | | | |
| 75 | 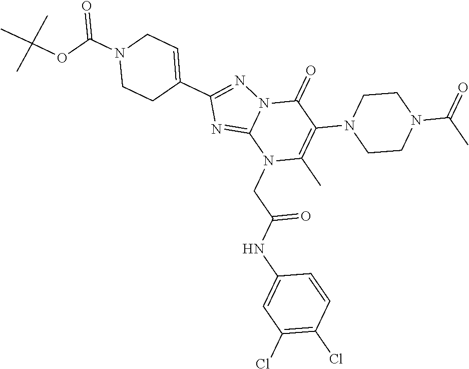 [SMILES: CC(=O)N1CCN(c2c(C)n(CC(=O)Nc3ccc(Cl)c(Cl)c3)c3nc(C4=CCN(C(=O)OC(C)(C)C)CC4)nn3c2=O)CC1] | 1.17 | 659.3 | UPLC- MS 15 |
| | tert-butyl 4-(6-(4-acetylpiperazin-1-yl)-4-(2-((3,4-dichlorophenyl)- | | | |
| | amino)-2-oxoethyl)-5-methyl-7-oxo-4,7-dihydro-[1,2,4]triazolo | | | |
| | [1,5-a]pyrimidin-2-yl)-5,6-dihydropyridine-1(2H)-carboxylate | | | |
| 76 | Intentionally blank | | | |
| 77 | 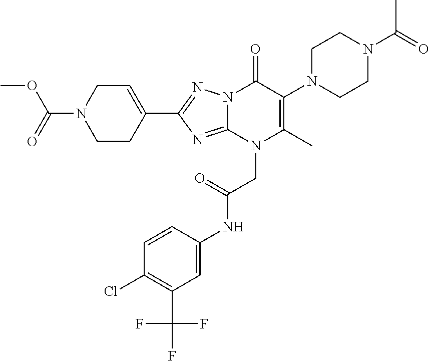 [SMILES: COC(=O)N1CC=C(c2nc3n(CC(=O)Nc4ccc(Cl)c(C(F)(F)F)c4)c(C)c(N4CCN(C(C)=O)CC4)c(=O)n3n2)CC1] | 1.04 | 651.3 | UPLC- MS 15 |
| | methyl 4-(6-(4-acetylpiperazin-1-yl)-4-(2-((4-chloro-3-(trifluoro- | | | |
| | methyl)phenyl)amino)-2-oxoethyl)-5-methyl-7-oxo-4,7-dihydro- | | | |
| | [1,2,4]triazolo[1,5-a]pyrimidin-2-yl)-5,6-dihydropyridine- | | | |
| | 1(2H)-carboxylate | | | |
| 78 | 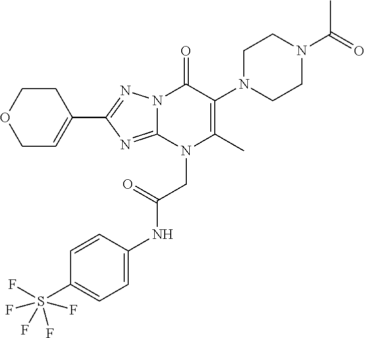 [SMILES: CC(=O)N1CCN(c2c(C)n(CC(=O)Nc3ccc(S(F)(F)(F)(F)F)cc3)c3nc(C4=CCOCC4)nn3c2=O)CC1] | 0.98 | 618.4 | UPLC- MS 8 |
| | 2-(6-(4-acetylpiperazin-1-yl)-2-(3,6-dihydro-2H-pyran-4- | | | |
| | yl)-5-methyl-7-oxo-[1,2,4]triazolo[1,5-a]pyrimidin-4(7H)- | | | |
| | yl)-N-(4-(pentafluoro-l6-sulfaneyl)phenyl)acetamide | | | |
| 79 | 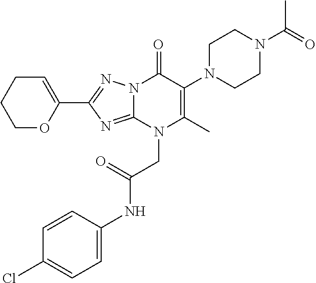 [SMILES: CC(=O)N1CCN(c2c(C)n(CC(=O)Nc3ccc(Cl)cc3)c3nc(C4=CCCCO4)nn3c2=O)CC1] | 0.93 | 526.2 | UPLC- MS 8 |
| | 2-(6-(4-acetylpiperazin-1-yl)-2-(3,4-dihydro-2H- | | | |
| | pyran-6-yl)-5-methyl-7-oxo-[1,2,4]triazolo[1,5-a] | | | |
| | pyrimidin-4(7H)-yl)-N-(4-chlorophenyl)acetamide | | | |
| 80 | 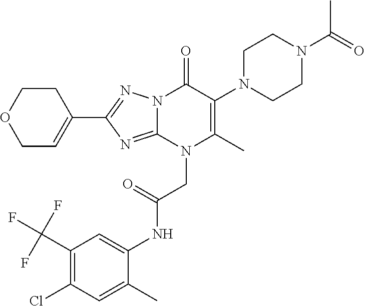 [SMILES: CC(=O)N1CCN(c2c(C)n(CC(=O)Nc3cc(C(F)(F)F)c(Cl)cc3C)c3nc(C4=CCOCC4)nn3c2=O)CC1] | 0.93 | 608.3 | UPLC- MS 3 |
| | 2-(6-(4-acetylpiperazin-1-yl)-2-(3,6-dihydro-2H- | | | |
| | pyran-4-yl)-5-methyl-7-oxo-[1,2,4]triazolo[1,5- | | | |
| | a]pyrimidin-4(7H)-yl)-N-(4-chloro-2-methyl-5- | | | |
| | (trifluoromethyl)phenyl)acetamide | | | |
| 81 | 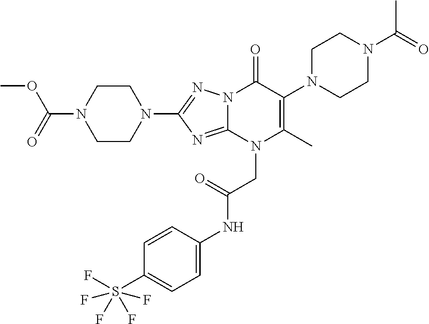 [SMILES: COC(=O)N1CCN(c2nc3n(CC(=O)Nc4ccc(S(F)(F)(F)(F)F)cc4)c(C)c(N4CCN(C(C)=O)CC4)c(=O)n3n2)CC1] | 0.98 | 678.1 | UPLC- MS 8 |
| | methyl 4-(6-(4-acetylpiperazin-1-yl)-5-methyl-7-oxo-4-(2- | | | |
| | oxo-2-((4-(pentafluoro-l6-sulfaneyl)phenyl)amino)ethyl)- | | | |
| | 4,7-dihydro-[1,2,4]triazolo[1,5-a]pyrimidin-2-yl)piper- | | | |
| | azine-1-carboxylate | | | |
| 82 | 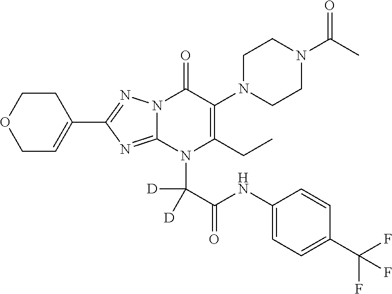 [SMILES: [2H]C([2H])(C(=O)Nc1ccc(C(F)(F)F)cc1)n1c(CC)c(N2CCN(C(C)=O)CC2)c(=O)n2nc(C3=CCOCC3)nc12] | 0.97 | 576.4 | UPLC- MS 1 |
| | 2-(6-(4-acetylpiperazin-1-yl)-2-(3,6-dihydro-2H-pyran-4- | | | |
| | yl)-5-ethyl-7-oxo-[1,2,4]triazolo[1,5-a]pyrimidin-4(7H)- | | | |
| | yl)-N-(4-(trifluoromethyl)phenyl)acetamide-2,2-d2 | | | |
| 83 | 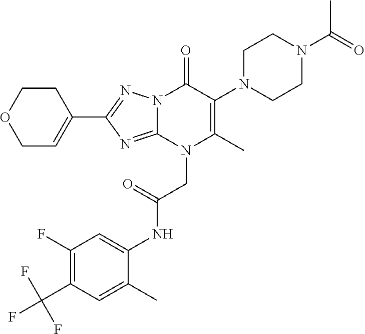 [SMILES: CC(=O)N1CCN(c2c(C)n(CC(=O)Nc3cc(F)c(C(F)(F)F)cc3C)c3nc(C4=CCOCC4)nn3c2=O)CC1] | 0.98 | 592.3 | UPLC- MS 4 |
| | 2-(6-(4-acetylpiperazin-1-yl)-2-(3,6-dihydro-2H-pyran-4-yl)- | | | |
| | 5-methyl-7-oxo-[1,2,4]triazolo[1,5-a]pyrimidin-4(7H)-yl)- | | | |
| | N-(5-fluoro-2-methyl-4-(trifluoromethyl)phenyl)acetamide | | | |
| 84 | 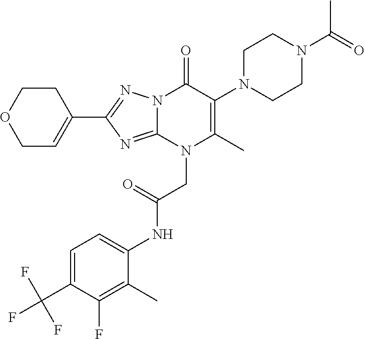 [SMILES: CC(=O)N1CCN(c2c(C)n(CC(=O)Nc3ccc(C(F)(F)F)c(F)c3C)c3nc(C4=CCOCC4)nn3c2=O)CC1] | 1.01 | 592.2 | UPLC- MS 3 |
| | 2-(6-(4-acetylpiperazin-1-yl)-2-(3,6-dihydro-2H-pyran- | | | |
| | 4-yl)-5-methyl-7-oxo-[1,2,4]triazolo[1,5-a]pyrimidin- | | | |
| | 4(7H)-yl)-N-(3-fluoro-2-methyl-4-(trifluoromethyl)- | | | |
| | phenyl)acetamide | | | |
| 85 | 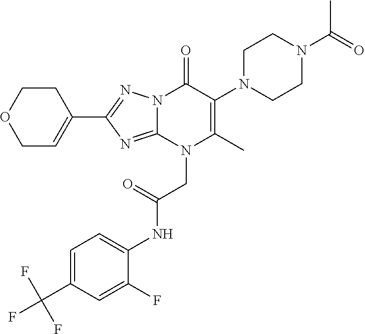 [SMILES: CC(=O)N1CCN(c2c(C)n(CC(=O)Nc3ccc(C(F)(F)F)cc3F)c3nc(C4=CCOCC4)nn3c2=O)CC1] | 0.96 | 578.3 | UPLC- MS 4 |
| | 2-(6-(4-acetylpiperazin-1-yl)-2-(3,6-dihydro-2H-pyran-4- | | | |
| | yl)-5-methyl-7-oxo-[1,2,4]triazolo[1,5-a]pyrimidin-4(7H)- | | | |
| | yl)-N-(2-fluoro-4-(trifluoromethyl)phenyl)acetamide | | | |
| 86 | 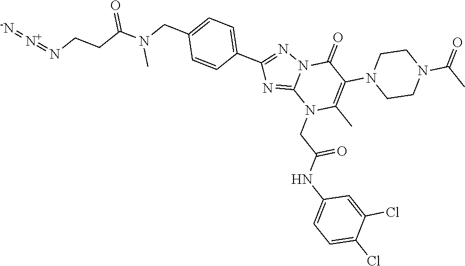 | 1.04 | 694.5 | UPLC- MS 8 |
| | N-(4-(6-(4-acetylpiperazin-1-yl)-4-(2-((3,4-dichlorophenyl)amino)- | | | |
| | 2-oxoethyl)-5-methyl-7-oxo-4,7-dihydro-[1,2,4]triazolo[1,5-a] | | | |
| | pyrimidin-2-yl)benzyl)-3-azido-N-methylpropanamide | | | |
| 87 | 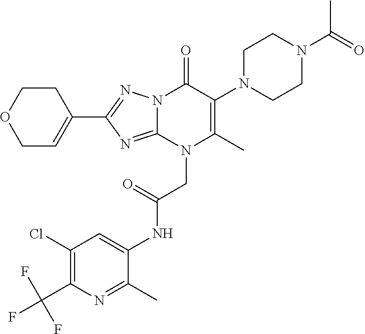 [SMILES: CC(=O)N1CCN(c2c(C)n(CC(=O)Nc3cc(Cl)c(C(F)(F)F)nc3C)c3nc(C4=CCOCC4)nn3c2=O)CC1] | 0.98 | 609.5 | UPLC- MS 3 |
| | 2-(6-(4-acetylpiperazin-1-yl)-2-(3,6-dihydro-2H-pyran- | | | |
| | 4-yl)-5-methyl-7-oxo-[1,2,4]triazolo[1,5-a]pyrimidin- | | | |
| | 4(7H)-yl)-N-(5-chloro-2-methyl-6-(trifluoromethyl)- | | | |
| | pyridin-3-yl)acetamide | | | |
| 88 | 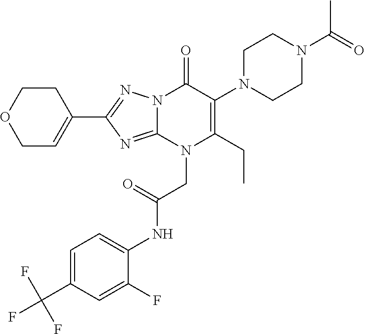 [SMILES: CCc1c(N2CCN(C(C)=O)CC2)c(=O)n2nc(C3=CCOCC3)nc2n1CC(=O)Nc1ccc(C(F)(F)F)cc1F] | 0.98 | 592.3 | UPLC- MS 4 |
| | 2-(6-(4-acetylpiperazin-1-yl)-2-(3,6-dihydro-2H-pyran-4- | | | |
| | yl)-5-ethyl-7-oxo-[1,2,4]triazolo[1,5-a]pyrimidin-4(7H)- | | | |
| | yl)-N-(2-fluoro-4-(trifluoromethyl)phenyl)acetamide | | | |
| 89 | 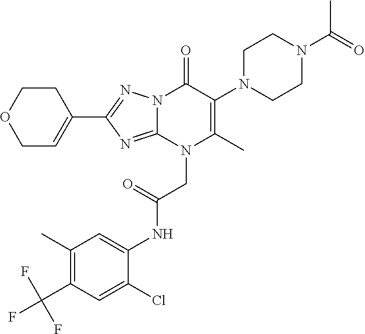 [SMILES: CC(=O)N1CCN(c2c(C)n(CC(=O)Nc3cc(C)c(C(F)(F)F)cc3Cl)c3nc(C4=CCOCC4)nn3c2=O)CC1] | 1.04 | 608.4 | UPLC- MS 1 |
| | 2-(6-(4-acetylpiperazin-1-yl)-2-(3,6-dihydro-2H-pyran- | | | |
| | 4-yl)-5-methyl-7-oxo-[1,2,4]triazolo[1,5-a]pyrimidin- | | | |
| | 4(7H)-yl)-N-(2-chloro-5-methyl-4-(trifluoromethyl)- | | | |
| | phenyl)acetamide | | | |
| 90 | 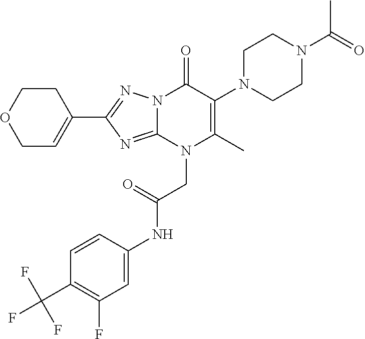 [SMILES: CC(=O)N1CCN(c2c(C)n(CC(=O)Nc3ccc(C(F)(F)F)c(F)c3)c3nc(C4=CCOCC4)nn3c2=O)CC1] | 0.98 | 578.6 | UPLC- MS 15 |
| | 2-(6-(4-acetylpiperazin-1-yl)-2-(3,6-dihydro-2H-pyran-4- | | | |
| | yl)-5-methyl-7-oxo-[1,2,4]triazolo[1,5-a]pyrimidin-4(7H)- | | | |
| | yl)-N-(3-fluoro-4-(trifluoromethyl)phenyl)acetamide | | | |
| 91 | 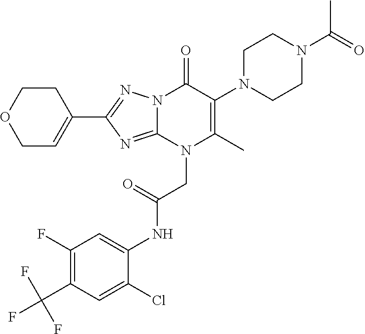 [SMILES: CC(=O)N1CCN(c2c(C)n(CC(=O)Nc3cc(F)c(C(F)(F)F)cc3Cl)c3nc(C4=CCOCC4)nn3c2=O)CC1] | 1.04 | 612.2 | UPLC- MS 3 |
| | 2-(6-(4-acetylpiperazin-1-yl)-2-(3,6-dihydro-2H-pyran- | | | |
| | 4-yl)-5-methyl-7-oxo-[1,2,4]triazolo[1,5-a]pyrimidin- | | | |
| | 4(7H)-yl)-N-(2-chloro-5-fluoro-4-(trifluoromethyl)- | | | |
| | phenyl)acetamide | | | |
| 92 | 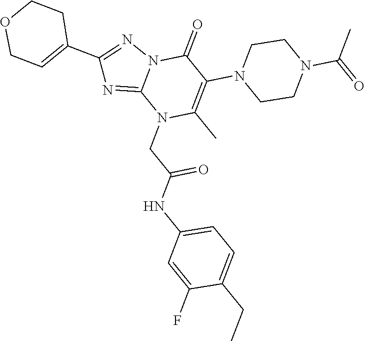 [SMILES: CCc1ccc(NC(=O)Cn2c(C)c(N3CCN(C(C)=O)CC3)c(=O)n3nc(C4=CCOCC4)nc23)cc1F] | 0.96 | 538.6 | UPLC- MS 15 |
| | 2-(6-(4-acetylpiperazin-1-yl)-2-(3,6-dihydro-2H-pyran-4- | | | |
| | yl)-5-methyl-7-oxo-[1,2,4]triazolo[1,5-a]pyrimidin- | | | |
| | 4(7H)-yl)-N-(4-ethyl-3-fluorophenyl)acetamide | | | |
| 93 | 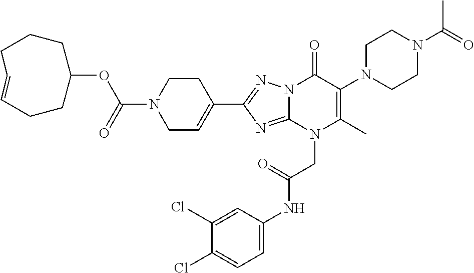 [SMILES: CC(=O)N1CCN(c2c(C)n(CC(=O)Nc3ccc(Cl)c(Cl)c3)c3nc(C4=CCN(C(=O)OC5CC/C=C/CCC5)CC4)nn3c2=O)CC1] | 1.27 | 711.3 | UPLC- MS 15 |
| | (E)-cyclooct-4-en-1-yl 4-(6-(4-acetylpiperazin-1-yl)-4-(2-((3,4- | | | |
| | dichlorophenyl)amino)-2-oxoethyl)-5-methyl-7-oxo-4,7-dihydro- | | | |
| | [1,2,4]triazolo[1,5-a]pyrimidin-2-yl)-3,6-dihydropyridine- | | | |
| | 1(2H)-carboxylate | | | |
| 94 | 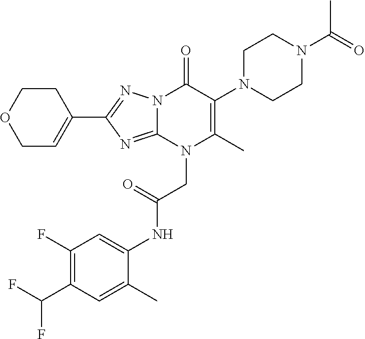 [SMILES: CC(=O)N1CCN(c2c(C)n(CC(=O)Nc3cc(F)c(C(F)F)cc3C)c3nc(C4=CCOCC4)nn3c2=O)CC1] | 0.83 | 574.4 | UPLC- MS 1 |
| | 2-(6-(4-acetylpiperazin-1-yl)-2-(3,6-dihydro-2H-pyran- | | | |
| | 4-yl)-5-methyl-7-oxo-[1,2,4]triazolo[1,5-a]pyrimidin- | | | |
| | 4(7H)-yl)-N-(4-(difluoromethyl)-5-fluoro-2-methyl- | | | |
| | phenyl)acetamide | | | |
| 95 | 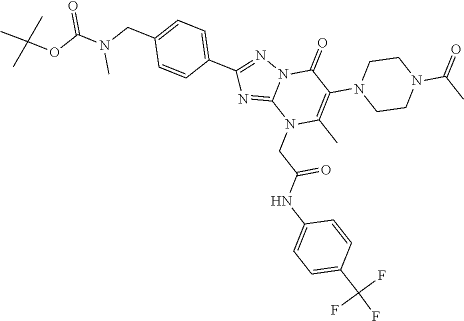 [SMILES: CC(=O)N1CCN(c2c(C)n(CC(=O)Nc3ccc(C(F)(F)F)cc3)c3nc(-c4ccc(CN(C)C(=O)OC(C)(C)C)cc4)nn3c2=O)CC1] | 1.17 | 697.7 | UPLC- MS 8 |
| | tert-butyl (4-(6-(4-acetylpiperazin-1-yl)-5-methyl-7-oxo-4-(2-oxo- | | | |
| | 2-((4-(trifluoromethyl)phenyl)amino)ethyl)-4,7-dihydro-[1,2,4] | | | |
| | triazolo[1,5-a]pyrimidin-2-yl)benzyl)(methyl)carbamate | | | |
| 96 | 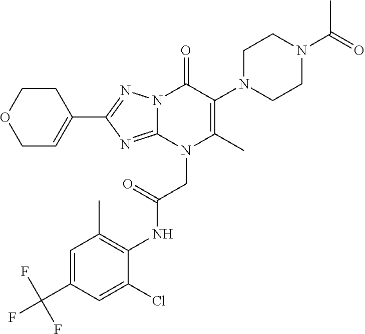 [SMILES: CC(=O)N1CCN(c2c(C)n(CC(=O)Nc3c(C)cc(C(F)(F)F)cc3Cl)c3nc(C4=CCOCC4)nn3c2=O)CC1] | 0.97 | 608.4 | UPLC- MS 3 |
| | 2-(6-(4-acetylpiperazin-1-yl)-2-(3,6-dihydro-2H-pyran- | | | |
| | 4-yl)-5-methyl-7-oxo-[1,2,4]triazolo[1,5-a]pyrimidin- | | | |
| | 4(7H)-yl)-N-(2-chloro-6-methyl-4-(trifluoromethyl)- | | | |
| | phenyl)acetamide | | | |
| 97 | 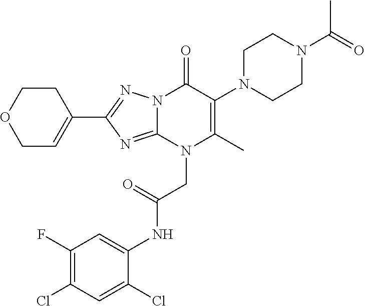 [SMILES: CC(=O)N1CCN(c2c(C)n(CC(=O)Nc3cc(F)c(Cl)cc3Cl)c3nc(C4=CCOCC4)nn3c2=O)CC1] | 0.96 | 578.3 | UPLC- MS 3 |
| | 2-(6-(4-acetylpiperazin-1-yl)-2-(3,6-dihydro-2H-pyran- | | | |
| | 4-yl)-5-methyl-7-oxo-[1,2,4]triazolo[1,5-a]pyrimidin- | | | |
| | 4(7H)-yl)-N-(2,4-dichloro-5-fluorophenyl)acetamide | | | |
| 98 | 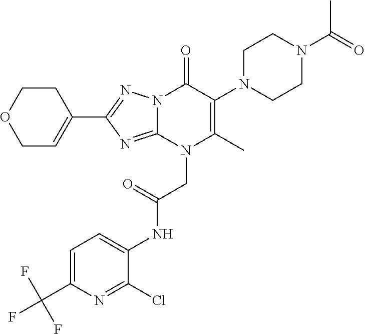 [SMILES: CC(=O)N1CCN(c2c(C)n(CC(=O)Nc3ccc(C(F)(F)F)nc3Cl)c3nc(C4=CCOCC4)nn3c2=O)CC1] | 0.89 | 595.3 | UPLC- MS 4 |
| | 2-(6-(4-acetylpiperazin-1-yl)-2-(3,6-dihydro-2H-pyran- | | | |
| | 4-yl)-5-methyl-7-oxo-[1,2,4]triazolo[1,5-a]pyrimidin- | | | |
| | 4(7H)-yl)-N-(2-chloro-6-(trifluoromethyl)pyridin-3- | | | |
| | yl)acetamide | | | |
| 99 | 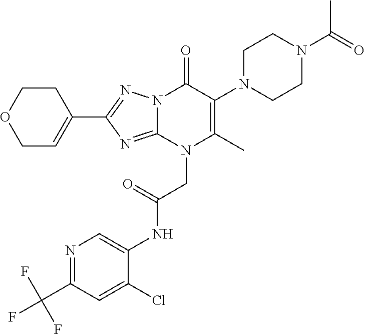 [SMILES: CC(=O)N1CCN(c2c(C)n(CC(=O)Nc3cnc(C(F)(F)F)cc3Cl)c3nc(C4=CCOCC4)nn3c2=O)CC1] | 0.87 | 595.4 | UPLC- MS 3 |
| | 2-(6-(4-acetylpiperazin-1-yl)-2-(3,6-dihydro-2H-pyran-4- | | | |
| | yl)-5-methyl-7-oxo-[1,2,4]triazolo[1,5-a]pyrimidin-4(7H)- | | | |
| | yl)-N-(4-chloro-6-(trifluoromethyl)pyridin-3-yl)acetamide | | | |
| 100 | 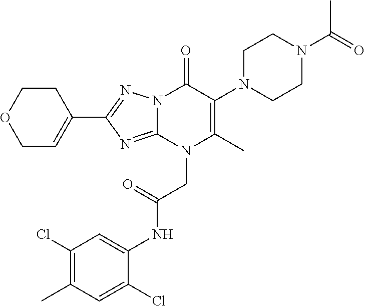 [SMILES: CC(=O)N1CCN(c2c(C)n(CC(=O)Nc3cc(Cl)c(C)cc3Cl)c3nc(C4=CCOCC4)nn3c2=O)CC1] | 0.96 | 574.3 | UPLC- MS 3 |
| | 2-(6-(4-acetylpiperazin-1-yl)-2-(3,6-dihydro-2H-pyran- | | | |
| | 4-yl)-5-methyl-7-oxo-[1,2,4]triazolo[1,5-a]pyrimidin- | | | |
| | 4(7H)-yl)-N-(2,5-dichloro-4-methylphenyl)acetamide | | | |
| 101 | 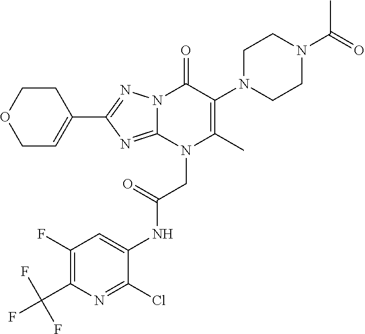 [SMILES: CC(=O)N1CCN(c2c(C)n(CC(=O)Nc3cc(F)c(C(F)(F)F)nc3Cl)c3nc(C4=CCOCC4)nn3c2=O)CC1] | 1.02 | 613.2 | UPLC- MS 3 |
| | 2-(6-(4-acetylpiperazin-1-yl)-2-(3,6-dihydro-2H-pyran-4- | | | |
| | yl)-5-methyl-7-oxo-[1,2,4]triazolo[1,5-a]pyrimidin-4(7H)- | | | |
| | yl)-N-(2-chloro-5-fluoro-6-(trifluoromethyl)pyridin-3-yl)- | | | |
| | acetamide | | | |
| 102 | 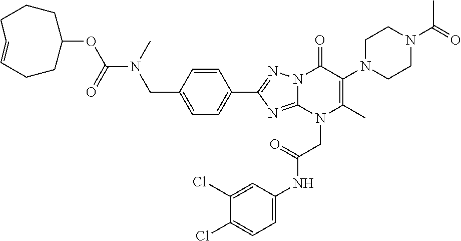 [SMILES: CC(=O)N1CCN(c2c(C)n(CC(=O)Nc3ccc(Cl)c(Cl)c3)c3nc(-c4ccc(CN(C)C(=O)OC5CC/C=C/CCC5)cc4)nn3c2=O)CC1] | 1.33 | 749.3 | UPLC- MS 15 |
| | (E)-cyclooct-4-en-1-yl (4-(6-(4-acetylpiperazin-1-yl)-4-(2-((3,4- | | | |
| | dichlorophenyl)amino)-2-oxoethyl)-5-methyl-7-oxo-4,7-dihydro- | | | |
| | [1,2,4]triazolo[1,5-a]pyrimidin-2-yl)benzyl)(methyl)carbamate | | | |
| 103 | 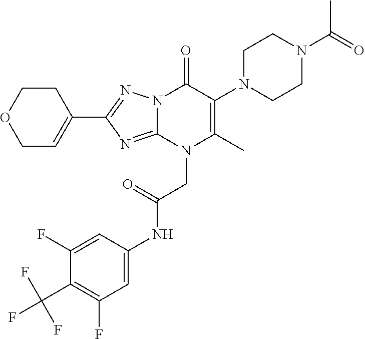 [SMILES: CC(=O)N1CCN(c2c(C)n(CC(=O)Nc3cc(F)c(C(F)(F)F)c(F)c3)c3nc(C4=CCOCC4)nn3c2=O)CC1] | 1.02 | 596.6 | UPLC- MS 15 |
| | 2-(6-(4-acetylpiperazin-1-yl)-2-(3,6-dihydro-2H-pyran-4- | | | |
| | yl)-5-methyl-7-oxo-[1,2,4]triazolo[1,5-a]pyrimidin-4(7H)- | | | |
| | yl)-N-(3,5-difluoro-4-(trifluoromethyl)phenyl)acetamide | | | |

### Route V

### Example 104: 2-(6-(4-acetylpiperazin-1-yl)-2-(5,6-dihydro-1,4-dioxin-2-yl)-5-methyl-7-oxo-[1,2,4]triazolo[1,5-a]pyrimidin-4(7H)-yl)-N-(4-(trifluoromethyl)phenyl)acetamide

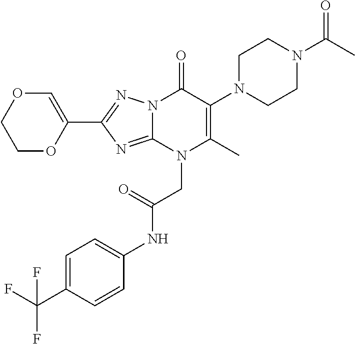 [SMILES: CC(=O)N1CCN(c2c(C)n(CC(=O)Nc3ccc(C(F)(F)F)cc3)c3nc(C4=COCCO4)nn3c2=O)CC1]

2-(6-(4-acetylpiperazin-1-yl)-2-bromo-5-methyl-7-oxo-[1,2,4]triazolo[1,5-a]pyrimidin-4(7H)-yl)-N-(4-(trifluoromethyl)phenyl)acetamide (Intermediate G) (370 mg, 665 μmol), 2-(5,6-dihydro-1,4-dioxin-2-yl)-4,4,5,5-tetramethyl-1,3,2-dioxaborolane (169 mg, 798 μmol), Pd X-Phos G3 (28.1 mg, 33.0 μmol) and K₃PO₄ 1 M in water (2.00 mL, 2.00 mmol) were mixed in dioxane (1 mL) and stirred at 65° C. for 1 hour. Water, aq sat NaHCO₃ and DCM were added. The aqueous layer was washed twice with DCM. The combined organic phases were dried through a phase separator and concentrated under reduced pressure. The residue was dissolved in DCM. Then ISOLUTE® Si-Thiol was added. The mixture was stirred at RT for 1 hour. Then it was filtered. The filtrate was concentrated down. The crude product was purified by column chromatography (Silica gel column: Silica 40 g, eluent DCM:DCM/MeOH (9/1) 100:0 to 0:100) to give the title compound.

LC-MS: Rt=0.92 min; MS m/z [M+H]⁺ 562.5, m/z [M−H]⁻ 560.5; UPLC-MS 8

Examples 105 to 135 were made using analogous methods to Example 104, using methods known to the skilled chemist and using starting materials in the public domain.

| | | Rt | MS | LC-MS |
|---|---|---|---|---|
| Ex. | Structure and name | [min] | [M + H]+ | method |
| 105 | 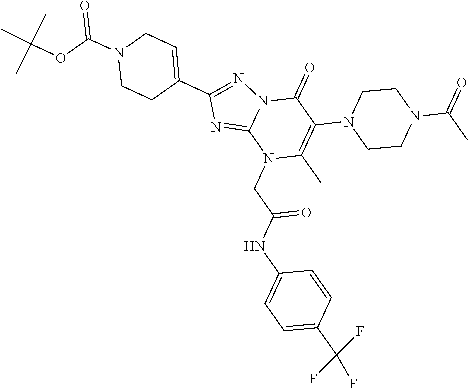 [SMILES: CC(=O)N1CCN(c2c(C)n(CC(=O)Nc3ccc(C(F)(F)F)cc3)c3nc(C4=CCN(C(=O)OC(C)(C)C)CC4)nn3c2=O)CC1] | 1.20 | 659.4 | UPLC- MS 1 |
| | tert-butyl 4-(6-(4-acetylpiperazin-1-yl)-5-methyl-7- | | | |
| | oxo-4-(2-oxo-2-((4- | | | |
| | (trifluoromethyl)phenyl)amino)ethyl)-4,7-dihydro- | | | |
| | [1,2,4]triazolo[1,5-a]pyrimidin-2-yl)-3,6- | | | |
| | dihydropyridine-1(2H)-carboxylate | | | |
| 106 | 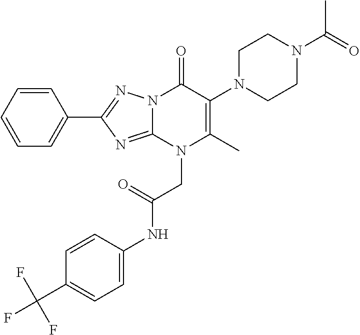 [SMILES: CC(=O)N1CCN(c2c(C)n(CC(=O)Nc3ccc(C(F)(F)F)cc3)c3nc(-c4ccccc4)nn3c2=O)CC1] | 1.12 | 555.2 | UPLC- MS 11 |
| | 2-(6-(4-acetylpiperazin-1-yl)-5-methyl-7-oxo-2- | | | |
| | (pyridin-3-yl)-[1,2,4]triazolo[1,5-a]pyrimidin-4(7H)- | | | |
| | yl)-N-(4-(trifluoromethyl)phenyl)acetamide | | | |
| 107 | 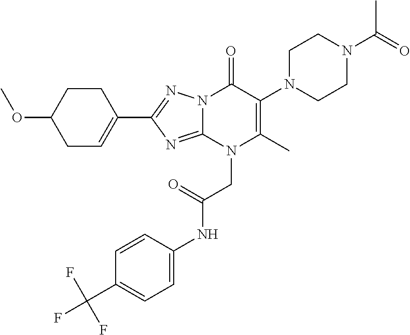 [SMILES: COC1CC=C(c2nc3n(CC(=O)Nc4ccc(C(F)(F)F)cc4)c(C)c(N4CCN(C(C)=O)CC4)c(=O)n3n2)CC1] | 1.48 | 588.4 | UPLC- MS 11 |
| | 2-(6-(4-acetylpiperazin-1-yl)-2-(4-methoxycyclohex- | | | |
| | 1-en-1-yl)-5-methyl-7-oxo-[1,2,4]triazolo[1,5- | | | |
| | a]pyrimidin-4(7H)-yl)-N-(4- | | | |
| | (trifluoromethyl)phenyl)acetamide | | | |
| 108 | 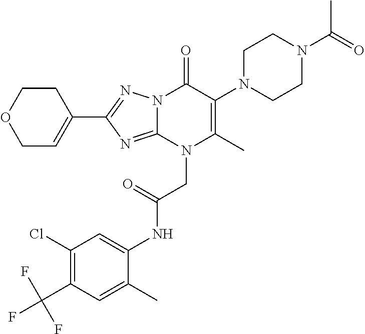 [SMILES: CC(=O)N1CCN(c2c(C)n(CC(=O)Nc3cc(Cl)c(C(F)(F)F)cc3C)c3nc(C4=CCOCC4)nn3c2=O)CC1] | 1.37 | 608.2 | UPLC- MS 11 |
| | 2-(6-(4-acetylpiperazin-1-yl)-2-(3,6-dihydro-2H- | | | |
| | pyran-4-yl)-5-methyl-7-oxo-[1,2,4]triazolo[1,5- | | | |
| | a]pyrimidin-4(7H)-yl)-N-(5-chloro-2-methyl-4- | | | |
| | (trifluoromethyl)phenyl)acetamide | | | |
| 109 | 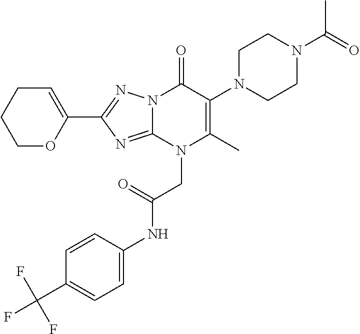 [SMILES: CC(=O)N1CCN(c2c(C)n(CC(=O)Nc3ccc(C(F)(F)F)cc3)c3nc(C4=CCCCO4)nn3c2=O)CC1] | 1.00 | 560.3 | UPLC- MS 4 |
| | 2-(6-(4-acetylpiperazin-1-yl)-2-(3,4-dihydro-2H- | | | |
| | pyran-6-yl)-5-methyl-7-oxo-[1,2,4]triazolo[1,5- | | | |
| | a]pyrimidin-4(7H)-yl)-N-(4- | | | |
| | (trifluoromethyl)phenyl)acetamide | | | |
| 110 | 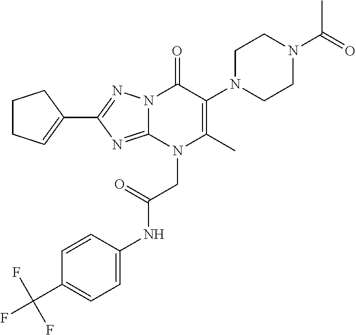 [SMILES: CC(=O)N1CCN(c2c(C)n(CC(=O)Nc3ccc(C(F)(F)F)cc3)c3nc(C4=CCCC4)nn3c2=O)CC1] | 1.06 | 544.3 | UPLC- MS 15 |
| | 2-(6-(4-acetylpiperazin-1-yl)-2-(cyclopent-1-en-1-yl)- | | | |
| | 5-methyl-7-oxo-[1,2,4]triazolo[1,5-a]pyrimidin-4(7H)- | | | |
| | yl)-N-(4-(trifluoromethyl)phenyl)acetamide | | | |
| 111 | 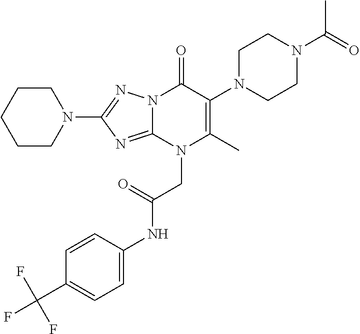 [SMILES: CC(=O)N1CCN(c2c(C)n(CC(=O)Nc3ccc(C(F)(F)F)cc3)c3nc(N4CCCCC4)nn3c2=O)CC1] | 1.06 | 561.6 | UPLC- MS 15 |
| | 2-(6-(4-acetylpiperazin-1-yl)-5-methyl-7-oxo-2- | | | |
| | (piperidin-1-yl)-[1,2,4]triazolo[1,5-a]pyrimidin-4(7H)- | | | |
| | yl)-N-(4-(trifluoromethyl)phenyl)acetamide | | | |
| 112 | 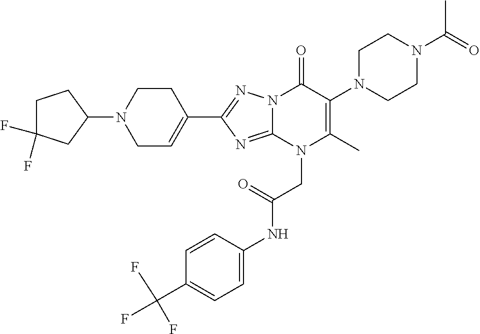 [SMILES: CC(=O)N1CCN(c2c(C)n(CC(=O)Nc3ccc(C(F)(F)F)cc3)c3nc(C4=CCN(C5CCC(F)(F)C5)CC4)nn3c2=O)CC1] | 0.86 | 663.4 | UPLC- MS 1 |
| | 2-(6-(4-acetylpiperazin-1-yl)-2-(4-(3,3- | | | |
| | difluoropyrrolidin-1-yl)cyclohex-1-en-1-yl)-5-methyl- | | | |
| | 7-oxo-[1,2,4]triazolo[1,5-a]pyrimidin-4(7H)-yl)-N-(4- | | | |
| | (trifluoromethyl)phenyl)acetamide | | | |
| 113 | 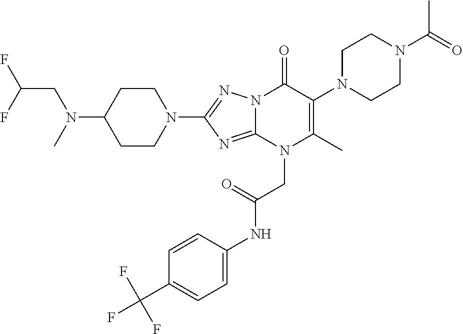 [SMILES: CC(=O)N1CCN(c2c(C)n(CC(=O)Nc3ccc(C(F)(F)F)cc3)c3nc(N4CCC(N(C)CC(F)F)CC4)nn3c2=O)CC1] | 0.85 | 654.3 | UPLC- MS 16 |
| | 2-(6-(4-acetylpiperazin-1-yl)-2-(4-((2,2- | | | |
| | difluoroethyl)(methyl)amino)piperidin-1-yl)-5-methyl- | | | |
| | 7-oxo-[1,2,4]triazolo[1,5-a]pyrimidin-4(7H)-yl)-N-(4- | | | |
| | (trifluoromethyl)phenyl)acetamide | | | |
| 114 | 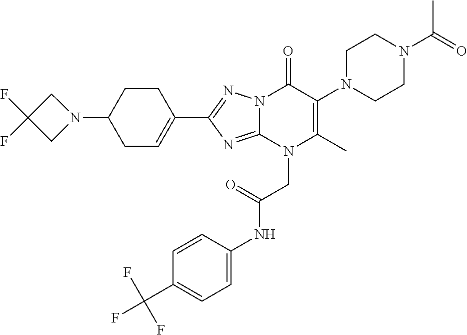 [SMILES: CC(=O)N1CCN(c2c(C)n(CC(=O)Nc3ccc(C(F)(F)F)cc3)c3nc(C4=CCC(N5CC(F)(F)C5)CC4)nn3c2=O)CC1] | 0.90 | 649.4 | UPLC- MS 1 |
| | 2-(6-(4-acetylpiperazin-1-yl)-2-(4-(3,3- | | | |
| | difluoroazetidin-1-yl)cyclohex-1-en-1-yl)-5-methyl-7- | | | |
| | oxo-[1,2,4]triazolo[1,5-a]pyrimidin-4(7H)-yl)-N-(4- | | | |
| | (trifluoromethyl)phenyl) acetamide | | | |
| 115 | 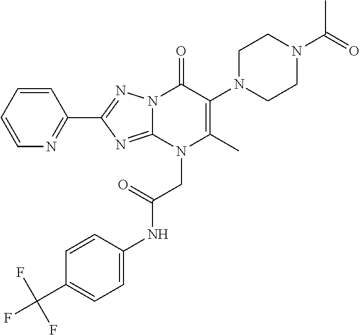 [SMILES: CC(=O)N1CCN(c2c(C)n(CC(=O)Nc3ccc(C(F)(F)F)cc3)c3nc(-c4ccccn4)nn3c2=O)CC1] | 1.14 | 555.2 | UPLC- MS 11 |
| | 2-(6-(4-acetylpiperazin-1-yl)-5-methyl-7-oxo-2- | | | |
| | (pyridin-2-yl)-[1,2,4]triazolo[1,5-a]pyrimidin-4(7H)- | | | |
| | yl)-N-(4-(trifluoromethyl)phenyl)acetamide | | | |
| 116 | 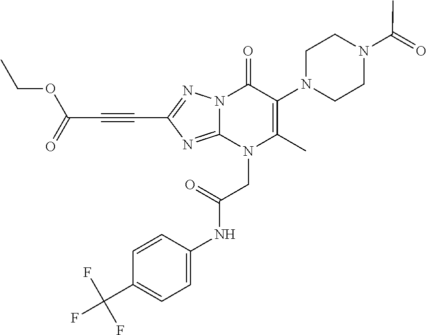 [SMILES: CCOC(=O)C#Cc1nc2n(CC(=O)Nc3ccc(C(F)(F)F)cc3)c(C)c(N3CCN(C(C)=O)CC3)c(=O)n2n1] | 1.04 | 574.4 | UPLC- MS 8 |
| | ethyl 3-(6-(4-acetylpiperazin-1-yl)-5-methyl-7-oxo-4- | | | |
| | (2-oxo-2-((4-(trifluoromethyl)phenyl)amino)ethyl)- | | | |
| | 4,7-dihydro-[1,2,4]triazolo[1,5-a]pyrimidin-2- | | | |
| | yl)propiolate | | | |
| 117 | 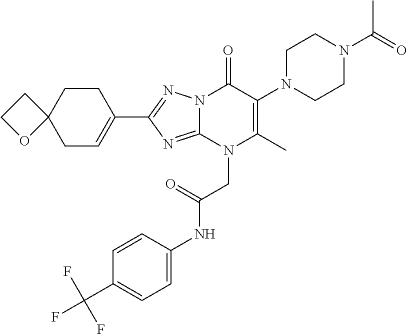 [SMILES: CC(=O)N1CCN(c2c(C)n(CC(=O)Nc3ccc(C(F)(F)F)cc3)c3nc(C4=CCC5(CCO5)CC4)nn3c2=O)CC1] | 0.99 | 600.3 | UPLC- MS 8 |
| | 2-(6-(4-acetylpiperazin-1-yl)-5-methyl-7-oxo-2-(1- | | | |
| | oxaspiro[3.5]non-6-en-7-yl)-[1,2,4]triazolo[1,5- | | | |
| | a]pyrimidin-4(7H)-yl)-N-(4- | | | |
| | (trifluoromethyl)phenyl)acetamide | | | |
| 118 | 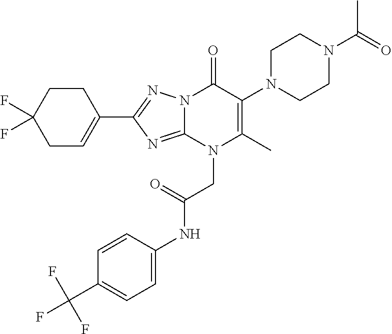 [SMILES: CC(=O)N1CCN(c2c(C)n(CC(=O)Nc3ccc(C(F)(F)F)cc3)c3nc(C4=CCC(F)(F)CC4)nn3c2=O)CC1] | 1.07 | 594.3 | UPLC- MS 8 |
| | 2-(6-(4-acetylpiperazin-1-yl)-2-(4,4-difluorocyclohex- | | | |
| | 1-en-1-yl)-5-methyl-7-oxo-[1,2,4]triazolo[1,5- | | | |
| | a]pyrimidin-4(7H)-yl)-N-(4- | | | |
| | (trifluoromethyl)phenyl)acetamide | | | |
| 119 | 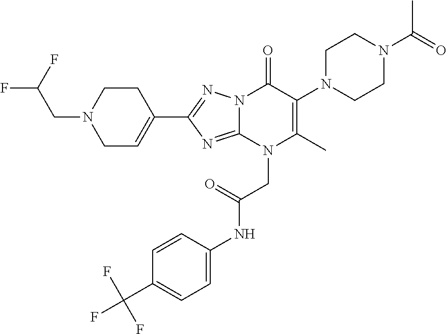 [SMILES: CC(=O)N1CCN(c2c(C)n(CC(=O)Nc3ccc(C(F)(F)F)cc3)c3nc(C4=CCN(CC(F)F)CC4)nn3c2=O)CC1] | 0.88 | 623.5 | UPLC- MS 1 |
| | 2-(6-(4-acetylpiperazin-1-yl)-2-(1-(2,2-difluoroethyl)- | | | |
| | 1,2,3,6-tetrahydropyridin-4-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 120 | 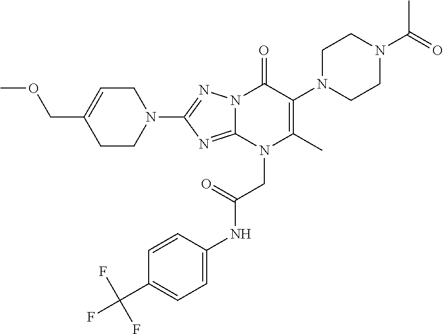 [SMILES: COCC1=CCN(c2nc3n(CC(=O)Nc4ccc(C(F)(F)F)cc4)c(C)c(N4CCN(C(C)=O)CC4)c(=O)n3n2)CC1] | 1.04 | 603.3 | UPLC- MS 16 |
| | 2-(6-(4-acetylpiperazin-1-yl)-2-(4-(methoxymethyl)- | | | |
| | 3,6-dihydropyridin-1(2H)-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 121 | 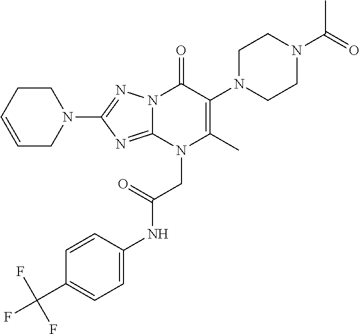 [SMILES: CC(=O)N1CCN(c2c(C)n(CC(=O)Nc3ccc(C(F)(F)F)cc3)c3nc(N4CC=CCC4)nn3c2=O)CC1] | 1.08 | 559.3 | UPLC- MS 16 |
| | 2-(6-(4-acetylpiperazin-1-yl)-2-(3,6-dihydropyridin- | | | |
| | 1(2H)-yl)-5-methyl-7-oxo-[1,2,4]triazolo[1,5- | | | |
| | a]pyrimidin-4(7H)-yl)-N-(4- | | | |
| | (trifluoromethyl)phenyl)acetamide | | | |
| 122 | 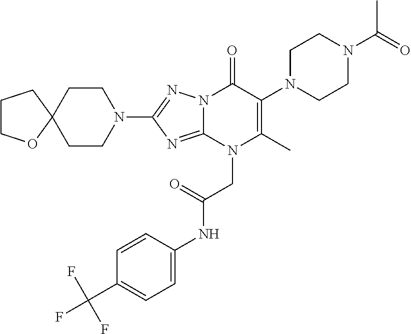 [SMILES: CC(=O)N1CCN(c2c(C)n(CC(=O)Nc3ccc(C(F)(F)F)cc3)c3nc(N4CCC5(CCCO5)CC4)nn3c2=O)CC1] | 1.07 | 617.3 | UPLC- MS 16 |
| | 2-(6-(4-acetylpiperazin-1-yl)-5-methyl-7-oxo-2-(1- | | | |
| | oxa-8-azaspiro[4.5]decan-8-yl)-[1,2,4]triazolo[1,5- | | | |
| | a]pyrimidin-4(7H)-yl)-N-(4- | | | |
| | (trifluoromethyl)phenyl)acetamide | | | |
| 123 | 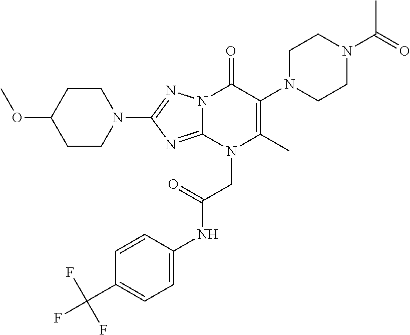 [SMILES: COC1CCN(c2nc3n(CC(=O)Nc4ccc(C(F)(F)F)cc4)c(C)c(N4CCN(C(C)=O)CC4)c(=O)n3n2)CC1] | 1.5 | 591.4 | UPLC- MS 12 |
| | 2-(6-(4-acetylpiperazin-1-yl)-2-(4-methoxypiperidin- | | | |
| | 1-yl)-5-methyl-7-oxo-[1,2,4]triazolo[1,5-a]pyrimidin- | | | |
| | 4(7H)-yl)-N-(4-(trifluoromethyl)phenyl)acetamide | | | |
| 124 | 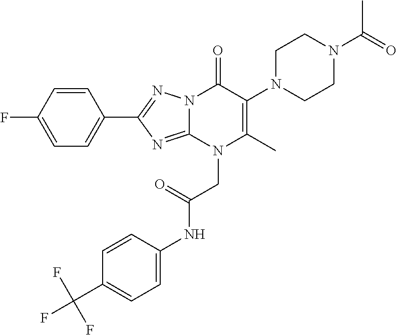 [SMILES: CC(=O)N1CCN(c2c(C)n(CC(=O)Nc3ccc(C(F)(F)F)cc3)c3nc(-c4ccc(F)cc4)nn3c2=O)CC1] | 1.48 | 572.8 | UPLC- MS 12 |
| | 2-(6-(4-acetylpiperazin-1-yl)-2-(4-fluorophenyl)-5- | | | |
| | methyl-7-oxo-[1,2,4]triazolo[1,5-a]pyrimidin-4(7H)- | | | |
| | yl)-N-(4-(trifluoromethyl)phenyl) acetamide | | | |
| 125 | 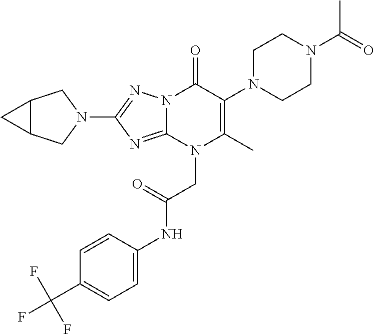 [SMILES: CC(=O)N1CCN(c2c(C)n(CC(=O)Nc3ccc(C(F)(F)F)cc3)c3nc(N4CC5CC5C4)nn3c2=O)CC1] | 1.06 | 559.3 | UPLC- MS 16 |
| | 2-(6-(4-acetylpiperazin-1-yl)-2-(3- | | | |
| | azabicyclo[3.1.0]hexan-3-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 126 | 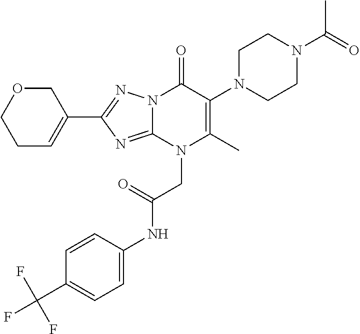 [SMILES: CC(=O)N1CCN(c2c(C)n(CC(=O)Nc3ccc(C(F)(F)F)cc3)c3nc(C4=CCCOC4)nn3c2=O)CC1] | 0.97 | 560.4 | UPLC- MS 1 |
| | 2-(6-(4-acetylpiperazin-1-yl)-2-(5,6-dihydro-2H- | | | |
| | pyran-3-yl)-5-methyl-7-oxo-[1,2,4]triazolo[1,5- | | | |
| | a]pyrimidin-4(7H)-yl)-N-(4- | | | |
| | (trifluoromethyl)phenyl)acetamide | | | |
| 127 | 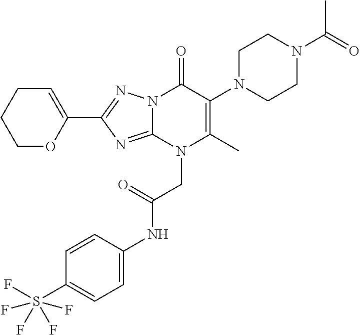 [SMILES: CC(=O)N1CCN(c2c(C)n(CC(=O)Nc3ccc(S(F)(F)(F)(F)F)cc3)c3nc(C4=CCCCO4)nn3c2=O)CC1] | 1.03 | 618.5 | UPLC- MS 8 |
| | 2-(6-(4-acetylpiperazin-1-yl)-2-(3,4-dihydro-2H- | | | |
| | pyran-6-yl)-5-methyl-7-oxo-[1,2,4]triazolo[1,5- | | | |
| | a]pyrimidin-4(7H)-yl)-N-(4-(pentafluoro-16- | | | |
| | sulfaneyl)phenyl)acetamide | | | |
| 128 | 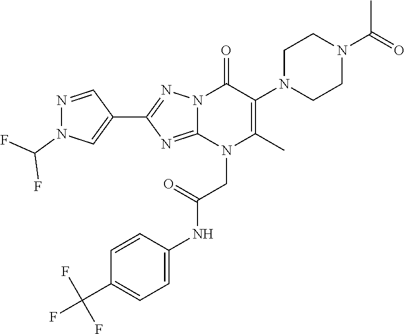 [SMILES: CC(=O)N1CCN(c2c(C)n(CC(=O)Nc3ccc(C(F)(F)F)cc3)c3nc(-c4cnn(C(F)F)c4)nn3c2=O)CC1] | 1.5 | 594.2 | UPLC- MS 11 |
| | 2-(6-(4-acetylpiperazin-1-yl)-2-(1-(difluoromethyl)- | | | |
| | 1H-pyrazol-4-yl)-5-methyl-7-oxo-[1,2,4]triazolo[1,5- | | | |
| | a]pyrimidin-4(7H)-yl)-N-(4- | | | |
| | (trifluoromethyl)phenyl)acetamide | | | |
| 129 | 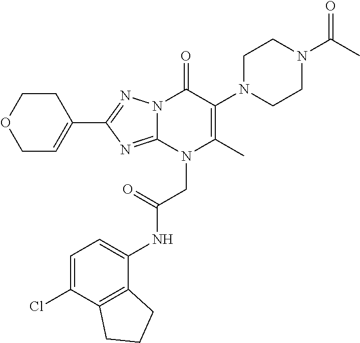 [SMILES: CC(=O)N1CCN(c2c(C)n(CC(=O)Nc3ccc(Cl)c4c3CCC4)c3nc(C4=CCOCC4)nn3c2=O)CC1] | 0.98 | 566.2 | UPLC- MS 16 |
| | 2-(6-(4-acetylpiperazin-1-yl)-2-(3,6-dihydro-2H- | | | |
| | pyran-4-yl)-5-methyl-7-oxo-[1,2,4]triazolo[1,5- | | | |
| | a]pyrimidin-4(7H)-yl)-N-(7-chloro-2,3-dihydro-1H- | | | |
| | inden-4-yl)acetamide | | | |
| 130 | 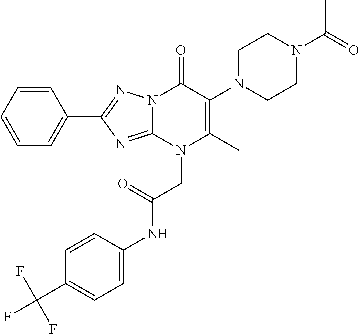 [SMILES: CC(=O)N1CCN(c2c(C)n(CC(=O)Nc3ccc(C(F)(F)F)cc3)c3nc(-c4ccccc4)nn3c2=O)CC1] | 1.52 | 554.3 | UPLC- MS 11 |
| | 2-(6-(4-acetylpiperazin-1-yl)-5-methyl-7-oxo-2- | | | |
| | phenyl-[1,2,4]triazolo[1,5-a]pyrimidin-4(7H)-yl)-N-(4- | | | |
| | (trifluoromethyl)phenyl)acetamide | | | |
| 131 | 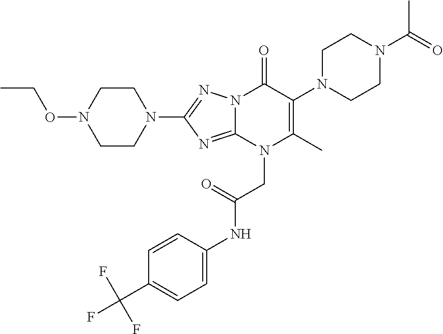 [SMILES: CCON1CCN(c2nc3n(CC(=O)Nc4ccc(C(F)(F)F)cc4)c(C)c(N4CCN(C(C)=O)CC4)c(=O)n3n2)CC1] | 1.08 | 606.3 | UPLC- MS 16 |
| | 2-(6-(4-acetylpiperazin-1-yl)-2-(4-ethoxypiperazin-1- | | | |
| | yl)-5-methyl-7-oxo-[1,2,4]triazolo[1,5-a]pyrimidin- | | | |
| | 4(7H)-yl)-N-(4-(trifluoromethyl)phenyl)acetamide | | | |
| 132 | 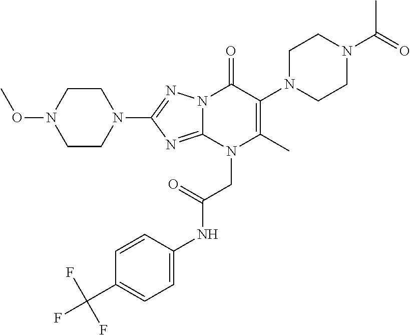 [SMILES: CON1CCN(c2nc3n(CC(=O)Nc4ccc(C(F)(F)F)cc4)c(C)c(N4CCN(C(C)=O)CC4)c(=O)n3n2)CC1] | 0.99 | 592.3 | UPLC- MS 16 |
| | 2-(6-(4-acetylpiperazin-1-yl)-2-(4-methoxypiperazin- | | | |
| | 1-yl)-5-methyl-7-oxo-[1,2,4]triazolo[1,5-a]pyrimidin- | | | |
| | 4(7H)-yl)-N-(4-(trifluoromethyl)phenyl)acetamide | | | |
| 133 | 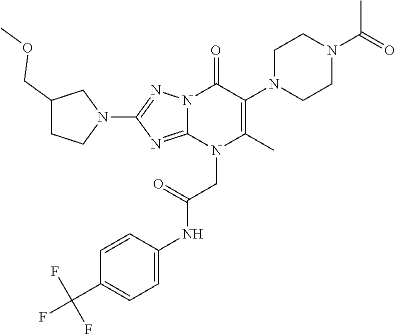 [SMILES: COCC1CCN(c2nc3n(CC(=O)Nc4ccc(C(F)(F)F)cc4)c(C)c(N4CCN(C(C)=O)CC4)c(=O)n3n2)C1] | 1.02 | 591.3 | UPLC- MS 16 |
| | 2-(6-(4-acetylpiperazin-1-yl)-2-(3- | | | |
| | (methoxymethyl)pyrrolidin-1-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 134 | 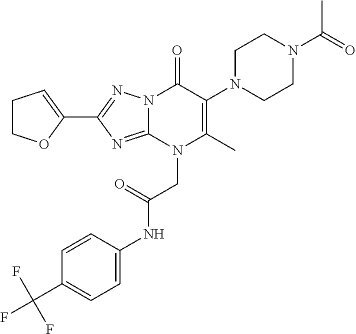 [SMILES: CC(=O)N1CCN(c2c(C)n(CC(=O)Nc3ccc(C(F)(F)F)cc3)c3nc(C4=CCCO4)nn3c2=O)CC1] | 1.32 | 546.2 | UPLC- MS 11 |
| | 2-(6-(4-acetylpiperazin-1-yl)-2-(4,5-dihydrofuran-2- | | | |
| | yl)-5-methyl-7-oxo-[1,2,4]triazolo[1,5-a]pyrimidin- | | | |
| | 4(7H)-yl)-N-(4-(trifluoromethyl)phenyl)acetamide | | | |
| 135 | 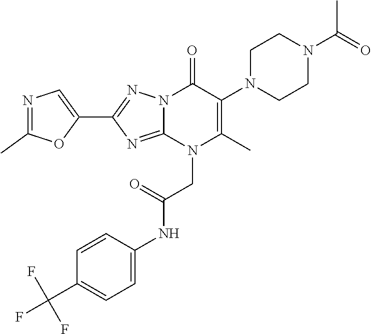 [SMILES: CC(=O)N1CCN(c2c(C)n(CC(=O)Nc3ccc(C(F)(F)F)cc3)c3nc(-c4cnc(C)o4)nn3c2=O)CC1] | 0.87 | 559.2 | UPLC- MS 4 |
| | 2-(6-(4-acetylpiperazin-1-yl)-5-methyl-2-(2- | | | |
| | methyloxazol-5-yl)-7-oxo-[1,2,4]triazolo[1,5- | | | |
| | a]pyrimidin-4(7H)-yl)-N-(4- | | | |
| | (trifluoromethyl)phenyl)acetamide | | | |

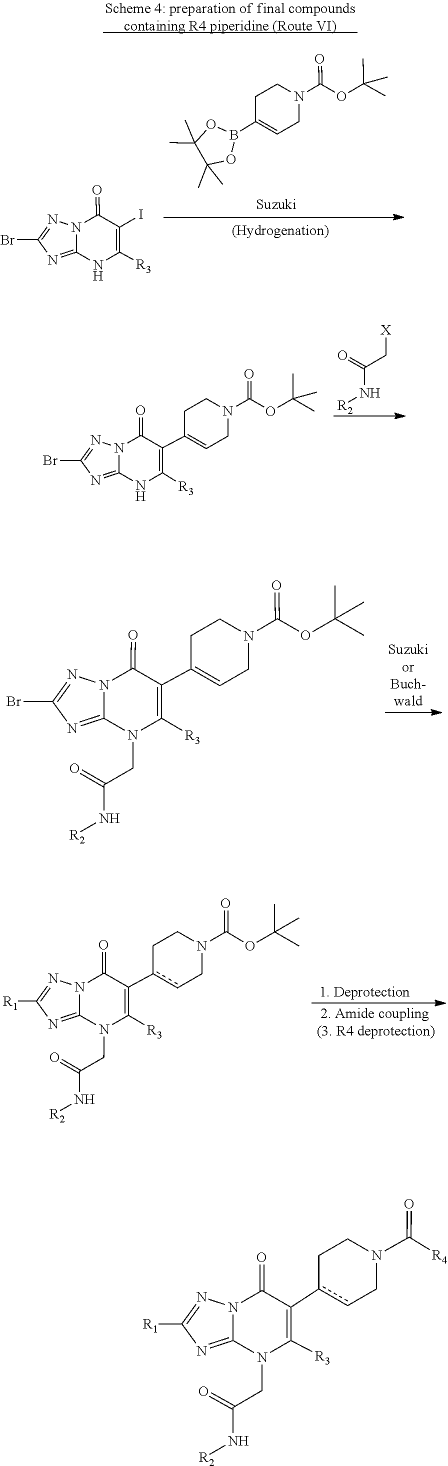

### Route VI

### Example 136: 2-(6-(1-(3,6-difluoro-2-hydroxybenzoyl)piperidin-4-yl)-2-(3,6-dihydro-2H-pyran-4-yl)-5-methyl-7-oxo-[1,2,4]triazolo[1,5-a]pyrimidin-4(7H)-yl)-N-(4-(trifluoromethyl)phenyl)acetamide

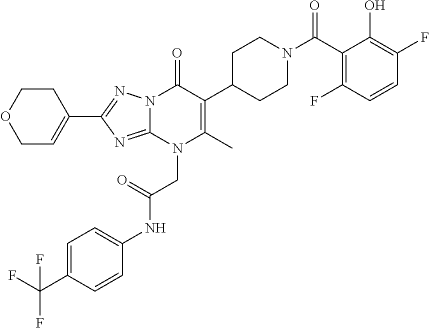 [SMILES: Cc1c(C2CCN(C(=O)c3c(F)ccc(F)c3O)CC2)c(=O)n2nc(C3=CCOCC3)nc2n1CC(=O)Nc1ccc(C(F)(F)F)cc1]

3,6-Difluoro-2-hydroxybenzoic acid (31.0 mg, 178 μmol) was dissolved in DCM (100 μL) and mixed with 1-Chloro-N,N,2-trimethyl-1-propenylamine (17.7 μl, 134 μmol). The RM was stirred at RT for 30 minutes. DIPEA (46.7 μL, 267 μmol) was added, followed by and 2-(2-(3,6-dihydro-2H-pyran-4-yl)-5-methyl-7-oxo-6-(piperidin-4-yl)-[1,2,4]triazolo[1,5-a]pyrimidin-4(7H)-yl)-N-(4-(trifluoromethyl)phenyl)acetamide (Intermediate C) (46.0 mg, 89.0 μmol). The RM was stirred at RT for 1 hour. The RM was concentrated under reduced pressure. The crude product was purified by reverse phase preparative HPLC (RP-HPLC acidic 1:5 to 100% B in 20 min), then by preparative chiral HPLC (instrument: Sepiatec prep SFC-100; column: LUX Amylose-1 (Chiralpak AD), 250 mm×30 mm 5 μm; eluent: A: 37% IPA+0.1% NH₃, B: 63% scCO₂; flow rate: 80.0 mL/min; detection: UV; injection volume: 2.3 mL; gradient: isocratic A: 37%, B: 63%; oven temperature: 40° C.; BPR: 120 bar) to give the title compound.

LC-MS: Rt=1.01 min; MS m/z [M+H]⁺ 673.4, m/z [M−H]⁻ 671.4; UPLC-MS 8

Examples 137 to 146 were made using analogous methods to Example 136, using methods known to the skilled chemist and using starting materials in the public domain. In certain cases, the step order was changed.

| | | Rt | MS | LC-MS |
|---|---|---|---|---|
| Ex. | Structure and name | [min] | [M + H]+ | method |
| 137 | 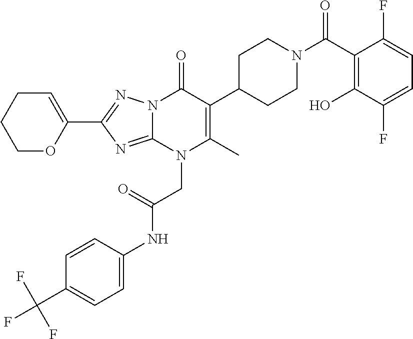 [SMILES: Cc1c(C2CCN(C(=O)c3c(F)ccc(F)c3O)CC2)c(=O)n2nc(C3=CCCCO3)nc2n1CC(=O)Nc1ccc(C(F)(F)F)cc1] | 1.06 | 673.2 | UPLC- MS 8 |
| | 2-(6-(1-(3,6-difluoro-2-hydroxybenzoyl)piperidin-4- | | | |
| | yl)-2-(3,4-dihydro-2H-pyran-6-yl)-5-methyl-7-oxo- | | | |
| | [1,2,4]triazolo[1,5-a]pyrimidin-4(7H)-yl)-N-(4- | | | |
| | (trifluoromethyl)phenyl) acetamide | | | |
| 138 | 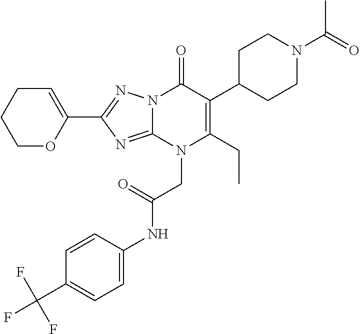 [SMILES: CCc1c(C2CCN(C(C)=O)CC2)c(=O)n2nc(C3=CCCCO3)nc2n1CC(=O)Nc1ccc(C(F)(F)F)cc1] | 1.00 | 573.3 | UPLC- MS 8 |
| | 2-(6-(1-acetylpiperidin-4-yl)-2-(3,4-dihydro-2H- | | | |
| | pyran-6-yl)-5-ethyl-7-oxo-[1,2,4]triazolo[1,5- | | | |
| | a]pyrimidin-4(7H)-yl)-N-(4- | | | |
| | (trifluoromethyl)phenyl)acetamide | | | |
| 139 | 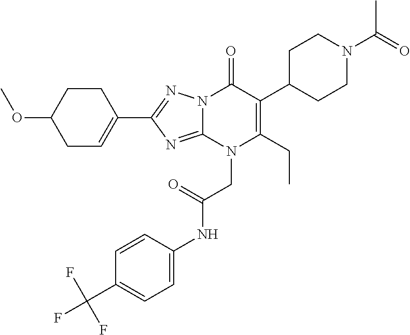 [SMILES: CCc1c(C2CCN(C(C)=O)CC2)c(=O)n2nc(C3=CCC(OC)CC3)nc2n1CC(=O)Nc1ccc(C(F)(F)F)cc1] | 1.03 | 601.3 | UPLC- MS 8 |
| | 2-(6-(1-acetylpiperidin-4-yl)-5-ethyl-2-(4- | | | |
| | methoxycyclohex-1-en-1-yl)-7-oxo- | | | |
| | [1,2,4]triazolo[1,5-a]pyrimidin-4(7H)-yl)-N-(4- | | | |
| | (trifluoromethyl)phenyl)acetamide | | | |
| 140 | 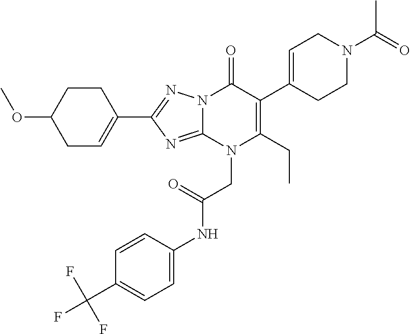 [SMILES: CCc1c(C2=CCN(C(C)=O)CC2)c(=O)n2nc(C3=CCC(OC)CC3)nc2n1CC(=O)Nc1ccc(C(F)(F)F)cc1] | 1.00 | 599.2 | UPLC- MS 8 |
| | 2-(6-(1-acetyl-1,2,3,6-tetrahydropyridin-4-yl)-5-ethyl- | | | |
| | 2-(4-methoxycyclohex-1-en-1-yl)-7-oxo- | | | |
| | [1,2,4]triazolo[1,5-a]pyrimidin-4(7H)-yl)-N-(4- | | | |
| | (trifluoromethyl)phenyl)acetamide | | | |
| 141 | 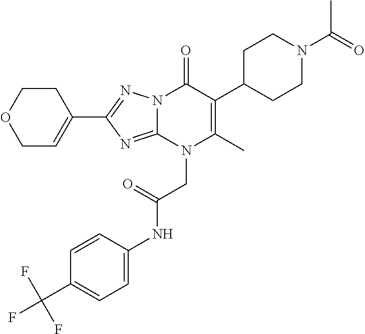 [SMILES: CC(=O)N1CCC(c2c(C)n(CC(=O)Nc3ccc(C(F)(F)F)cc3)c3nc(C4=CCOCC4)nn3c2=O)CC1] | 0.92 | 559.2 | UPLC- MS 8 |
| | 2-(6-(1-acetylpiperidin-4-yl)-2-(3,6-dihydro-2H- | | | |
| | pyran-4-yl)-5-methyl-7-oxo-[1,2,4]triazolo[1,5- | | | |
| | a]pyrimidin-4(7H)-yl)-N-(4- | | | |
| | (trifluoromethyl)phenyl)acetamide | | | |
| 142 | 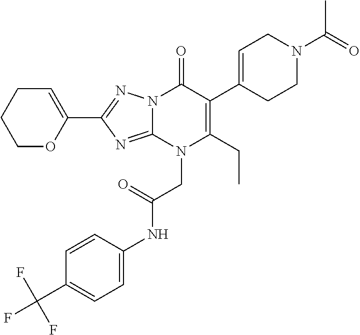 [SMILES: CCc1c(C2=CCN(C(C)=O)CC2)c(=O)n2nc(C3=CCCCO3)nc2n1CC(=O)Nc1ccc(C(F)(F)F)cc1] | 0.98 | 571.3 | UPLC- MS 8 |
| | 2-(6-(1-acetyl-1,2,3,6-tetrahydropyridin-4-yl)-2-(3,4- | | | |
| | dihydro-2H-pyran-6-yl)-5-ethyl-7-oxo- | | | |
| | [1,2,4]triazolo[1,5-a]pyrimidin-4(7H)-yl)-N-(4- | | | |
| | (trifluoromethyl)phenyl)acetamide | | | |
| 143 | 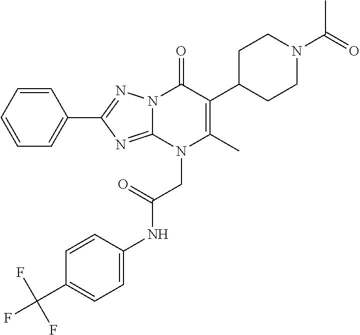 [SMILES: CC(=O)N1CCC(c2c(C)n(CC(=O)Nc3ccc(C(F)(F)F)cc3)c3nc(-c4ccccc4)nn3c2=O)CC1] | 1.06 | 553.5 | UPLC- MS 15 |
| | 2-(6-(1-acetylpiperidin-4-yl)-5-methyl-7-oxo-2- | | | |
| | phenyl-[1,2,4]triazolo[1,5-a]pyrimidin-4(7H)-yl)-N-(4- | | | |
| | (trifluoromethyl)phenyl) acetamide | | | |
| 144 | 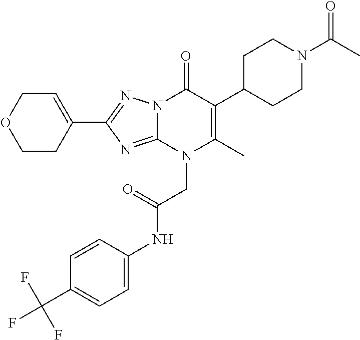 | 1.00 | 553.4 | UPLC- MS 8 |
| | 2-(6-(1-acetylpiperidin-4-yl)-2-(3,6-dihydro-2H- | | | |
| | pyran-4-yl)-5-methyl-7-oxo-[1,2,4]triazolo[1,5- | | | |
| | a]pyrimidin-4(7H)-yl)-N-(3-chloro-4- | | | |
| | ethylphenyl)acetamide | | | |
| 145 | 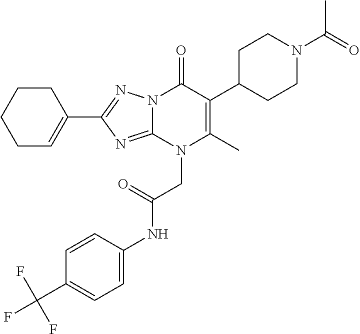 [SMILES: CC(=O)N1CCC(c2c(C)n(CC(=O)Nc3ccc(C(F)(F)F)cc3)c3nc(C4=CCCCC4)nn3c2=O)CC1] | 1.09 | 557.4 | UPLC- MS 8 |
| | 2-(6-(1-acetylpiperidin-4-yl)-2-(cyclohex-1-en-1-yl)- | | | |
| | 5-methyl-7-oxo-[1,2,4]triazolo[1,5-a]pyrimidin-4(7H)- | | | |
| | yl)-N-(4-(trifluoromethyl)phenyl)acetamide | | | |
| 146 | 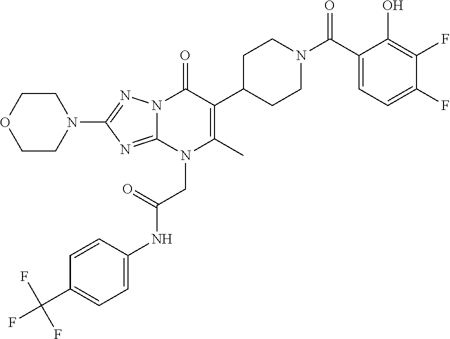 [SMILES: Cc1c(C2CCN(C(=O)c3ccc(F)c(F)c3O)CC2)c(=O)n2nc(N3CCOCC3)nc2n1CC(=O)Nc1ccc(C(F)(F)F)cc1] | 1.51 | 676.2 | UPLC- MS 11 |
| | 2-(6-(1-(3,4-difluoro-2-hydroxybenzoyl)piperidin-4- | | | |
| | yl)-5-methyl-2-morpholino-7-oxo-[1,2,4]triazolo[1,5- | | | |
| | a]pyrimidin-4(7H)-yl)-N-(4- | | | |
| | (trifluoromethyl)phenyl)acetamide | | | |

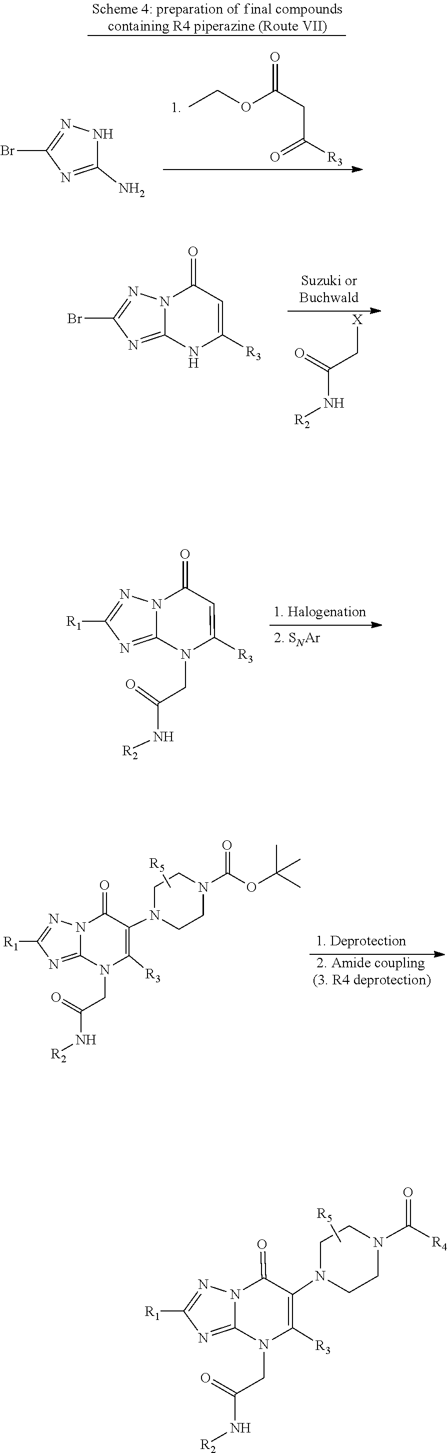

### Example 147: 2-(6-(4-acetylpiperazin-1-yl)-5-cyclopropyl-2-(3,6-dihydro-2H-pyran-4-yl)-7-oxo-[1,2,4]triazolo[1,5-a]pyrimidin-4(7H)-yl)-N-(4-(trifluoromethyl)phenyl)acetamide

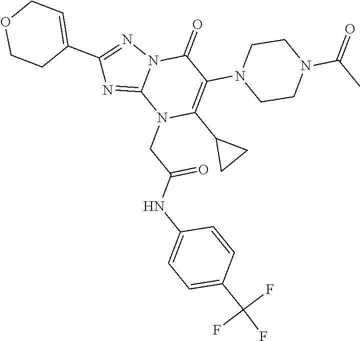 [SMILES: CC(=O)N1CCN(c2c(C3CC3)n(CC(=O)Nc3ccc(C(F)(F)F)cc3)c3nc(C4=CCOCC4)nn3c2=O)CC1]

2-(5-cyclopropyl-2-(3,6-dihydro-2H-pyran-4-yl)-7-oxo-6-(piperazin-1-yl)-[1,2,4]triazolo[1,5-a]pyrimidin-4(7H)-yl)-N-(4-(trifluoromethyl)phenyl)acetamide (Intermediate B) (45.0 mg, 83.0 μmol) was dissolved in THF (1.65 mL) under argon. Et₃N (34.4 μL, 248 μmol) was added, followed by acetic anhydride (9.30 mg, 91.0 μmol). The RM was stirred at RT for 2 hours. Et₃N (30.0 μL, 216 μmol) and acetic anhydride (9.30 mg, 91.0 μmol) were added again and the RM was stirred at RT for 2.25 hours. The RM was diluted with DCM and washed with aq sat NH4Cl and water. The organic phase was dried through a phase separator and concentrated under reduced pressure. The crude product was purified by SFC (SFC 8). The product containing fractions were combined, concentrated under reduced pressure and dried under HV to give the title compound as a white solid.

LC-MS: Rt=0.97 min; MS m/z [M+H]⁺ 586.4, m/z [M−H]⁻ 584.3; UPLC-MS 4

Examples 148 to 166 were made using analogous methods to Example 147, using methods known to the skilled chemist and using starting materials in the public domain.

| | | Rt | MS | LC-MS |
|---|---|---|---|---|
| Ex. | Structure and name | [min] | [M + H]+ | method |
| 148 | 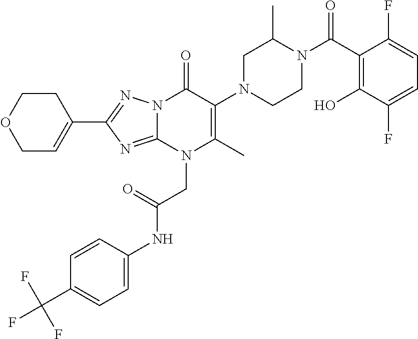 [SMILES: Cc1c(N2CCN(C(=O)c3c(F)ccc(F)c3O)C(C)C2)c(=O)n2nc(C3=CCOCC3)nc2n1CC(=O)Nc1ccc(C(F)(F)F)cc1] | 1.04 | 688.2 | UPLC- MS 8 |
| | 2-(6-(4-(3,6-difluoro-2-hydroxybenzoyl)-3- | | | |
| | methylpiperazin-1-yl)-2-(3,6-dihydro-2H-pyran-4-yl)- | | | |
| | 5-methyl-7-oxo-[1,2,4]triazolo[1,5-a]pyrimidin-4(7H)- | | | |
| | yl)-N-(4-(trifluoromethyl)phenyl)acetamide | | | |
| 149 | 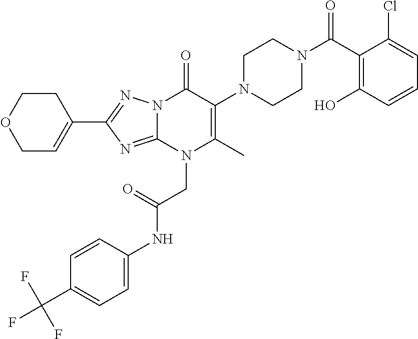 [SMILES: Cc1c(N2CCN(C(=O)c3c(O)cccc3Cl)CC2)c(=O)n2nc(C3=CCOCC3)nc2n1CC(=O)Nc1ccc(C(F)(F)F)cc1] | 1.03 | 672.3 | UPLC- MS 8 |
| | 2-(6-(4-(2-chloro-6-hydroxybenzoyl)piperazin-1-yl)- | | | |
| | 2-(3,6-dihydro-2H-pyran-4-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 150 | 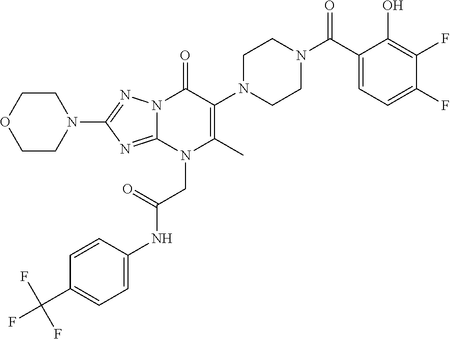 [SMILES: Cc1c(N2CCN(C(=O)c3ccc(F)c(F)c3O)CC2)c(=O)n2nc(N3CCOCC3)nc2n1CC(=O)Nc1ccc(C(F)(F)F)cc1] | 1.18 | 677.2 | UPLC- MS 11 |
| | 2-(6-(4-(3,4-difluoro-2-hydroxybenzoyl)piperazin-1- | | | |
| | yl)-5-methyl-2-morpholino-7-oxo-[1,2,4]triazolo[1,5- | | | |
| | a]pyrimidin-4(7H)-yl)-N-(4- | | | |
| | (trifluoromethyl)phenyl)acetamide | | | |
| 151 | 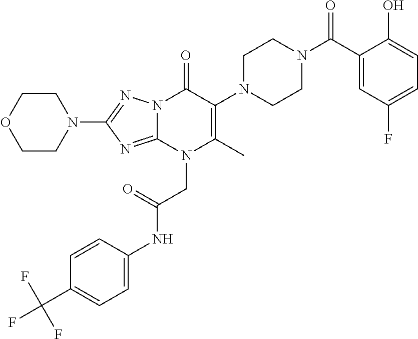 [SMILES: Cc1c(N2CCN(C(=O)c3cc(F)ccc3O)CC2)c(=O)n2nc(N3CCOCC3)nc2n1CC(=O)Nc1ccc(C(F)(F)F)cc1] | 1.17 | 659.2 | UPLC- MS 11 |
| | 2-(6-(4-(5-fluoro-2-hydroxybenzoyl)piperazin-1-yl)-5- | | | |
| | methyl-2-morpholino-7-oxo-[1,2,4]triazolo[1,5- | | | |
| | a]pyrimidin-4(7H)-yl)-N-(4- | | | |
| | (trifluoromethyl)phenyl)acetamide | | | |
| 152 | 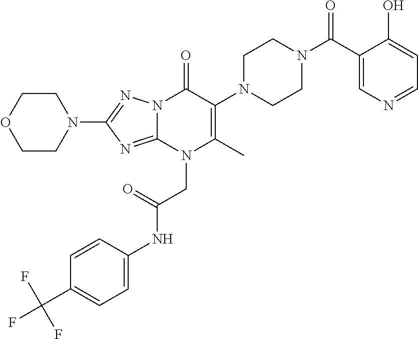 [SMILES: Cc1c(N2CCN(C(=O)c3cnccc3O)CC2)c(=O)n2nc(N3CCOCC3)nc2n1CC(=O)Nc1ccc(C(F)(F)F)cc1] | 1.38 | 642.4 | UPLC- MS 11 |
| | 2-(5-methyl-2-morpholino-7-oxo-6-(4-(4-oxo-1,4- | | | |
| | dihydropyridine-3-carbonyl)piperazin-1-yl)- | | | |
| | [1,2,4]triazolo[1,5-a]pyrimidin-4(7H)-yl)-N-(4- | | | |
| | (trifluoromethyl)phenyl)acetamide | | | |
| 153 | 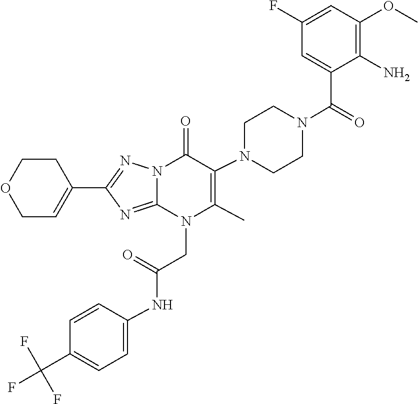 [SMILES: COc1cc(F)cc(C(=O)N2CCN(c3c(C)n(CC(=O)Nc4ccc(C(F)(F)F)cc4)c4nc(C5=CCOCC5)nn4c3=O)CC2)c1N] | 1.07 | 685.7 | UPLC- MS 8 |
| | 2-(6-(4-(2-amino-5-fluoro-3- | | | |
| | methoxybenzoyl)piperazin-1-yl)-2-(3,6-dihydro-2H- | | | |
| | pyran-4-yl)-5-methyl-7-oxo-[1,2,4]triazolo[1,5- | | | |
| | a]pyrimidin-4(7H)-yl)-N-(4- | | | |
| | (trifluoromethyl)phenyl)acetamide | | | |
| 154 | 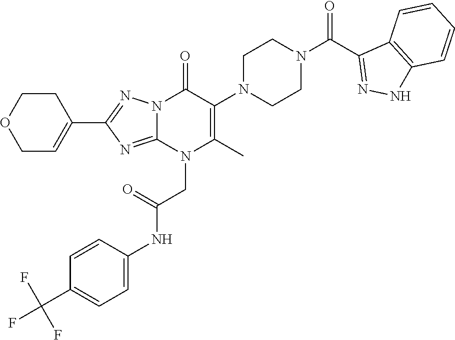 [SMILES: Cc1c(N2CCN(C(=O)c3n[nH]c4ccccc34)CC2)c(=O)n2nc(C3=CCOCC3)nc2n1CC(=O)Nc1ccc(C(F)(F)F)cc1] | 1.04 | 662.7 | UPLC- MS 15 |
| | 2-(6-(4-(1H-indazole-3-carbonyl)piperazin-1-yl)-2- | | | |
| | (3,6-dihydro-2H-pyran-4-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 155 | 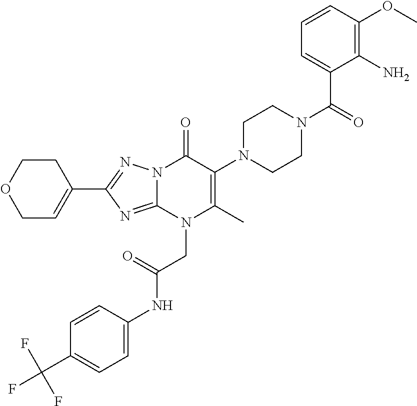 [SMILES: COc1cccc(C(=O)N2CCN(c3c(C)n(CC(=O)Nc4ccc(C(F)(F)F)cc4)c4nc(C5=CCOCC5)nn4c3=O)CC2)c1N] | 1.07 | 667.7 | UPLC- MS 15 |
| | 2-(6-(4-(2-amino-3-methoxybenzoyl)piperazin-1-yl)- | | | |
| | 2-(3,6-dihydro-2H-pyran-4-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 156 | 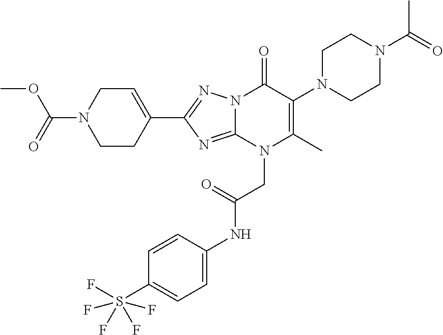 [SMILES: COC(=O)N1CC=C(c2nc3n(CC(=O)Nc4ccc(S(F)(F)(F)(F)F)cc4)c(C)c(N4CCN(C(C)=O)CC4)c(=O)n3n2)CC1] | 1.02 | 675.3 | UPLC- MS 15 |
| | methyl 4-(6-(4-acetylpiperazin-1-yl)-5-methyl-7-oxo- | | | |
| | 4-(2-oxo-2-((4-(pentafluoro-16- | | | |
| | sulfaneyl)phenyl)amino)ethyl)-4,7-dihydro- | | | |
| | [1,2,4]triazolo[1,5-a]pyrimidin-2-yl)-3,6- | | | |
| | dihydropyridine-1(2H)-carboxylate | | | |
| 157 | 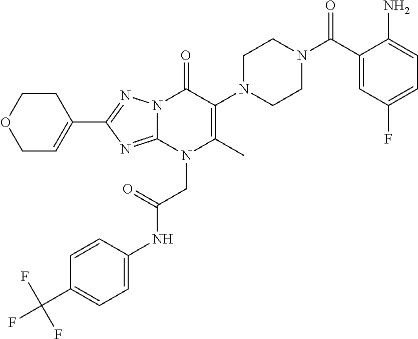 [SMILES: Cc1c(N2CCN(C(=O)c3cc(F)ccc3N)CC2)c(=O)n2nc(C3=CCOCC3)nc2n1CC(=O)Nc1ccc(C(F)(F)F)cc1] | 1.05 | 655.7 | UPLC- MS 15 |
| | 2-(6-(4-(2-amino-5-fluorobenzoyl)piperazin-1-yl)-2- | | | |
| | (3,6-dihydro-2H-pyran-4-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 158 | 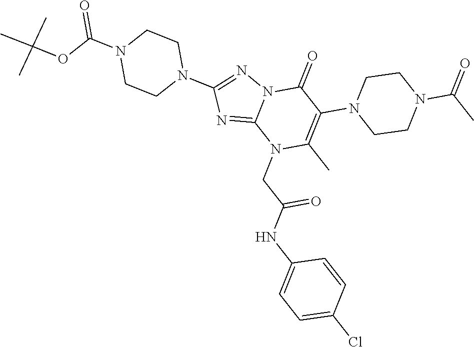 [SMILES: CC(=O)N1CCN(c2c(C)n(CC(=O)Nc3ccc(Cl)cc3)c3nc(N4CCN(C(=O)OC(C)(C)C)CC4)nn3c2=O)CC1] | 1.05 | 628.4 | UPLC- MS 15 |
| | tert-butyl 4-(6-(4-acetylpiperazin-1-yl)-4-(2-((4- | | | |
| | chlorophenyl)amino)-2-oxoethyl)-5-methyl-7-oxo- | | | |
| | 4,7-dihydro-[1,2,4]triazolo[1,5-a]pyrimidin-2- | | | |
| | yl)piperazine-1-carboxylate | | | |
| 159 | 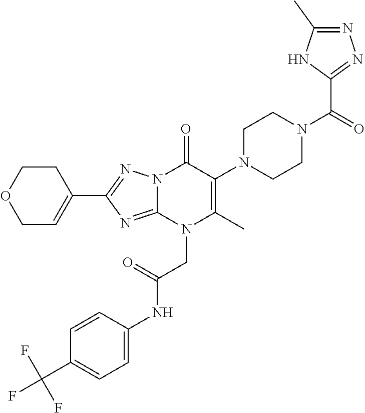 [SMILES: Cc1nnc(C(=O)N2CCN(c3c(C)n(CC(=O)Nc4ccc(C(F)(F)F)cc4)c4nc(C5=CCOCC5)nn4c3=O)CC2)[nH]1] | 0.88 | 627.6 | UPLC- MS 8 |
| | 2-(2-(3,6-dihydro-2H-pyran-4-yl)-5-methyl-6-(4-(5- | | | |
| | methyl-4H-1,2,4-triazole-3-carbonyl)piperazin-1-yl)- | | | |
| | 7-oxo-[1,2,4]triazolo[1,5-a]pyrimidin-4(7H)-yl)-N-(4- | | | |
| | (trifluoromethyl)phenyl)acetamide | | | |
| 160 | 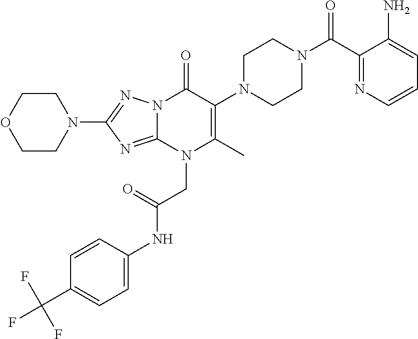 [SMILES: Cc1c(N2CCN(C(=O)c3ncccc3N)CC2)c(=O)n2nc(N3CCOCC3)nc2n1CC(=O)Nc1ccc(C(F)(F)F)cc1] | 1.41 | 641.4 | UPLC- MS 11 |
| | 2-(6-(4-(3-aminopicolinoyl)piperazin-1-yl)-5-methyl- | | | |
| | 2-morpholino-7-oxo-[1,2,4]triazolo[1,5-a]pyrimidin- | | | |
| | 4(7H)-yl)-N-(4-(trifluoromethyl)phenyl)acetamide | | | |
| 161 | 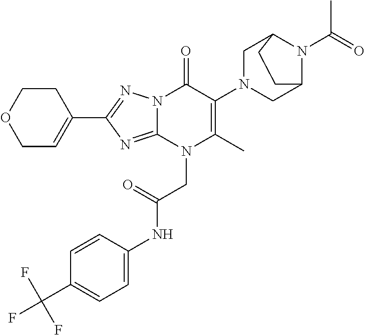 [SMILES: CC(=O)N1C2CCC1CN(c1c(C)n(CC(=O)Nc3ccc(C(F)(F)F)cc3)c3nc(C4=CCOCC4)nn3c1=O)C2] | 0.98 | 586.4 | UPLC- MS 8 |
| | 2-(6-(8-acetyl-3,8-diazabicyclo[3.2.1]octan-3-yl)-2- | | | |
| | (3,6-dihydro-2H-pyran-4-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 162 | 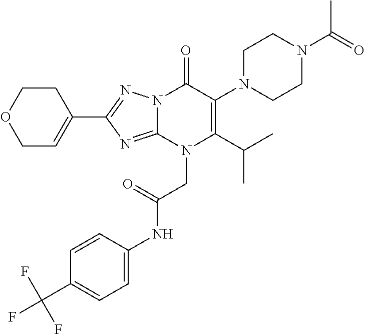 [SMILES: CC(=O)N1CCN(c2c(C(C)C)n(CC(=O)Nc3ccc(C(F)(F)F)cc3)c3nc(C4=CCOCC4)nn3c2=O)CC1] | 1.01 | 588.4 | UPLC- MS 4 |
| | 2-(6-(4-acetylpiperazin-1-yl)-2-(3,6-dihydro-2H- | | | |
| | pyran-4-yl)-5-isopropyl-7-oxo-[1,2,4]triazolo[1,5- | | | |
| | a]pyrimidin-4(7H)-yl)-N-(4- | | | |
| | (trifluoromethyl)phenyl)acetamide | | | |
| 163 | 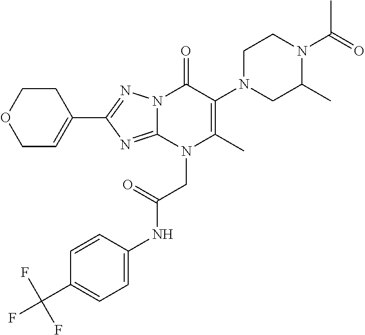 [SMILES: CC(=O)N1CCN(c2c(C)n(CC(=O)Nc3ccc(C(F)(F)F)cc3)c3nc(C4=CCOCC4)nn3c2=O)CC1C] | 0.97 | 574.6 | UPLC- MS 15 |
| | 2-(6-(4-acetyl-3-methylpiperazin-1-yl)-2-(3,6- | | | |
| | dihydro-2H-pyran-4-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 164 | 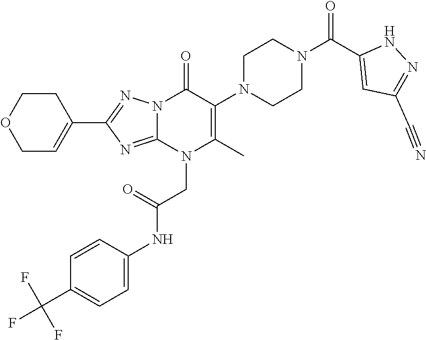 [SMILES: Cc1c(N2CCN(C(=O)c3cc(C#N)n[nH]3)CC2)c(=O)n2nc(C3=CCOCC3)nc2n1CC(=O)Nc1ccc(C(F)(F)F)cc1] | 0.99 | 637.6 | UPLC- MS 8 |
| | 2-(6-(4-(3-cyano-1H-pyrazole-5-carbonyl)piperazin- | | | |
| | 1-yl)-2-(3,6-dihydro-2H-pyran-4-yl)-5-methyl-7-oxo- | | | |
| | [1,2,4]triazolo[1,5-a]pyrimidin-4(7H)-yl)-N-(4- | | | |
| | (trifluoromethyl)phenyl)acetamide | | | |
| 165 | 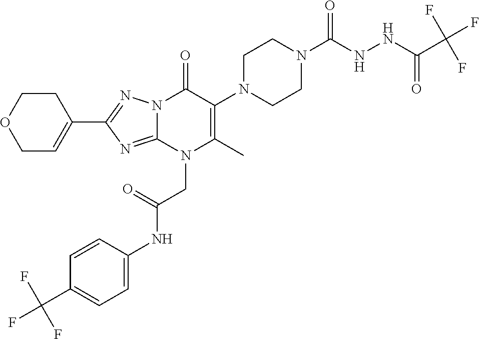 [SMILES: Cc1c(N2CCN(C(=O)NNC(=O)C(F)(F)F)CC2)c(=O)n2nc(C3=CCOCC3)nc2n1CC(=O)Nc1ccc(C(F)(F)F)cc1] | 0.95 | 672.3 | UPLC- MS 8 |
| | 2-(2-(3,6-dihydro-2H-pyran-4-yl)-5-methyl-7-oxo-6- | | | |
| | (4-(2-(2,2,2-trifluoroacetyl)hydrazine-1- | | | |
| | carbonyl)piperazin-1-yl)-[1,2,4]triazolo[1,5- | | | |
| | a]pyrimidin-4(7H)-yl)-N-(4- | | | |
| | (trifluoromethyl)phenyl)acetamide | | | |
| 166 | 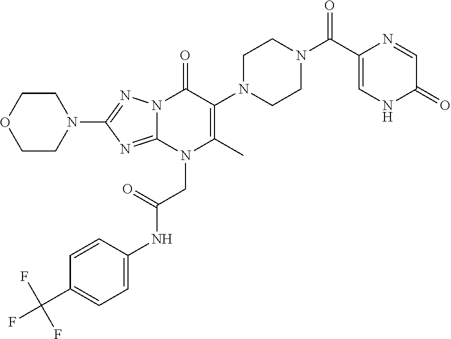 [SMILES: Cc1c(N2CCN(C(=O)c3c[nH]c(=O)cn3)CC2)c(=O)n2nc(N3CCOCC3)nc2n1CC(=O)Nc1ccc(C(F)(F)F)cc1] | 1.44 | 643.2 | UPLC- MS 11 |
| | 2-(5-methyl-2-morpholino-7-oxo-6-(4-(5-oxo-4,5- | | | |
| | dihydropyrazine-2-carbonyl)piperazin-1-yl)- | | | |
| | [1,2,4]triazolo[1,5-a]pyrimidin-4(7H)-yl)-N-(4- | | | |
| | (trifluoromethyl)phenyl)acetamide | | | |

### Intermediates

### Intermediate A: 2-(2-(3,6-dihydro-2H-pyran-4-yl)-5-methyl-7-oxo-6-(piperazin-1-yl)-[1,2,4]triazolo[1,5-a]pyrimidin-4(7H)-yl)-N-(4-trifluoromethyl)phenyl)acetamide

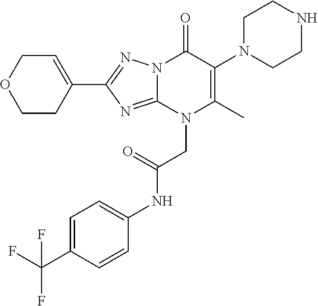 [SMILES: Cc1c(N2CCNCC2)c(=O)n2nc(C3=CCOCC3)nc2n1CC(=O)Nc1ccc(C(F)(F)F)cc1]

### Step 1: tert-butyl 4-(2-bromo-5-methyl-7-oxo-4-(2-oxo-2-((4-(trifluoromethyl)phenyl)amino)ethyl)-4,7-dihydro-[1,2,4]triazolo[1,5-a]pyrimidin-6-yl)piperazine-1-carboxylate

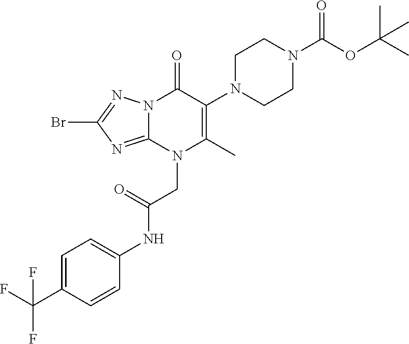 [SMILES: Cc1c(N2CCN(C(=O)OC(C)(C)C)CC2)c(=O)n2nc(Br)nc2n1CC(=O)Nc1ccc(C(F)(F)F)cc1]

Tert-butyl 4-(2-bromo-5-methyl-7-oxo-4,7-dihydro-[1,2,4]triazolo[1,5-a]pyrimidin-6-yl)piperazine-1-carboxylate (Intermediate J) (7.38 g, 17.9 mmol), 2-bromo-N-(4-(trifluoromethyl)phenyl)acetamide (6.04 g, 21.4 mmol) and DIPEA (9.36 mL, 53.6 mmol) were dissolved in DMF (50 mL) and the RM was stirred at 80° C. for 2 hours. The RM was cooled to RT, diluted with DCM, and the organic phase was extracted with aq sat NaHCO₃ and brine, dried over Na₂SO₄ and concentrated under reduced pressure. The crude product was purified by column chromatography (eluent heptane:EtOAc/MeOH (9/1) 100:0 to 30:70). The product containing fractions were combined and concentrated under reduced pressure and then crystallized from TBME to give the title compound.

LC-MS: Rt=1.15 min; MS m/z [M+H]⁺ 614.0, m/z [M−H]⁻ 612.0; UPLC-MS 4

### Step 2: tert-butyl 4-(2-(3,6-dihydro-2H-pyran-4-yl)-5-methyl-7-oxo-4-(2-oxo-2-((4-(trifluoromethyl)phenyl)amino)ethyl)-4,7-dihydro-[1,2,4]triazolo[1,5-a]pyrimidin-6-yl)piperazine-1-carboxylate

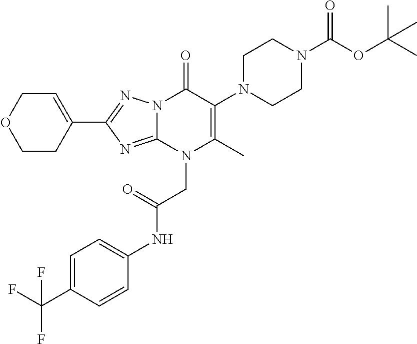 [SMILES: Cc1c(N2CCN(C(=O)OC(C)(C)C)CC2)c(=O)n2nc(C3=CCOCC3)nc2n1CC(=O)Nc1ccc(C(F)(F)F)cc1]

Tert-butyl 4-(2-bromo-5-methyl-7-oxo-4-(2-oxo-2-((4-(trifluoromethyl)phenyl)amino)ethyl)-4,7-dihydro-[1,2,4]triazolo[1,5-a]pyrimidin-6-yl)piperazine-1-carboxylate (7.70 g, 12.5 mmol), 2-(3,6-dihydro-2H-pyran-4-yl)-4,4,5,5-tetramethyl-1,3,2-dioxaborolane (3.95 g, 18.8 mmol), K₃PO₄ 1.5N (20.9 mL, 31.3 mmol) and XPhos Pd G3 (1.06 g, 1.25 mmol) were dissolved in 1,4-dioxane (50 mL). The RM was stirred at 90° C. for 1 hour and after cooling diluted with EtOAc. The organic phase was washed with aq sat NaHCO₃ and brine, dried over Na₂SO₄ and concentrated under reduced pressure. This material was dissolved in DCM/MeOH (1:1) and ISOLUTE® Si-Thiol (258 mg) was added. After stirring for 30 minutes, the mixture was filtered and concentrated. The crude product was crystallized from DCM and TBME to give the title compound.

LC-MS: Rt=1.13 min; MS m/z [M+H]⁺ 618.2, m/z [M−H]⁻ 616.1; UPLC-MS 4

### Step 3: 2-(2-(3,6-dihydro-2H-pyran-4-yl)-5-methyl-7-oxo-6-(piperazin-1-yl)-[1,2,4]triazolo[1,5-a]pyrimidin-4(7H)-yl)-N-(4-(trifluoromethyl)phenyl)acetamide

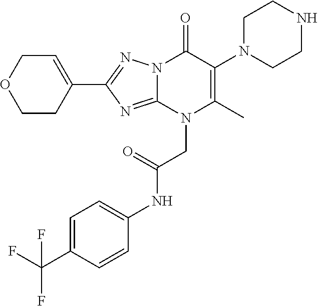 [SMILES: Cc1c(N2CCNCC2)c(=O)n2nc(C3=CCOCC3)nc2n1CC(=O)Nc1ccc(C(F)(F)F)cc1]

Tert-butyl 4-(2-(3,6-dihydro-2H-pyran-4-yl)-5-methyl-7-oxo-4-(2-oxo-2-((4-(trifluoromethyl)phenyl)amino)ethyl)-4,7-dihydro-[1,2,4]triazolo[1,5-a]pyrimidin-6-yl)piperazine-1-carboxylate (5.95 g, 9.63 mmol) was dissolved in DCM (50 mL) and TFA (22.3 mL, 289 mmol) was added. The RM was stirred at RT for 1 hour and then concentrated under reduced pressure. Toluene was added and removed again, and this procedure was repeated. The residue was dissolved in EtOAc and washed with aq sat NaHCO₃ and brine. During the extraction the product crystallized, and the solids were collected and dried to give the title compound.

LC-MS: Rt=0.84 min; MS m/z [M+H]⁺ 518.2, m/z [M−H]⁻ 516.0; UPLC-MS 4

### Intermediate B: 2-(5-cyclopropyl-2-(3,6-dihydro-2H-pyran-4-yl)-7-oxo-6-(piperazin-1-yl)-[1,2,4]triazolo[1,5-a]pyrimidin-4(7H)-yl)-N-(4-(trifluoromethyl)phenyl)acetamide

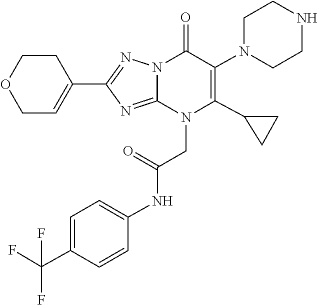 [SMILES: O=C(Cn1c(C2CC2)c(N2CCNCC2)c(=O)n2nc(C3=CCOCC3)nc12)Nc1ccc(C(F)(F)F)cc1]

### Step 1: 2-bromo-5-cyclopropyl-[1,2,4]triazolo[1,5-a]pyrimidin-7(4H)-one

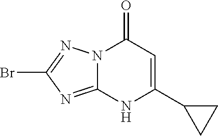 [SMILES: O=c1cc(C2CC2)[nH]c2nc(Br)nn12]

5-Bromo-4H-1,2,4-triazol-3-amine (5.00 g, 29.1 mmol) and ethyl 3-cyclopropyl-3-oxopropanoate (6.83 g, 43.7 mmol) were mixed in 1-butanol (40 mL). H₃PO₄ (8.40 g, 72.9 mmol) was added and the RM was stirred at 100° C. for 20 hours. Ethyl 3-cyclopropyl-3-oxopropanoate (1.00 g, 6.40 mmol) was added and the RM was stirred at 100° C. for 22.5 hours. Ethyl 3-cyclopropyl-3-oxopropanoate (1.00 g, 6.40 mmol) was added and the RM was stirred at 100° C. for 23.5 hours. The RM was cooled to RT and the yellow suspension was filtered. The cake was washed with a small amount of EtOH and the filtrate was concentrated under reduced pressure. The cake was washed with hot EtOH and the filtrate was concentrated under reduced pressure. The cake and the filtrate contained product, so both were combined again and concentrated under reduced pressure. The resulting oil was stood at RT over the weekend. A solid crystallized out which was filtered off and washed with Et₂O to give a white solid (2.62 g). The crude product was adsorbed onto Isolute and purified by column chromatography (Silica gel column: Silica 40 g, eluent DCM:MeOH 100:0 to 85:15). The product containing fractions were combined and concentrated under reduced pressure to give the title compound as a white solid (923 mg, 99% pure, yield: 12%). The mother liquid was concentrated, adsorbed onto Isolute and purified by column chromatography (Silica gel column: Silica 120 g, eluent DCM:MeOH 100:0 to 85:15). The product containing fractions were combined and concentrated under reduced pressure to give the title compound as a beige solid (1.14 g, 99% pure, yield: 15%).

Total: 2.06 g, 99% pure, yield: 27%).

LC-MS: Rt=0.50 min; MS m/z [M+H]⁺ 255.0/257.0, m/z [M−H]⁻ 252.9/254.9; UPLC-MS 4

### Step 2: 2-(2-bromo-5-cyclopropyl-7-oxo-[1,2,4]triazolo[1,5-a]pyrimidin-4(7H)-yl)-N-(4-(trifluoromethyl)phenyl)acetamide

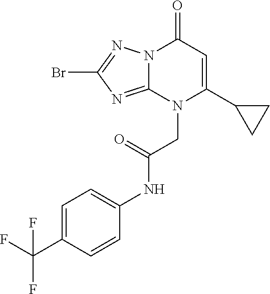 [SMILES: O=C(Cn1c(C2CC2)cc(=O)n2nc(Br)nc12)Nc1ccc(C(F)(F)F)cc1]

2-Bromo-5-cyclopropyl-[1,2,4]triazolo[1,5-a]pyrimidin-7(4H)-one (1.19 g, 4.67 mmol) and 2-bromo-N-(4-(trifluoromethyl)phenyl)acetamide (1.73 g, 5.84 mmol) were mixed in DMF (12 mL). DIPEA (2.50 mL, 14.0 mmol) was added and the RM was stirred at 65° C. for 6 hours, then at RT overnight. Water was added and the RM was continued stirring at RT. A resin collapsed. The suspension was filtered and the cake was washed with water. The cake (1.78 g) was suspended in DCM and MeOH and filtered off. Then it was washed and dried under Hv to give the title compound as a beige solid (829 mg, 85% pure, yield: 33%).

LC-MS: Rt=1.03 min; MS m/z [M+H]⁺ 456.1/458.1, m/z [M−H]⁻ 453.9/455.9; UPLC-MS 4

### Step 3: 2-(5-cyclopropyl-2-(3,6-dihydro-2H-pyran-4-yl)-7-oxo-[1,2,4]triazolo[1,5-a]pyrimidin-4(7H)-yl)-N-(4-(trifluoromethyl)phenyl)acetamide

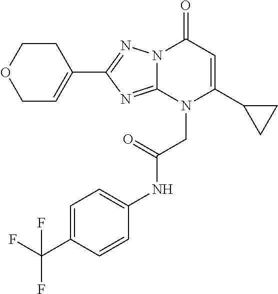 [SMILES: O=C(Cn1c(C2CC2)cc(=O)n2nc(C3=CCOCC3)nc12)Nc1ccc(C(F)(F)F)cc1]

2-(2-Bromo-5-cyclopropyl-7-oxo-[1,2,4]triazolo[1,5-a]pyrimidin-4(7H)-yl)-N-(4-(trifluoromethyl)phenyl)acetamide (1.34 g, 2.94 mmol), 2-(3,6-dihydro-2H-pyran-4-yl)-4,4,5,5-tetramethyl-1,3,2-dioxaborolane (926 mg, 4.41 mmol) and Pd X-Phos G3 (124 mg, 147 μmol) were mixed in dioxane (15 mL) and 1M K₃PO₄ in water (8.81 mL, 8.81 mmol) was added. The RM was vacuumed and backfilled with argon several times, then it was stirred at 90° C. for 1.5 hours. The RM was cooled to RT. The RM was extracted with EtOAc (3×70 mL) and water (2×20 mL). The organic layer was dried through a phase separator and concentrated under reduced pressure. The aqueous layer was a suspension which was filtered. The aqueous layer was extracted three times with DCM, dried through a phase separator and concentrated under reduced pressure. All organics were combined with the cake and were suspended in hot EtOH (500 mL). Then it was filtered, and the cake was dissolved in warm ACN and Si-Thiol (2.00 g) was added. The mixture was stirred at 45° C. for 5 minutes, then it was filtered. The filtrate was concentrated under reduced pressure to give the title compound as a grey solid (650 mg, 99% pure, yield: 48%). The mother liquor was mixed with Si-Thiol (2.00 g) and stirred at 45° C. for 5 minutes, then it was filtered. The filtrate was concentrated under reduced pressure to give the title compound as a bright brown solid (610 mg, 79% pure, yield: 36%).

Total: 1.26 g, 89% pure, yield: 84%.

LC-MS: Rt=0.98 min; MS m/z [M+H]⁺ 460.3, m/z [M−H]⁻ 458.3; UPLC-MS 4

### Step 4: 2-(6-bromo-5-cyclopropyl-2-(3,6-dihydro-2H-pyran-4-yl)-7-oxo-[1,2,4]triazolo[1,5-a]pyrimidin-4(7H)-yl)-N-(4-(trifluoromethyl)phenyl)acetamide

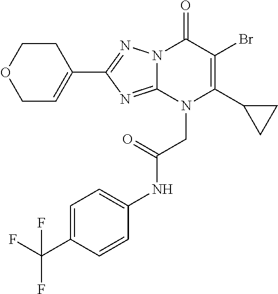 [SMILES: O=C(Cn1c(C2CC2)c(Br)c(=O)n2nc(C3=CCOCC3)nc12)Nc1ccc(C(F)(F)F)cc1]

2-(5-Cyclopropyl-2-(3,6-dihydro-2H-pyran-4-yl)-7-oxo-[1,2,4]triazolo[1,5-a]pyrimidin-4(7H)-yl)-N-(4-(trifluoromethyl)phenyl)acetamide (610 mg, 1.33 mmol) and NBS (295 mg, 1.66 mmol) were mixed in DMF (28 mL). The RM was stirred at 60° C. for 5.5 hours, then it was stood at RT over 2 days. NBS (125 mg, 703 μmol) was added and the RM was stirred at 60° C. for 5 hours. NBS (50 mg, 281 μmol) was added and the RM was stirred at 60° C. for 1.5 hours. Then it was cooled to RT and stood at RT overnight. The RM was diluted with DCM and aq sat NaHCO₃. Most of the DMF was removed under reduced pressure. The solid residue was extracted with EtOAc (3×40 mL), water (2×20 mL) and brine (25 mL). The organic layer was dried through a phase separator and concentrated under reduced pressure. The brown solid residue was mixed with hexane and the suspension was filtered. The cake was mixed again with hexane and filtered again. The cake was dried under HV to give the title compound as a bright brown solid (508 mg, 74% pure, yield: 53%).

LC-MS: Rt=1.04 min; MS m/z [M+H]⁺ 538.1/540.1, m/z [M−H]⁻ 536.2/538.2; UPLC-MS 4

### Step 5: 2-(5-cyclopropyl-2-(3,6-dihydro-2H-pyran-4-yl)-7-oxo-6-(piperazin-1-yl)-[1,2,4]triazolo[1,5-a]pyrimidin-4(7H)-yl)-N-(4-(trifluoromethyl)phenyl)acetamide

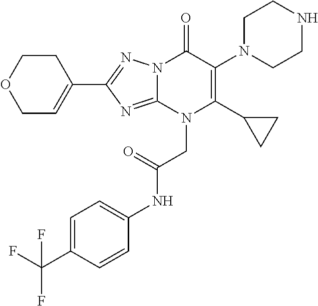 [SMILES: O=C(Cn1c(C2CC2)c(N2CCNCC2)c(=O)n2nc(C3=CCOCC3)nc12)Nc1ccc(C(F)(F)F)cc1]

2-(6-bromo-5-cyclopropyl-2-(3,6-dihydro-2H-pyran-4-yl)-7-oxo-[1,2,4]triazolo[1,5-a]pyrimidin-4(7H)-yl)-N-(4-(trifluoromethyl)phenyl)acetamide (548 mg, 743 μmol) and piperazine (2.50 g, 29.0 mmol) were mixed with DMSO (5 mL) and the RM was stirred at 140° C. under argon for 5 hours. The RM was cooled to RT and stood at RT overnight, then it was combined with another batch. The RM was extracted with EtOAc (3×80 mL), aq sat NaHCO₃ (2×30 mL) and water (2×30 mL). The organic layer was washed with 1 N HCl (4×25 mL) and water (2×20 mL). The organic layer was concentrated a bit and extracted twice with 1N HCl. The combined aqueous layers were basified with solid NaHCO₃ and extracted three times with EtOAc. The organic layer was dried through a phase separator and concentrated under reduced pressure. The solid residue was suspended in DCM and MeOH and filtered. The cake was washed well with DCM and the filtrate was concentrated down to give a bright brown solid (254 mg). The crude product was adsorbed onto Isolute and purified by column chromatography (Silica gel column: Silica 24 g, eluent DCM:MeOH/Et₃N (95/5) 90:10 to 50:50). The product containing fractions were combined and concentrated under reduced pressure to give the title compound as a solid (97.0 mg, 87% pure, yield: 21%).

LC-MS: Rt=0.86 min; MS m/z [M+H]⁺ 544.3, m/z [M−H]⁻ 542.3; UPLC-MS 3

### Intermediate C: 2-(2-(3,6-dihydro-2H-pyran-4-yl)-5-methyl-7-oxo-6-(piperidin-4-yl)-[1,2,4]triazolo[1,5-a]pyrimidin-4(7H)-yl)-N-(4-(trifluoromethyl)phenyl)acetamide

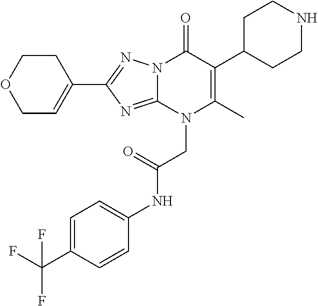 [SMILES: Cc1c(C2CCNCC2)c(=O)n2nc(C3=CCOCC3)nc2n1CC(=O)Nc1ccc(C(F)(F)F)cc1]

### Step 1: 2-bromo-5-methyl-[1,2,4]triazolo[1,5-a]pyrimidin-7(4H)-one

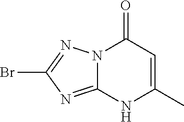

To a solution of 3-bromo-1H-1,2,4-triazol-5-amine (Intermediate R) (60.0 g, 60.6 mmol) in AcOH (380 mL) was added ethyl 3-oxobutanoate (86.6 g, 66.6 mmol). The RM was stirred at 80° C. overnight. The mixture was filtrate and washed with AcOH (160 mL). The wet filter cake was dried to give the title compound (80.0 g, 80%).

### Step 2: 2-bromo-6-iodo-5-methyl-[1,2,4]triazolo[1,5-a]pyrimidin-7(4H)-one

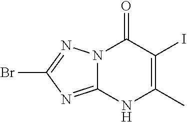

2-Bromo-5-methyl-[1,2,4]triazolo[1,5-a]pyrimidin-7(4H)-one (2.00 g, 8.73 μmol) was added in a flame dried flask under nitrogen. Acetic acid (29.1 mL) was added, followed by NIS (2.16 g, 9.61 mmol). The RM was stirred at 60° C. for 1 hour. The RM was cooled to RT. The solid was filtered off and washed 3 times with EtOH. The solid was dried under Hv to give the title compound (2.76 g, 95% pure, yield: 89%).

LC-MS: Rt=0.56 min; MS m/z [M+H]⁺ 354.9/356.9, m/z [M−H]⁻ 353.0/355.0; UPLC-MS 8

### Step 3: tert-butyl 4-(2-bromo-5-methyl-7-oxo-4,7-dihydro-[1,2,4]triazolo[1,5-a]pyrimidin-6-yl)-3,6-dihydropyridine-1(2H)-carboxylate

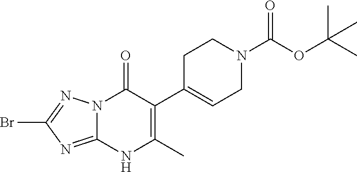 [SMILES: Cc1[nH]c2nc(Br)nn2c(=O)c1C1=CCN(C(=O)OC(C)(C)C)CC1]

Palladium G3-Tricyclohexylphosphine, [(Tricyclohexylphosphine)-2-(2-aminobiphenyl)]palladium(II) methanesulfonate (183 mg, 282 μmol) was purged with nitrogen. A solution of 2-bromo-6-iodo-5-methyl-[1,2,4]triazolo[1,5-a]pyrimidin-7(4H)-one (1.00 g, 2.82 mmol) and tert-butyl 4-(4,4,5,5-tetramethyl-1,3,2-dioxaborolan-2-yl)-3,6-dihydropyridine-1(2H)-carboxylate (894 mg, 2.89 mmol) in n-butanol (100 μL) was added, followed by K₃PO₄ 1.5M in water (5.63 mL, 8.45 mmol). The RM was stirred at 70° C. for 1 hour. Tert-butyl 4-(4,4,5,5-tetramethyl-1,3,2-dioxaborolan-2-yl)-3,6-dihydropyridine-1(2H)-carboxylate (50.0 mg, 162 μmol) was added and the RM was stirred at 70° C. overnight. N-butanol was removed under reduced pressure. EtOAc was added and the mixture was washed with NH₄Cl. The organic layer was dried through a phase separator. ISOLUTE® Si-TMT (6.50 g, 2.82 mmol) was added and the mixture was stirred at 40° C. for 1 hour. The solid was filtered off and washed with EtOAc. The filtrate was concentrated under reduced pressure. The crude product was purified by column chromatography (Silica gel column: Silica 40 g, eluent DCM:DCM/MeOH (8/2) 100:0 to 50:50) to give the title compound as a white powder (734 mg, 90% pure, yield: 57%).

LC-MS: Rt=0.86 min; MS m/z [M−H]⁻ 408.2/410.2; UPLC-MS 14

### Step 4: tert-butyl 4-(2-bromo-5-methyl-7-oxo-4,7-dihydro-[1,2,4]triazolo[1,5-a]pyrimidin-6-yl)piperidine-1-carboxylate

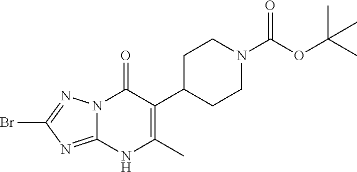 [SMILES: Cc1[nH]c2nc(Br)nn2c(=O)c1C1CCN(C(=O)OC(C)(C)C)CC1]

To a solution of tert-butyl 4-(2-bromo-5-methyl-7-oxo-4,7-dihydro-[1,2,4]triazolo[1,5-a]pyrimidin-6-yl)-3,6-dihydropyridine-1(2H)-carboxylate (734 mg, 1.79 mmol) in MeOH (10 mL) was added PtO₂ (40.6 mg, 179 μmol) under N2 atmosphere. The flask was purged with hydrogen for 2 minutes. The RM was stirred at RT for 2 hours. PtO₂ (66.0 mg, 291 μmol) was added and the RM was stirred at RT for 2 hours. PtO₂ (103 mg, 454 μmol) was added and the RM was stirred at RT overnight. PtO₂ (122 mg, 543 μmol) was added and the RM was stirred at RT. The RM was filtered through a pad of celite. The crude product was purified by column chromatography (Silica gel column: Silica 12 g, eluent DCM:DCM/MeOH (8/2) 100:0 to 50:50) to give the title compound (580 mg, 80% pure, yield: 63%).

LC-MS: Rt=0.89 min; MS m/z [M+H]⁺ 412.2/414.2, m/z [M−H]⁻ 410.3/412.3; UPLC-MS 8

### Step 5: tert-butyl 4-(2-(3,6-dihydro-2H-pyran-4-yl)-5-methyl-7-oxo-4,7-dihydro-[1,2,4]triazolo[1,5-a]pyrimidin-6-yl)piperidine-1-carboxylate

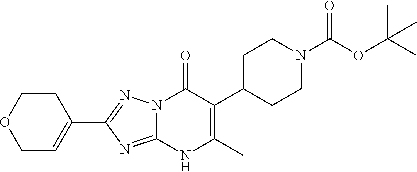 [SMILES: Cc1[nH]c2nc(C3=CCOCC3)nn2c(=O)c1C1CCN(C(=O)OC(C)(C)C)CC1]

Tert-butyl 4-(2-bromo-5-methyl-7-oxo-4,7-dihydro-[1,2,4]triazolo[1,5-a]pyrimidin-6-yl)piperidine-1-carboxylate (580 mg, 1.41 mmol), 2-(3,6-dihydro-2H-pyran-4-yl)-4,4,5,5-tetramethyl-1,3,2-dioxaborolane (443 mg, 2.11 mmol) and X-Phos Pd G3 (71.4 mg, 84.0 μmol) were mixed. Under N2 atmosphere were added DMF (1.4 mL) and K₃PO₄ 1M in water (2.81 mL, 1.82 mmol). The RM was stirred at 80° C. for 1 hour. X-Phos Pd G3 (10.0 mg, 11.8 μmol) and 2-(3,6-dihydro-2H-pyran-4-yl)-4,4,5,5-tetramethyl-1,3,2-dioxaborolane (60.0 mg, 286 μmol) were added and the RM was stirred for 3 hours to give the title compound as RM.

LC-MS: Rt=1.25 min; MS m/z [M+H]⁺ 416.3, m/z [M−H]⁻ 414.4; UPLC-MS 8

### Step 6: tert-butyl 4-(2-(3,6-dihydro-2H-pyran-4-yl)-5-methyl-7-oxo-4-(2-oxo-2-((4-(trifluoromethyl)phenyl)amino)ethyl)-4,7-dihydro-[1,2,4]triazolo[1,5-a]pyrimidin-6-yl)piperidine-1-carboxylate

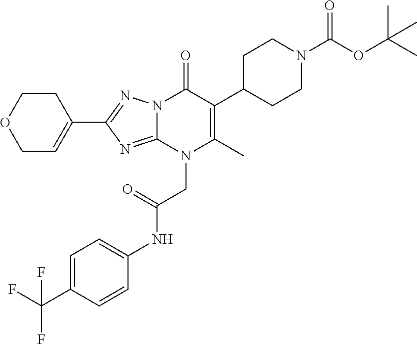 [SMILES: Cc1c(C2CCN(C(=O)OC(C)(C)C)CC2)c(=O)n2nc(C3=CCOCC3)nc2n1CC(=O)Nc1ccc(C(F)(F)F)cc1]

To the RM containing tert-butyl 4-(2-(3,6-dihydro-2H-pyran-4-yl)-5-methyl-7-oxo-4,7-dihydro-[1,2,4]triazolo[1,5-a]pyrimidin-6-yl)piperidine-1-carboxylate was added 2-bromo-N-(4-(trifluoromethyl)phenyl)acetamide (397 mg, 1.41 mmol). The RM was stirred at 80° C. for 40 minutes. 2-Bromo-N-(4-(trifluoromethyl)phenyl)acetamide (88.0 mg, 313 μmol) was added and the RM was stirred at 80° C. for 30 minutes. Most of the DMF was removed under reduced pressure. EtOAc was added and the mixture was washed with NaHCO₃. The organic layer was dried through a phase separator and concentrated under reduced pressure. The mixture was treated with Si TMT. The solvent was removed, the residue was adsorbed onto Isolute and purified by column chromatography (Silica gel column: Silica 24 g, eluent DCM:DCM/MeOH (8/2) 100:0 to 70:30), then in 5 portions by reverse phase preparative HPLC (5×RP-HPLC acidic 1:20 to 95% B in 20 min), to give the title compound (185 mg, 60% pure, yield: 18%).

LC-MS: Rt=1.18 min; MS m/z [M+H]⁺ 617.4, m/z [M−H]⁻ 615.5; UPLC-MS 8

### Step 7: 2-(2-(3,6-dihydro-2H-pyran-4-yl)-5-methyl-7-oxo-6-(piperidin-4-yl)-[1,2,4]triazolo[1,5-a]pyrimidin-4(7H)-yl)-N-(4-(trifluoromethyl)phenyl)acetamide

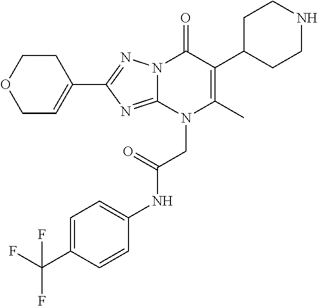 [SMILES: Cc1c(C2CCNCC2)c(=O)n2nc(C3=CCOCC3)nc2n1CC(=O)Nc1ccc(C(F)(F)F)cc1]

To tert-butyl 4-(2-(3,6-dihydro-2H-pyran-4-yl)-5-methyl-7-oxo-4-(2-oxo-2-((4-(trifluoromethyl)phenyl)amino)ethyl)-4,7-dihydro-[1,2,4]triazolo[1,5-a]pyrimidin-6-yl)piperidine-1-carboxylate (145 mg, 198 μmol) was added TFA (306 μL, 3.97 mmol) in DCM (2 mL). The RM was stirred at RT for 1 hour. DCM was added and the crude product was washed with NaOH solution. The aqueous layer was washed with EtOAc. The organic layer was dried through a phase separator and concentrated under reduced pressure to give the title compound (130 mg, 79% pure, quantitative).

LC-MS: Rt=0.74 min; MS m/z [M+H]⁺ 517.3, m/z [M−H]⁻ 515.4; UPLC-MS 8

### Intermediate D: N-(2-chloro-4-(trifluoromethyl)phenyl)-2-(2-(3,6-dihydro-2H-pyran-4-yl)-5-ethyl-7-oxo-6-(piperazin-1-yl)-[1,2,4]triazolo[1,5-a]pyrimidin-4(7H)-yl)acetamide

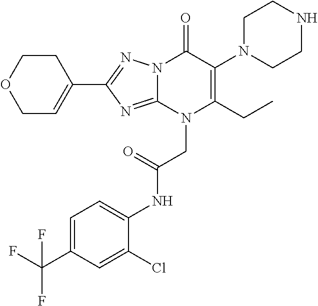 [SMILES: CCc1c(N2CCNCC2)c(=O)n2nc(C3=CCOCC3)nc2n1CC(=O)Nc1ccc(C(F)(F)F)cc1Cl]

### Step 1: tert-butyl 4-(2-bromo-5-ethyl-7-oxo-4,7-dihydro-[1,2,4]triazolo[1,5-a]pyrimidin-6-yl)piperazine-1-carboxylate

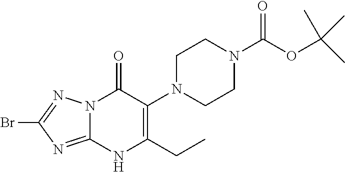 [SMILES: CCc1[nH]c2nc(Br)nn2c(=O)c1N1CCN(C(=O)OC(C)(C)C)CC1]

3-Bromo-1H-1,2,4-triazol-5-amine (Intermediate R) (82.6 g, 507 mmol) and tert-butyl 4-(1-methoxy-1,3-dioxopentan-2-yl)piperazine-1-carboxylate (Intermediate N) (175 g, 557 mmol) were mixed in EtOH (465 mL). H₃PO₄ (49.7 g, 507 mmol) was added. The mixture was stirred at 80° C. for 12 hours under nitrogen. The mixture was concentrated in vacuo to remove EtOH, then quenched by addition of aq sat NaHCO₃ (1 L), and extracted with DCM (3×1 L). The combined organic layers were washed with brine (3×1 L), dried over Na₂SO₄, filtered and concentrated under reduced pressure to give a residue. The residue was purified by column chromatography (Silica column, eluent DCM:MeOH 1:0 to 10:1). The product containing fractions were combined and concentrated under reduced pressure to give the title compound as a yellow solid.

LC-MS: Rt=0.91 min; MS m/z [M+H-Boc]⁺327.1/329.1, m/z [M+H]⁺ 427.2/429.2, m/z [M−H]⁻ 425.2/427.2; UPLC-MS 1

LC-MS: Rt=4.53 min; MS m/z [M+H−Boc]⁺327.1/329.1, m/z [M−H]⁻ 425.2/427.2; UPLC-MS 2

¹H NMR (400 MHz, DMSO-d₆) δ 13.27 (s, 1H), 3.91 (m, 2H), 3.31 (m, 2H), 2.88 (m, 2H), 2.75 (m, 2H), 2.61 (m, 2H), 1.42 (s, 9H), 1.17 (t, J=7.4 Hz, 3H)

### Step 2: tert-butyl 4-(2-(3,6-dihydro-2H-pyran-4-yl)-5-ethyl-7-oxo-4,7-dihydro-[1,2,4]triazolo[1,5-a]pyrimidin-6-yl)piperazine-1-carboxylate

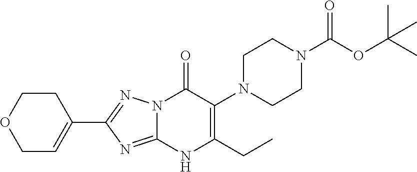 [SMILES: CCc1[nH]c2nc(C3=CCOCC3)nn2c(=O)c1N1CCN(C(=O)OC(C)(C)C)CC1]

To the stirred solution of tert-butyl 4-(2-bromo-5-ethyl-7-oxo-4,7-dihydro-[1,2,4]triazolo[1,5-a]pyrimidin-6-yl)piperazine-1-carboxylate (15.0 g, 35.1 mmol) in 1,4-dioxane (150 mL) and water (50 mL) was added 2-(3,6-dihydro-2H-pyran-4-yl)-4,4,5,5-tetramethyl-1,3,2-dioxaborolane (11.1 g, 52.7 mmol) and Na₂CO₃ (7.44 g, 70.2 mmol). The RM was degassed with nitrogen for 15 minutes. Pd(dppf)Cl₂.DCM (1.43 g, 1.76 mmol) was added and the RM was stirred at 100° C. for 14 hours. Water (300 mL) was added and the RM was extracted with 10% MeOH in DCM (2×500 mL). The organic layer was washed with brine (300 mL), dried over Na₂SO₄ and concentrated under reduced pressure. The crude product was purified by column chromatography (Silica gel column: Silica 40 g, eluent DCM:MeOH 100:0 to 97:3). The product containing fractions were combined, concentrated under vacuum and dried under HV to give the title compound.

LC-MS: Rt=0.96 min; MS m/z [M+H]⁺ 431.4, m/z [M−H]⁻ 429.3; UPLC-MS 3
¹H NMR (400 MHz, DMSO-d₆) δ 13.00 (s, br, 1H), 6.81 (m, 1H), 4.28 (m, 2H), 3.92 (m, 2H), 3.82 (m, 2H), 3.37 (m, 2H), 2.89 (m, 2H), 2.76 (m, 2H), 2.62 (m, 2H), 2.51 (m, 2H), 1.43 (s, 9H), 1.19 (t, J=7.3 Hz, 3H)

### Step 3: tert-butyl 4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazine-1-carboxylate

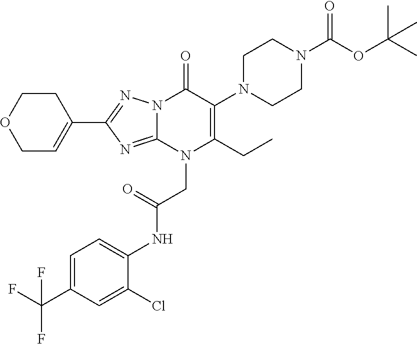 [SMILES: CCc1c(N2CCN(C(=O)OC(C)(C)C)CC2)c(=O)n2nc(C3=CCOCC3)nc2n1CC(=O)Nc1ccc(C(F)(F)F)cc1Cl]

Tert-butyl 4-(2-(3,6-dihydro-2H-pyran-4-yl)-5-ethyl-7-oxo-4,7-dihydro-[1,2,4]triazolo[1,5-a]pyrimidin-6-yl)piperazine-1-carboxylate (6.50 g, 15.1 mmol) and N-(2-chloro-4-(trifluoromethyl)phenyl)-2-iodoacetamide (Intermediate S) (6.04 g, 16.6 mmol) were mixed in DMF (72 mL) at 0° C. DIPEA (7.91 mL, 45.3 mmol) was added and the RM was stirred at 45° C. for 3.5 hours. The RM was cooled to RT. Water (70 mL) was added and the suspension was stirred at RT overnight. The suspension was sonicated for 25 minutes and filtered. The cake was washed with a small amount of water and dried. The filtrate was filtered again. The second filtrate was extracted with EtOAc (2×400 mL), washed with brine (2×50 mL), dried through a phase separator and concentrated under reduced pressure. The 2 cakes were adsorbed onto Isolute and purified by column chromatography (RediSep Column: Silica 220 g, eluent DCM:DCM/MeOH (1/1) 100:0 to 80:20). The pure product containing fractions were combined and concentrated under reduced pressure. The beige solid foam was dissolved in Et₂O and the resulting crystals were sonicated. The suspension was left standing overnight, filtered, washed with a small amount of Et₂O and dried under HV to give cake 1 as a white solid. The impure fractions were combined and concentrated under reduced pressure. Then they were combined with the concentrated organic layer from the extraction and purified by column chromatography again (RediSep Column: Silica 120 g Gold, eluent DCM:DCM/MeOH (1/1) 100:0 to 85:15). The product containing fractions were combined, concentrated under reduced pressure and dried under HV. The beige solid foam was crystallized out of Et₂O to give cake 2 as a white solid. Cake 1 and cake 2 were combined to give the title compound.

LC-MS: Rt=1.33 min; MS m/z [M+H-Boc]⁺566.0/568.0, m/z [M+H]⁺ 666.0/668.0, m/z [M−H]⁻ 664.1/666.1; UPLC-MS 1

### Step 4: N-(2-chloro-4-(trifluoromethyl)phenyl)-2-(2-(3,6-dihydro-2H-pyran-4-yl)-5-ethyl-7-oxo-6-(piperazin-1-yl)-[1,2,4]triazolo[1,5-a]pyrimidin-4(7H)-yl)acetamide

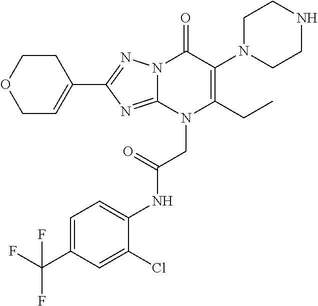 [SMILES: CCc1c(N2CCNCC2)c(=O)n2nc(C3=CCOCC3)nc2n1CC(=O)Nc1ccc(C(F)(F)F)cc1Cl]

Tert-butyl 4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazine-1-carboxylate (6.41 g, 9.62 mmol) was dissolved in DCM (70 mL) and TFA (11.1 mL, 144 mmol) was added. The RM was stirred at RT for 1 hour. The RM was concentrated under reduced pressure. The residue was dissolved in DCM and concentrated under reduced pressure again. This was performed three times. The resulting oil was dried under HV to result in a pale rose solid foam. The foam was suspended in Et₂O and sonicated. The suspension was filtered, washed with Et₂O and dried under HV to give the title compound as a white solid.

LC-MS: Rt=0.78 min; MS m/z [M+H]⁺ 566.4/568.4, m/z [M−H]⁻ 564.2/566.2; UPLC-MS 1

### Intermediate D: N-(2-chloro-4-(trifluoromethyl)phenyl)-2-(2-(3,6-dihydro-2H-pyran-4-yl)-5-ethyl-7-oxo-6-(piperazin-1-yl)-[1,2,4]triazolo[1,5-a]pyrimdin-4(7H)-yl)acetamide

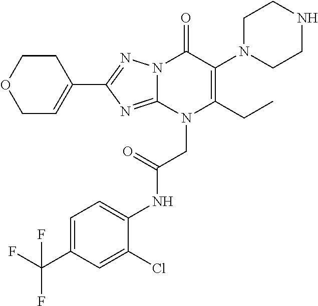 [SMILES: CCc1c(N2CCNCC2)c(=O)n2nc(C3=CCOCC3)nc2n1CC(=O)Nc1ccc(C(F)(F)F)cc1Cl]

### Step 1: tert-butyl 4-(3-amino-6-ethyl-2-imino-4-oxo-1,2,3,4-tetrahydropyrimidin-5-yl)piperazine-1-carboxylate

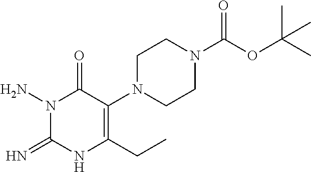 [SMILES: CCc1[nH]c(=N)n(N)c(=O)c1N1CCN(C(=O)OC(C)(C)C)CC1]

Hydrazinecarboximidamide.HCl (35.0 g, 317 mmol) was mixed with EtOH (400 mL), followed by aqueous tetrabutylammonium hydroxide solution, 40 wt % in water (206 g, 318 mmol). The mixture was stirred at 55° C. for 80 minutes. Tert-butyl 4-(1-methoxy-1,3-dioxopentan-2-yl)piperazine-1-carboxylate (Intermediate N) (50.0 g, 159 mmol) was added. The mixture was stirred at reflux for 6 hours, and then it was cooled to RT. The solvent was removed under vacuum until ¼ of the solvent volume remained. The resulting suspension was stirred for 1-2 hours, then filtered. The cake was dried under vacuum to give the title compound as a white solid.

MS m/z [M+H]⁺ 339.2

### Step 2: tert-butyl 4-(2-(3,6-dihydro-2H-pyran-4-yl)-5-ethyl-7-oxo-4,7-dihydro-[1,2,4]triazolo[1,5-a]pyrimidin-6-yl)piperazine-1-carboxylate

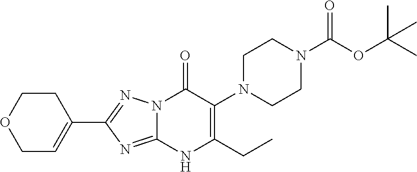 [SMILES: CCc1[nH]c2nc(C3=CCOCC3)nn2c(=O)c1N1CCN(C(=O)OC(C)(C)C)CC1]

A solution of 3,6-dihydro-2H-pyran-4-carbaldehyde (Intermediate Q) (20.0 g, 178 mmol) in NMP and tert-butyl 4-(3-amino-6-ethyl-2-imino-4-oxo-1,2,3,4-tetrahydropyrimidin-5-yl)piperazine-1-carboxylate (52.9 g, 149 mmol) in NMP (400 mL), followed by FeCl₃ (48 g, 297 mmol). The dark solution was heated to 50° C. open to air and stirred for 48 hours. The dark RM was cooled to RT. Water (1.2 L) was added slowly (exothermic). The suspension was filtered and washed with water (400 mL). The resulting wet cake was added to acetone (400 mL), stirred at RT for 4 hours. The suspension was filtered and washed with acetone (100 mL). The cake was added to EtOH (400 mL) and heated to 70° C., stirred for 4 hours. Then the mixture was cooled to RT, filtered and washed with EtOH (100 mL) to give the title compound as brown solid.

MS m/z [M+H]⁺ 430.2

### Step 3: tert-butyl 4-(4-(2-(tert-butoxy)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazine-1-carboxylate

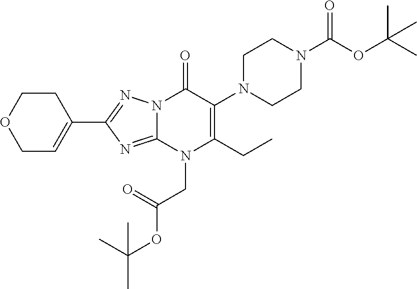 [SMILES: CCc1c(N2CCN(C(=O)OC(C)(C)C)CC2)c(=O)n2nc(C3=CCOCC3)nc2n1CC(=O)OC(C)(C)C]

Tert-butyl 4-(2-(3,6-dihydro-2H-pyran-4-yl)-5-ethyl-7-oxo-4,7-dihydro-[1,2,4]triazolo[1,5-a]pyrimidin-6-yl)piperazine-1-carboxylate (3.00 g, 6.97 mol) and tert-butyl 2-bromoacetate (1.46 g, 7.32 mmol) were mixed in DMF (20 mL) under argon. DIPEA (3.65 mL, 20.9 mmol) was added and the RM was stirred at 55° C. for 4.5 hours. Water (50 mL) was added and the RM was stirred at RT overnight. The suspension was sonicated for 10 minutes, filtered and washed with water. The cake was dried under HV, mixed with Et₂O and sonicated for 5 minutes. The suspension was stirred at reflux, filtered and the solid washed with Et₂O. A second crop of solid precipitates from the filtrate which was filtered. Both cakes were combined to give the title compound as a beige solid.

LC-MS: Rt=1.16 min; MS m/z [M+H-Boc]⁺445.4, UPLC-MS 1

### Step 4: 2-(6-(4-(tert-butoxycarbonyl)piperazin-1-yl)-2-(3,6-dihydro-2H-pyran-4-yl)-5-ethyl-7-oxo-[1,2,4]triazolo[1,5-a]pyrimidin-4(7H)-yl)acetic acid

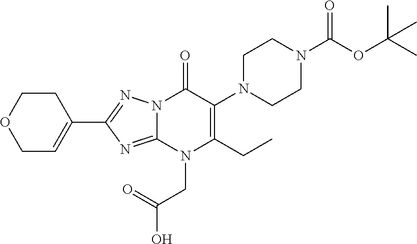 [SMILES: CCc1c(N2CCN(C(=O)OC(C)(C)C)CC2)c(=O)n2nc(C3=CCOCC3)nc2n1CC(=O)O]

Tert-butyl 4-(4-(2-(tert-butoxy)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazine-1-carboxylate (2.75 g, 5.00 mmol) was dissolved in THF (50 mL) and MeOH (20 mL). 1M NaOH (7.50 mL, 7.50 mmol) was added and the RM was stirred at RT for 18.5 hours. The solvent was removed, water was added and the RM was extracted with Et₂O (3×70 mL) and water (3×15 mL). The aqueous layer was cooled to 0° C. and 4M HCl (1.87 mL, 7.50 mmol) was added until pH 3. The resulting suspension was extracted with EtOAc (3×200 mL) and twice with brine, then twice with EtOAc. The combined organic layers were eluted through a phase separator and concentrated under reduced pressure to give the title compound as a bright brown solid.

LC-MS: Rt=0.76 min; MS m/z [M+H-Boc]⁺389.5, m/z [M−H]⁻ 487.2; UPLC-MS 1

### Step 5: tert-butyl 4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazine-1-carboxylate

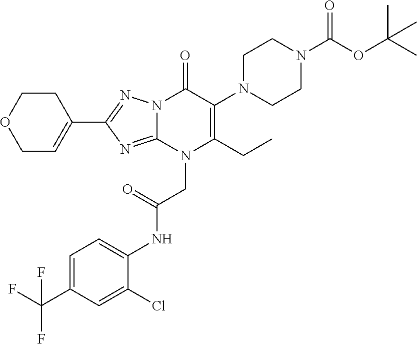 [SMILES: CCc1c(N2CCN(C(=O)OC(C)(C)C)CC2)c(=O)n2nc(C3=CCOCC3)nc2n1CC(=O)Nc1ccc(C(F)(F)F)cc1Cl]

2-(6-(4-(Tert-butoxycarbonyl)piperazin-1-yl)-2-(3,6-dihydro-2H-pyran-4-yl)-5-ethyl-7-oxo-[1,2,4]triazolo[1,5-a]pyrimidin-4(7H)-yl)acetic acid (4.20 g, 8.60 mmol) was dissolved in EtOAc (50 mL) and Et₃N (4.77 mL, 34.4 mmol). 2-Chloro-4-(trifluoromethyl)aniline (1.68 g, 8.60 mmol) and T₃P 50% in DMF (10.2 mL, 17.2 mmol) were added and the RM was stirred at RT for 1 hour. The RM was adsorbed onto Isolute and purified by column chromatography (RediSep Column: Silica 120 g, eluent cyclohexane:EtOAc 100:0 to 20:80). The product containing fractions were combined and concentrated to give the title compound.

LC-MS: Rt=1.35 min; MS m/z [M+H-Boc]⁺566.3/568.3, m/z [M−H]⁻ 664.4/666.4; UPLC-MS

### Step 2: N-(2-chloro-4-(trifluoromethyl)phenyl)-2-(2-(3,6-dihydro-2H-pyran-4-yl)-5-ethyl-7-oxo-6-(piperazin-1-yl)-[1,2,4]triazolo[1,5-a]pyrimidin-4(7H)-yl)acetamide

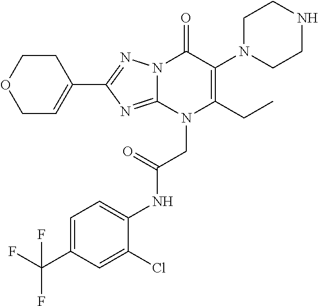 [SMILES: CCc1c(N2CCNCC2)c(=O)n2nc(C3=CCOCC3)nc2n1CC(=O)Nc1ccc(C(F)(F)F)cc1Cl]

To a solution of tert-butyl 4-(4-(2-((2-chloro-4-(trifluoromethyl)phenyl)amino)-2-oxoethyl)-2-(3,6-dihydro-2H-pyran-4-yl)-5-ethyl-7-oxo-4,7-dihydro-[1,2,4]triazolo[1,5-a]pyrimidin-6-yl)piperazine-1-carboxylate (4.82 g, 7.24 mmol) in DCM (50 mL) was added TFA (8.36 mL, 109 mmol) and the RM was stirred at RT for 2 hours. The RM was concentrated under reduced pressure. DCM was added and the mixture was extracted with aq NaHCO₃, adjusted to pH 10, extracted three times with DCM, dried through a phase separator and concentrated under reduced pressure to give the title compound.

LC-MS: Rt=0.79 min; MS m/z [M+H]⁺ 566.3/568.3, m/z [M−H]⁻ 564.4/566.4; UPLC-MS 1

### Intermediate E: tert-butyl 4-(2-(3,6-dihydro-2H-pyran-4-yl)-5-methyl-7-oxo-4,7-dihydro-[1,2,4]triazolo[1,5-a]pyrimidin-6-yl)piperazine-1-carboxylate

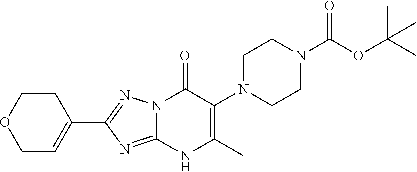 [SMILES: Cc1[nH]c2nc(C3=CCOCC3)nn2c(=O)c1N1CCN(C(=O)OC(C)(C)C)CC1]

### Step 1: tert-butyl 4-(5-methyl-7-oxo-4,7-dihydro-[1,2,4]triazolo[1,5-a]pyrimidin-6-yl)piperazine-1-carboxylate

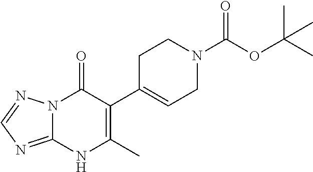 [SMILES: Cc1[nH]c2ncnn2c(=O)c1C1=CCN(C(=O)OC(C)(C)C)CC1]

4H-1,2,4-triazol-3-amine (13.6 g, 162 mmol) and tert-butyl 4-(1-ethoxy-1,3-dioxobutan-2-yl)piperazine-1-carboxylate (Intermediate M) (50.9 g, 162 mmol) were heated in AcOH (139 mL, 2.43 mol) at 100° C. for 70 minutes. The majority of the AcOH was removed in vacuo. The mixture was diluted with EtOH (100 mL) and heated at 88° C. for 18 hours. The mixture was allowed to cool to RT and filtered. The solid was washed with EtOH (120 mL) and dried in vacuo at 50° C. overnight to give the title compound as an off-white solid.

LC-MS: Rt=0.79 min; MS m/z [M+H]⁺ 335.5, m/z [M−H]⁻ 333.4; UPLC-MS 8

### Step 2: tert-butyl 4-(2-(3,6-dihydro-2H-pyran-4-yl)-5-methyl-7-oxo-4,7-dihydro-[1,2,4]triazolo[1,5-a]pyrimidin-6-yl)piperazine-1-carboxylate

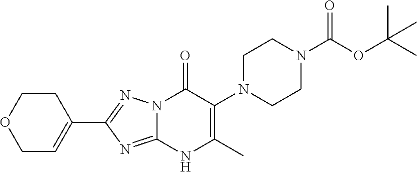 [SMILES: Cc1[nH]c2nc(C3=CCOCC3)nn2c(=O)c1N1CCN(C(=O)OC(C)(C)C)CC1]

A flask was flame-dried under reduced pressure and backfilled with argon. Tert-butyl 4-(5-methyl-7-oxo-4,7-dihydro-[1,2,4]triazolo[1,5-a]pyrimidin-6-yl)piperazine-1-carboxylate (5.00 g, 14.7 mmol) was suspended in anhydrous THF (14.7 mL). To the white suspension was added at RT zinc chloro 2,2,6,6-tetramethylpiperidide lithium chloride complex solution (35.9 mL, 32.2 mmol) with dropwise 1 mL/min. The RM was stirred for 1.5 hours. Then it was stored in the freezer under argon overnight. The next morning it was stirred at RT for 4 hours. 4-bromo-3,6-dihydro-2H-pyran (3.18 mL, 29.3 mmol), CPhos (392 mg, 879 μmol) and CPhos Pd G3 (746 mg, 879 μmol) were introduced under argon. The RM was stirred at RT for 68.5 hours. The reaction was partitioned between THF (100 mL) and aq. NH₄Cl 5N (100 mL). The aqueous layer was backextracted with EtOAc (3×50 mL). The organic layers were combined, washed with aq. 10% Na₂S₂O₃ (150 mL), brine (150 mL) and dried through a phase separator. The solvent was collected and treated with ISOLUTE® Si-TMT (36.6 g, 17.6 mmol). The suspension was stirred at 40° C. for 1 hour and was filtered off over a pad of celite. Removal of volatiles under pressure. The residue was adsorbed onto Isolute and purified by column chromatography (FlashPure® EcoFlex silica cartridge 330 g, eluent DCM:MeOH 100:0 to 95:5). The product containing fractions were combined and concentrated to give the title compound as an off-white solid.

LC-MS: Rt=0.89 min; MS m/z [M+H]⁺ 417.3, m/z [M−H]⁻ 415.2; UPLC-MS 8

### Intermediate F: 6-(4-acetylpiperazin-1-yl)-2-(3-fluoropiperidin-1-yl)-5-methyl-[1,2,4]triazolo[1,5-a]pyrimidin-7(4H)-one

### Step 1: 3-(3-fluoropiperidin-1-yl)-1H-1,2,4-triazol-5-amine

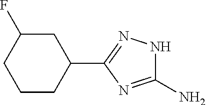 [SMILES: Nc1nc(C2CCCC(F)C2)n[nH]1]

3-Fluoropiperidine hydrochloride (1.00 g, 7.30 mmol) was suspended in ACN (5 mL). Dimethyl cyanocarbonimidodithioate (1.07 g, 7.30 mmol) and DIPEA (1.27 mL, 7.30 mmol) were added and the RM was stirred at 80° C. for 14 hours. Hydrazine hydrate (11.6 mL, 7.30 mmol) was added and the RM was stirred at 80° C. for 14 hours. The RM was concentrated under reduced pressure. Water was added and it was extracted with 10% MeOH in DCM.

The organic phase was dried over Na₂SO₄ and concentrated under reduced pressure to give the title compound.

### Step 2: 6-(4-acetylpiperazin-1-yl)-2-(3-fluoropiperidin-1-yl)-5-methyl-[1,2,4]triazolo[1,5-a]pyrimidin-7(4H)-one

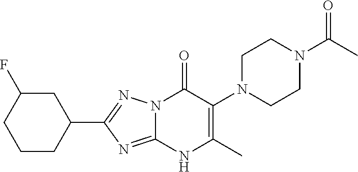 [SMILES: CC(=O)N1CCN(c2c(C)[nH]c3nc(C4CCCC(F)C4)nn3c2=O)CC1]

3-(3-Fluoropiperidin-1-yl)-1H-1,2,4-triazol-5-amine (800 mg, 4.32 mmol) was suspended in EtOH (25 mL). Ethyl 2-(4-acetylpiperazin-1-yl)-3-oxobutanoate (Intermediate L) (1.45 g, 5.62 mmol) and acetic acid (200 μL, 3.49 mmol) were added and the RM was stirred at 100° C. for 12 hours. The RM was concentrated under reduced pressure. The crude product was purified by column chromatography (Combiflash column: 12 g, eluent DCM:MeOH 100:0 to 95:5). The product containing fractions were combined, concentrated, and dried under HV to give the title compound.

Intermediate G: 2-(6-(4-acetylpiperazin-1-yl)-2-bromo-5-methyl-7-oxo-[1,2,4]triazolo[1,5-a]pyrimidin-4(7H)-yl)-N-(4-(trifluoromethyl)phenyl)acetamide

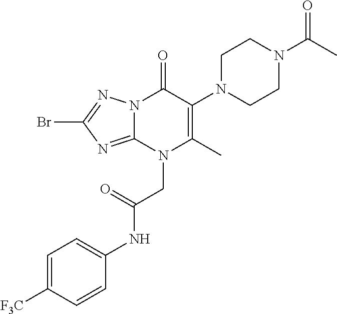 [SMILES: CC(=O)N1CCN(c2c(C)n(CC(=O)Nc3ccc(C(F)(F)F)cc3)c3nc(Br)nn3c2=O)CC1]

### Step 1: 2-(6-(4-acetylpiperazin-1-yl)-2-bromo-5-methyl-7-oxo-[1,2,4]triazolo[1,5-a]pyrimidin-4(7H)-yl)acetic acid

To a solution of tert-butyl 2-(6-(4-acetylpiperazin-1-yl)-2-bromo-5-methyl-7-oxo-[1,2,4]triazolo[1,5-a]pyrimidin-4(7H)-yl)acetate (Intermediate I) (1.5 g, 3.2 mmol) in DCM (5 mL) was added TFA (3.7 g, 32.4 mmol) dropwise. The RM was stirred at 40° C. for 5 hours. The RM was concentrated under reduced pressure to give the title compound as a brown residue.

LC-MS: Rt=0.42 min; MS m/z [M+H]⁺ 413.2; UPLC-MS 8

Step 2: 2-(6-(4-acetylpiperazin-1-yl)-2-bromo-5-methyl-7-oxo-[1,2,4]triazolo[1,5-a]pyrimidin-4(7H)-yl)acetic acid (1.37 g, 3.32 mmol), 4-(trifluoromethyl)aniline (374 mg, 2.32 mmol), T3P 50% in EtOAc (3.95 mL, 6.63 mmol) and Et3N (1.85 mL, 13.3 mmol) were mixed in DCM (5 mL) and stirred at RT for 3 hours. Water, aq sat NaHCO₃ and DCM were added to the RM. The aqueous layer was washed twice with DCM. The combined organic layer was dried through a phase separator and concentrated under reduced pressure. The mixture was suspended in MeOH and filtered. The cake was dried under HV.

LC-MS: Rt=0.97 min; MS m/z [M+H]⁺ 556.4, m/z; UPLC-MS 8

### Intermediate H: 2-(6-(4-acetylpiperazin-1-yl)-2-(3,6-dihydro-2H-pyran-4-yl)-5-methyl-7-oxo-[1,2,4]triazolo[1,5-a]pyrimidin-4(7H)-yl)acetic acid

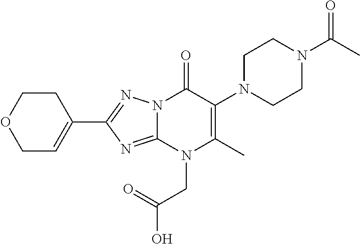 [SMILES: CC(=O)N1CCN(c2c(C)n(CC(=O)O)c3nc(C4=CCOCC4)nn3c2=O)CC1]

### Step 1: tert-butyl 2-(6-(4-acetylpiperazin-1-yl)-2-(3,6-dihydro-2H-pyran-4-yl)-5-methyl-7-oxo-[1,2,4]triazolo[1,5-a]pyrimidin-4(7H)-yl)acetate

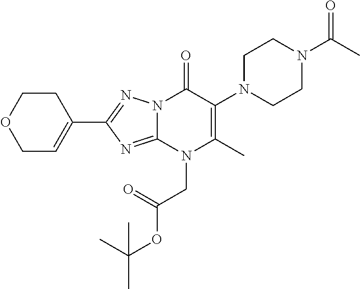 [SMILES: CC(=O)N1CCN(c2c(C)n(CC(=O)OC(C)(C)C)c3nc(C4=CCOCC4)nn3c2=O)CC1]

Tert-butyl 2-(6-(4-acetylpiperazin-1-yl)-2-bromo-5-methyl-7-oxo-[1,2,4]triazolo[1,5-a]pyrimidin-4(7H)-yl)acetate (Intermediate I) (200 mg, 426 μmol), 3,6-Dihydro-2H-pyran-4-boronic acid pinacol ester (112 mg, 533 μmol) and X-Phos G3 (18.0 mg, 21.0 μmol) were introduced in a MW vial, which was then several times purged/vacuumed with argon. DMF (3 mL) and K₃PO₄ 1 M in water (852 μL, 852 μmol) were added. The RM was stirred at 60° C. 3.75 hours. The RM was diluted with DCM and washed three times with aq sat NaHCO₃. The combined organic phases were dried through a phase separator and concentrated under reduced pressure. The crude product was purified by column chromatography (RediSep Column: Silica 12 g, eluent DCM:DCM/MeOH (9/1) 100:0 to 50:50) to give the title compound.

LC-MS: Rt=0.88 min; MS m/z [M+H]⁺ 473.4, m/z [M−H]⁻ 471.4; UPLC-MS 8

### Step 2: 2-(6-(4-acetylpiperazin-1-yl)-2-(3,6-dihydro-2H-pyran-4-yl)-5-methyl-7-oxo-[1,2,4]triazolo[1,5-a]pyrimidin-4(7H)-yl)acetic acid

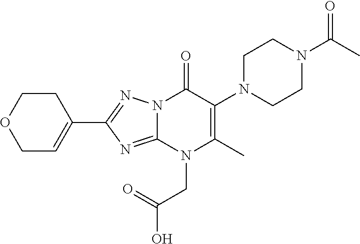 [SMILES: CC(=O)N1CCN(c2c(C)n(CC(=O)O)c3nc(C4=CCOCC4)nn3c2=O)CC1]

To a solution of tert-butyl 2-(6-(4-acetylpiperazin-1-yl)-2-(3,6-dihydro-2H-pyran-4-yl)-5-methyl-7-oxo-[1,2,4]triazolo[1,5-a]pyrimidin-4(7H)-yl)acetate (195 mg, 392 μmol) in DCM (600 μL) was added TFA (604 μL, 7.84 mmol) dropwise. The RM was stirred at RT for 20.5 hours. The RM was concentrated under reduced pressure to give the title compound as a sticky brown residue.

LC-MS: Rt=0.47 min; MS m/z [M+H]⁺ 417.3, m/z [M−H]⁻ 415.4; UPLC-MS 8

### Intermediate I: tert-butyl 2-(6-(4-acetylpiperazin 1 yl)-2-bromo-5-methyl-7-oxo-[1,2,4]triazolo[1,5-a]pyrimidin-4(7H)-yl)acetate

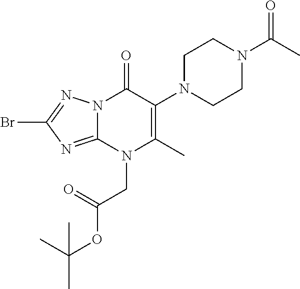 [SMILES: CC(=O)N1CCN(c2c(C)n(CC(=O)OC(C)(C)C)c3nc(Br)nn3c2=O)CC1]

6-(4-Acetylpiperazin-1-yl)-2-bromo-5-methyl-[1,2,4]triazolo[1,5-a]pyrimidin-7(4H)-one (Intermediate K) (3.25 g, 8.68 mmol) and tert-butyl 2-bromoacetate (1.90 g, 9.55 mmol) were mixed in DMF (20 mL). K₂CO₃ (2.40 g, 17.4 mmol) was added and the RM was stirred at 80° C. for 4.5 hours. The RM was concentrated under reduced pressure. The residue was dissolved in DCM and extracted twice with water (2×25 mL) and three times with DCM (3×150 mL). The combined organic phases were dried through a phase separator and concentrated under reduced pressure. The crude product was adsorbed onto Isolute and purified by column chromatography (RediSep Column: Silica 120 g, eluent DCM:DCM/MeOH (9/1) 100:0 to 50:50) to give the title compound as a beige solid.

LC-MS: Rt=0.90 min; MS m/z [M+H]⁺ 469.2/471.2, m/z [M−H]⁻ 467.1/469.1; UPLC-MS 8

### Intermediate J: tert-butyl 4-(2-bromo-5-methyl-7-oxo-4,7-dihydro-[1,2,4]triazolo[1,5-a]pyrimidin-6-yl)piperazine-1-carboxylate

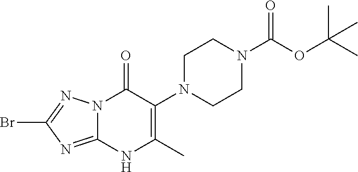 [SMILES: Cc1[nH]c2nc(Br)nn2c(=O)c1N1CCN(C(=O)OC(C)(C)C)CC1]

3-Bromo-1H-1,2,4-triazol-5-amine (Intermediate R) (4.00 g, 24.5 mmol), tert-butyl 4-(1-ethoxy-1,3-dioxobutan-2-yl)piperazine-1-carboxylate (Intermediate M) (8.49 g, 27.0 mmol) and H₃PO₄ (2.97 g, 25.8 mmol) were mixed in EtOH (25 mL) and stirred at reflux for 18 hours. The RM was cooled to RT, DIPEA (12.9 mL, 73.6 mmol) and Boc₂O (1.71 mL, 7.36 mmol) were added, and the RM was stirred at RT for 1 hour. The RM was quenched with aq NH₄Cl, diluted with DCM, extracted twice with DCM, dried over Na₂SO₄, concentrated and dried. The crude product was crystallized from DCM and TBME to give the title compound.

LC-MS: Rt=0.87 min; MS m/z [M+H]⁺ 413.1, m/z [M−H]⁻ 411.0; UPLC-MS 4

### Intermediate K: 6-(4-acetylpiperazin-1-yl)-2-bromo-5-methyl-[1,2,4]triazolo[1,5-a]pyrimidin-7(4H)-one

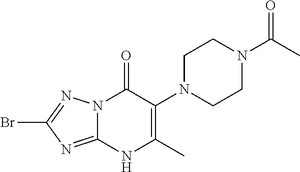 [SMILES: CC(=O)N1CCN(c2c(C)[nH]c3nc(Br)nn3c2=O)CC1]

5-Bromo-1H-1,2,4-triazol-3-amine (Intermediate R) (41.0 g, 239 mmol) and ethyl 2-(4-acetylpiperazin-1-yl)-3-oxobutanoate (Intermediate L) (68.1 g, 239 mmol) were suspended in AcOH (137 mL, 2.39 mol) and heated at 100° C. for 3 hours. The RM was cooled down to RT and it crystallized. Water (200 mL) was added and the mixture was stirred at RT for 2 hours. The solid was filtered off and the white powder was dried under HV at 30° C. to give the title compound.

LC-MS: Rt=0.53 min; MS m/z [M+H]⁺ 355.1/357.1, m/z [M−H]⁻ 353.0/355.1; UPLC-MS 8

### Intermediate L: ethyl 2-(4-acetylpiperazin-1-yl)-3-oxobutanoate

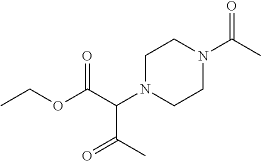 [SMILES: CCOC(=O)C(C(C)=O)N1CCN(C(C)=O)CC1]

To a yellow solution of 1-Acetylpiperazine (105 g, 808 mmol) in toluene (808 mL) was added ethyl 2-chloroacetatoacetate (58.8 mL, 404 mmol). The solution was stirred at 100° C. for 2 hours. The RM was filtered through hyflo and the residue was washed with toluene. The filtrate was evaporated. The brown oil was stirred in DCM (200 mL) for 1 hour, filtered and the residue was washed with DCM. The filtrate was concentrated under reduced pressure. The crude product was purified by column chromatography (RediSep Column: Silica 330 g, eluent EtOAc). The product containing fractions were combined, concentrated and dried under HV to give the title compound.

LC-MS: Rt=0.60/0.87 min; MS m/z [M+H]⁺ 257.2, m/z [M−H]⁻ 255.1; UPLC-MS 8

### Intermediate M: tert-butyl 4-(1-ethoxy-1,3-dioxobutan-2-yl)piperazine-1-carboxylate

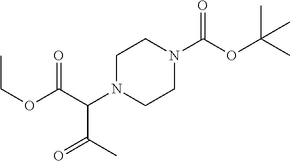 [SMILES: CCOC(=O)C(C(C)=O)N1CCN(C(=O)OC(C)(C)C)CC1]

To a stirred solution of tert-butyl piperazine-1-carboxylate (150 g, 805 mmoL) in ACN (1.5 L) at RT was added K₂CO₃ (223 g, 1.61 mol) and the RM was stirred for 15 minutes. Then ethyl 2-chloro-3-oxobutanoate (112 mL, 809 mmol) was added slowly at the same temperature. The resulting RM was stirred at RT for 16 hours. The RM was filtered through celite pad. The celite pad was washed with EtOAc (2 L). The combined organic layers were concentrated under reduced pressure to get crude residue. The residue was dissolved in EtOAc (3 L) and then washed with ice cold water, brine, dried over Na₂SO₄ and concentrated under reduced pressure to get crude product as pale brown liquid. The crude product was purified by column chromatography (silica gel, 60-120 mesh, eluent petroleum ether:EtOAc 100:0 to 85:15). The pure product containing fractions were combined and concentrated under reduced pressure to give the title compound as a liquid. The impure fractions were combined and concentrated under reduced pressure. Then they were purified again by column chromatography (silica gel, 60-120 mesh, eluent petroleum ether:EtOAc 100:0 to 85:15). The pure product containing fractions were combined and concentrated under reduced pressure to give the title compound as a liquid. Both liquids were mixed, dissolved in DCM and concentrated under reduced pressure to get the title compound as as a brown liquid. The liquid was again dissolved in DCM, concentrated under reduced pressure. The process was repeated three times and then dried under vacuum to give the title compound as a brown liquid.

HPLC: Rt=11.763 min; HPLC 6

### Intermediate N: tert-butyl 4-(1-methoxy-1,3-dioxopentan-2-yl)piperazine-1-carboxylate

### Step 1: methyl 2-chloro-3-oxopentanoate

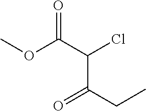

To a solution of methyl 3-oxopentanoate (10.4 kg, 80.0 mol) in DCM (67 L) was added SO₂Cl₂ (14.0 kg, 104 mol) at RT over 2.5 hours. The reaction was allowed to warm to RT and stirred for 16 hours. The RM was concentrated under reduced pressure and the residue was dissolved in DCM (20 L) and washed with water (10 L), brine (10 L), dried over Na₂SO₄ and filtered. The filtrate was concentrated under reduced pressure to give the title compound as a light yellow liquid.

¹H NMR (400 MHz, CDCl₃-d) δ 4.65 (s, 1H), 3.68 (s, 3H), 2.59 (m, 2H), 0.96 (t, 3H)

### Step 2: tert-butyl 4-(1-methoxy-1,3-dioxopentan-2-yl)piperazine-1-carboxylate

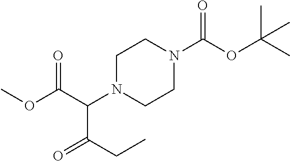 [SMILES: CCC(=O)C(C(=O)OC)N1CCN(C(=O)OC(C)(C)C)CC1]

To a solution of methyl 2-chloro-3-oxopentanoate (12.1 kg, 53.7 mol) in dry ACN (53 L) was added Et₃N (22.3 L, 161 mol) over 1.5 hours, followed by dropwise addition of tert-butyl piperazine-1-carboxylate (10.0 kg, 53.7 mol) in ACN (50 L) over 2.5 hours. The reaction was stirred at 60° C. for 16 hours. The RM was filtered and washed with EtOAc (10 L). The filtrate was then concentrated under reduced pressure and the residue was dissolved in EtOAc (45 L) and washed with water (45 L), dried over Na₂SO₄ and filtered. The filtrate was concentrated under reduced pressure and the residue was purified by column chromatography (Silica column 150 mm×800 mm×70 mm, eluent heptane:EtOAc 100:0 to 90:10) to give the title compound.

HPLC: Rt=3.681/5.513 min; HPLC 4

### Intermediate 0: 6-hydroxypyrazolo[1,5-a]pyridine-7-carboxylic acid

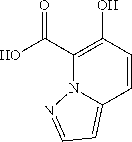 [SMILES: O=C(O)c1c(O)ccc2ccnn12]

### Step 1: 5-bromoimidazo[1,2-a]pyridin-6-ol

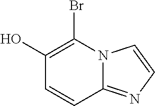 [SMILES: Oc1ccc2nccn2c1Br]

The flask was flame-dried in vacuo and backfilled with argon. The dry argon flushed flask was charged with 5-bromo-6-methoxyimidazo[1,2-a]pyridine (1.00 g, 4.18 mmol) and anhydrous DCM (14 mL). The resulting mixture was cooled to −78° C. and then 1 M BBr₃ in DCM (20.9 mL, 20.9 mmol) was added dropwise. After the complete addition the resulting deep brown suspension was warmed to RT. The RM was stirred at RT for 18 hours. The RM was cooled to −78° C. and then anhydrous MeOH (3.42 mL, 84.0 mmol) was added slowly. The RM was concentrated to dryness. MeOH (5 mL) was added followed by Et₂O (100 mL). The brown suspension was sonicated and the solid was filtered off. The solid was washed with Et₂O (2×50 mL) and dried under vacuum to give the title compound as a pink powder (1.19 g, 98% pure, yield: 95%).

LC-MS: Rt=0.23 min; MS m/z [M+H]⁺ 213.2/215.2, MS m/z [M−H]⁻ 210.9/212.9; UPLC-MS 7

### Step 2: 6-(benzyloxy)-5-bromoimidazo[1,2-a]pyridine

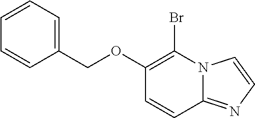 [SMILES: Brc1c(OCc2ccccc2)ccc2nccn12]

The flask was flame-dried in vacuo and backfilled with argon. The dry argon flushed flask was charged with 5-bromoimidazo[1,2-a]pyridin-6-ol (1.19 g, 3.97 mmol) and potassium carbonate (1.66 g, 11.9 mmol). The contents were suspended in anhydrous DMF (9.92 mL) and the resulting mixture was treated with (bromomethyl)benzene (626 μL, 5.16 mmol). The RM was stirred at RT for 21 hours. The reaction was partitioned between water (35 mL) and EtOAc (30 mL). The organic layer was collected and the aqueous layer was back-extracted with EtOAc (3×25 mL). The organic layers were combined, washed with brine (50 mL) and dried through a phase separator. The solvent was reduced to dryness to provide a brownish oil (668 mg). The crude product was purified by normal phase chromatography (Buchi® FlashPure ID HP silica cartridge 24 g, eluent heptane:EtOAc 100:0 to 15:85). The product containing fractions were combined and concentrated to give the title compound as a beige powder (94.6 mg, 98% pure, yield: 8%).

LC-MS: Rt=0.80 min; MS m/z [M+H]⁺ 303.1/305.1; UPLC-MS 1

### Step 3: ethyl 6-(benzyloxy)imidazo[1,2-a]pyridine-5-carboxylate

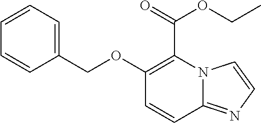 [SMILES: CCOC(=O)c1c(OCc2ccccc2)ccc2nccn12]

The reactor was charged with 6-(benzyloxy)-5-bromoimidazo[1,2-a]pyridine (94.0 mg, 304 μmol), PdCl₂(dppf).DCM adduct (12.4 mg, 15.0 μmol), Et₃N (128 μL, 912 μmol) and anhydrous EtOH (10 mL). The autoclave was subjected to three cycles of evacuation-backfilling with argon. Subsequently it was filled with 20 bars of CO at RT and then heated at 80° C. for 20 hours. PdCl₂(dppf).DCM adduct (12.4 mg, 15.0 μmol) and Et₃N (128 μL, 912 μmol) were introduced and the RM was heated at 90° C. for 20 hours. ISOLUTE® Si-TMT (62.0 mg, 30.0 μmol) was introduced to the mixture and it was left to stir at RT. The mixture was filtered off over a pad of celite. Removal of volatiles under pressure to give a yellow-brown solid (175 mg). A dry argon flushed vial was charged with the yellow-brown solid (175 mg) from the previous step and K₂CO₃ (42.4 mg, 304 μmol). The contents were suspended with anhydrous DMF (2 mL) and (bromomethyl)benzene (26.0 μL, 213 μmol) was introduced. The vial was sealed and the resulting mixture was left to react at RT for 16 hours. (Bromomethyl)benzene (13.0 μL, 106 μmol) was introduced and the RM was stirred at RT for 22.5 hours. The reaction was partitioned between water (10 mL) and EtOAc (5 mL). The organic layer was collected and the aqueous layer was back-extracted with EtOAc (3×5 mL). The organic layers were combined, washed with brine (20 mL) and dried through a phase separator. The solvent was reduced to dryness to give a brownish oil (107 mg). The crude product was adsorbed onto Isolute and purified by normal phase chromatography (Buchi® FlashPure ID HP silica cartridge 12 g, eluent heptane:EtOAc 100:0 to 0:100). The product containing fractions were combined and concentrated to give the title compound as a beige solid (37.3 mg, 98% pure, yield:41%).

LC-MS: Rt=0.79 min; MS m/z [M+H]⁺ 297.4; UPLC-MS 1

### Step 4: 6-(benzyloxy)imidazo[1,2-a]pyridine-5-carboxylic acid

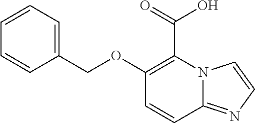 [SMILES: O=C(O)c1c(OCc2ccccc2)ccc2nccn12]

Ethyl 6-(benzyloxy)imidazo[1,2-a]pyridine-5-carboxylate (36.0 mg, 119 μmol) was dissolved in ACN (1.19 mL) and the mixture was treated with 1M NaOH in water (119 μL, 119 μmol). The resulting mixture was stirred at RT for 23.5 hours. 1M NaOH in water (11.9 mL, 119 μmol) was added and the RM was stirred at RT for 21.5 hours. The RM was frozen in a mixture of dryice/acetone and lyophilized to give the title compound as a white powder (33.7 mg, 98% pure, yield: 95%).

LC-MS: Rt=0.56 min; MS m/z [M+H]⁺ 269.3, MS m/z [M−H]⁻ 267.1; UPLC-MS 10

### Step 5: 6-hydroxypyrazolo[1,5-a]pyridine-7-carboxylic acid

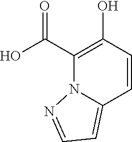 [SMILES: O=C(O)c1c(O)ccc2ccnn12]

To a purple/brown solution of 6-(benzyloxy)imidazo[1,2-a]pyridine-5-carboxylic acid (53 mg, 0.186 mmol) in 4 ml MeOH/THF 1:1 vacuumed and purged with argon several times was added Pd—C(5 mg, 4.70 μmol). The resulting dark green/black mixture was vacuumed and purged with hydrogen several times then stirred for 1.5 h at 20° C. The reaction mixture was filtered through a Millipore filter (PTFE Membrane Filter 0.2 im) and the filtrate was concentrated and dried under vacuum (40° C.) to give the title compound as a dark purple residue (36 mg, 0.186 mmol, 100% yield).

LC-MS: Rt=0.49 min; MS m/z [M+H]⁺ 179.0; UPLC-MS 1

### Intermediate P: 7-(methoxymethoxy)-2,3-dihydrofuro[3,2-c]pyridine-6-carboxylic acid

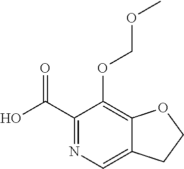 [SMILES: COCOc1c(C(=O)O)ncc2c1OCC2]

### Step 1: 6-bromofuro[3,2-c]pyridin-7-ol

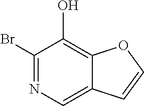 [SMILES: Oc1c(Br)ncc2ccoc12]

The flask was flame-dried in vacuo and backfilled with argon. The dry argon flushed flask was charged with 6-bromo-7-methoxyfuro[3,2-c]pyridine (5.00 g, 20.8 mmol) and anhydrous DCM (69.4 mL). The RM was cooled to −78° C. and then 1M BBr₃ in DCM (125 mL, 125 mmol) was added dropwise. After the complete addition the resulting yellow suspension was warmed to RT and stirred for 13.5 hours. The RM was cooled to −78° C. and then anhydrous MeOH (17.0 mL, 417 mmol) was added dropwise. The reaction was concentrated to dryness. MeOH (10 mL) was added followed by Et₂O (150 mL). The brown suspension was sonicated and filtered. The solid was washed with Et₂O (2×100 mL) and dried at 40° C. under vacuum overnight to give the title compound as a white solid (4.58 g, 98% pure, yield: 73%).

LC-MS: Rt=0.36 min; MS m/z [M+H]⁺ 214.0/216.0, m/z [M−H]⁻ 212.1/214.0; UPLC-MS 1

### Step 2: ethyl 7-hydroxyfuro[3,2-c]pyridine-6-carboxylate

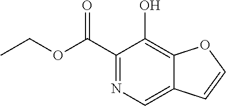

The reactor was charged with 6-bromofuro[3,2-c]pyridin-7-ol (4.58 g, 15.3 mmol), PdCl₂(dppf).DCM (624 mg, 764 μmol), Et₃N (8.60 mL, 61.1 mmol) and anhydrous EtOH (50 mL). The autoclave was subjected to three cycles of evacuation-backfilling with argon. Subsequently it was filled with 10 bar of CO at RT and then heated at 80° C. for 24 hours. ISOLUTE® Si-TMT (15.6 g, 7.64 mmol) was introduced and the suspension was stirred at 40° C. for 1 hour. The mixture was filtered over a pad of celite. The filtrate was concentrated under reduced pressure. The residue was adsorbed onto Isolute and purified by normal phase chromatography (Buchi® FlashPure ID HP silica cartridge 120 g, eluent heptane:DCM/MeOH (8:2) 100:0 to 20:80). The product containing fractions were combined and concentrated under reduced pressure to give the title compound as a brown solid (2.06 g, 98% pure, yield: 64%).

LC-MS: Rt=0.54 min; MS m/z [M+H]⁺ 208.2; UPLC-MS 1

### Step 3: ethyl 7-hydroxy-2,3-dihydrofuro[3,2-c]pyridine-6-carboxylate

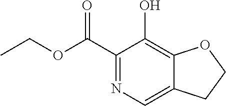

The reactor was charged with Pd—C 10 wt % (518 mg, 487 μmol) and a solution of ethyl 7-hydroxyfuro[3,2-c]pyridine-6-carboxylate (2.06 g, 9.74 mmol) in anhydrous EtOH (32.5 mL). The autoclave was subjected to three cycles of evacuation-backfilling with nitrogen. Subsequently it was filled with 5 bar of hydrogen (20.0 mg, 9.74 mmol) at RT and stirred for 5 days. The RM was filtered over a pad of celite. The cake was washed with EtOH (25 mL). The filtrate was concentrated under reduced pressure. The residue was adsorbed onto Isolute and purified by normal phase chromatography (Buchi® FlashPure ID HP silica cartridge 80 g, eluent DCM:DCM/MeOH (8:2) 100:0 to 70:30). The product containing fractions were combined and concentrated under reduced pressure to give the title compound as a white powder (1.37 g, 98% pure, yield: 66%).

LC-MS: Rt=0.38 min; MS m/z [M+H]⁺ 210.2; UPLC-MS 1

### Step 4: ethyl 7-(methoxymethoxy)-2,3-dihydrofuro[3,2-c]pyridine-6-carboxylate

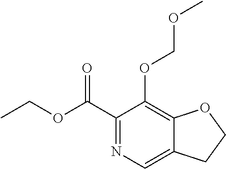 [SMILES: CCOC(=O)c1ncc2c(c1OCOC)OCC2]

To a colourless solution of ethyl 7-hydroxy-2,3-dihydrofuro[3,2-c]pyridine-6-carboxylate (1.12 g, 5.23 mmol) and DMAP (130 mg, 1.05 mmol) in anhydrous DCM (52.3 mL), previously purged/vacuumed with argon was added with DIPEA (1.38 mL, 7.84 mmol). The colourless mixture was cooled down to 0° C. then MOMCI (662 μL, 7.84 mmol) was added dropwise (the colourless solution turned progressively into a dark orange solution). The dark orange RM was stirred at 0° C. for 1.5 hours, the ice bath was removed and the resulting RM was stirred at RT for 3.25 hours. The RM was quenched with aqueous NaHCO₃ (100 mL). The aqueous layer was extracted twice with DCM. The combined organic layers were dried through a phase separator, concentrated and dried under high vacuum. The residue was adsorbed onto Isolute and purified by normal phase chromatography (RediSep Column: Silica 40 g, eluent heptane:EtOAc 100:0 to 10:90). The product containing fractions were combined and concentrated to give the title compound as a white solid (1.19 g, 73% pure, yield: 66%).

LC-MS: Rt=0.48 min; MS m/z [M+H]⁺ 254.2; UPLC-MS 1

### Step 5: 7-(methoxymethoxy)-2,3-dihydrofuro[3,2-c]pyridine-6-carboxylic acid

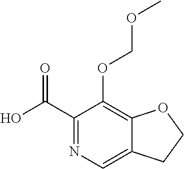 [SMILES: COCOc1c(C(=O)O)ncc2c1OCC2]

To a colourless solution of ethyl 7-(methoxymethoxy)-2,3-dihydrofuro[3,2-c]pyridine-6-carboxylate (1.19 g, 3.42 mmol) in ACN (46 mL) was added 1M NaOH in water (4.60 mL, 4.60 mmol). The resulting colourless RM was stirred at RT for 73 hours. The white suspension was filtered. The cake was washed with a bit of water and dried under vacuum. The filtrate was freezed and lyophilized. The resulting white solid was dissolved in water and extracted twice with DCM. The combined organic layers were dried trough a phase separator, concentrated and dried under vacuum to give the title compound as a sodium salt (871 mg, 95% pure, yield: 97%).

LC-MS: Rt=0.25 min; MS m/z [M+H]⁺ 226.3; UPLC-MS 10

### Intermediate Q: 3,6-dihydro-2H-pyran-4-carbaldehyde

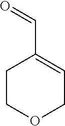

According to ref. Org. Lett. 2014, 16, 4142-4145.

To a mixture of tetrahydro-4H-pyran-4-one (30.0 g, 300 mmol) and water (300 mL), was added NaCN (15.4 g, 315 mmol) at 5° C. followed by NaHSO₄ until a pH=4-5 was reached. The reaction was stirred at 10° C. for 1 hour, then NaCl (17.5 g, 300 mmol) was added at 25° C. followed by 2-MeTHF. The organic layers were separated and the aqueous layer was extracted twice with 2-MeTHF. The combined organic layers were washed with brine, dried over Na2SO4, concentrated under reduced pressure, and switched the solvent to toluene (300 mL) to give 4-hydroxytetrahydro-2H-pyran-4-carbonitrile. Pyridine (48.5 mL, 599 mmol) was added at 65° C., followed by a slow addition of POCl₃ (27.9 mL, 300 mmol). The RM was stirred at 65° C. for 1 hour, then it was cooled to RT and water was added. The layers were separated, and the aqueous layer was extracted twice with toluene. The combined organic layers were washed with brine, dried over Na₂SO₄ and concentrated under reduced pressure to give 3,6-dihydro-2H-pyran-4-carbonitrile. The residue was mixed with toluene (300 mL) and DIBAL-H (46.9 g, 330 mmol) was added at −10° C. The reaction was stirred at −10° C. for 1 hour, then HCl 4M was added. The two layers were separated, and the aqueous layer was extracted twice with DCM. The combined organic layers were washed with brine and dried over Na₂SO₄. The organic solution was then concentrated under reduced pressure to give the title compound as solution in toluene (not stable when concentrated).

1H NMR (400 MHz, DMSO-d6) δ 9.46 (s, 1H), 7.05 (m, 1H), 4.33 (m, 2H), 3.69 (m, 2H), 2.16 (m, 2H).

### Intermediate R: 3-bromo-1H-1,2,4-triazol-5-amine

### Step 1: 3,5-dibromo-1-(methoxymethyl)-1H-1,24-triazol

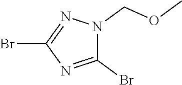

To a solution of NaH (846 g, 21.2 mol, 60%) in DMF (12 L) was added 3,5-dibromo-1H-1,2,4-triazole (4.00 kg, 17.6 mol) at 10° C. The resulting solution was stirred at 10° C. for 1 hour. This was followed by the addition of chloro(methoxy)methane (1.70 kg, 21.2 mol) dropwise at 20° C. The mixture was stirred at RT overnight. The reaction was quenched with H₂O (20 L). The resulting solution was extracted with TBME (2×7 L). The combined organic layers were washed with 10% NaCl (2×7 L), dried over Na₂SO₄ and concentrated under reduced pressure at 40° C. The residue was triturated with heptane (3 L) to give the title compound as a white solid.

HPLC: Rt=3.042 min; HPLC 4

### Step 2: 3-bromo-1-(methoxymethyl)-1H-1,2,4-triazol-5-amine

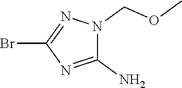

3,5-Dibromo-1-(methoxymethyl)-1H-1,2,4-triazole (800 g, 2.78 mol) was dissolved in 25% NH₃—H₂O (2.89 L, 16.3 mol) and MeOH (80 mL) at RT. The mixture was stirred at 120° C. for 18 hours. The mixture was cooled to 5~10° C., the solids were collected by filtration and washed with water (200 mL). The cake was dried under vacuum at 60° C. to give the title compound as a white solid.

HPLC: Rt=1.701 min; HPLC 4

### Step 3: 3-bromo-1H-1,2,4-triazol-5-amine

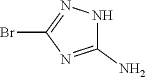

To a solution of 3-bromo-1-(methoxymethyl)-1H-1,2,4-triazol-5-amine (329 g, 1.45 mol) in MeOH (1.5 L) was added HBr (4.39 kg, 21.7 mol) at RT. The mixture was stirred at 100° C. for 18 hours. The mixture was adjusted to pH=7.0~7.5 with 10% NaOH at 20-30° C. and extracted with EtOAc (10×3 L). The combined organic phase was dried over Na₂SO₄ and concentrated under reduced pressure at 50° C. to give the title compound as a white solid.

HPLC: Rt=0.702 min; HPLC 4

HPLC: Rt=1.902 min; HPLC 5

### Intermediate R: 3-bromo-1H-1,2,4-triazol-5-amine

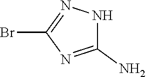

To a solution of 1H-1,2,4-triazole-3,5-diamine (300 g, 3.03 mol) in HBr/H₂O (2.4 L) was added dropwise NaNO₂ (313 g, 4.54 mol) in water (782 mL) at 0° C. over 1.5 hours. The reaction was allowed to warm to RT and stirred for 1 hour. The reaction was stirred at 100° C. and for 16 hours. The RM was cooled to RT, filtered and the pH of the mixture (66 batches combined) was adjusted to 4 by addition of 10% NaOH. The mixture was extracted with EtOAc (2×55 L), dried over Na₂SO₄ and filtered. The organic phase was concentrated under reduced pressure to give the title compound. The pH of the aqueous phase was adjusted to 7-7.5 by 10% NaOH. Then it was extracted with EtOAc (10×35 L), dried over Na₂SO₄ and filtered. The organic phase was concentrated under reduced pressure to give the title compound.

HPLC: Rt=1.933 min; HPLC 5

### Intermediate S: N-(2-chloro-4-(trifluoromethyl)phenyl)-2-iodoacetamide

### Step 1: 2-chloro-N-(2-chloro-4-(trifluoromethyl)phenyl)acetamide

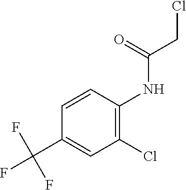 [SMILES: O=C(CCl)Nc1ccc(C(F)(F)F)cc1Cl]

2-Chloro-4-(trifluoromethyl)aniline (18.5 g, 95.0 mmol) was dissolved in DCM (180 mL) at 0° C. A solution of 2-chloroacetyl chloride (10.7 g, 95.0 mmol) in DCM (40 mL) was added dropwise over 15 minutes. After 30 minutes at 0° C. the RM was warmed to RT. The white suspension was stirred at RT overnight. The suspension was filtered and washed with DCM. The filtrate was concentrated under reduced pressure and dried under HV to give the title compound as a white solid.

LC-MS: Rt=1.14 min; MS m/z [M−H]⁻ 270.1/272.1/274.0; UPLC-MS 1

### Step 2: N-(2-chloro-4-(trifluoromethyl)phenyl)-2-iodoacetamide

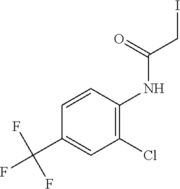 [SMILES: O=C(CI)Nc1ccc(C(F)(F)F)cc1Cl]

2-Chloro-N-(2-chloro-4-(trifluoromethyl)phenyl)acetamide (15.8 g, 58.2 mmol) was dissolved in acetone (215 mL), KI (10.6 g, 64.0 mmol) was added and the RM was stirred at reflux for 2.25 hours. The RM was cooled to RT and the suspension was filtered. The cake was washed with acetone and DCM. The filtrate was concentrated under reduced pressure and dried under HV to give the title compound.

LC-MS: Rt=1.13 min; MS m/z [M−H]⁻ 362.0/364.0; UPLC-MS 1

### Synthetic Schemes for the Compounds of Formulae 1b, 1c, 1d, 1e and 1f

Processes are provided to make to compounds of formulae 1b, 1c, 1d, 1e and 1f. Unless otherwise stated, the groups of the process schemes are as defined in the embodiments and preferences herein. The syntheses can be modified to make variants under formula (I), according to procedures known to the skilled chemist.

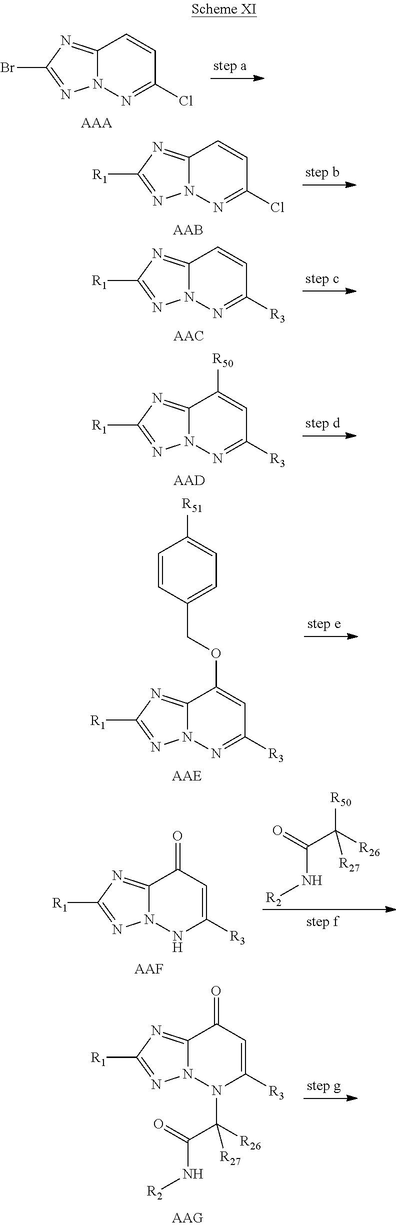

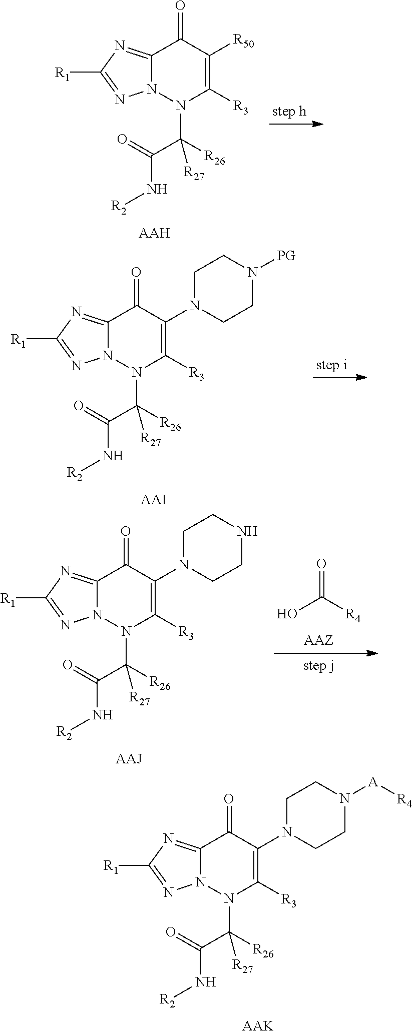

A process is provided for preparing a compound of formula AAK (Scheme XI) comprising steps a, b, c, d, e, f, g, h, i, and j. It is understood that the order of process steps a, b, c, d, e, f, g, h, i and j may be changed to optimize the synthesis as necessary. The compound of formula AAK can be obtained via coupling reaction step j by reacting compound of formula AAJ with compound AAZ wherein R₄ is defined above. The coupling reaction can be an amide formation. The coupling reaction step can be carried out with for example HATU ((1-[Bis(dimethylamino)methylene]-1H-1,2,3-triazolo[4,5-b]pyridinium 3-oxide hexafluorophosphate) or alternatively Ghosez reagent (1-Chloro-N,N,2-trimethylpropenylamine), preferably in a one or two step procedure. Alternatively, further alternative amide coupling methods are known in the art. For examples of amide bond formations, see Mantalbetti, C.A.G.N and Falque, V., Amide bond formation and peptide coupling, Tetrahedron, 2005, 61(46), pp10827-10852 and references cited therein.

Compound of formula AAJ can be prepared comprising step i of deprotecting PG from the compound of formula AAI, wherein PG represents a suitable protecting group, preferably a BOC group, and wherein the other substituents are as defined above. There are many known ways of deprotecting BOC groups. The deprotection step can be carried out with for example TFA or HCl in a solvent for example dichloromethane or dioxane.

Compound of formula AAI can be prepared comprising step h starting from a compound of formula AAH wherein R₅₀ represents halo, particularly bromo and wherein PG represents a suitable protecting group for example a BOC group and the other substituents are as defined above. Step h can be a nucleophilic aromatic substitution reaction and can be carried out by combining compound of formula AAH with an amine for example tert-butyl piperazine-1-carboxylate or alternatively piperazine. A stoichiometric excess of the amine can be used, preferably between 2 and 50 mole equivalents in an organic solvent for example DMSO or NMP. The reaction is preferably stirred at a temperature of approximately 80-140° C. and can be carried out in a capped tube. An alternative method for step h can use Buchwald-Hartwig conditions using a amine for example tert-butyl piperazine-1-carboxylate, a ligand such as Brettphos or RuPhos or RuPhos hybrid with a palladium catalyst such as RuPhos Pd G1, RuPhos Pd G4 or [PdCl(allyl)]2 in the presence of a base such as K2CO3 or Cs2CO3 or tert-BuONa in an organic solvent such as dioxane or THF. The reaction is preferably stirred at a temperature of approximately 80-120° C. The reaction is preferably carried out under an inert gas such as nitrogen or argon. Alternatively, further alternative Buchwald-Hartwig coupling methods are known in the art, for examples of methods, see B. T. Ingoglia et al Biaryl monophosphine ligands in palladium-catalyzed C—N coupling: An updated User's guide, Tetrahedron, 2019, 75(32), pp4199-4211 and references cited therein.

Compound of formula AAH can be prepared comprising step g wherein compound of formula AAG, wherein the substituents are defined as herein, is halogenated. Step g can be carried out using a halogenating reagent such as N-bromosuccinimide or N-iodosuccinimide in a solvent for example DMF or acetonitrile. The reaction is preferably stirred at a temperature of approximately 20-80° C.

Compound of formula AAG can be prepared comprising step f wherein compound of formula AAF, wherein the substituents are defined as herein, is alkylated by reacting compound of formula AAY wherein R₅₀ represents halo, particularly bromo, iodo or chloro and the other substituents are defined as above. Step f can be carried out in the presence of a base such as K₂CO₃ or N,N-diisopropylethylamine in a solvent for example DMF or dioxane. The reaction is preferably stirred at a temperature of approximately 20-80° C.

Compound of formula AAF can be prepared comprising step e starting from compound of formula AAE wherein R₅₁ represents H or methoxy and the other substituents are as defined above. Many methods of cleaving benzyl or para-methoxybenzyl groups are known in the art. Step e can be carried out in the presence of an acid such as TFA or HCl or HBr, preferably in stoichiometric excess in a solvent such as dichloromethane or dioxane and is preferably stirred at a temperature of approximately 20-80° C. An alternative method for step e can use hydrogenation conditions in the presence of a hydrogen atmosphere and a catalyst such as Pd/C or palladium hydroxide/C. The reaction is preferably stirred at a temperature of approximately 20-50° C. in an organic solvent such as ethanol or methanol.

Compound of formula AAE can be prepared comprising step d starting from compound of formula AAD wherein R₅₀ represents halo, particularly bromo and the other substituents are as defined above. Step d comprises reacting 2-10 mol equivalents of an alcohol for example benzyl alcohol or para-methoxybenzyl alcohol with 2-5 mol equivalents of a base such as sodium hydride in an organic solvent such as THF or dioxane with stirring at a temperature of approximately 20-40° C., preferably 20° C. for approximately 10-60 minutes. A compound of formula AAD is then added and stirring is continued at a temperature of approximately 20-100° C., preferably 20-60° C. The reaction is preferably carried out under an inert gas such as nitrogen or argon. An example method is described in WO2021/222522, 2021, A1 page 574.

Compound of formula AAD wherein R₅₀ represents halo, particularly bromo or iodo, and the other substituents are as defined herein, can be prepared comprising step c starting from compound of formula AAC. Step c comprises reacting a compound of formula AAC with a base such as LiTMP (lithium tetramethylpiperidide) or LDA (lithium diisopropylamide) with stirring in a solvent such as THF at a temperature of approximately −78° C. to 20° C. under an inert gas such as nitrogen or argon. After stirring for an appropriate time, approximately 30 minutes to 3 hours, a halogenating reagent such as bromine or iodine is then added at a temperature of approximately −78° C. to 20° C. and stirring is continued. Other suitable halogenating agents are known in the art.

Compound of formula AAC wherein R₁ and R₃ are as defined above can be prepared comprising step b starting from compound of formula AAB. Step b can be a Suzuki or Negishi or Stille or Kumada cross-coupling reaction and comprises reacting a compound of formula AAB with R₃n-MX wherein R₃ is as defined above, n is 1,2,3 or 4 and MX represents for example B(OH)2, BPin (Pin represents boronic acid pinacol ester) BF3K, B(MIDA), Sn, Zn, Mg-Halo. Example Negishi cross-coupling conditions comprise reacting compound of formula AAD with an alkyl zincate for example dimethylzinc or diethylzinc, preferably in stoichiometric excess for example 2-10 mol equivalents, in the presence of a catalyst such as PdCl2(dppf) or Pd(PPh3)₄ in a suitable solvent such as THF at a temperature of approximately 20-120° C., preferably 20-80° C. under an inert gas such as nitrogen or argon.

Compound of formula AAB wherein R₁ is as defined above can be prepared comprising step a from compound AAA. Step a can be a Suzuki or Negishi or Stille or Kumada cross-coupling reaction and comprises reacting a compound of formula AAA with R1 n-MX wherein R₁ is as defined above, n is 1,2,3 or 4 and MX represents for example B(OH)₂, BPin (Pin represents boronic acid pinacol ester) BF₃K, Sn, Zn, Mg-Halo. Example Suzuki cross-coupling conditions comprise reacting compound of formula AAA with R₁-BPin in the presence of a catalyst such as PdCl₂(dppf) or Pd(PPh₃)₄ and a base such as K₃PO₄ or potassium carbonate in a suitable solvent mixture such as DMF, THF or dioxane or water at a temperature of approximately 20-120° C., preferably 20-80° C. under an inert gas such as nitrogen or argon. An example method is described in CN112707908 A page 32.

Compound of formula AAA can be prepared according to the method described in CN112707908 A page 31.

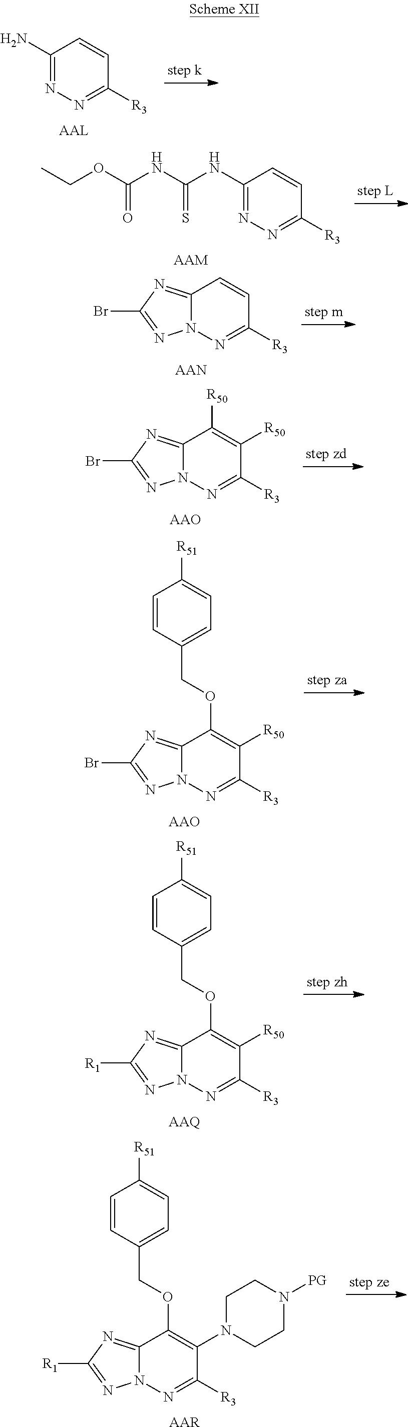

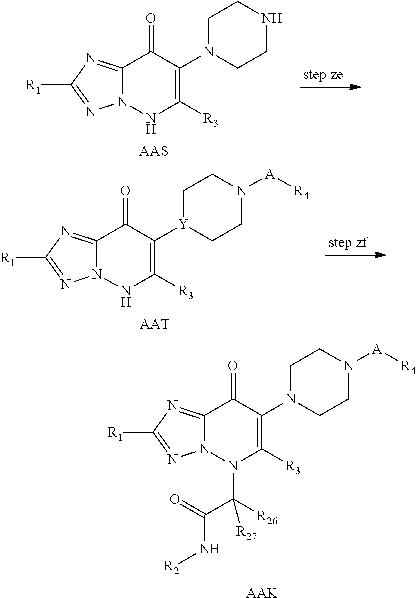

Alternatively compound of formula AAK can be prepared according to the route shown in Scheme XII comprising steps k, L, m, zd, za, zh, ze, zj and zf. Methods comprising steps zd, za, zh, ze, zj and zf to prepare compounds of formulas AAP, AAQ, AAR, AAS and AAT can be performed using analogous conditions as those described above for steps d, a, h, e, j and f for Scheme XI. It is understood that the order of process steps k, L, m, zd, za, zh, ze, zj and zf may be changed to optimize the synthesis as necessary.

Compound of formula AAO wherein R₅₀ represents halo, particularly bromo or chloro, and the other substituents are as defined above, can be prepared comprising step m starting from compound of formula AAN. Step m comprises reacting a compound of formula AAN with a halogenating agent such as PCl₅ or PBr₃ in stoichiometric excess for example 2-10 mol equivalents in a sealed tube at a temperature of approximately 200-270° C., preferably 250-270° C. for approximately 1-10 hours. An example method is described in J. Org. Chem., Vol. 39, No. 15, 1974, page 2146.

Compound of formula AAN wherein R₃ is as defined above, can be prepared comprising step L starting from compound of formula AAM. Step L comprises reacting a compound of formula AAM with hydroxylamine in the presence of a base such as triethylamine and a solvent such as ethanol or methanol at a temperature of approximately 60-100° C. The product of this reaction is then reacted with tert-butyl nitrite in the presence of CuBr₂ in a solvent such as acetonitrile at a temperature of approximately 20-50° C. An example method is described in CN112707908 A page 31.

Compound of formula AAM wherein R₃ is as defined above, can be prepared comprising step k starting from AAL. Step k comprises reacting a compound of formula AAL with ethoxycarbonyl isothiocyanate in a solvent such as dichloromethane at a temperature of approximately 0-20° C. for 2-18 hours.

Compound of formula AAL wherein R₃ is as defined above are commercially available, or methods for their preparation are known in the art.

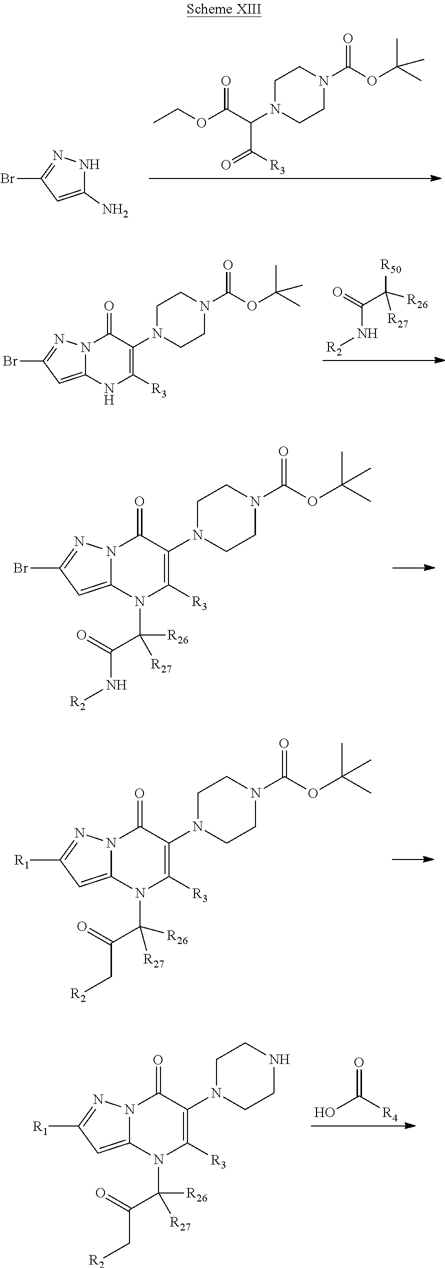

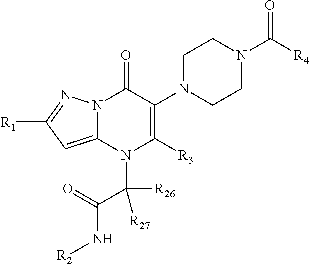

Compound of formula 1d can be prepared according to the example route shown in Scheme XIII using analogous methods to those described herein. Analogous methods to chemists skilled in the art can be adapted accordingly. It is understood that the order of process steps shown in Scheme XIII may change to optimize the synthesis as necessary.

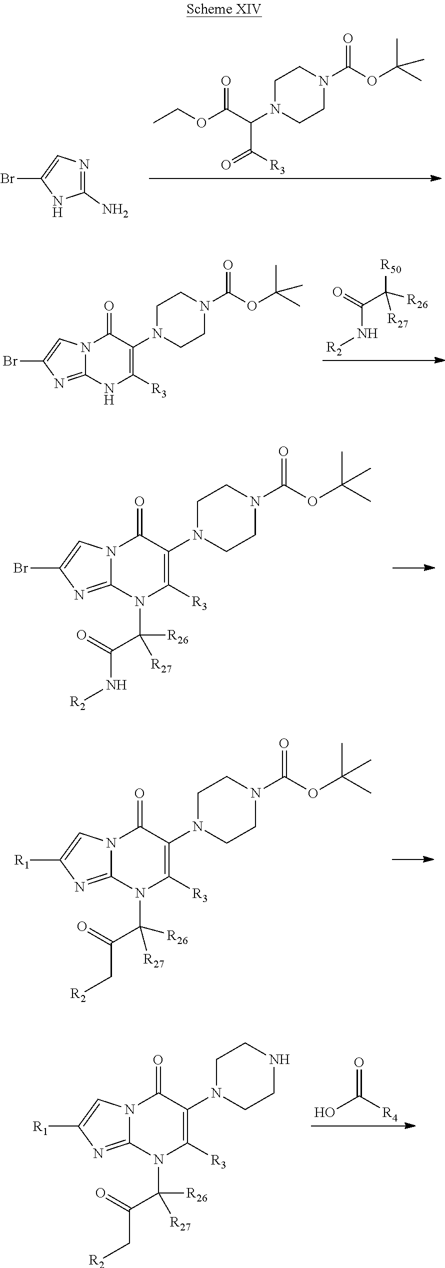

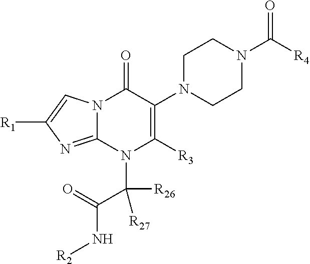

Compound of formula 1e can be prepared according to the example route shown in Scheme XIV using analogous methods to those described herein. Analogous methods to chemists skilled in the art can be adapted accordingly. It is understood that the order of process steps shown in Scheme XIV may change to optimize the synthesis as necessary.

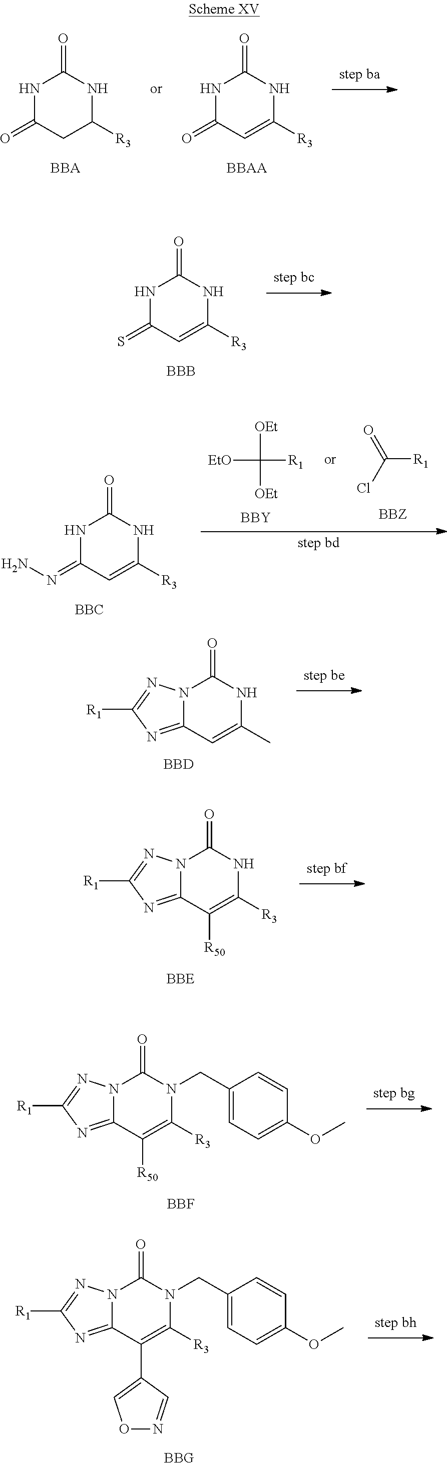

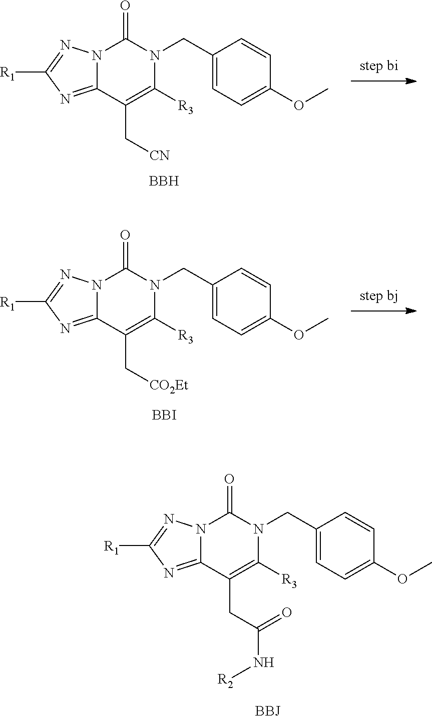

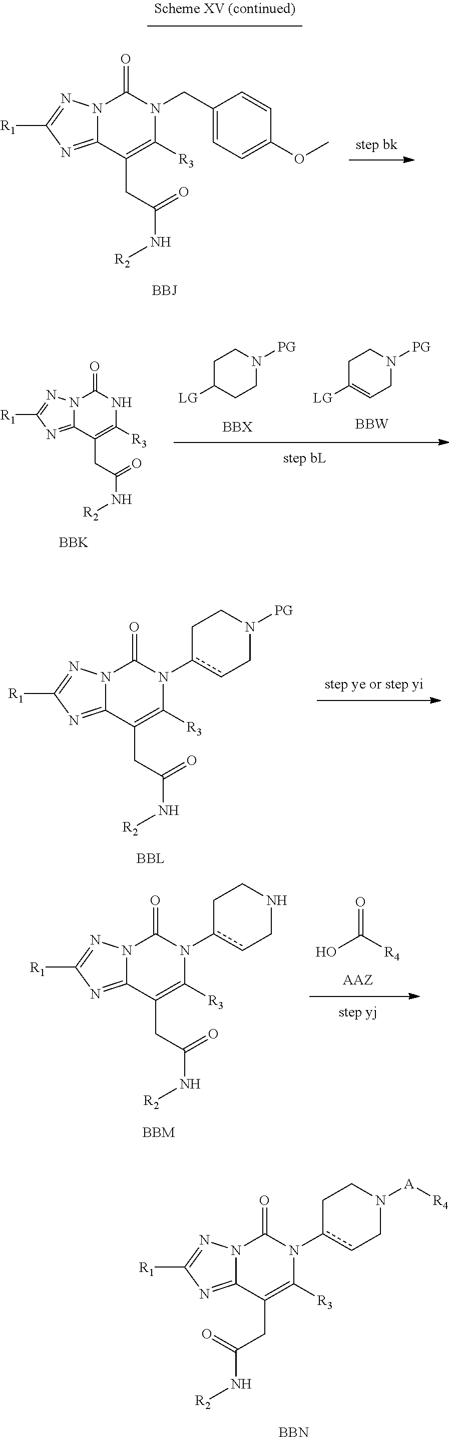

A process for preparing compound of formula BBN (Scheme XV) comprising steps ba, bc, bd, be, bf, bg, bh, bj, bk, bL, bm, ye or yi and yj. It is understood that the order of process ba, bc, bd, be, bf, bg, bh, bj, bk, bL, bm, ye or yi and yj may change to optimize the synthesis as necessary. The compound of formula BBN can be obtained via coupling reaction step yj by reacting compound of formula BBM wherein the substituents are as defined above with compound AAZ wherein R₄ is as defined above using analogous methods to those described herein.

Compound of formula BBM wherein the substituents are as defined above can be prepared deprotecting compound of formula BBL wherein PG represents a suitable protecting group such as BOC or para-methoxybenzyl or benzyl and the other substituents are as defined above comprising step ye or step yi using analogous methods to those described for step e or step i for Scheme XI.

Compound of formula BBL can be prepared comprising step bL starting from compound BBK wherein the substituents are as defined above with either compound BBX or compound BBW wherein PG is as defined above and LG is represented by halo, particularly iodo or bromo or OH or OMs (methanesulfonate) or OTs (p-toluenesulfonate) or OTf (trifluoromethanesulfonate) or B(OH)2, BPin (Pin represents boronic acid pinacol ester) BF3K. Step bL can be performed by combining compound of formula BBK with compound BBX in the presence of a base such as sodium hydride or K2CO3 or DBU or NaOtBu or phosphazene base P2-Et. A stoichiometric excess BBX can be used, preferably between 2 and 50 mole equivalents in an organic solvent for example DMF or NMP. The reaction is preferably stirred at a temperature of approximately 80-150° C. and can be carried out in a capped tube. An alternative method for step bL can use Ullmann-type reaction. Example Ullmann-type cross-coupling conditions comprise reacting compound of formula BBK with compound BBW in the presence of a catalyst such as copper(I)iodide, a ligand such as N-(2-cyanophenyl)pyridine-2-carboxamide or 4,7-dimethoxy-1,10-phenanthroline or N1,N2-dibenzylethane-1,2-diamine and a base such as K3PO4 or K2CO3 in a suitable solvent mixture such as DMSO or DMF at an approximate temperature of 80-150° C. A stoichiometric excess BBX can be used, preferably between 2 and 50 mole equivalents. An alternative method for step bL can comprise reacting compound of formula BBK with compound BBW using Buchwald-Hartwig conditions using for example using an analogous method to those described for step h (Scheme XI). The product of the reaction between the compound of formula BBK and the compound of formula BBW can optionally be hydrogenated using methods known in the art, to give a compound of formula BBL wherein the piperidine ring is saturated. Alternative cross-coupling conditions are known in the art, for examples of methods, see De Meijere et aL. Metal-Catalyzed Cross-Coupling Reactions, Wiley, 2014 and references cited therein.

Compound of formula BBK can be prepared comprising step bk starting from compound of formula BBJ and the substituents are as defined above. Step bk comprises reacting a compound of formula BBJ with a stoichiometric excess of L-methionine for example 3 to 5 mol equivalents in a solvent such as methanesulfonic acid at approximate temperature of 20-80° C.

Compound of formula BBJ can be prepared comprising step bj starting from compound of formula BBI wherein the substituents are as defined above. Step bj comprises reacting a compound of formula BBI with a stoichiometric excess of R2-NH2 wherein R₂ is as defined above for example 3 to 5 mol equivalents in the presence of a stoichiometric excess of trimethylaluminium for example 3-5 mol equivalents in a solvent such as toluene at approximate temperature of 20-80° C.

Compound of formula BBI can be prepared comprising step bi starting from compound of formula BBH wherein the substituents are as defined above. Step bj comprises reacting a compound of formula BBH with a stoichiometric excess of gaseous hydrogen chloride for example 20-100 mol equivalents in ethanol at approximate temperature of 20-100° C. preferably in a sealed tube.

Compound of formula BBH can be prepared comprising step bh starting from compound of formula BBG wherein the substituents are as defined above. Step bh comprises reacting a compound of formula BBH with a stoichiometric excess of potassium fluoride for example 3 to 5 mol equivalents in water in the presence of an additional solvent such as DMF or methanol at an approximate temperature of 20-100° C. preferably 60-100° C.

Compound of formula BBG can be prepared comprising step bg starting from compound of formula BBF wherein R₅₀ is represented by halo, particularly iodo or bromo and the other substituents are as defined above. Step bg comprises reacting a compound of formula BBF with 4-(4,4,5,5-tetramethyl-1,3,2-dioxaborolan-2-yl)isoxazole in the presence of a catalyst such as XPhos Pd G3 and a base such as potassium carbonate in a suitable solvent such as DMF at a temperature of approximately 20-120° C., preferably 60-100° C. under an inert gas such as nitrogen or argon.

Compound of formula BBF can be prepared comprising step bf starting from compound of formula BBE wherein the substituents are as defined above. Step bf comprises reacting a compound of formula BBE with a base such as sodium hydride at an approximate temperature of 0-20° C. in a solvent such as DMF for approximately 5 to 30 minutes under an inert gas such as nitrogen or argon. 1-(bromomethyl)-4-methoxybenzene is then added and the reaction is stirred at a temperature of approximately 0-20° C.

Compound of formula BBE can be prepared comprising step be wherein compound of formula BBD wherein the substituents are defined as above is halogenated. Step be can be carried out using a halogenating reagent such as N-bromosuccinimide or N-iodosuccinimide in a solvent for example DMF or acetonitrile. The reaction is preferably stirred at a temperature of approximately 20-80° C.

Compound of formula BBD can be prepared comprising step bd starting from compound of formula BBC wherein the substituents are as defined above. Step bd comprises reacting a compound of formula BBC with a compound of formula BBY wherein R1 is defined as above in a solvent such as DMF or toluene or dioxane at a temperature of approximately 80-150° C. An alternative method for step bd can comprise reacting compound of formula BBC with a compound of formula BBZ wherein R1 is defined as above in a solvent such as dichloroethane or DMF or toluene or dioxane at a temperature of approximately 0-20° C. A base such as triethylamine may be added. The reaction is then stirred at an approximate temperature of 80-150° C.

Compound of formula BBC can be prepared comprising step bc starting from compound of formula BBB wherein the substituents are defined as above. Step bc comprises reacting a compound of formula BBB with a stoichiometric excess of hydrazine hydrate for example 2 to 5 mol equivalents in a solvent such as ethanol. The reaction is preferably stirred at a temperature of approximately 60-100° C.

Compound of formula BBB can be prepared comprising step ba starting from compound of formula BBA or BBAA wherein the substituents are defined as above. Compound of formula BBA or BBAA are commercially available, or methods for their preparation are known in the art. Step ba comprises reacting a compound of formula BBA or BBAA with P2S5 or lawessons reagent in a solvent such as dioxane or pyridine. The reaction is preferably stirred at a temperature of approximately 80-120° C.

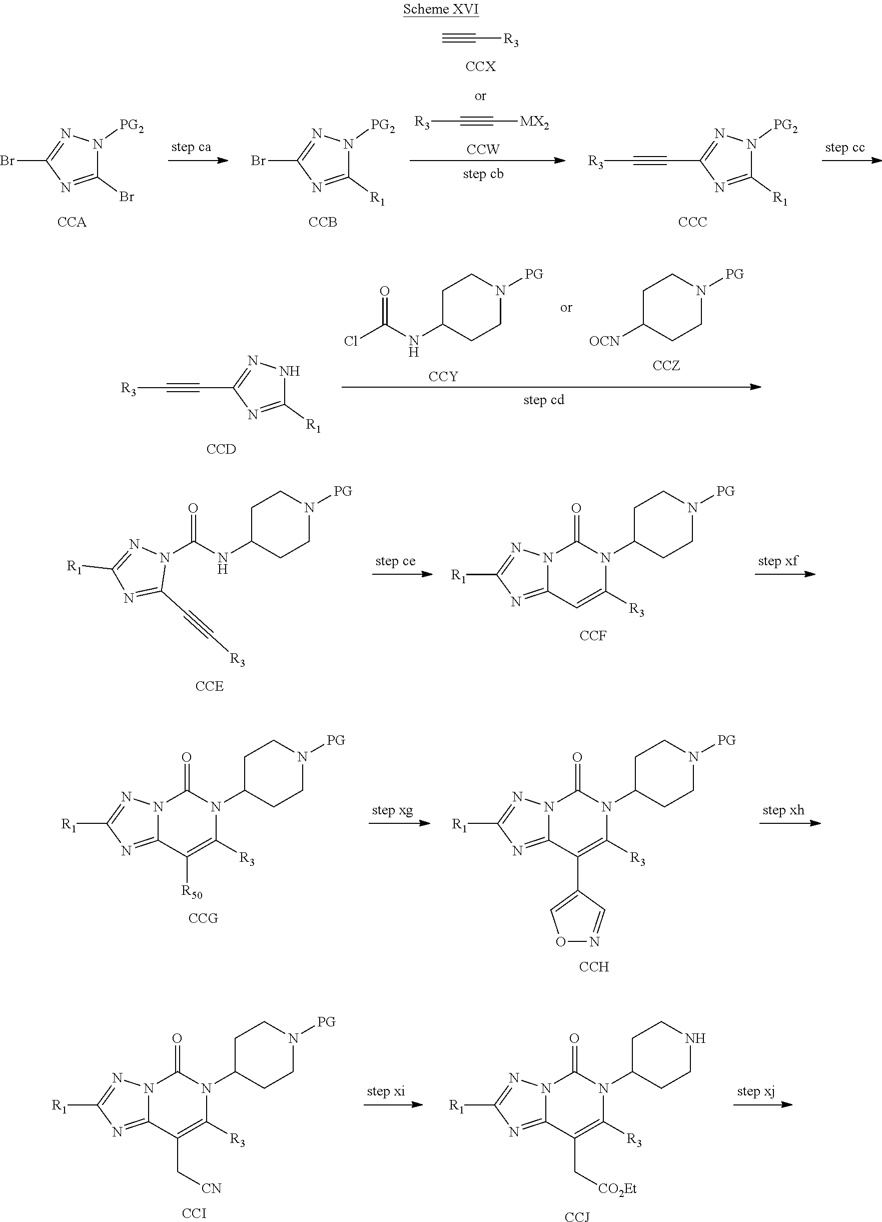

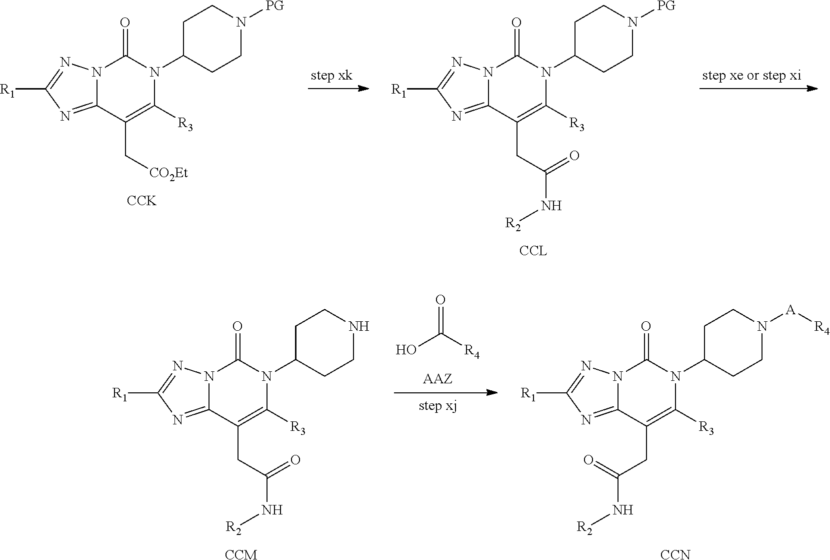

Compound of formula CCN can be prepared according to the route shown in Scheme XVI comprising steps ca, cb, cc, cd, ce, cf, xg, xh, xi, xj, xk, xe or xi and xj. It is understood that the order of process ca, cb, cc, cd, ce, cf, xg, xh, xi, xj, xk, xe or xi and xj may change to optimize the synthesis as necessary. Methods comprising steps xg, xh, xi, xj, xk, xe or xi and xj to prepare compounds of formulas CCG, CCH, CCI, CCJ, CCL, CCM and CCN can be performed using analogous conditions as those described above for steps bf, bg, bh, bi, e, i and j for Scheme XI and Scheme XV.

Compound of formula CCK can be prepared comprising step xj starting from compound of formula CCJ wherein the substituents are defined as above. Step xj comprises reacting a compound of formula CCJ with di-tert-butyl dicarbonate or para-methoxybenzyl bromide or benzyl bromide in the presence of a base such as triethylamine in a solvent such as dichloromethane or dioxane at a temperature of approximately 0-20° C.

Compound of formula CCF wherein PG represents a suitable protecting group such as BOC or para-methoxybenzyl or benzyl and the other substituents are as defined above can be prepared comprising step ce starting from compound of formula CCE wherein substituents are as defined above. Step ce comprises reacting a compound of formula CCE with Echavarren's gold(I) catalyst in a solvent such as THF at a temperature of approximately 60-140° C., preferably 80-120° C. in a sealed tube. An example method is described in Org. Lett. 2013, 15, 11, 2616-2619. An alternative method of preparing compound of formula CCF comprises reacting compound of formula CCE with a base such as sodium hydride in a solvent such as DMF or THF or dioxane at a temperature of approximately 60-140° C., preferably 80-120° C. under an inert gas such as nitrogen or argon in a sealed tube.

Compound of formula CCE can be prepared comprising step cd starting from compound CCD wherein the substituents are defined as above. Step cd comprises reacting a compound of formula CCD with compound of formula CCY or CCZ wherein PG represents a suitable protecting group such as BOC or para-methoxybenzyl or benzyl in the presence of a base such as triethylamine or N-ethyl-N,N-diisopropylamine in a solvent such as THF or dioxane or DMF at a temperature of approximately 20-140° C. preferably 60-120° C. Compound of formula CCY or CCZ are commercially available, or methods for their preparation are known in the art.

Compound of formula CCD can be prepared comprising step cc starting from compound CCC wherein PG2 represents a protecting group such as MOM (methoxymethyl) or SEM (trimethylsilyl)ethoxymethyl) and the other substituents are as defined above. Step cc comprises reacting a compound of formula CCC with an acid such as HCl or TFA in a solvent such as dioxane or dichloromethane at a temperature of approximately 0-80° C. preferably 20-60° C. Alternative methods for deprotection of SEM or MOM groups are known in the art.

Compound of formula CCC can be prepared comprising step cb starting from compound of formula CCB wherein the substituents are as defined above. Step cb can be a Sonogashira reaction reacting a compound of formula CCB with a compound of formula CCX wherein R₃ is as defined above in the presence of a catalyst such as Pd(PPh₃)₄ and a copper catalyst such as copper(I) iodide and a base such as triethylamine or lithium carbonate in a solvent such as dioxane or DMF or acetonitrile THF at a temperature of approximately 20-120° C., preferably 80-120° C. under an inert gas such as nitrogen or argon. Step cb can alternatively be a Suzuki or Stille cross-coupling reaction and comprises reacting a compound of formula CCB with a compound of formula CCW wherein MX2 represents B(OH)₂, BPin (Pin represents boronic acid pinacol ester) BF₃K, B(MIDA), tributyltin and R₃ is as defined above. Methods for Sonogashira, Suzuki or Stille reactions are known in the art. for examples of methods, see Molnar et aL. Palladium-Catalyzed Coupling Reactions, Wiley, 2013 and references cited therein.

Compound of formula CCB can be prepared comprising step ca starting from compound of formula CCA wherein the substituents are defined as above. Step a can be a Suzuki cross-coupling reaction and comprises reacting a compound of formula CCA with R₁ₙ-MX wherein R1 is as defined above, n is 1,2,3 or 4 and MX represents for example B(OH)₂, BPin (Pin represents boronic acid pinacol ester) BF₃K. Example Suzuki cross-coupling conditions comprise reacting compound of formula CCA with R₁—BPin in the presence of a catalyst such as PdCl₂(dppf) or Pd(PPh₃)₄ and a base such as K₃PO₄ or potassium carbonate in a suitable solvent mixture such as DMF, THF or dioxane or water at a temperature of approximately 20-120° C., preferably 60-120° C. under an inert gas such as nitrogen or argon. Compound of formula CCA are commercially available, or methods for their preparation are known in the art.

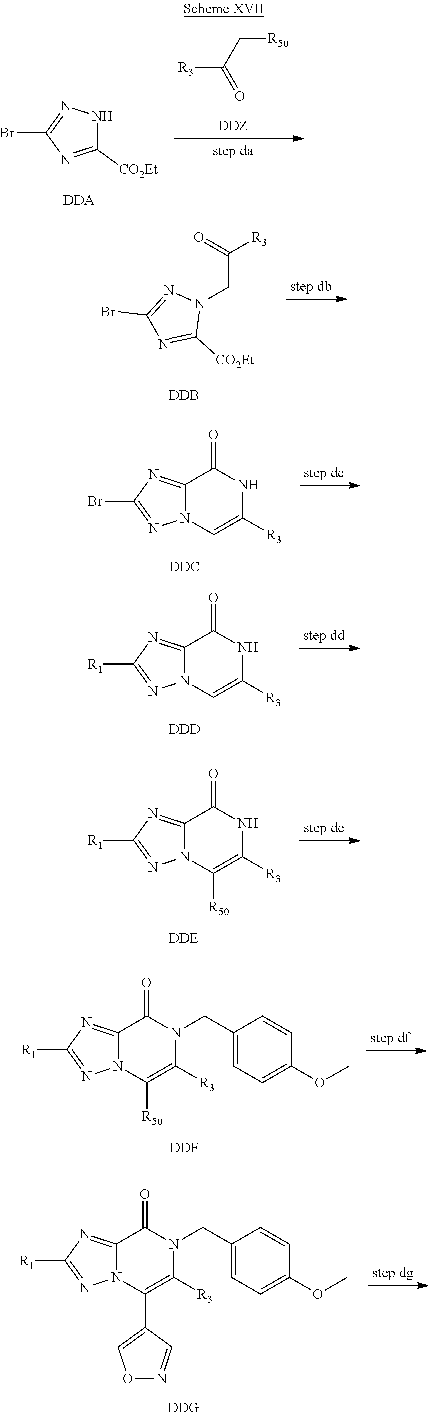

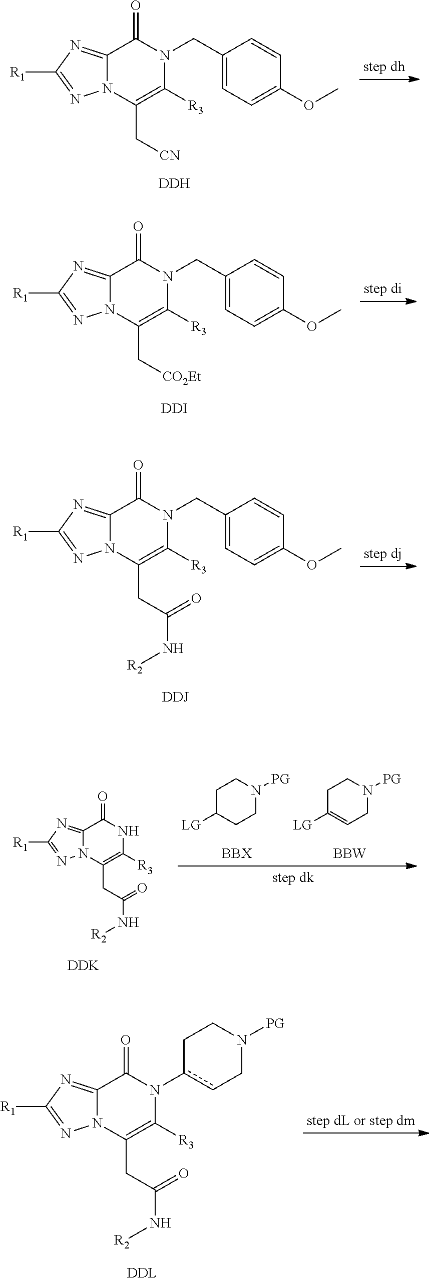

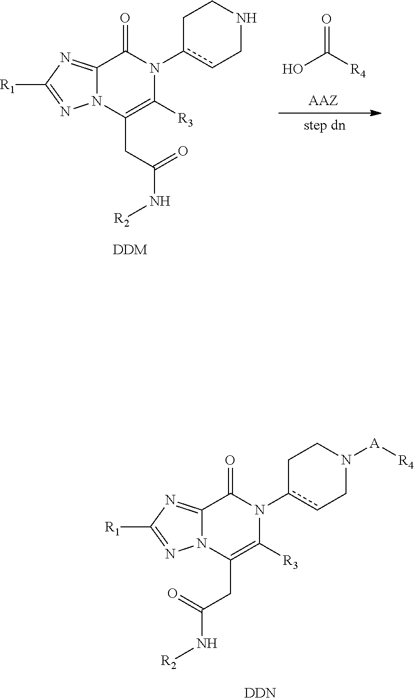

Compound of formula DDN can be prepared according to the route shown in Scheme XVII comprising steps da, db, dc, dd, de, df, dg, dh, di, dj, dk, dL or dM and dn. It is understood that the order of process da, db, dc, dd, de, df, dg, dh, di, dj, dk, dL or dM and dn may change to optimize the synthesis as necessary. Methods comprising steps de, df, dg, dh, di, dj, dk, dL, dM and dn to prepare compounds of formulas DDF, DDG, DDH, DDI, DDJ, DDK, DDL, DDM and DDN can be performed using analogous conditions as those described herein, including optional hydrogenation steps, for example step dk.

Compound of formula DDE wherein R₅₀ represents halo particularly bromo or iodo and the other substituents are as defined above can be prepared comprising step dd starting from compound of formula DDD wherein the substituents are as defined above. Step dd can be carried out using a halogenating reagent such as N-bromosuccinimide or N-iodosuccinimide in a solvent for example DMF or acetonitrile or acetic acid. The reaction is preferably stirred at a temperature of approximately 20-110° C.

Compound of formula DDD wherein the substituents are as defined above can be prepared comprising step dc starting from compound of formula DDC wherein the substituents are as defined above. Step dc can be performed using analogous conditions as those described above for step a for Scheme XI.

Compound of formula DDC wherein the substituents are as defined above can be prepared comprising step db starting from compound of formula DDB wherein the substituents are as defined above. Step db comprises reacting compound of formula DDB with a stoichiometric excess of an ammonium salt such as ammonium acetate for example 3 to 100 mol equivalents particularly 10 to 20 mol equivalents in a solvent such as acetic acid at an approximate temperature of 60-130° C., preferably 80-120° C.

Compound of formula DDB wherein the substituents are as defined above can be prepared comprising step da starting from compound of formula DDA. Step da comprises reacting compound of formula DDA with compound of formula DDZ wherein R₅₀ represents halo particularly chloro or bromo and the other the substituents are as defined above in the presence of a base such as potassium carbonate in a solvent such as acetone or acetonitrile at an approximate temperature of 0-50° C., preferably 0-20° C. Compound of formula DDZ are commercially available, or methods for their preparation are known in the art.

### “Protecting Group”:

In the methods describe above, functional groups which are present in the starting materials and are not intended to take part in the reaction, are present in protected form if necessary, and protecting groups that are present are cleaved, whereby said starting compounds may also exist in the form of salts provided that a salt-forming group is present and a reaction in salt form is possible. In additional process steps, carried out as desired, functional groups of the starting compounds which should not take part in the reaction may be present in unprotected form or may be protected for example by one or more protecting groups. The protecting groups are then wholly or partly removed according to one of the known methods. Protecting groups, and the manner in which they are introduced and removed are described, for example, in “Protective Groups in Organic Chemistry”, Plenum Press, London, New York 1973, and in “Methoden der organischen Chemie”, Houben-Weyl, 4th edition, Vol. 15/1, Georg-Thieme-Verlag, Stuttgart 1974 and in Theodora W. Greene, “Protective Groups in Organic Synthesis”, John Wiley & Sons, New York 1981. A characteristic of protecting groups is that they can be removed readily, i.e. without the occurrence of undesired secondary reactions, for example by solvolysis, reduction, photolysis or alternatively under physiological conditions

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure antipodes.

Compounds of the invention and intermediates can also be converted into each other according to methods generally known to those skilled in the art.

Intermediates and final products can be worked up and/or purified according to standard methods, e.g. using chromatographic methods, distribution methods, (re-) crystallization, and the like.

The following applies in general to all processes mentioned herein before and hereinafter.

All the above-mentioned process steps can be carried out under reaction conditions that are known to those skilled in the art, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g. in the H+ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described herein above.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, methycyclohexane, or mixtures of those solvents, for example aqueous solutions, unless otherwise indicated in the description of the processes. Such solvent mixtures may also be used in working up, for example by chromatography or partitioning.

Sulfonimidamides, and their synthesis, are described in Chem. Eur. J. 2017, 23, 15189-15193 DOI:10.1002/chem.201703272.

1. A compound of formula (I) or a pharmaceutically acceptable salt thereof, 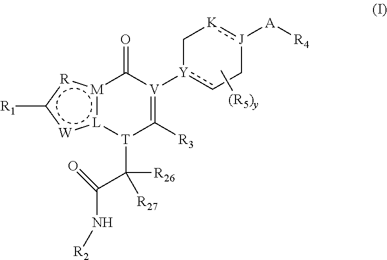
wherein
R, M, W, L, V and T are independently selected from C, CH and N,
to form subformulae 1a, 1b, 1c, 1d, 1e and 1f: 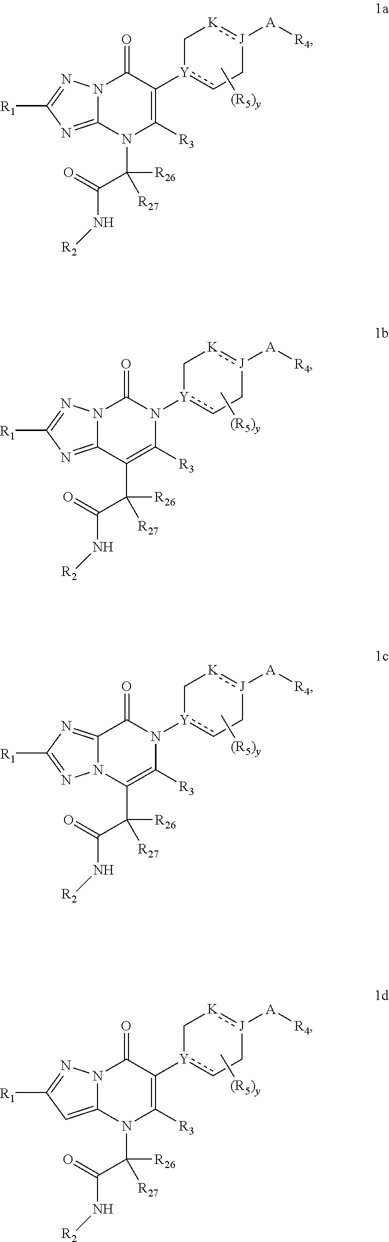 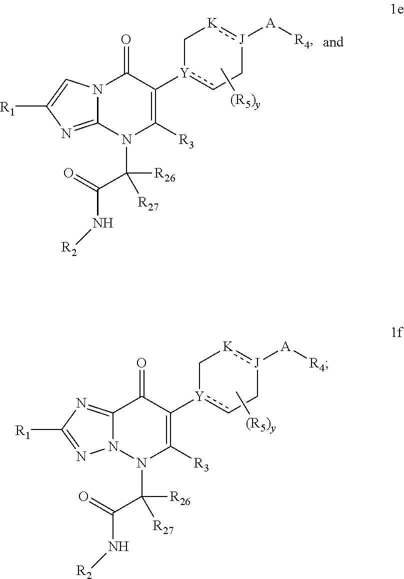
A is a linker which is —C(O)—;
Y is N, C or CH;
y is 0, 1, 2, 3 or 4;
Y [symbol] means Y is linked via a single bond to the adjacent carbon atom when Y is CH, or Y is linked via a double bond to the adjacent atom when Y is C, and when Y [symbol] is a single bond, Y is carbon unsubstituted or substituted by OH or F;
when Y is N, Y [symbol] is a single bond;
K [symbol] means K is linked via a single or double bond to the adjacent atom;
wherein:
when K [symbol] is a double bond, Y [symbol] is a single bond, K is CH and J is C, or
when K [symbol] is a single bond, K is selected from —CH₂—, —CH₂CH₂—, —NH— and a
bond (to form a 5-membered ring: 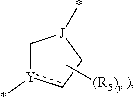
and J is N;
R₅ is independently selected from:
—(C₁-C₄)alkyl,
—(C₃-C₅)cycloalkyl,
and wherein two R₅ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S,
when K [symbol] J is a carbon-nitrogen single bond, a R₅ substituent on K and on the adjacent carbon atom may join to form ring C: 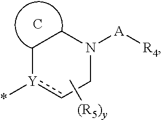
wherein ring C is a fused (C₃-C₆)cycloalkyl ring, a fused (C₃-C₆)heterocyclyl ring or a fused phenyl ring, wherein said fused (C₃-C₆)heterocyclyl ring contains ring carbon atoms and one ring heteroatom selected from O, N and S,
and wherein when ring C is a fused (C₃-C₆)cycloalkyl ring, said fused (C₃-C₆)cycloalkyl ring is unsubstituted or substituted with 1 or 2 R₄₀ groups, wherein said R₄₀ is selected from:
(C₁-C₂)alkyl, wherein each (C₁-C₂)alkyl is independently unsubstituted or substituted by OH or 1, 2 or 3 halo,
halo, in particular F,
or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
or wherein two R₄₀ substituents on adjacent carbon atoms join together with the carbon atoms to which they are attached, to form a fused cyclopropyl ring;
and wherein when K is —CH₂— and J is N, two R₅ substituents may join to form a (C₁-C₃)alkylene bridge or a heteroalkylene bridge, wherein said heteroalkylene bridge is one heteroatom selected from N and O, or is —CH₂—O—CH₂—;
R₁ is:
cycloalkenyl, wherein said cycloalkenyl is a partially unsaturated monocyclic ring containing 5 or 6 ring carbon atoms, and said cycloalkenyl is unsubstituted or substituted by 1, 2, 3 or 4, preferably 1 or 2, R₃₃, wherein R₃₃ is halo, and wherein said cycloalkenyl or halo-substituted cycloalkenyl is substituted by 0, 1 or 2 R₁₅ substituents, or said cycloalkenyl or halo-substituted cycloalkenyl has 2 substitutents at the same ring carbon atom which join to form an oxetanyl spiro ring,
or R₁ is heterocyclyl, wherein said heterocyclyl is a 5 or 6 membered fully saturated or partially unsaturated group comprising ring carbon atoms and 1 or 2 ring heteroatoms independently selected from N, O and S, and wherein said heterocyclyl is unbridged or bridged, and said bridge is 1 or 2 carbon atoms, wherein said heterocyclyl is unsubstituted or substituted by 1, 2, 3 or 4, preferably 1 or 2, R₃₃, wherein R₃₃ is halo, and wherein said heterocyclyl or halo-substituted heterocyclyl is substituted by 0, 1 or 2 substituents independently selected from R₁₅, R₁₆, R₁₇, R₁₈, R₁₉, R₂₀, R₂₂ and R₂₃,
or said heterocyclyl or halo-substituted heterocyclyl is fused to a cyclopropyl ring, wherein said cyclopropyl ring is unsubstituted or substituted by 1, 2 or 3 F,
or said heterocyclyl or halo-substituted heterocyclyl has 2 substitutents at the same ring carbon atom which join to form a cyclopropyl spiro ring, or tetrahydrofuranyl spiro ring,
or said heterocyclyl or halo-substituted heterocyclyl is fused with a (C₃-C₅)heterocycloalkyl ring, wherein said (C₃-C₅)heterocycloalkyl ring contains ring carbon atoms and 1 ring O atom;
or R₁ is heteroaryl, wherein said heteroaryl is a 5 or 6 membered fully unsaturated monocyclic group comprising ring carbon atoms and 1, 2, 3 or 4 ring heteroatoms independently selected from N, O and S, preferably 1 or 2 ring heteraoms, preferably wherein the total number of ring S atoms does not exceed 1 and preferably the total number of ring 0 atoms does not exceed 1,
and wherein said heteroaryl is unsubstituted or substituted by 1, 2 or 3 substituents independently selected from R₂₁ and R₃₀, wherein R₂₁ and R₃₀ are independently selected from halo and (C₁-C₄)alkyl, wherein said (C₁-C₄)alkyl is unsubstituted or substituted by 1, 2 or 3 halo,
or R₁ is phenyl, wherein said phenyl is unsubstituted or substituted by 1, 2, 3 or 4, preferably 1 or 2, R₃₃, wherein R₃₃ is halo, and wherein said phenyl or halo-substituted phenyl is substituted by 0, 1 or 2 R₁₅ substituents,
or R₁ is (C₂-C₄)alkynyl or (C₂-C₄)alkenyl, wherein said (C₂-C₄)alkynyl and (C₂-C₄)alkenyl are unsubstituted or substituted by (C₁-C₄)alkyl-O—C(O)—, or morpholinyl;
each R₁₅, R₁₆, R₁₇, R₁₈, Rig, R₂₀, R₂₂ and R₂₃ is independently selected from:
halo
(C₁-C₄)alkyl-O—(CH₂)ₙ unsubstituted or substituted by 1, 2 or 3 halo;
(C₁-C₄)alkyl unsubstituted or substituted by OH, —O—(C₁-C₂)alkyl or 1, 2 or 3 halo,
HOC(O)—(CH₂)ₙ—,
(C₁-C₄)alkyl-C(O)(CH₂)ₙ—,
(E)-cyclooct-4-en-1-yl-O—C(O)—,
(C₁-C₄)alkyl-O—C(O)(CH₂)ₙ,
═O
azetidinyl or pyrrolidinyl, wherein said azetidinyl and pyrrolidinyl are linked to the rest of the molecule via the N atom, and are each unsubstituted or substituted by 1 or 2 F,
R₂₅(R₂₄)N—(CH₂)ₙ, wherein R₂₄ is H or (C₁-C₄)alkyl unsubstituted or substituted by 1, 2 or 3 halo, and R₂₅ is:
H,
(C₁-C₄)alkyl-C(O)(CH₂)ₙ—, wherein said (C₁-C₄)alkyl of (C₁-C₄)alkyl-C(O)(CH₂)ₙ— is unsubstituted or substituted by halo or —N₃,
(C₁-C₄)alkyl-O—C(O)(CH₂)ₙ,
(C₁-C₄)alkyl unsubstituted or substituted by 1, 2 or 3 halo, or
(E)-cyclooct-4-en-1-yl-O—C(O)—,
OH
wherein n is 0, 1 or 2,
R₂₆ is CH₃, H or deuterium;
R₂₇ is CH₃, H or deuterium;
or R₂₆ and R₂₇ join, together with the carbon atom to which they are attached, to form a cyclopropyl ring;
R₂ is the moiety: 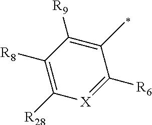
R₆ is selected from:
H,
halo,
(C₁-C₄)alkyl unsubstituted or substituted by 1, 2 or 3 halo,
(C₃-C₅)cycloalkyl unsubstituted or substituted by 1, 2 or 3 halo,
—O—(C₁-C₄)alkyl unsubstituted or substituted by 1, 2 or 3 halo,
OH, and
CN;
R₈ is selected from H, halo, and (C₁-C₄)alkyl unsubstituted or substituted by 1, 2 or 3 halo,
R₉ is selected from H, O—CH₃, OH, CN, CH₃ and halo;
R₂₈ is selected from:
SF₅,
H,
—C(O)H,
halo,
(C₁-C₄)alkyl unsubstituted or substituted by 1, 2 or 3 halo,
(C₁-C₄)alkynyl,
(C₁-C₄)alkenyl,
(C₃-C₅)cycloalkyl unsubstituted or substituted by 1, 2 or 3 halo, and
OCF₃;
X is selected from C—R₇ and N, wherein R₇ is H, CF₃ or halo, or R₇ can join, together with R₂₈ or R₆, and the atoms to which they are attached, to form a fused (C₄-C₆)cycloalkyl ring, wherein said fused (C₄-C₆)cycloalkyl ring is unsubstituted or substituted by 1, 2 or 3 halo,
or
R₂ is selected from: 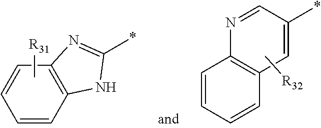
wherein
R₃₁ is selected from H, halo and CH₃,
R₃₂ is selected from H, halo and CH₃,
R₃ is:
cyclopropyl,
O—CH₃,
N(CH₃)₂,
S—CH₃,
(C₁-C₄)alkyl unsubstituted or substituted by 1, 2 or 3 substituents independently selected from halo and OH;
R₄ is selected from:
—(C₁-C₄)alkyl, unsubstituted or substituted by NH₂;
—O—CH₂phenyl;
—O—CH₂CH₂phenyl;
—NH—NH—C(O)—CF₃;
heteroaryl1, wherein said heteroaryl1 is a 5 or 6 membered, fully unsaturated, monocyclic ring comprising ring carbon atoms and 1, 2, 3 or 4 ring heteroatoms independently selected from N, O and S;
heteroaryl2, wherein said heteroaryl2 is a 9 or 10 membered fused bicyclic ring comprising ring carbon atoms and 1, 2, 3 or 4 ring heteroatoms independently selected from N, O and S, and wherein both rings are fully unsaturated, or one ring is fully unsaturated, and the other is saturated or partially unsaturated, and wherein the heteroatoms may be in one or both rings;
-phenyl;
-heterocyclyl2, wherein said heterocyclyl2 is a 5 or 6 membered fully saturated or partially unsaturated group comprising ring carbon atoms and 1 or 2 ring heteroatoms independently selected from N, O and S,
and 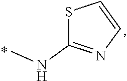
wherein heteroaryl1, heteroaryl2 and phenyl are each substituted by 1, 2 or 3 substituents independently selected from R₁₀, R₁₁, R₁₂, R₁₃ and R₁₄, wherein each R₁₀, R₁₁, R₁₂, R₁₃ and R₁₄ is independently selected from:
H,
halo,
(C₁-C₄)alkyl unsubstituted or substituted by 1, 2 or 3 halo substituents,
(C₁-C₂)alkyl substituted by —O—(C₁-C₂)alkyl or OH,
—S—(C₁-C₃)alkyl,
—O—(C₁-C₄)alkyl unsubstituted or substituted by 1, 2 or 3 halo substituents,
OH,
(C₃-C₅)cycloalkyl, wherein said (C₃-C₅)cycloalkyl is unsubstituted or substituted by 1 or 2 halo,
—O—(C₃-C₅)cycloalkyl,
—NR₃₄R₃₅ wherein R₃₄ and R₃₅ are independently selected from:
H,
(C₁-C₄)alkyl, wherein said (C₁-C₄)alkyl is unsubstituted or substituted by OH or —O(C₁-C₂)alkyl,
and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
CN,
—(C₂-C₄)alkenyl,
—(C₂-C₄)alkynyl,
═O
—C(O)H, and
—C(O)(C₁-C₄)alkyl;
with the proviso that R₄ is not: 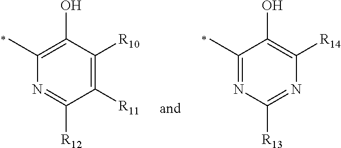
wherein
R₁₀, R₁₁, R₁₂, R₁₃ and R₁₄ are independently selected from:
H,
halo,
(C₁-C₄)alkyl unsubstituted or substituted by 1, 2 or 3 halo substituents,
(C₁-C₂)alkyl substituted by —O—(C₁-C₂)alkyl or OH,
—S—(C₁-C₃)alkyl,
—O—(C₁-C₄)alkyl unsubstituted or substituted by 1, 2 or 3 halo substituents,
OH,
(C₃-C₅)cycloalkyl, wherein said (C₃-C₅)cycloalkyl is unsubstituted or substituted by 1 or 2 halo,
—O—(C₃-C₅)cycloalkyl,
—NR₃₄R₃₅ wherein R₃₄ and R₃₅ are independently selected from:
H,
(C₁-C₄)alkyl, wherein said (C₁-C₄)alkyl is unsubstituted or substituted by OH or —O(C₁-C₂)alkyl,
and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
CN,
—(C₂-C₄)alkenyl,
—(C₂-C₄)alkynyl,
—C(O)H, and
—C(O)(C₁-C₄)alkyl;
and
* indicates a point of attachment.

2. A compound of formula (I) or a pharmaceutically acceptable salt thereof, according to claim 1, wherein when R₁ is a ring, then:
each R₁ ring atom adjacent to the R₁ ring atom to which said R₁ ring is joined to the remainder of the molecule, is independently unsubstituted or substituted by halo only, in particular, independently unsubstituted or substituted with one F substituent, and
preferably, said R₁ ring is linked to the remainder of the molecule via a R₁ ring nitrogen atom, or a R₁ ring carbon atom which is double-bonded to an adjacent R₁ ring atom.

3. A compound of formula (I) or a pharmaceutically acceptable salt thereof, according to claims 1 or 2, wherein R₁ is:
cycloalkenyl, wherein said cycloalkenyl is a partially unsaturated monocyclic ring containing 5 or 6 ring carbon atoms, and said cycloalkenyl is unsubstituted or substituted by 1, 2, 3 or 4, preferably 1 or 2, R₃₃, wherein R₃₃ is halo, and wherein said cycloalkenyl or halo-substituted cycloalkenyl is substituted by 0, 1 or 2 R₁₅ substituents, preferably 1 substituent, or said cycloalkenyl or halo-substituted cycloalkenyl has 2 substitutents at the same ring carbon atom which join to form an oxetanyl spiro ring,
or R₁ is heterocyclyl, wherein said heterocyclyl is a 5 or 6 membered fully saturated or partially unsaturated group comprising ring carbon atoms and 1 or 2 ring heteroatoms independently selected from N, NH, O and S, and wherein said heterocyclyl is unbridged or bridged, and said bridge is 1 or 2 carbon atoms, wherein said heterocyclyl is unsubstituted or substituted by 1, 2, 3 or 4, for example 1, 2 or 3, in particular 1 or 2 R₃₃, wherein R₃₃ is halo, and wherein said heterocyclyl or halo-substituted heterocyclyl is substituted by 0, 1 or 2 substituents, preferably 0 or 1 substituent, independently selected from R₁₅, R₁₆, R₁₇, R₁₈, R₁₉, R₂₀, R₂₂ and R₂₃,
or said heterocyclyl or halo-substituted heterocyclyl is fused to a cyclopropyl ring, wherein said cyclopropyl ring is unsubstituted or substituted by 1, 2 or 3 F,
or said heterocyclyl or halo-substituted heterocyclyl has 2 substitutents at the same ring carbon atom which join to form a tetrahydrofuranyl spiro ring,
or R₁ is heteroaryl, wherein said heteroaryl is a 5 or 6 membered fully unsaturated monocyclic group comprising ring carbon atoms and 1, 2, 3 or 4 ring heteroatoms independently selected from N, O and S, preferably 1 or 2 ring heteraoms, wherein the total number of ring S atoms does not exceed 1, and the total number of ring 0 atoms does not exceed 1, wherein said heteroaryl is unsubstituted or substituted by 1, 2 or 3 substituents independently selected from R₂₁ and R₃₀, wherein R₂₁ and R₃₀ are independently selected from halo and (C₁-C₄)alkyl, wherein said (C₁-C₄)alkyl is unsubstituted or substituted by 1, 2 or 3 halo,
or R₁ is phenyl, wherein said phenyl is unsubstituted or substituted by 1, 2, 3 or 4, preferably 1 or 2, R₃₃, wherein R₃₃ is halo, and wherein said phenyl or halo-substituted phenyl is substituted by 0 or 1 R₁₅ substituents,
or R₁ is (C₂-C₄)alkynyl, unsubstituted or substituted by (C₁-C₄)alkyl-O—C(O)—;
and each R₁₅, R₁₆, R₁₇, R₁₈, Rig, R₂₀, R₂₂ and R₂₃ is independently selected from:
halo
(C₁-C₄)alkyl-O—(CH₂)ₙ unsubstituted or substituted by 1, 2 or 3 halo;
(C₁-C₄)alkyl unsubstituted or substituted by OH, —O—(C₁-C₂)alkyl or 1, 2 or 3 halo,
HOC(O)—(CH₂)ₙ—,
H₃C—C(O)(CH₂)ₙ—,
(C₁-C₄)alkyl-O—C(O)(CH₂)ₙ,
═O
azetidinyl or pyrrolidinyl, wherein said azetidinyl and pyrrolidinyl are linked to the rest of the molecule via the N atom, and are each unsubstituted or substituted by 1 or 2 F,
R₂₅(R₂₄)N—(CH₂)ₙ, wherein R₂₄ is H or (C₁-C₂)alkyl unsubstituted or substituted by 1, 2 or 3 halo, R₂₅ is H, (C₁-C₄)alkyl-C(O)—, (C₁-C₄)alkyl-O—C(O)—, or (C₁-C₄)alkyl unsubstituted or substituted by 1, 2 or 3 halo,
OH
wherein n is 0, 1 or 2,

4. A compound of formula (I) or a pharmaceutically acceptable salt thereof, according to any of claims 1, 2 or 3, wherein R₁ is:
cycloalkenyl, wherein said cycloalkenyl is a partially unsaturated monocyclic ring containing 5 or 6 ring carbon atoms, and said cycloalkenyl is unsubstituted or substituted by 1 or 2 R₃₃, wherein R₃₃ is halo, preferably F, and wherein said cycloalkenyl or halo-substituted cycloalkenyl is substituted by 0 or 1 R₁₅ substituents, wherein R₁₅ is selected from:
o) (C₁-C₂)alkyl-O— unsubstituted or substituted by 1, 2 or 3 halo;
p) (C₁-C₂)alkyl unsubstituted or substituted by 1, 2 or 3 halo,
q) HOC(O)—(CH₂)ₙ—,
r) H₃C—C(O)(CH₂)ₙ—,
S) H₃C—O—C(O)(CH₂)ₙ,
t) ═O, and
u) R₂₅(R₂₄)N—, H, wherein R₂₄ is H or (C₁-C₂)alkyl unsubstituted or substituted by 1, 2 or 3 halo, R₂₅ is H or (C₁-C₂)alkyl unsubstituted or substituted by 1, 2 or 3 halo,
n is 0 or 1,
wherein
the R₁₅ substituent a) to g) of said cycloalkenyl or halo-substituted cycloalkenyl is not present on the ring atoms adjacent to the ring atom to which the cycloalkenyl or halo-substituted cycloalkenyl is joined to the remainder of the molecule, and preferably, said cycloalkenyl or halo-substituted cycloalkenyl is a 6 membered ring, with 1 R₁₅ substituent in the ring para position relative to the remainder of the molecule; and
said cycloalkenyl or halo-substituted cycloalkenyl is linked to the remainder of the compound via a R₁ ring carbon atom which is double bonded to an adjacent R₁ ring carbon atom;
or R₁ is heterocyclyl, wherein said heterocyclyl is a 5 or 6 membered fully saturated or partially unsaturated group comprising ring carbon atoms and 1 or 2 ring heteroatoms independently selected from N, NH, O and S, and wherein said heterocyclyl is unbridged or bridged, and said bridge is 1 or 2 carbon atoms, wherein said heterocyclyl is unsubstituted or substituted by 1 or 2 R₃₃, wherein R₃₃ halo, is preferably F, and wherein said heterocyclyl or halo-substituted heterocyclyl is substituted by 0 or 1 substituents independently selected from R₁₅, R₁₆, R₁₇, R₁₈, Rig, R₂₀, R₂₂ and R₂₃, wherein said R₁₅, R₁₆, R₁₇, R₁₈, Rig, R₂₀, R₂₂ and R₂₃ are independently selected from:
q) (C₁-C₄)alkyl-O— unsubstituted or substituted by 1, 2 or 3 halo;
r) (C₁-C₄)alkyl unsubstituted or substituted by OH, —O—(C₁-C₂)alkyl or 1, 2 or 3 halo,
s) HOC(O)—(CH₂)ₙ—,
t) H₃C—C(O)(CH₂)ₙ—,
u) H₃C—O—C(O)(CH₂)ₙ,
v) ═O
w) R₂₅(R₂₄)N—, wherein R₂₄ is H, (C₁-C₂)alkyl unsubstituted or substituted by 1, 2 or 3 halo, R₂₅ is H, (C₁-C₂)alkyl unsubstituted or substituted by 1, 2 or 3 halo,
x) OH
wherein n is 0 or 1,
and wherein:
substituent a) to h) of said heterocyclyl or halo-substituted heterocyclyl is not present on the ring atoms adjacent to the ring atom to which the heterocyclyl or halo-substituted heterocyclyl is joined to the remainder of the molecule, and preferably, when said heterocyclyl or halo-substituted heterocyclyl is a 6 membered ring, it has 0 or 1 substituent selected from a) to h) in the meta or para position, preferably para, relative to the remainder of the molecule; and
said heterocyclyl is linked to the remainder of the compound via a R₁ ring nitrogen atom, or a R₁ ring carbon atom which is double bonded to an adjacent ring atom;
or R₁ is heteroaryl, wherein said heteroaryl is a 5 or 6 membered fully unsaturated monocyclic group comprising ring carbon atoms and 1 or 2 ring heteroatoms independently selected from N, O and S, preferably N, wherein the total number of ring S atoms does not exceed 1, and the total number of ring O atoms does not exceed 1, wherein said heteroaryl is unsubstituted or substituted by 1 or 2 substituents independently selected from R₂₁ and R₃₀, wherein R₂₁ and R₃₀ are independently selected from (C₁-C₂)alkyl, and said (C₁-C₂)alkyl is unsubstituted or substituted by 1, 2 or 3 halo, and wherein preferably, said alkyl or halo-alkyl substituent is not present on the R₁ ring atoms adjacent to the R₁ ring atom to which the heteroaryl is joined to the remainder of the molecule, and more preferably, when heteroaryl is a 6-membered ring, said alkyl or halo-alkyl substituent is in the ring para position relative to the rest of the molecule.

5. A compound of formula (I) or a pharmaceutically acceptable salt thereof, according to any of claims 1 to 4, wherein R₁ is selected from: 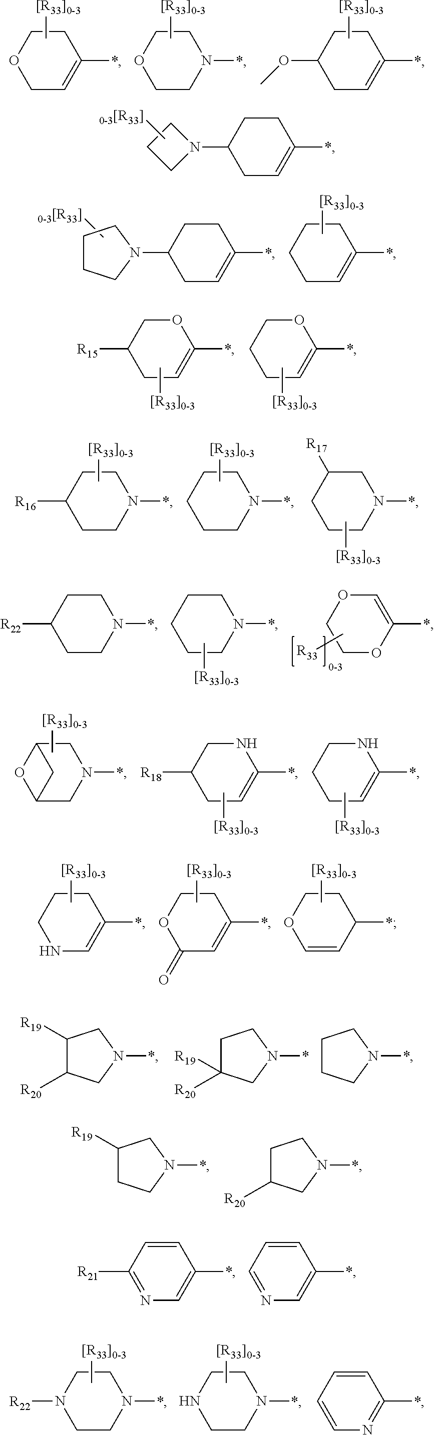 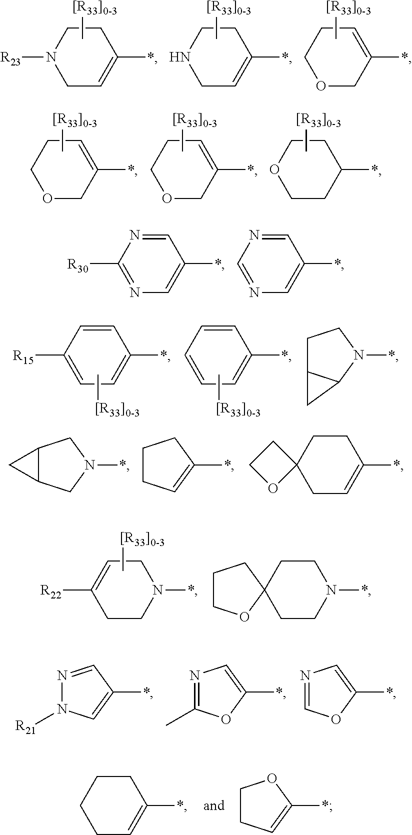
alternatively, there are 0-2 R₃₃ substituents, in each of the moieties above,
R₃₃ is F;
R₁₅ is:
halo,
R₂₅(R₂₄)N—(CH₂)ₙ, wherein R₂₄ is H or CH₃ unsubstituted or substituted by 1, 2 or 3 halo, R₂₅ is H, (C₁-C₄)alkyl-C(O)—, (C₁-C₄)alkyl-O—C(O)—, or (C₁-C₄)alkyl unsubstituted or substituted by 1, 2 or 3 halo, or
azetidinyl or pyrrolidinyl, wherein said azetidinyl and pyrrolidinyl are linked to the rest of the molecule via the N atom, and are unsubstituted or substituted by 1 or 2 F;
R₁₆ is R₂₅(R₂₄)N—, wherein R₂₄ is H or (C₁-C₂)alkyl, R₂₅ is H or (C₁-C₂)alkyl unsubstituted or substituted by 1, 2 or 3 halo, in particular F;
R₁₇ is halo
R₁₈ is halo;
R₁₉ is:
halo
(C₁-C₄)alkyl unsubstituted or substituted by 1, 2 or 3 halo,
(C₁-C₄)alkyl-O—(CH₂)ₙ—;
R₂₀ is halo;
R₂₁ is (C₁-C₂)alkyl, unsubstituted or substituted by 1, 2 or 3 F;
R₂₂ and R₂₃ are each independently selected from:
(C₁-C₄)alkyl unsubstituted or substituted by 1, 2 or 3 halo,
(C₁-C₄)alkyl-O—(CH₂)ₙ—
HOC(O)—(CH₂)ₙ—,
H₃C—C(O)(CH₂)ₙ—,
(H₃C)₃C—O—C(O)(CH₂)ₙ—;
wherein n is 0, 1 or 2;
and
R₃₀ is CH₃.

6. A compound of formula (I) or a pharmaceutically acceptable salt thereof, according to any of claims 1 to 5, wherein R₁ is selected from: 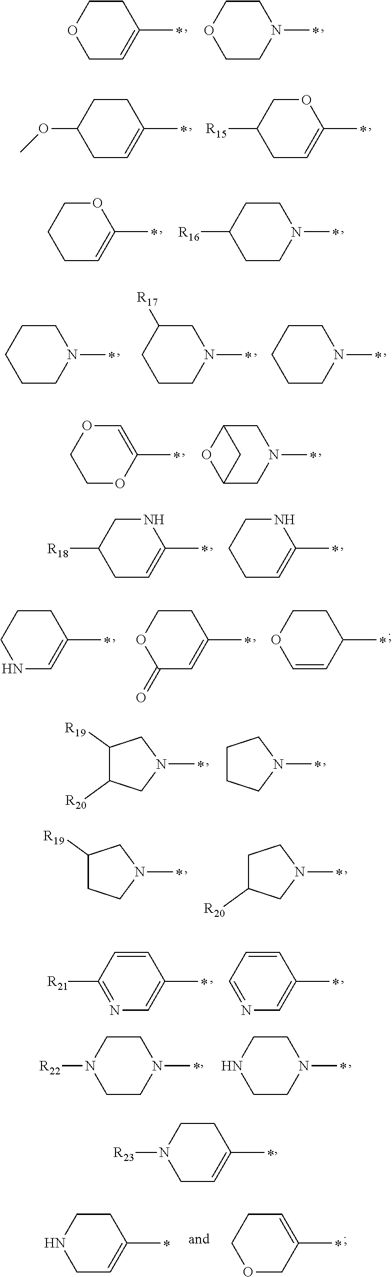
R₁₅ is F;
R₁₆ is R₂₅(R₂₄)N—;
R₁₇ is F;
R₁₈ is F;
R₁₉ is F;
R₂₀ is F;
R₂₁ is CH₃;
R₂₂ is CF₃, CHF₂CH₂, HOC(O)—CH₂—, H₃C—C(O)—, (H₃C)₃C—O—C(O)—;
R₂₃ is CF₃, CHF₂CH₂—, (H₃C)₃C—O—C(O)—;
R₂₄ is CH₃; and
R₂₅ is CHF₂CH₂—.

7. A compound of formula (I), or a pharmaceutically acceptable salt thereof, according to any of claims 1 to 6, wherein R₁ is selected from:

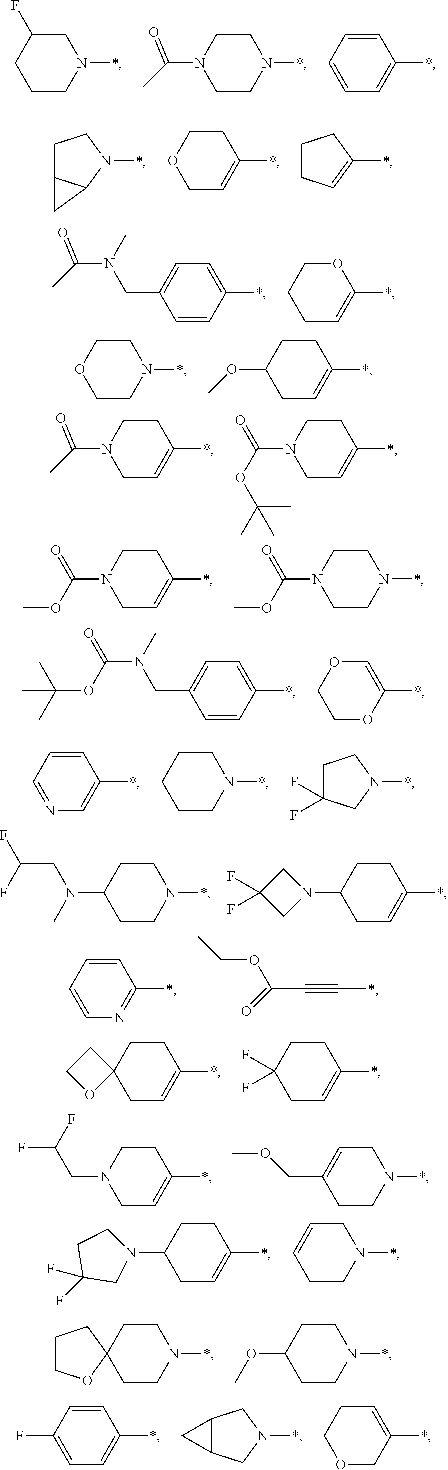

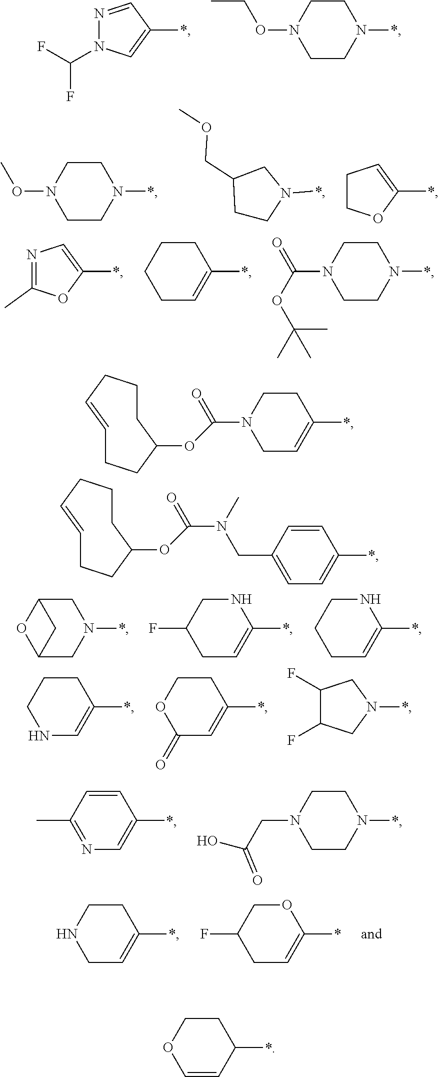

8. A compound of formula (I) or a pharmaceutically acceptable salt thereof, according to any of claims 1 to 7, wherein R₂ is the moiety: 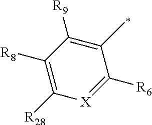
wherein
R₆ is selected from H, halo, (C₁-C₄)alkyl unsubstituted or substituted by 1, 2 or 3 halo;
R₈ is selected from H, halo, (C₁-C₄)alkyl unsubstituted or substituted by 1, 2 or 3 halo;
R₉ is selected from H, O—CH₃, OH, CN, CH₃ and halo;
R₂₈ is selected from SF₅, halo, (C₁-C₄)alkyl unsubstituted or substituted by 1, 2 or 3 halo, and —C(O)H;
X is selected from C—R₇ and N; and
R₇ is selected from H and halo.

9. A compound of formula (I), or a pharmaceutically acceptable salt thereof, according to any of claims 1 to 8, wherein R₂ is selected from

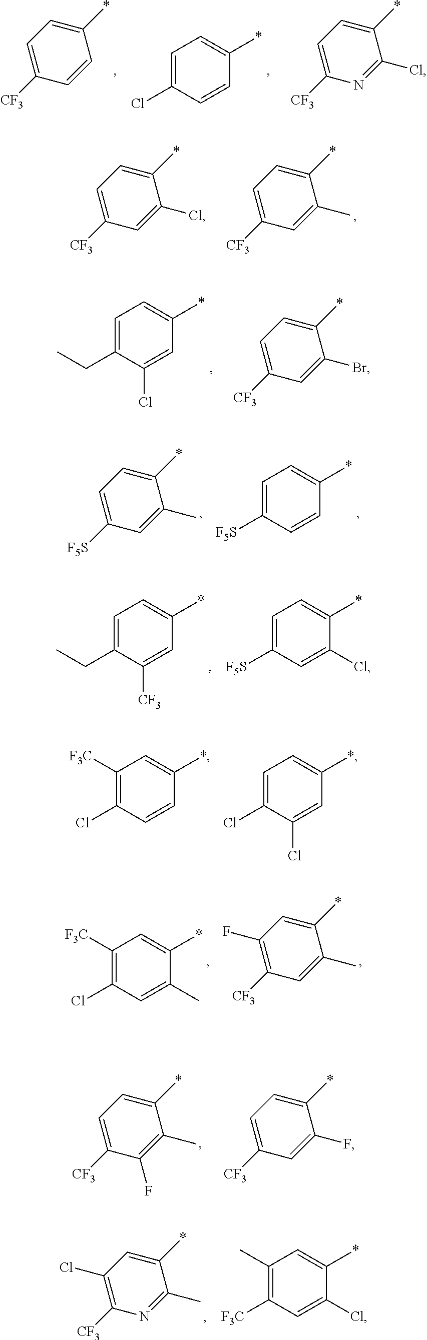

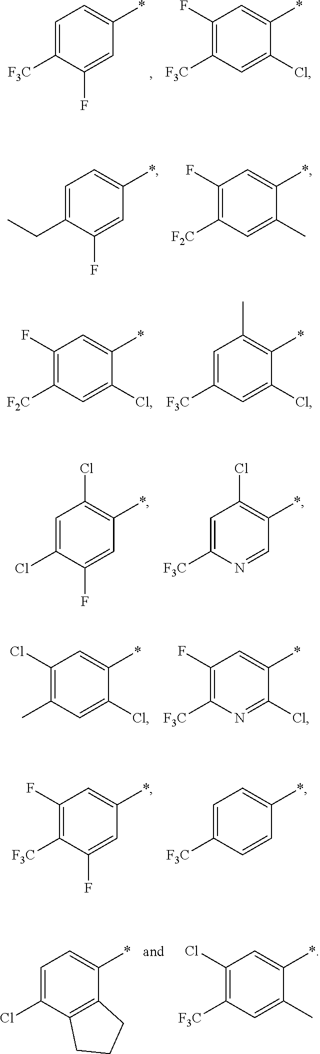

10. A compound of formula (I) or a pharmaceutically acceptable salt thereof, according to any of claims 1 to 9, wherein R₃ is (C₁-C₄)alkyl unsubstituted or substituted by 1, 2 or 3 substituents independently selected from halo and OH.

11. A compound of formula (I) or a pharmaceutically acceptable salt thereof, according to any of claims 1 to 10, wherein R₄ is selected from:
CH₃, 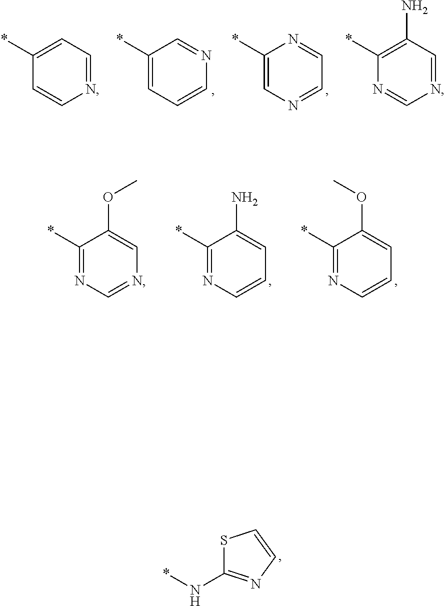
-heteroaryl1, wherein said heteroaryl1 is a 5 membered, fully unsaturated, monocyclic ring comprising ring carbon atoms and 1, 2, 3 or 4 ring heteroatoms independently selected from N, O and S;
-heteroaryl2, wherein said heteroaryl2 is a 9 or 10 membered fused bicyclic ring comprising ring carbon atoms and 1, 2, 3 or 4 ring heteroatoms independently selected from N, O and S, and wherein both rings are fully unsaturated, or one ring is fully unsaturated, and the other is saturated or partially unsaturated, and wherein the heteroatoms may be in one or both rings;
-phenyl; or
-heterocyclyl2, wherein said heterocyclyl2 is a 5 or 6 membered fully saturated or partially unsaturated group comprising ring carbon atoms and 1 or 2 ring heteroatoms independently selected from N, O and S,
wherein heteroary1, heteroaryl2, phenyl, and the moiety selected from: 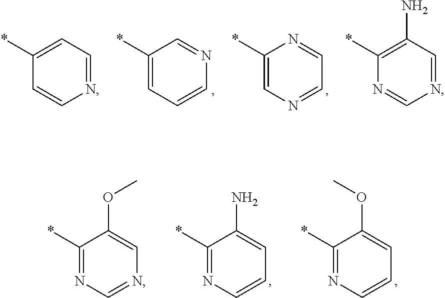
are each substituted by 1, 2 or 3 substituents, in particular 1 or 2 substituents, independently selected from R₁₀, R₁₁, R₁₂, R₁₃ and R₁₄, wherein each R₁₀, R₁₁, R₁₂, R₁₃ and R₁₄ is independently selected from:
H,
halo,
(C₁-C₄)alkyl unsubstituted or substituted by 1, 2 or 3 halo substituents,
(C₁-C₂)alkyl substituted by —O—(C₁-C₂)alkyl or OH,
—S—(C₁-C₃)alkyl,
—O—(C₁-C₄)alkyl unsubstituted or substituted by 1, 2 or 3 halo substituents,
OH,
(C₃-C₅)cycloalkyl, wherein said (C₃-C₅)cycloalkyl is unsubstituted or substituted by 1 or 2 halo,
—O—(C₃-C₅)cycloalkyl,
—NR₃₄R₃₅ wherein R₃₄ and R₃₅ are independently selected from:
H,
(C₁-C₄)alkyl, wherein said (C₁-C₄)alkyl is unsubstituted or substituted by OH or —O(C₁-C₂)alkyl,
and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
CN,
—(C₂-C₄)alkenyl,
—(C₂-C₄)alkynyl,
═O
—C(O)H, and
—C(O)(C₁-C₄)alkyl;

12. A compound of formula (I) or a pharmaceutically acceptable salt thereof, according to any of claims 1 to 11, wherein R₄ is as described in claim 1 or claim 11, with the proviso that at least one OH, CN, ═O, or NH₂ substituent is present on each heteroaryl1, heteroaryl2, phenyl, 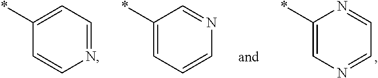
and the remaining R₁₀, R₁₁, R₁₂, R₁₃ and R₁₄ are as defined herein.

13. A compound of formula (I) or a pharmaceutically acceptable salt thereof, according to any of claims 1 to 12, wherein R₄ is selected from:

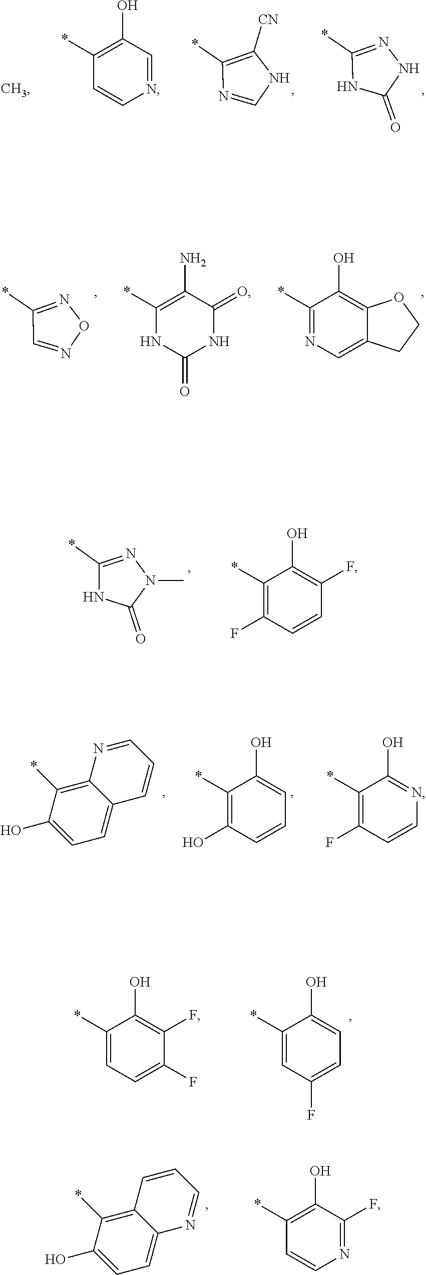

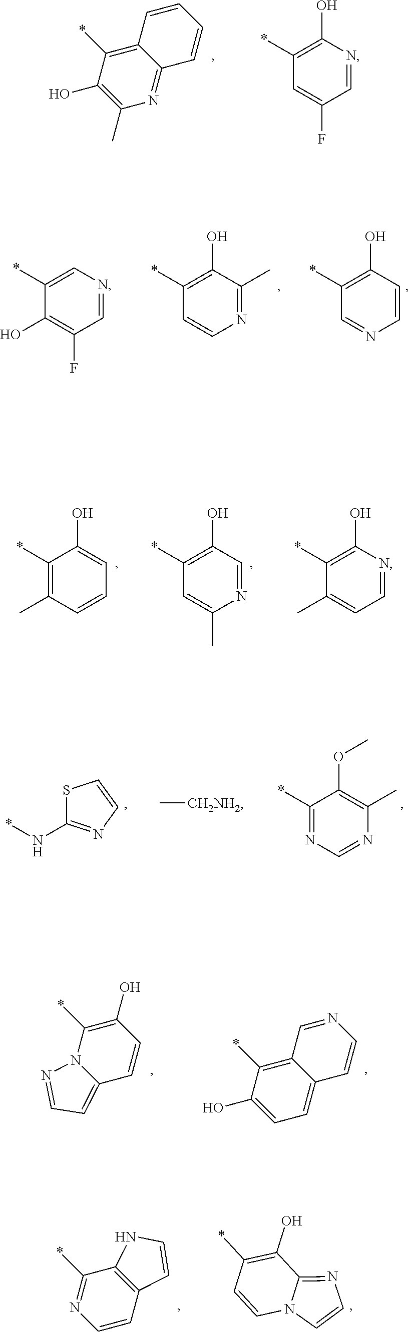

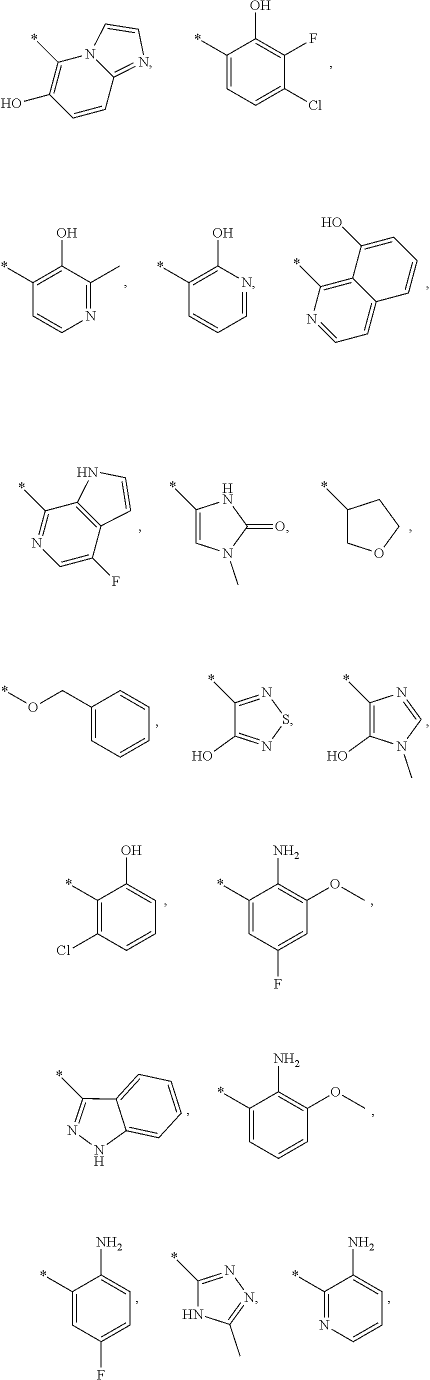

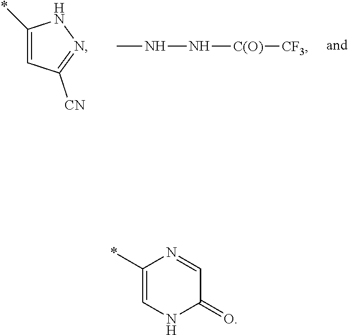

14. A compound of formula (I) or a pharmaceutically acceptable salt thereof, according to any of claims 1 to 13, wherein K [symbol] is K linked by a single bond, K is —OH₂— and J is N.

15. A compound of formula (I) or a pharmaceutically acceptable salt thereof, according to any of claims 1 to 14, wherein R₅ is independently selected from:
—(C₁-C₄)alkyl, preferably methyl,
and wherein two R₅ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S,
when K [symbol] J is a carbon-nitrogen single bond, a R₅ substituent on K and on the adjacent carbon atom may join to form ring C: 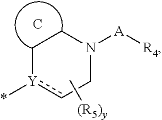
wherein ring C is a fused (C₃-C₆)cycloalkyl ring, in particular a fused cyclobutyl ring, a fused (C₃-C₆)heterocyclyl ring or a fused phenyl ring, wherein said fused (C₃-C₆)heterocyclyl ring contains ring carbon atoms and one ring heteroatom selected from O, N and S,
and wherein when ring C is a fused (C₃-C₆)cycloalkyl ring, in particular fused cyclobutyl ring, said fused (C₃-C₆)cycloalkyl ring is unsubstituted or substituted with 1 or 2 R₄₀ groups, wherein said R₄₀ is selected from:
(C₁-C₂)alkyl, wherein each (C₁-C₂)alkyl is independently unsubstituted or substituted by OH or 1, 2 or 3 halo,
halo, in particular F,
or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
or wherein two R₄₀ substituents on adjacent carbon atoms join together with the carbon atoms to which they are attached, to form a fused cyclopropyl ring;
and wherein when K is —CH₂— and J is N, two R₅ substituents may join to form a (C₁-C₃)alkylene bridge or a heteroalkylene bridge, wherein said heteroalkylene bridge is one heteroatom selected from N and O, or is —CH₂—O—CH₂—.

16. A compound of formula (I) or a pharmaceutically acceptable salt thereof, according to any of claims 1 to 15, wherein R₅ is independently selected from:
—(C₁-C₂)alkyl, preferably methyl, and
when K [symbol] J is a carbon-nitrogen single bond, a R₅ substituent on K and on the adjacent carbon atom may join to form ring C: 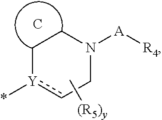
wherein ring C is a fused (C₃-C₄)cycloalkyl ring, in particular a fused cyclobutyl ring, and said fused (C₃-C₄)cycloalkyl ring, in particular fused cyclobutyl ring, is unsubstituted or substituted with 1 or 2 R₄₀ groups as described in the embodiments herein.

17. A compound of formula (I) or a pharmaceutically acceptable salt thereof, according to any of claims 1 to 16, wherein y is 0, 1, 2 or 3, preferably 0, 1, or 2.

18. A compound of formula (I) or a pharmaceutically acceptable salt thereof, according to any of claims 1 to 17, wherein the compound of formula (I) includes the moiety: 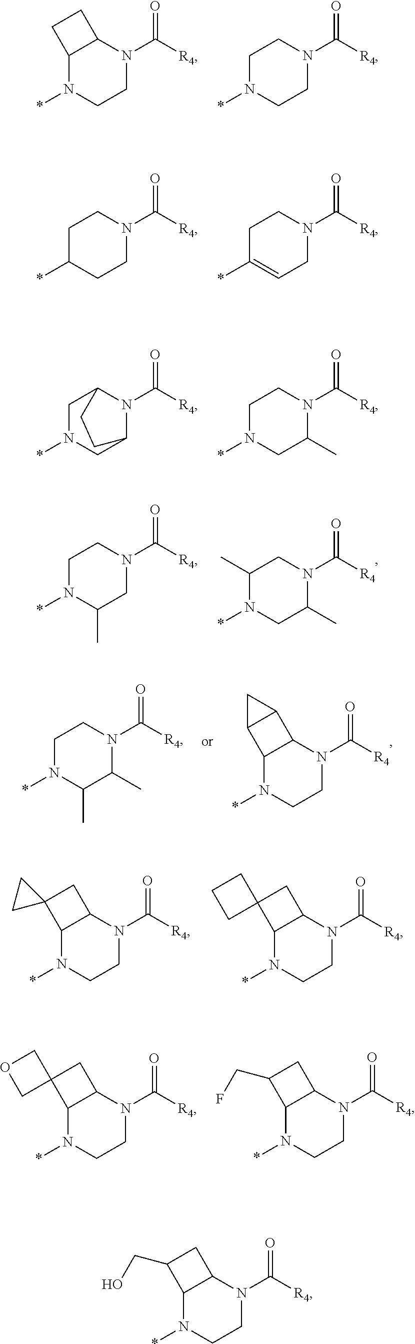
in particular
A: 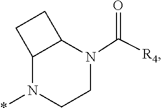
more particularly 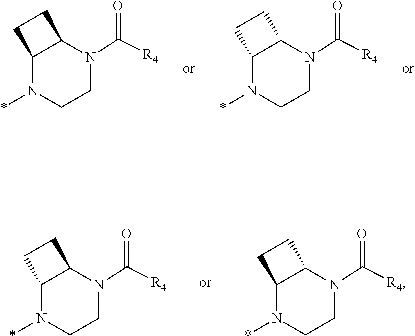
or B: 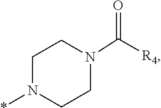
or C:

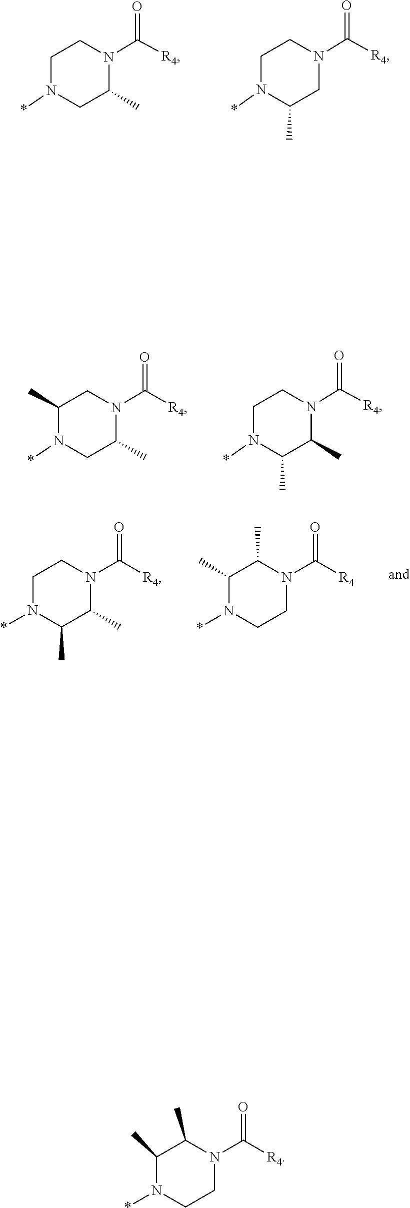

19. A compound of formula (I) or a pharmaceutically acceptable salt thereof, according to any of claims 1 to 18, wherein formula (I) is formula 1a, wherein A is —C(O)—:

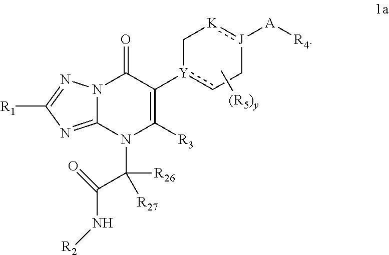

20. A compound of Formula (I) or a pharmaceutically acceptable salt thereof, selected from an exemplified compound herein.

21. A combination comprising a compound of formula (I), or a pharmaceutically acceptable salt thereof, according to any of claims 1 to 20, and one or more additional therapeutically active agents.

22. A combination according to claim 21, wherein an additional therapeutically active agent is an anti-cancer agent.

23. A combination according to claim 21, wherein an additional therapeutically active agent is a chemotherapy selected from anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®), in particular irinotecan.

24. A combination according to claim 21, wherein an additional therapeutically active agent is a PD-1 inhibitor.

25. A combination according to claim 24, wherein an additional therapeutically active agent is a PD-1 inhibitor selected from PDR001 (Novartis), Nivolumab (Bristol-Myers Squibb), Pembrolizumab (Merck & Co), Pidilizumab (CureTech), MEDI0680 (Medimmune), Cemiplimab (REGN2810, Regeneron), Dostarlimab (TSR-042, Tesaro), PF-06801591 (Pfizer), Tislelizumab (BGB-A317, Beigene), BGB-108 (Beigene), INCSHR1210 (Incyte), Balstilimab (AGEN2035, Agenus), Sintilimab (InnoVent), Toripalimab (Shanghai Junshi Bioscience), Camrelizumab (Jiangsu Hengrui Medicine Co.), and AMP-224 (Amplimmune), in particular PDR001, Tislelizumab and Pembrolizumab, more particularly Tislelizumab.

26. A pharmaceutical composition comprising a compound of formula (I) or a pharmaceutically acceptable salt thereof, according to any of claims 1 to 20, and one or more pharmaceutically acceptable carriers.

27. A compound of formula (I), or a pharmaceutically acceptable salt thereof, according to any of claims 1 to 20, for use as a medicament.

28. A compound of formula (I), or a pharmaceutically acceptable salt thereof, according to any of claims 1 to 20, for use according to claim 27, wherein the use is for the treatment of a disease that is treated by WRN inhibition.

29. A compound of formula (I), or a pharmaceutically acceptable salt thereof, according to any of claims 1 to 20, for use according to claim 27, wherein the use is for the treatment of cancer.

30. A compound of formula (I), or a pharmaceutically acceptable salt thereof, according to any of claims 1 to 20, for use according to claim 29, wherein the cancer is characterized as microsatellite instability-high (MSI-H) or mismatch repair deficient (dMMR).

31. A compound of formula (I), or a pharmaceutically acceptable salt thereof, according to any of claims 1 to 20, for use according to claim 30, wherein the cancer characterized as microsatellite instability-high (MSI-H) or mismatch repair deficient (dMMR) is selected from colorectal, gastric, and endometrial, adrenocortical, uterine, cervical, esophageal, breast, kidney and ovarian cancer.

32. A compound of formula (I), or a pharmaceutically acceptable salt thereof, according to any of claims 1 to 20, for use according to claim 31, wherein the cancer characterized as microsatellite instability-high (MSI-H) or mismatch repair deficient (dMMR) is selected from colorectal, gastric and endometrial cancer.

33. A compound of formula (I), or a pharmaceutically acceptable salt thereof, according to any of claims 1 to 20, for use according to claim 30, wherein the cancer characterized as microsatellite instability-high (MSI-H) or mismatch repair deficient (dMMR) is selected from uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma and ovarian serous cystadenocarcinoma.

34. A method of modulating WRN activity in a subject, wherein the method comprises administering to the subject a therapeutically effective amount of the compound of formula (I), or a pharmaceutically acceptable salt thereof, according to any of claims 1 to 20.

35. A method of treating a disorder or disease which can be treated by WRN inhibition in a subject, comprising administering to the subject a therapeutically effective amount of the compound of formula (I), or a pharmaceutically acceptable salt thereof, according to any of claims 1 to 20.

36. A method of treating cancer in a subject, comprising administering to the subject a therapeutically effective amount of the compound of formula (I), or a pharmaceutically acceptable salt thereof, according to any of claims 1 to 20.

37. A method of treating cancer in a subject, comprising administering a compound of formula (I), or a pharmaceutically acceptable salt thereof, according to any of claims 1 to 20, wherein the cancer is characterized as microsatellite instability-high (MSI-H) or mismatch repair deficient (dMMR).

38. The use of a compound, or pharmaceutically acceptable salt thereof, according to any of claims 1 to 20, in the manufacture of a medicament for the treatment of cancer.

39. The use of a compound of Formula (I) as described in any of claims 1 to 20, or salt thereof, as a research chemical, a chemical probe, or as a tool compound.

40. A process or intermediate as defined herein.
